SEQUENCE LISTING (151 sequences)

SEQ ID NO: 1 - protein, 19 aa, synthetic construct
MDWIWRILFLVGAATGAHS

SEQ ID NO: 2 - DNA, 57 bp, synthetic construct
  source 1..57: mol_type = other DNA
atggactggatttggcggatcctgtttctcgtgggagccgccacaggcgcccattct

SEQ ID NO: 3 - protein, 19 aa, Homo sapiens
MDWIWRILFLVGAATGAHS

SEQ ID NO: 4 - DNA, 57 bp, Homo sapiens
  source 1..57: mol_type = genomic DNA
atggactggatctggcggattctgttcctcgtgggagccgccacaggcgctcactca

SEQ ID NO: 5 - DNA, 57 bp, Homo sapiens
  source 1..57: mol_type = genomic DNA
atggactggatctggcgcatcctcttcctcgtcggcgctgctaccggcgctcattct

SEQ ID NO: 6 - DNA, 57 bp, Homo sapiens
  source 1..57: mol_type = genomic DNA
atggactggatctggcgcatcctgtttcttgtgggagccgccacaggcgcccattct

SEQ ID NO: 7 - protein, 21 aa, Homo sapiens
MALPVTALLLPLALLLHAARP

SEQ ID NO: 8 - DNA, 63 bp, Homo sapiens
  source 1..63: mol_type = genomic DNA
atggccttaccagtgaccgccttgctcctgccgctggccttgctgctccacgccgccaggccg

SEQ ID NO: 9 - protein, 15 aa, synthetic construct
GGGGSGGGGSGGGGS

SEQ ID NO: 10 - DNA, 45 bp, synthetic construct
  source 1..45: mol_type = other DNA
ggaggcggaggatcaggcggcggaggaagtgggggaggcggatct

SEQ ID NO: 11 - DNA, 45 bp, synthetic construct
  source 1..45: mol_type = other DNA
ggaggcggaggaagtggagggggaggatcaggcggcggaggcagc

SEQ ID NO: 12 - protein, 98 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRD

SEQ ID NO: 13 - DNA, 294 bp, synthetic construct
  source 1..294: mol_type = other DNA
atacaacgcacgcctaaaatccaagtctatagtcggcacccggcggaaaatgggaaatctaatttccttaactgctatgtgtccggtttccacccatccgacattgaggtagacctgctgaaaaacggggaacggatagaaaaggtcgaacacagtgatctgtcctttagtaaggattggtctttttatctgctctactacacagagtttactccgacagaaaaagacgagtacgcgtgccgcgtaaaccacgtaacactgagccaaccgaagattgtgaagtgggatagagat

SEQ ID NO: 14 - protein, 229 aa, synthetic construct
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - DNA, 687 bp, synthetic construct
  source 1..687: mol_type = other DNA
gagtctaagtacggccctccttgtcctagctgccccgctcctgaatttgaaggcggcccttccgtgttcctgtttcctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagcagactgaccgtggacaagagcagatggcaagagggcaacgtgttcagctgcagcgtgatgcacgaggccctgcacaaccactacacccagaagtctctgagcctgagcctcggcaag

SEQ ID NO: 16 - protein, 74 aa, synthetic construct
AGSGGSGGSGGSPVPSTPPTNSSSTPPTPSPSPVPSTPPTNSSSTPPTPSPSPVPSTPPTNSSSTPPTPSPSAS

SEQ ID NO: 17 - DNA, 222 bp, synthetic construct
  source 1..222: mol_type = other DNA
gcaggcagtggtggaagtggtgggtcaggcggttccccggttccatcaacaccaccaaccaatagctcatctaccccgccaacaccaagtccctctcccgtccccagcacacccccgacgaactcatcctccacaccgccaaccccgtctccttccccggttccctctacacctccgacaaactctagttcaacgcctcccactccatcaccttccgcatca

SEQ ID NO: 18 - DNA, 45 bp, synthetic construct
  source 1..45: mol_type = other DNA
ggcggcggcggatcaggcggcggaggaagcggaggtggggggtcc

SEQ ID NO: 19 - DNA, 45 bp, synthetic construct
  source 1..45: mol_type = other DNA
ggaggcggaggttcaggtggcggaggaagtggcggcggaggatca

SEQ ID NO: 20 - protein, 5 aa, synthetic construct
GGGGS

SEQ ID NO: 21 - DNA, 15 bp, synthetic construct
  source 1..15: mol_type = other DNA
ggaggcggaggttca

SEQ ID NO: 22 - protein, 10 aa, synthetic construct
GGGGSGGGGS

SEQ ID NO: 23 - protein, 20 aa, synthetic construct
GGGGSGGGGSGGGGSGGGGS

SEQ ID NO: 24 - protein, 18 aa, synthetic construct
KESGSVSSEQLAQFRSLD

SEQ ID NO: 25 - protein, 14 aa, synthetic construct
EGKSSGSGSESKST

SEQ ID NO: 26 - protein, 15 aa, synthetic construct
EGKSSGSGSESKSTQ

SEQ ID NO: 27 - protein, 14 aa, synthetic construct
GSTSGSGKSSEGKG

SEQ ID NO: 28 - protein, 25 aa, synthetic construct
SSADDAKKDDAKKDDAKKDDAKKDG

SEQ ID NO: 29 - protein, 14 aa, synthetic construct
EGKSSGSGSESKVD

SEQ ID NO: 30 - protein, 16 aa, synthetic construct
ESGSVSSEELAFRSLD

SEQ ID NO: 31 - protein, 20 aa, Homo sapiens
DLEPKSCDKTHTCPPCPDPK

SEQ ID NO: 32 - DNA, 60 bp, Homo sapiens
  source 1..60: mol_type = genomic DNA
gatctcgagcccaaatcttgtgacaaaactcacacatgcccaccgtgcccggatcccaag

SEQ ID NO: 33 - protein, 231 aa, Homo sapiens
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGS

SEQ ID NO: 34 - DNA, 693 bp, Homo sapiens
  source 1..693: mol_type = genomic DNA
gagtctaagtacggccctccttgtcctagctgccccgctcctgaatttgaaggcggcccttccgtgttcctgtttcctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagcagactgaccgtggacaagagcagatggcaagagggcaacgtgttcagctgcagcgtgatgcacgaggccctgcacaaccactacacccagaagtctctgagcctgagcctcggcaagggctcc

SEQ ID NO: 35 - protein, 45 aa, Homo sapiens
TTTPAPRPPTPAPTIASQPLSLRPEACRPAAGGAVHTRGLDFACD

SEQ ID NO: 36 - DNA, 135 bp, Homo sapiens
  source 1..135: mol_type = genomic DNA
accaccacaccagctcctcggcctccaactcctgctcctacaatagccagccagcctctgtctctgaggcccgaagcatgcagacctgctgctggcggagccgtgcatacaagaggactggatttcgcctgcgac

SEQ ID NO: 37 - protein, 39 aa, Homo sapiens
IEVMYPPPYLDNEKSNGTIIHVKGKHLCPSPLFPGPSKP

SEQ ID NO: 38 - DNA, 116 bp, Homo sapiens
  source 1..116: mol_type = genomic DNA
atcgaagtgatgtacccgcctccttacctggacaacgagaagtccaacggcaccatcatccacgtgaagggaaagcacctgtgtccttctccactgttccccggacctagcaagcc

SEQ ID NO: 39 - protein, 15 aa, Homo sapiens
EPKSCDKTHTCPPCP

SEQ ID NO: 40 - protein, 12 aa, Homo sapiens
ERKCCVECPPCP

SEQ ID NO: 41 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 42 - protein, 12 aa, Homo sapiens
ESKYGPPCPSCP

SEQ ID NO: 43 - protein, 27 aa, Homo sapiens
FWVLVVVGGVLACYSLLVTVAFIIFWV

SEQ ID NO: 44 - DNA, 82 bp, Homo sapiens
  source 1..82: mol_type = genomic DNA
tttctgggtgctcgttgttgttggcggcgtgctggcctgttacagcctgctggttaccgtggccttcatcatcttttgggtc

SEQ ID NO: 45 - DNA, 81 bp, Homo sapiens
  source 1..81: mol_type = genomic DNA
ttctgggtgctggtggtcgtgggcggagtgctggcctgttacagcctgctcgtgaccgtggccttcatcatcttttgggtg

SEQ ID NO: 46 - protein, 36 aa, Homo sapiens
FWVLVVVGGVLACYSLLVTVAFIIFWVRSKRSRLLH

SEQ ID NO: 47 - DNA, 108 bp, Homo sapiens
  source 1..108: mol_type = genomic DNA
ttctgggtgctggtggtcgtgggcggagtgctggcctgttacagcctgctcgtgaccgtggccttcatcatcttttgggtgcgcagcaagcggagccggctgctgcac

SEQ ID NO: 48 - protein, 24 aa, Homo sapiens
IYIWAPLAGTCGVLLLSLVITLYC

SEQ ID NO: 49 - DNA, 72 bp, Homo sapiens
  source 1..72: mol_type = genomic DNA
atctacatctgggcccctctggctggaacatgtggcgttctgctgctgagcctggtcatcaccctgtactgc

SEQ ID NO: 50 - protein, 21 aa, Homo sapiens
LCYLLDGILFIYGVILTALFL

SEQ ID NO: 51 - DNA, 63 bp, Homo sapiens
  source 1..63: mol_type = genomic DNA
ctgtgctacctgctggacggcatcctgttcatctacggcgtgatcctgaccgccctgttcctg

SEQ ID NO: 52 - protein, 66 aa, Homo sapiens
IEVMYPPPYLDNEKSNGTIIHVKGKHLCPSPLFPGPSKPFWVLVVVGGVLACYSLLVTVAFIIFWV

SEQ ID NO: 53 - DNA, 198 bp, Homo sapiens
  source 1..198: mol_type = genomic DNA
atcgaagtgatgtacccgcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtc

SEQ ID NO: 54 - protein, 41 aa, Homo sapiens
RSKRSRLLHSDYMNMTPRRPGPTRKHYQPYAPPRDFAAYRS

SEQ ID NO: 55 - DNA, 123 bp, Homo sapiens
  source 1..123: mol_type = genomic DNA
cgaagcaagcggagccggctgctgcacagcgactacatgaacatgacccctagacggcccggaccaaccagaaagcactaccagccttacgctcctcctagagatttcgccgcctaccggtcc

SEQ ID NO: 56 - DNA, 123 bp, Homo sapiens
  source 1..123: mol_type = genomic DNA
cgcagcaagcggagccggctgctgcacagcgactacatgaacatgacccccagacggcctggccccaccagaaagcactaccagccttacgcccctcccagagacttcgccgcctaccggtcc

SEQ ID NO: 57 - protein, 42 aa, unidentified
KRGRKKLLYIFKQPFMRPVQTTQEEDGCSCRFPEEEEGGCEL

SEQ ID NO: 58 - DNA, 126 bp, unidentified
  source 1..126: mol_type = unassigned DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 59 - protein, 36 aa, unidentified
RDQRLPPDAHKPPGGGSFRTPIQEEQADAHSTLAKI

SEQ ID NO: 60 - DNA, 108 bp, unidentified
  source 1..108: mol_type = unassigned DNA
agggaccagaggctgccccccgatgcccacaagccccctgggggaggcagtttccggacccccatccaagaggagcaggccgacgcccactccaccctggccaagatc

SEQ ID NO: 61 - protein, 171 aa, unidentified
AAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDI

SEQ ID NO: 62 - DNA, 513 bp, unidentified
  source 1..513: mol_type = unassigned DNA
gctgctggcggacctggcgccggatctgctgctcctgtgtctagcacaagcagcctgcctctggccgccctgaacatgagagtgcggagaaggctgagcctgttcctgaacgtgcggacacaggtggccgccgattggacagccctggccgaggaaatggacttcgagtacctggaaatccggcagctggaaacccaggccgaccctacaggcagactgctggatgcttggcagggcagaccaggcgcttctgtgggaaggctgctggaactgctgaccaagctgggcagggacgacgtgctgctggaactgggccctagcatcgaagaggactgccagaagtacatcctgaagcagcagcaggaagaggccgagaagcctctgcaggtggcagccgtggatagcagcgtgccaagaacagccgagctggccggcatcaccaccctggatgatcctctgggccacatgcccgagagattcgacgccttcatctgctactgccccagcgacatc

SEQ ID NO: 63 - protein, 172 aa, unidentified
MAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDI

SEQ ID NO: 64 - DNA, 516 bp, unidentified
  source 1..516: mol_type = unassigned DNA
atggccgctgggggcccaggcgccggatcagctgctcccgtatcttctacttcttctttgccgctggctgctctgaacatgcgcgtgagaagacgcctctccctgttccttaacgttcgcacacaagtcgctgccgattggaccgcccttgccgaagaaatggactttgaatacctggaaattagacaacttgaaacacaggccgaccccactggcagactcctggacgcatggcagggaagacctggtgcaagcgttggacggctcctggatctcctgacaaaactgggacgcgacgacgtactgcttgaactcggacctagcattgaagaagactgccaaaaatatatcctgaaacaacaacaagaagaagccgaaaaacctctccaagtcgcagcagtggactcatcagtaccccgaacagctgagcttgctgggattactacactcgacgacccactcggacatatgcctgaaagattcgacgctttcatttgctattgcccctctgacata

SEQ ID NO: 65 - protein, 171 aa, unidentified
AAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDI

SEQ ID NO: 66 - DNA, 513 bp, unidentified
  source 1..513: mol_type = unassigned DNA
gccgctgggggcccaggcgccggatcagctgctcccgtatcttctacttcttctttgccgctggctgctctgaacatgcgcgtgagaagacgcctctccctgttccttaacgttcgcacacaagtcgctgccgattggaccgcccttgccgaagaaatggactttgaatacctggaaattagacaacttgaaacacaggccgaccccactggcagactcctggacgcatggcagggaagacctggtgcaagcgttggacggctcctggatctcctgacaaaactgggacgcgacgacgtactgcttgaactcggacctagcattgaagaagactgccaaaaatatatcctgaaacaacaacaagaagaagccgaaaaacctctccaagtcgcagcagtggactcatcagtaccccgaacagctgagcttgctgggattactacactcgacgacccactcggacatatgcctgaaagattcgacgctttcatttgctattgcccctctgacata

SEQ ID NO: 67 - protein, 62 aa, unidentified
KKVAKKPTNKAPHPKQEPQEINFPDDLPGSNTAAPVQETLHGCQPVTQEDGKESRISVQERQ

SEQ ID NO: 68 - DNA, 186 bp, unidentified
  source 1..186: mol_type = unassigned DNA
aagaaggtggccaagaagcccaccaacaaggccccccaccccaagcaggaaccccaggaaatcaacttccccgacgacctgcccggcagcaatactgctgcacccgtgcaggaaaccctgcacggctgtcagcctgtgacccaggaagatggcaaagaaagccggatctctgtgcaggaacgccag

SEQ ID NO: 69 - protein, 112 aa, unidentified
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - DNA, 336 bp, unidentified
  source 1..336: mol_type = unassigned DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 71 - DNA, 336 bp, unidentified
  source 1..336: mol_type = unassigned DNA
agagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaaga

SEQ ID NO: 72 - protein, 116 aa, unidentified
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRA

SEQ ID NO: 73 - DNA, 348 bp, unidentified
  source 1..348: mol_type = unassigned DNA
agagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagcc

SEQ ID NO: 74 - protein, 18 aa, unidentified
EGRGSLLTCGDVEENPGP

SEQ ID NO: 75 - DNA, 54 bp, unidentified
  source 1..54: mol_type = unassigned DNA
gaaggcagaggctctctgctgacatgtggcgacgtggaagagaatcctggacct

SEQ ID NO: 76 - DNA, 54 bp, unidentified
  source 1..54: mol_type = unassigned DNA
gagggcagaggcagcctgctgacatgtggcgacgtggaagagaacccaggcccc

SEQ ID NO: 77 - protein, 19 aa, unidentified
AEGRGSLLTCGDVEENPGP

SEQ ID NO: 78 - protein, 21 aa, unidentified
GSGEGRGSLLTCGDVEENPGP

SEQ ID NO: 79 - protein, 63 aa, unidentified
GSGSRVTELLYRMKRAETYCPRPLLAIHPTEARHKQKIVAPVKQLLNFDLLKLAGDVESNPGP

SEQ ID NO: 80 - protein, 19 aa, unidentified
LLCFLLLLLSGDVELNPGP

SEQ ID NO: 81 - protein, 20 aa, unidentified
HHFMFLLLLLAGDIELNPGP

SEQ ID NO: 82 - protein, 20 aa, Saccoglossus kowalevskii
WFLVLLSFILSGDIEVNPGP

SEQ ID NO: 83 - protein, 20 aa, Branchiostoma floridae
KNCAMYMLLLSGDVETNPGP

SEQ ID NO: 84 - protein, 20 aa, Branchiostoma floridae
MVISQLMLKLAGDVEENPGP

SEQ ID NO: 85 - protein, 22 aa, unidentified
GSGATNFSLLKQAGDVEENPGP

SEQ ID NO: 86 - protein, 23 aa, Equine rhinitis A virus
GSGQCTNYALLKLAGDVESNPGP

SEQ ID NO: 87 - protein, 8 aa, synthetic construct
  VARIANT 2: note = Any amino acid
  VARIANT 4: note = Any amino acid
DXEXNPGP

SEQ ID NO: 88 - protein, 333 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 89 - DNA, 999 bp, synthetic construct
  source 1..999: mol_type = other DNA
atgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttc

SEQ ID NO: 90 - DNA, 999 bp, synthetic construct; identical to SEQ ID NO: 89
  source 1..999: mol_type = other DNA

SEQ ID NO: 91 - DNA, 1002 bp, synthetic construct
  source 1..1002: mol_type = other DNA
atgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttctaa

SEQ ID NO: 92 - protein, 13 aa, synthetic construct
CTVALPGGYVRVC

SEQ ID NO: 93 - DNA, 39 bp, synthetic construct
  source 1..39: mol_type = other DNA
tgtactgtggcccttcctggtggatacgttagagtgtgc

SEQ ID NO: 94 - protein, 115 aa, synthetic construct
QVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSS

SEQ ID NO: 95 - DNA, 345 bp, synthetic construct
  source 1..345: mol_type = other DNA
caggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagc

SEQ ID NO: 96 - protein, 108 aa, synthetic construct
DVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKS

SEQ ID NO: 97 - DNA, 324 bp, synthetic construct
  source 1..324: mol_type = other DNA
gatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagc

SEQ ID NO: 98 - protein, 238 aa, synthetic construct
QVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKS

SEQ ID NO: 99 - DNA, 714 bp, synthetic construct
  source 1..714: mol_type = other DNA
caggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagcggaggcggaggatcaggcggcggaggaagtgggggaggcggatctgatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagc

SEQ ID NO: 100 - protein, 329 aa, synthetic construct
MDWIWRILFLVGAATGAHSCTVALPGGYVRVCEFGGGGSGGGGSGGGGSAAAQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKSIEVMYPPPYLDNEKSNGTIIHVKGKHLCPSPLFPGPSKP

SEQ ID NO: 101 - DNA, 986 bp, synthetic construct
  source 1..986: mol_type = other DNA
atggactggatttggcggatcctgtttctcgtgggagccgccacaggcgcccattcttgtactgtggcccttcctggtggatacgttagagtgtgcgaattcggaggcggaggatcaggcggcggaggaagtgggggaggcggatctgcggccgctcaggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagcggaggcggaggatcaggcggcggaggaagtgggggaggcggatctgatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagcatcgaagtgatgtacccgcctccttacctggacaacgagaagtccaacggcaccatcatccacgtgaagggaaagcacctgtgtccttctccactgttccccggacctagcaagcc

SEQ ID NO: 102 - protein, 412 aa, synthetic construct
MDWIWRILFLVGAATGAHSCTVALPGGYVRVCEFIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDAAAQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKSIEVMYPPPYLDNEKSNGTIIHVKGKHLCPSPLFPGPSKP

SEQ ID NO: 103 - DNA, 1235 bp, synthetic construct
  source 1..1235: mol_type = other DNA
atggactggatttggcggatcctgtttctcgtgggagccgccacaggcgcccattcttgtactgtggcccttcctggtggatacgttagagtgtgcgaattcatacaacgcacgcctaaaatccaagtctatagtcggcacccggcggaaaatgggaaatctaatttccttaactgctatgtgtccggtttccacccatccgacattgaggtagacctgctgaaaaacggggaacggatagaaaaggtcgaacacagtgatctgtcctttagtaaggattggtctttttatctgctctactacacagagtttactccgacagaaaaagacgagtacgcgtgccgcgtaaaccacgtaacactgagccaaccgaagattgtgaagtgggatagagatgcggccgctcaggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagcggaggcggaggatcaggcggcggaggaagtgggggaggcggatctgatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagcatcgaagtgatgtacccgcctccttacctggacaacgagaagtccaacggcaccatcatccacgtgaagggaaagcacctgtgtccttctccactgttccccggacctagcaagcc

SEQ ID NO: 104 - protein, 543 aa, synthetic construct
MDWIWRILFLVGAATGAHSCTVALPGGYVRVCEFESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAAAQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKSIEVMYPPPYLDNEKSNGTIIHVKGKHLCPSPLFPGPSKP

SEQ ID NO: 105 - DNA, 1628 bp, synthetic construct
  source 1..1628: mol_type = other DNA
atggactggatttggcggatcctgtttctcgtgggagccgccacaggcgcccattcttgtactgtggcccttcctggtggatacgttagagtgtgcgaattcgagtctaagtacggccctccttgtcctagctgccccgctcctgaatttgaaggcggcccttccgtgttcctgtttcctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagcagactgaccgtggacaagagcagatggcaagagggcaacgtgttcagctgcagcgtgatgcacgaggccctgcacaaccactacacccagaagtctctgagcctgagcctcggcaaggcggccgctcaggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagcggaggcggaggatcaggcggcggaggaagtgggggaggcggatctgatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagcatcgaagtgatgtacccgcctccttacctggacaacgagaagtccaacggcaccatcatccacgtgaagggaaagcacctgtgtccttctccactgttccccggacctagcaagcc

SEQ ID NO: 106 - protein, 388 aa, synthetic construct
MDWIWRILFLVGAATGAHSCTVALPGGYVRVCEFAGSGGSGGSGGSPVPSTPPTNSSSTPPTPSPSPVPSTPPTNSSSTPPTPSPSPVPSTPPTNSSSTPPTPSPSASAAAQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKSIEVMYPPPYLDNEKSNGTIIHVKGKHLCPSPLFPGPSKP

SEQ ID NO: 107 - DNA, 1163 bp, synthetic construct
  source 1..1163: mol_type = other DNA
atggactggatttggcggatcctgtttctcgtgggagccgccacaggcgcccattcttgtactgtggcccttcctggtggatacgttagagtgtgcgaattcgcaggcagtggtggaagtggtgggtcaggcggttccccggttccatcaacaccaccaaccaatagctcatctaccccgccaacaccaagtccctctcccgtccccagcacacccccgacgaactcatcctccacaccgccaaccccgtctccttccccggttccctctacacctccgacaaactctagttcaacgcctcccactccatcaccttccgcatcagcggccgctcaggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagcggaggcggaggatcaggcggcggaggaagtgggggaggcggatctgatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagcatcgaagtgatgtacccgcctccttacctggacaacgagaagtccaacggcaccatcatccacgtgaagggaaagcacctgtgtccttctccactgttccccggacctagcaagcc

SEQ ID NO: 108 - protein, 153 aa, synthetic construct
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - DNA, 459 bp, synthetic construct
  source 1..459: mol_type = other DNA
cgaagcaagcggagccggctgctgcacagcgactacatgaacatgacccctagacggcccggaccaaccagaaagcactaccagccttacgctcctcctagagatttcgccgcctaccggtccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 110 - protein, 509 aa, synthetic construct
MDWIWRILFLVGAATGAHSCTVALPGGYVRVCEFGGGGSGGGGSGGGGSAAAQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - DNA, 1527 bp, synthetic construct
  source 1..1527: mol_type = other DNA
atggactggatttggcggatcctgtttctcgtgggagccgccacaggcgcccattcttgtactgtggcccttcctggtggatacgttagagtgtgcgaattcggaggcggaggatcaggcggcggaggaagtgggggaggcggatctgcggccgctcaggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagcggaggcggaggatcaggcggcggaggaagtgggggaggcggatctgatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagcatcgaagtgatgtacccgcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagatttcgccgcctaccggtccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 112 - protein, 592 aa, synthetic construct
MDWIWRILFLVGAATGAHSCTVALPGGYVRVCEFIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDAAAQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - DNA, 1776 bp, synthetic construct
  source 1..1776: mol_type = other DNA
atggactggatttggcggatcctgtttctcgtgggagccgccacaggcgcccattcttgtactgtggcccttcctggtggatacgttagagtgtgcgaattcatacaacgcacgcctaaaatccaagtctatagtcggcacccggcggaaaatgggaaatctaatttccttaactgctatgtgtccggtttccacccatccgacattgaggtagacctgctgaaaaacggggaacggatagaaaaggtcgaacacagtgatctgtcctttagtaaggattggtctttttatctgctctactacacagagtttactccgacagaaaaagacgagtacgcgtgccgcgtaaaccacgtaacactgagccaaccgaagattgtgaagtgggatagagatgcggccgctcaggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagcggaggcggaggatcaggcggcggaggaagtgggggaggcggatctgatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagcatcgaagtgatgtacccgcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagatttcgccgcctaccggtccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 114 - protein, 723 aa, synthetic construct
MDWIWRILFLVGAATGAHSCTVALPGGYVRVCEFESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAAAQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - DNA, 2169 bp, synthetic construct
  source 1..2169: mol_type = other DNA
atggactggatttggcggatcctgtttctcgtgggagccgccacaggcgcccattcttgtactgtggcccttcctggtggatacgttagagtgtgcgaattcgagtctaagtacggccctccttgtcctagctgccccgctcctgaatttgaaggcggcccttccgtgttcctgtttcctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagcagactgaccgtggacaagagcagatggcaagagggcaacgtgttcagctgcagcgtgatgcacgaggccctgcacaaccactacacccagaagtctctgagcctgagcctcggcaaggcggccgctcaggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagcggaggcggaggatcaggcggcggaggaagtgggggaggcggatctgatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagcatcgaagtgatgtacccgcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagatttcgccgcctaccggtccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 116 - protein, 568 aa, synthetic construct
MDWIWRILFLVGAATGAHSCTVALPGGYVRVCEFAGSGGSGGSGGSPVPSTPPTNSSSTPPTPSPSPVPSTPPTNSSSTPPTPSPSPVPSTPPTNSSSTPPTPSPSASAAAQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - DNA, 1704 bp, synthetic construct
  source 1..1704: mol_type = other DNA
atggactggatttggcggatcctgtttctcgtgggagccgccacaggcgcccattcttgtactgtggcccttcctggtggatacgttagagtgtgcgaattcgcaggcagtggtggaagtggtgggtcaggcggttccccggttccatcaacaccaccaaccaatagctcatctaccccgccaacaccaagtccctctcccgtccccagcacacccccgacgaactcatcctccacaccgccaaccccgtctccttccccggttccctctacacctccgacaaactctagttcaacgcctcccactccatcaccttccgcatcagcggccgctcaggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagcggaggcggaggatcaggcggcggaggaagtgggggaggcggatctgatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagcatcgaagtgatgtacccgcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagatttcgccgcctaccggtccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 118 - protein, 867 aa, synthetic construct
MDWIWRILFLVGAATGAHSCTVALPGGYVRVCEFGGGGSGGGGSGGGGSAAAQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRSGVDSGEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 119 - DNA, 2604 bp, synthetic construct
  source 1..2604: mol_type = other DNA
atggactggatttggcggatcctgtttctcgtgggagccgccacaggcgcccattcttgtactgtggcccttcctggtggatacgttagagtgtgcgaattcggaggcggaggatcaggcggcggaggaagtgggggaggcggatctgcggccgctcaggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagcggaggcggaggatcaggcggcggaggaagtgggggaggcggatctgatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagcatcgaagtgatgtacccgcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagatttcgccgcctaccggtccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcagatctggcgtcgactctggcgaaggcagaggctctctgctgacatgtggcgacgtggaagagaatcctggacctatgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttctaa

SEQ ID NO: 120 - protein, 950 aa, synthetic construct
MDWIWRILFLVGAATGAHSCTVALPGGYVRVCEFIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDAAAQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRSGVDSGEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 121 - DNA, 2853 bp, synthetic construct
  source 1..2853: mol_type = other DNA
atggactggatttggcggatcctgtttctcgtgggagccgccacaggcgcccattcttgtactgtggcccttcctggtggatacgttagagtgtgcgaattcatacaacgcacgcctaaaatccaagtctatagtcggcacccggcggaaaatgggaaatctaatttccttaactgctatgtgtccggtttccacccatccgacattgaggtagacctgctgaaaaacggggaacggatagaaaaggtcgaacacagtgatctgtcctttagtaaggattggtctttttatctgctctactacacagagtttactccgacagaaaaagacgagtacgcgtgccgcgtaaaccacgtaacactgagccaaccgaagattgtgaagtgggatagagatgcggccgctcaggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagcggaggcggaggatcaggcggcggaggaagtgggggaggcggatctgatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagcatcgaagtgatgtacccgcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagatttcgccgcctaccggtccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcagatctggcgtcgactctggcgaaggcagaggctctctgctgacatgtggcgacgtggaagagaatcctggacctatgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttctaa

SEQ ID NO: 122 - protein, 1081 aa, synthetic construct
MDWIWRILFLVGAATGAHSCTVALPGGYVRVCEFESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAAAQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRSGVDSGEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 123 - DNA, 3246 bp, synthetic construct
  source 1..3246: mol_type = other DNA
atggactggatttggcggatcctgtttctcgtgggagccgccacaggcgcccattcttgtactgtggcccttcctggtggatacgttagagtgtgcgaattcgagtctaagtacggccctccttgtcctagctgccccgctcctgaatttgaaggcggcccttccgtgttcctgtttcctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagcagactgaccgtggacaagagcagatggcaagagggcaacgtgttcagctgcagcgtgatgcacgaggccctgcacaaccactacacccagaagtctctgagcctgagcctcggcaaggcggccgctcaggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagcggaggcggaggatcaggcggcggaggaagtgggggaggcggatctgatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagcatcgaagtgatgtacccgcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagatttcgccgcctaccggtccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcagatctggcgtcgactctggcgaaggcagaggctctctgctgacatgtggcgacgtggaagagaatcctggacctatgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttctaa

SEQ ID NO: 124 - protein, 926 aa, synthetic construct
MDWIWRILFLVGAATGAHSCTVALPGGYVRVCEFAGSGGSGGSGGSPVPSTPPTNSSSTPPTPSPSPVPSTPPTNSSSTPPTPSPSPVPSTPPTNSSSTPPTPSPSASAAAQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRSGVDSGEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 125 - DNA, 2781 bp, synthetic construct
  source 1..2781: mol_type = other DNA
atggactggatttggcggatcctgtttctcgtgggagccgccacaggcgcccattcttgtactgtggcccttcctggtggatacgttagagtgtgcgaattcgcaggcagtggtggaagtggtgggtcaggcggttccccggttccatcaacaccaccaaccaatagctcatctaccccgccaacaccaagtccctctcccgtccccagcacacccccgacgaactcatcctccacaccgccaaccccgtctccttccccggttccctctacacctccgacaaactctagttcaacgcctcccactccatcaccttccgcatcagcggccgctcaggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagcggaggcggaggatcaggcggcggaggaagtgggggaggcggatctgatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagcatcgaagtgatgtacccgcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagatttcgccgcctaccggtccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcagatctggcgtcgactctggcgaaggcagaggctctctgctgacatgtggcgacgtggaagagaatcctggacctatgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttctaa

SEQ ID NO: 126 - protein, 14 aa, synthetic construct
  SITE 1: note = Biotin-6-aminohexanoic acid
XCTVALPGGYVRVC

SEQ ID NO: 127 - protein, 244 aa, synthetic construct
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELK

SEQ ID NO: 128 - DNA, 732 bp, synthetic construct
  source 1..732: mol_type = other DNA
cagattcagctggtgcagtctggccccgagctgaagaaacccggcgagacagtgaagatcagctgcaaggccagcggctacatcttcaccaactacggcatgaactgggtcaagcaggcccctggcaagagcttcaagtggatgggctggatcaacacctacaccggcgagagcacctacagcgccgacttcaagggcagattcgccttcagcctggaaaccagcgccagcaccgcctacctgcacatcaacgacctgaagaacgaggacaccgccacctacttttgcgccagaagcggcggctacgaccctatggattattggggccagggcaccagcgtgaccgtgtctagcggaggcggaggaagtggcggcggaggatctgggggaggcggatctgatatcgtgctgacccagagccctgccagcctggctgtgtctctgggacagagagccaccatcagctgtcgggccagcgagagcgtggacaattacggcaacaccttcatgcactggtatcagcagaagcccggccagccccccaagctgctgatctacagagccagcaacctggaaagcggcatccccgccagattttccggcagcggcagcagaaccgacttcaccctgaccatcaaccccgtggaagccgacgacgtggccacctattactgccagcagagcaacgaggacccccctacctttggagccggcaccaagctggaactgaag

SEQ ID NO: 129 - DNA, 363 bp, synthetic construct
  source 1..363: mol_type = other DNA
gaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctct

SEQ ID NO: 130 - protein, 121 aa, synthetic construct
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVS

SEQ ID NO: 131 - DNA, 321 bp, synthetic construct
  source 1..321: mol_type = other DNA
gatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaag

SEQ ID NO: 132 - protein, 107 aa, synthetic construct
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 133 - DNA, 732 bp, synthetic construct
  source 1..732: mol_type = other DNA
gaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaag

SEQ ID NO: 134 - protein, 244 aa, synthetic construct
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 135 - DNA, 21 bp, synthetic construct
  source 1..21: mol_type = other DNA
agatctggcgtcgactctggc

SEQ ID NO: 136 - protein, 7 aa, synthetic construct
RSGVDSG

SEQ ID NO: 137 - DNA, 207 bp, Homo sapiens
  source 1..207: mol_type = genomic DNA
accaccacaccagctcctcggcctccaactcctgctcctacaattgccagccagcctctgtctctgaggcccgaagcttgtagacctgctgctggcggagccgtgcatacaagaggactggatttcgcctgcgacatctacatctgggcccctctggctggaacatgtggcgttctgctgctgagcctggtcatcaccctgtactgc

SEQ ID NO: 138 - DNA, 123 bp, Homo sapiens
  source 1..123: mol_type = genomic DNA
cggtccaagagaagcagactgctgcacagcgactacatgaacatgacccctagacggcccggacctaccagaaagcactaccagccttacgctcctcctagagatttcgccgcctaccggtcc

SEQ ID NO: 139 - DNA, 336 bp, Homo sapiens
  source 1..336: mol_type = genomic DNA
agagtgaagttctccagatccgccgatgctcccgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 140 - DNA, 888 bp, synthetic construct
  source 1..888: mol_type = other DNA
atggactggatttggcggatcctgtttctcgtgggagccgccacaggcgcccattcttgtactgtggcccttcctggtggatacgttagagtgtgcgaattcggaggcggaggatcaggcggcggaggaagtgggggaggcggatctgcggccgctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaag

SEQ ID NO: 141 - protein, 296 aa, synthetic construct
MDWIWRILFLVGAATGAHSCTVALPGGYVRVCEFGGGGSGGGGSGGGGSAAAEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 142 - DNA, 1536 bp, synthetic construct
  source 1..1536: mol_type = other DNA
cgaagcaagcggagccggctgctgcacagcgactacatgaacatgacccctagacggcccggaccaaccagaaagcactaccagccttacgctcctcctagagatttcgccgcctaccggtccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcagatctggcgtcgactctggcgaaggcagaggctctctgctgacatgtggcgacgtggaagagaatcctggacctatgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttctaa

SEQ ID NO: 143 - protein, 511 aa, synthetic construct
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRSGVDSGEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 144 - DNA, 2622 bp, synthetic construct
  source 1..2622: mol_type = other DNA
atggactggatttggcggatcctgtttctcgtgggagccgccacaggcgcccattcttgtactgtggcccttcctggtggatacgttagagtgtgcgaattcggaggcggaggatcaggcggcggaggaagtgggggaggcggatctgcggccgctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaagatcgaagtgatgtacccgcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagatttcgccgcctaccggtccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcagatctggcgtcgactctggcgaaggcagaggctctctgctgacatgtggcgacgtggaagagaatcctggacctatgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttctaa

SEQ ID NO: 145 - protein, 873 aa, synthetic construct
MDWIWRILFLVGAATGAHSCTVALPGGYVRVCEFGGGGSGGGGSGGGGSAAAEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRSGVDSGEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 146 - DNA, 879 bp, synthetic construct
  source 1..879: mol_type = other DNA
atggactggatttggcggatcctgtttctcgtgggagccgccacaggcgcccattcttgtactgtggcccttcctggtggatacgttagagtgtgcgaattcggaggcggaggatcaggcggcggaggaagtgggggaggcggatctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaag

SEQ ID NO: 147 - protein, 293 aa, synthetic construct
MDWIWRILFLVGAATGAHSCTVALPGGYVRVCEFGGGGSGGGGSGGGGSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 148 - DNA, 462 bp, synthetic construct
  source 1..462: mol_type = other DNA
cggtccaagagaagcagactgctgcacagcgactacatgaacatgacccctagacggcccggacctaccagaaagcactaccagccttacgctcctcctagagatttcgccgcctaccggtccagagtgaagttctccagatccgccgatgctcccgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataa

SEQ ID NO: 149 - protein, 153 aa, synthetic construct; identical to SEQ ID NO: 108

SEQ ID NO: 150 - DNA, 1548 bp, synthetic construct
  source 1..1548: mol_type = other DNA
atggactggatttggcggatcctgtttctcgtgggagccgccacaggcgcccattcttgtactgtggcccttcctggtggatacgttagagtgtgcgaattcggaggcggaggatcaggcggcggaggaagtgggggaggcggatctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaagaccaccacaccagctcctcggcctccaactcctgctcctacaattgccagccagcctctgtctctgaggcccgaagcttgtagacctgctgctggcggagccgtgcatacaagaggactggatttcgcctgcgacatctacatctgggcccctctggctggaacatgtggcgttctgctgctgagcctggtcatcaccctgtactgccggtccaagagaagcagactgctgcacagcgactacatgaacatgacccctagacggcccggacctaccagaaagcactaccagccttacgctcctcctagagatttcgccgcctaccggtccagagtgaagttctccagatccgccgatgctcccgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataa

SEQ ID NO: 151 - protein, 515 aa, synthetic construct
MDWIWRILFLVGAATGAHSCTVALPGGYVRVCEFGGGGSGGGGSGGGGSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2025/0195658 A1
(43) Pub. Date: Jun. 19, 2025

(54) BISPECIFIC CHIMERIC ANTIGEN RECEPTORS TARGETING GRP78 AND CD123 OR GRP78 AND B7H3

(71) Applicant: ST. JUDE CHILDREN'S RESEARCH HOSPITAL, INC., Memphis, TN (US)

(72) Inventors: Mireya VELASQUEZ, Memphis, TN (US); Stephen GOTTSCHALK, Germantown, TN (US); Jaquelyn ZOINE, Memphis, TN (US); Nikhil HEBBAR, Cordova, TN (US)

(73) Assignee: ST. JUDE CHILDREN'S RESEARCH HOSPITAL, INC., Memphis, TN (US)

(21) Appl. No.: 18/727,752

(22) PCT Filed: Jan. 11, 2023
(86) PCT No.: PCT/US2023/010607

§ 371 Date: Jul. 10, 2024

Related U.S. Application Data
(60) Provisional application No. 63/298,817, filed on Jan. 12, 2022.

(51) Int. Cl. A61K 40/42; A61K 40/11; A61K 40/31; A61P 35/00; C07K 14/725; C07K 16/28; C07K 16/40

(52) U.S. Cl. CPC A61K 40/4217; A61K 40/11; A61K 40/31; A61K 40/4244; A61P 35/00; C07K 14/7051; C07K 16/2866; C07K 16/40; C12Y 306/0401; C07K 2317/31; C07K 2317/569

(57) ABSTRACT

The application provides bispecific chimeric antigen receptors (CARs) targeting glucose-regulated-protein 78 (GRP78) and Cluster of Differentiation 123 (CD123) or GRP78 and B7-homolog 3 (B7H3). The application further provides polynucleotides and recombinant vectors encoding the CARs, as well isolated host cells and methods for preparing isolated host cells that express the CARs. The application further provides pharmaceutical compositions comprising the CAR modified cells and methods for treating a tumor using the CAR modified cells.

### GOVERNMENT FUNDING STATEMENT

This invention was made with government support under CA236748 awarded by National Institutes of Health. The government has certain rights in the invention.

### FIELD OF THE INVENTION

The application relates to bispecific chimeric antigen receptors (CARs) targeting glucose-regulated-protein 78 (GRP78) and Cluster of Differentiation 123 (CD123) or GRP78 and B7-homolog 3 (B7H3). The application further relates to polynucleotides and recombinant vectors encoding the CARs, as well as to isolated host cells and methods for preparing isolated host cells that express the CARs. The application further relates to pharmaceutical compositions comprising the CAR modified cells and to methods for treating a tumor using the CAR modified cells.

### BACKGROUND

Adoptive immunotherapy has shown encouraging results treating patients with B cell acute lymphoblastic leukemia (ALL), which has led to preclinical and clinical exploration of chimeric antigen (CAR) T cell therapy for acute myeloid leukemia (AML).¹⁻³ In many cases of AML, tumor cells survive induction and consolidation chemotherapy, leading to relapsed disease. There are two major patterns in AML relapse: i) the initial clone gains mutations; or ii) a subclone survives initial treatments.⁴ However, in the majority of cases the relapse clone is characterized as a leukemia stem cell (LSC) based on CD34+ CD38− phenotypes.^{5,6} These minor LSC clones are largely responsible for relapse cases of AML.^{7,8} Given the aggressive nature of AML and its capacity to relapse it is imperative to find new therapies capable of sustaining remission.

CAR T cells consist of an antigen recognition domain (generally a single chain variable fragment [scFv]), a hinge (H), transmembrane (TM) domain, and signaling domain which provides costimulation and signals through CD3ζ to activate the T cell in an antigen-dependent manner.^{9,10} The pursuit of effective CAR T cells for AML has been challenging due to the heterogeneity of AML and the overlapping expression of antigens on healthy tissues such as hematopoietic progenitor cells (HPCs) and AML blasts.¹¹ Preclinical studies using CAR T cells targeting AML antigens such as CD33, CLL1 and CD123 have had encouraging results and clinical trials are ongoing.¹²

CD123 is highly expressed on AML blasts and leukemia stem cells, and exhibits low levels of expression on endothelial cells, normal IPCs and mature myeloid lineages.¹³ In addition, the inventors have generated a glucose related protein 78 (GRP78)-specific CAR with potent anti-AML activity in vitro and in vivo.²⁹ Surface expression of GRP78 is limited to cancer cells and is associated with tumor cell proliferation, survival, and chemoresistance.¹⁸ B7-homolog 3 (B7H3), also referred to as CD276, is a coreceptor belonging to the B7 family of immune checkpoint molecules and is an attractive target for immunotherapy as it is expressed on several malignancies, including solid tumors and leukemic blasts but not on normal hematopoietic stem cells (HSCs).

Clinical experience with CD19-CAR T cells treating CD19+ malignancies have shown a high incidence of CD19-negative relapse due to loss of CD19 surface expression.^{14,15} It is hypothesized that the selective pressure of single antigen directed targeted therapy results in immune escape by the cancer. Immune escape can occur through antigen escape (e.g., alternative splicing, genetic variants, loss of heterozygosity) or lineage switch. ¹⁶⁻¹⁸.

### SUMMARY OF THE INVENTION

As specified in the Background section above, there is a great need in the art for strategies to circumvent immune escape by targeting more than one antigen, in particular, via targeting two antigens by designing a bispecific antigen-binding domain for CAR T cells. The present application addresses these and other needs.

Disclosed herein are anti-glucose-regulated-protein 78 (GRP78) bispecific CARs targeting GRP78 and Cluster of Differentiation 123 (CD123) and GRP78 and B7-homolog 3 (B7H3). Further disclosed are related polynucleotides, vectors, and cell compositions comprising the same, as well as compositions (e.g., pharmaceutical compositions) comprising the polypeptides, polynucleotides, vectors, or cell compositions, and methods of using such compositions in treating a cancer in a subject.

In one aspect, the present disclosure provides a polynucleotide encoding a chimeric antigen receptor (CAR) comprising:
a) an extracellular antigen-binding domain comprising one or more GRP78-binding moieties and a CD123-binding moiety;
b) a transmembrane domain; and
c) a cytoplasmic domain comprising a signaling domain.

In some embodiments, the GRP78-binding moiety may comprise a GRP78-binding peptide.

In some embodiments, the GRP78-binding peptide may comprise the amino acid sequence CTVALPGGYVRVC (SEQ ID NO: 92), or a variant thereof.

In some embodiments, the nucleotide sequence encoding the GRP78-binding peptide may comprise the sequence TGTACTGTGGCCCTTCCTGGTGGATACGTTAGAGTGTGC (SEQ ID NO: 93), or a nucleotide sequence having at least 80% sequence identity thereof.

In some embodiments, the extracellular antigen-binding domain may comprise one, two or three GRP78-binding moieties.

In some embodiments, when more than one GRP78-binding moiety may be used, each GRP78-binding moiety may be linked via a linker sequence.

In some embodiments, the extracellular antigen-binding domain may comprise one GRP78-binding moiety.

In some embodiments, the CD123-binding moiety may be an antibody or antibody fragment.

In some embodiments, the CD123-binding moiety may be a single chain variable fragment (scFv).

In some embodiments, the anti-CD123 scFv may be derived from antibody 26292 (scFV (292)).

In some embodiments, scFV (292) may comprise a heavy chain variable domain (VH) comprising the amino acid sequence of SEQ ID NO: 94, or an amino acid sequence having at least 80% identity thereof, and/or a light chain variable domain (VL) which may comprise the amino acid sequence of SEQ ID NO: 96, or an amino acid sequence having at least 80% identity thereof.

In some embodiments, the nucleotide sequence encoding scFV (292) may comprise a nucleotide encoding the VH and comprising the nucleotide sequence of SEQ ID NO: 95, or a nucleotide having at least 80% identity thereof, and/or a nucleotide encoding the VL and comprising the nucleotide sequence of SEQ ID NO: 97, or a nucleotide sequence having at least 80% identity thereof.

In some embodiments, the VH and the VL may be linked via a linker sequence.

In some embodiments, scFV (292) comprises three heavy chain complementarity determining regions (CDRs) (HCDR1, HCDR2 and HCDR3) contained within the VH sequence of SEQ ID NO: 94; and/or three light chain CDRs (LCDR1, LCDR2 and LCDR3) contained within the VL sequence of SEQ ID NO: 96.

In some embodiments, scFV (292) may comprise an amino acid sequence of SEQ ID NO: 98, or an amino acid sequence having at least 80% identity thereof.

In some embodiments, the nucleotide sequence encoding scFV (292) may comprise the nucleotide sequence of SEQ ID NO: 99, or a nucleotide sequence having at least 80% identity thereof.

In some embodiments of any of the polynucleotides disclosed herein, the extracellular antigen-binding domain may further comprise a linker sequence between the one or more GRP78-binding moieties and the CD123-binding moiety.

In some embodiments, the linker sequence may comprise any one of the linker sequences selected from Table 2 disclosed herein, or an amino acid sequence having at least 80% identity thereof.

In some embodiments, the linker sequence may comprise a (G4S)3 linker (SEQ ID NO: 9), a β2M linker (SEQ ID NO: 12), a mutated IgG4 linker (SEQ ID NO: 14), or a GPcPcPc linker (SEQ ID NO: 16), or an amino acid sequence having at least 80% identity thereof.

In some embodiments, the linker sequence may be encoded by any one of SEQ ID NOs: 10, 11, 13, 15, or 17, or a nucleotide sequence having at least 80% sequence identity thereof.

In some embodiments, the linker sequence may comprise a (G4S)3 linker (SEQ ID NO: 9), or is encoded by SEQ ID NOs: 10 or 11.

In some embodiments of any of the polynucleotides disclosed herein, the extracellular antigen-binding domain may further comprise a leader sequence.

In some embodiments, the leader sequence may be derived from human immunoglobulin (IgG) heavy chain variable region of CD8a.

In some embodiments, the IgG-derived leader sequence may comprise the amino acid sequence of SEQ ID NO: 1, or an amino acid sequence having at least 80% sequence identity thereof.

In some embodiments, the nucleotide sequence encoding the IgG-derived leader sequence may comprise the sequence of SEQ ID NO: 2, or a nucleotide sequence having at least 80% sequence identity thereof.

In some embodiments of any of the polynucleotides disclosed herein, the CAR may further comprise a hinge domain between the extracellular target binding domain and the transmembrane domain.

In some embodiments, the hinge domain may be derived from CD8a, CD28, or an IgG.

In some embodiments, the hinge domain may be derived from CD28.

In some embodiments, the CD28 hinge domain may comprise the amino acid sequence of SEQ ID NO: 37, or an amino acid sequence having at least 80% sequence identity thereof.

In some embodiments, the nucleotide sequence encoding the CD28 hinge domain may comprise the sequence of SEQ ID NO: 38, or a nucleotide sequence having at least 80% sequence identity thereof.

In some embodiments of any of the polynucleotides disclosed herein, the transmembrane domain may be derived from CD8a, CD28, CD8, CD4, CD3ζ, CD40, CD134 (OX-40), NKG2A/C/D/E or CD7.

In some embodiments of any of the polynucleotides disclosed herein, the transmembrane domain may be derived from CD28.

In some embodiments, the CD28 transmembrane domain may comprise the amino acid sequence SEQ ID NO: 43, or an amino acid sequence having at least 80% sequence identity thereof.

In some embodiments, the nucleotide sequence encoding the CD28 transmembrane domain may comprise the sequence SEQ ID NO: 44 or 45, or a nucleotide sequence having at least 80% sequence identity thereof.

In some embodiments of any of the polynucleotides disclosed herein, the signaling domain may be derived from CD3ζ, DAP10, DAP12, Fc ε receptor I γ chain (FCER1G), CD3δ, CD3ε, CD3γ, CD226, NKG2D, or CD79A.

In some embodiments, the signaling domain may be derived from CD3ζ.

In some embodiments, the CD3ζ signaling domain may comprise the amino acid sequence SEQ ID NO: 69, or an amino acid sequence having at least 80% sequence identity thereof.

In some embodiments, the nucleotide sequence encoding the CD3ζ signaling domain may comprise the sequence SEQ ID NO: 70 or 71, or a nucleotide sequence having at least 80% sequence identity thereof.

In some embodiments of any of the polynucleotides disclosed herein, the cytoplasmic domain may further comprise one or more costimulatory domains.

In some embodiments, the one or more costimulatory domains may be derived from CD28, CD27, CD40, CD134, CD226, CD79A, ICOS, 4-1BB, OX40 or MyD88, or any combination thereof.

In some embodiments, the cytoplasmic domain may comprise a CD28 costimulatory domain.

In some embodiments, the CD28 costimulatory domain may comprise the amino acid sequence of SEQ ID NO: 54, or an amino acid sequence having at least 80% sequence identity thereof.

In some embodiments, the nucleotide sequence encoding the CD28 costimulatory domain may comprise the sequence of SEQ ID NO: 55 or 56, or a nucleotide sequence having at least 80% sequence identity thereof.

In some embodiments of any of the polynucleotides disclosed herein, the CAR may comprise the amino acid sequence of any one of SEQ ID NOs: 110, 112, 114, or 116, or an amino acid sequence having at least 80% sequence identity thereof.

In some embodiments, the nucleotide sequence encoding the CAR may comprise the sequence of any one of SEQ ID NOs: 113, 115, 117 or 119, or a nucleotide sequence having at least 80% sequence identity thereof.

In some embodiments of any of the polynucleotides disclosed herein, polynucleotide further encodes at least one additional polypeptide.

In some embodiments, the at least one polypeptide may be a transduced host cell selection marker, an in vivo tracking marker, a cytokine, or a safety switch gene, dimerization moiety, or degradation moiety.

In some embodiments, the transduced host cell selection marker may be a truncated CD19 (tCD19) polypeptide.

In some embodiments, the tCD19 may comprise the amino acid sequence SEQ ID NO: 88, or an amino acid sequence having at least 80% sequence identity thereof.

In some embodiments, the nucleotide sequence encoding the tCD19 may comprise the nucleotide sequence SEQ ID NO: 89, or a nucleotide sequence having at least 80% sequence identity thereof.

In some embodiments of any of the polynucleotides disclosed herein, the sequence encoding the CAR may be operably linked to the sequence encoding at least an additional polypeptide sequence via a sequence encoding a self-cleaving peptide and/or an internal ribosomal entry site (IRES).

In some embodiments, the self-cleaving peptide may be a 2A peptide.

In some embodiments, the 2A peptide may be T2A, P2A, E2A, or F2A peptide.

In some embodiments, the 2A peptide may be a T2A peptide.

In some embodiments, the T2A peptide may comprise the amino acid sequence SEQ ID NO: 74, or an amino acid sequence having at least 80% sequence identity thereof.

In some embodiments, the sequence encoding the T2A peptide may comprise the nucleotide sequence SEQ ID NO: 75 or 76, or a nucleotide sequence having at least 80% sequence identity thereof.

In some embodiments, any of the polynucleotides disclosed herein may encode the amino acid sequence of any one of SEQ ID NOs: 118, 120, 122, or 124, or an amino acid sequence having at least 80% sequence identity thereof.

In some embodiments, any of the polynucleotides disclosed herein may comprise the nucleotide sequence of any one of SEQ ID NOs: 119, 121, 123 or 125, or a nucleotide sequence having at least 80% sequence identity thereof.

In some embodiments, any of the polynucleotides disclosed herein may be a DNA molecule.

In some embodiments, any of the polynucleotides disclosed herein may be an RNA molecule.

In some embodiments of any of the polynucleotides disclosed herein, the polynucleotide may be expressed in an inducible fashion, achieved with an inducible promoter, an inducible expression system, an artificial signaling circuit, and/or drug induced splicing.

In some embodiments, the promoter may be a T cell-specific promoter or an NK cell-specific promoter.

In another aspect, the present disclosure provides a chimeric antigen receptor (CAR) encoded by any of the polynucleotides disclosed herein.

In another aspect, the present disclosure provides a recombinant vector comprising any of the polynucleotides disclosed herein.

In some embodiments, the vector may be a viral vector.

In some embodiments, the viral vector may be a retroviral vector, a lentiviral vector, an adenoviral vector, an adeno-associated virus vector, an alphaviral vector, a herpes virus vector, a baculoviral vector, or a vaccinia virus vector.

In some embodiments, the viral vector may be a retroviral vector.

In some embodiments, the vector may be a non-viral vector.

In some embodiments, the non-viral vector may be a minicircle plasmid, a Sleeping Beauty transposon, a piggyBac transposon, or a single or double stranded DNA molecule that is used as a template for homology directed repair (HDR) based gene editing.

In another aspect, the present disclosure provides an isolated host cell comprising any of the polynucleotides disclosed herein or any of the recombinant vectors disclosed herein.

In another aspect, the present disclosure provides an isolated host cell comprising a chimeric antigen receptor (CAR) encoded by any of the polynucleotide disclosed herein.

In some embodiments, the host cell may be an immune cell.

In some embodiments of any of the isolated host cells disclosed herein, the host cell may be a T cell, a natural killer (NK) cell, a mesenchymal stem cell (MSC), or a macrophage.

In some embodiments, the host cell may be a T cell.

In some embodiments, the host cell may be a CD8+ T cell, a CD4+ T cell, a cytotoxic T cell, an αβ T cell receptor (TCR) T cell, an invariant natural killer T (iNKT) cell, a γδ T cell, a memory T cell, a memory stem T cell (TSCM), a naïve T cell, an effector T cell, a T-helper cell, or a regulatory T cell (Treg).

In some embodiments, the host cell may be a natural killer (NK) cell.

In some embodiments, the NK cell may be derived from peripheral, cord blood, IPSCs, and/or a cell line (e.g., NK-92 cells).

In some embodiments, the immune cell may be derived from an induced pluripotent stem (IPS) cell.

In some embodiments, any of the isolated host cells of the present disclosure may further genetically modified to enhance its function by expressing one or more additional genes (e.g., transcription factors (e.g., c-Jun) or cytokines (e.g., IL-15); or deleting one or more inhibitory genes (e.g., REGNASE-1, CISH, DNMT3A) with gene editing technologies (e.g., CRISPR-Cas9, base editors, or transcription activator-like effector nucleases (TALENs)).

In some embodiments, the host cell has been activated and/or expanded ex vivo.

In some embodiments, the host cell may be an allogeneic cell.

In some embodiments, the host cell may be an autologous cell.

In some embodiments, the host cell may be isolated from a subject having a cancer, wherein one or more cells of the cancer express GRP78 and/or CD123.

In some embodiments, the cancer may be a hematologic malignancy.

In some embodiments, the hematologic malignancy is acute myeloid leukemia (AML), acute lymphoblastic leukemia (ALL), B-cell acute lymphoblastic leukemia (B-ALL), T cell acute lymphoblastic leukemia (T-ALL), a blastic plasmacytoid dendritic neoplasm (BPCDN), a hairy cell leukemia, or lymphoma.

In some embodiments, the host cell may be derived from a blood, marrow, tissue, or a tumor sample.

In another aspect, the present disclosure provides a pharmaceutical composition comprising any of the host cell disclosed herein and a pharmaceutically acceptable carrier and/or excipient.

In another aspect, the present disclosure provides a method of generating any of the isolated host cells disclosed herein, said method comprising genetically modifying the host cell with any of the polynucleotides disclosed herein or any of the recombinant vectors disclosed herein.

In some embodiments, the genetic modifying step may be conducted via viral gene delivery.

In some embodiments, the genetic modifying step may be conducted via non-viral gene delivery.

In some embodiments, the genetic modification may be conducted ex vivo.

In some embodiments of any of the methods disclosed herein, the method further may comprise activation and/or expansion of the host cell ex vivo before, after and/or during said genetic modification.

In another aspect, the present disclosure provides a method for killing a cancer cell expressing GRP78 and/or CD123, said method comprising contacting said cell with any of the host cells disclosed herein or the pharmaceutical composition disclosed herein.

In another aspect, the present disclosure provides a method for treating a cancer in a subject in need thereof, wherein one or more cells of the tumor express GRP78 and/or CD123, said method comprising administering to the subject a therapeutically effective amount of any of the host cells disclosed herein or the pharmaceutical composition disclosed herein.

In some embodiments, the cancer may be a hematologic malignancy.

In some embodiments, the hematologic malignancy may be acute myeloid leukemia (AML), acute lymphoblastic leukemia (ALL), B-cell acute lymphoblastic leukemia (B-ALL), T cell acute lymphoblastic leukemia (T-ALL), a blastic plasmacytoid dendritic neoplasm (BPCDN), a hairy cell leukemia, or lymphoma.

In some embodiments of any of the methods disclosed, the method may comprise:
a) isolating T cells, NK cells, mesenchymal stem cells or macrophages from the subject;
b) genetically modifying said T cells, NK cells, mesenchymal stem cells, or macrophages ex vivo with any of the polynucleotides disclosed herein or any of the vectors disclosed herein;
c) optionally, expanding and/or activating said T cells, NK cells, mesenchymal stem cells, or macrophages before, after or during step (b); and
d) introducing the genetically modified T cells, NK cells, mesenchymal stem cells, or macrophages into the subject.

In some embodiments of any of the methods disclosed herein, the subject may be human.

In one aspect, the present disclosure provides a polynucleotide encoding a chimeric antigen receptor (CAR) comprising:
a) an extracellular antigen-binding domain comprising one or more GRP78-binding moieties and a B7H3-binding moiety;
b) a transmembrane domain; and
c) a cytoplasmic domain comprising a signaling domain.

In some embodiments, the GRP78-binding moiety may comprise a GRP78-binding peptide.

In some embodiments, the GRP78-binding peptide may comprise the amino acid sequence CTVALPGGYVRVC (SEQ ID NO: 92), or a variant thereof.

In some embodiments, the nucleotide sequence encoding the GRP78-binding peptide may comprise the sequence TGTACTGTGGCCCTTCCTGGTGGATACGTTAGAGTGTGC (SEQ ID NO: 93), or a nucleotide sequence having at least 80% sequence identity thereof.

In some embodiments, the extracellular antigen-binding domain may comprise one, two or three GRP78-binding moieties.

In some embodiments, when more than one GRP78-binding moiety may be used, each GRP78-binding moiety may be linked via a linker sequence.

In some embodiments, the extracellular antigen-binding domain may comprise one GRP78-binding moiety.

In some embodiments, the B7H3-binding moiety may be an antibody or antibody fragment.

In some embodiments, the B7H3-binding moiety may be a single chain variable fragment (scFv).

In some embodiments, the B7H3-binding moiety may be a single chain variable fragment (scFv) derived from humanized B7-H3-specific monoclonal antibody (mAb) MGA271.

In some embodiments, the B7H3 scFV may comprise a heavy chain variable domain (VH) comprising the amino acid sequence of SEQ ID NO: 130, or an amino acid sequence having at least 80% identity thereof, and/or a light chain variable domain (VL) which may comprise the amino acid sequence of SEQ ID NO: 132, or an amino acid sequence having at least 80% identity thereof.

In some embodiments, the nucleotide sequence encoding the B7H3 scFV may comprise a nucleotide encoding the VH and comprising the nucleotide sequence of SEQ ID NO: 129, or a nucleotide having at least 80% identity thereof, and/or a nucleotide encoding the VL and comprising the nucleotide sequence of SEQ ID NO: 131, or a nucleotide sequence having at least 80% identity thereof.

In some embodiments, the VH and the VL may be linked via a linker sequence.

In some embodiments, the B7H3 scFv may comprise three heavy chain complementarity determining regions (CDRs) (HCDR1, HCDR2 and HCDR3) contained within the VH sequence of SEQ ID NO: 94; and/or three light chain CDRs (LCDR1, LCDR2 and LCDR3) contained within the VL sequence of SEQ ID NO: 96.

In some embodiments, the B7H3 scFV may comprise an amino acid sequence of SEQ ID NO: 134, or an amino acid sequence having at least 80% identity thereof.

In some embodiments, the nucleotide sequence encoding the B7H3 scFV may comprise the nucleotide sequence of SEQ ID NO: 133, or a nucleotide sequence having at least 80% identity thereof.

In some embodiments of any of the polynucleotides disclosed herein, the extracellular antigen-binding domain may further comprise a linker sequence between the one or more GRP78-binding moieties and the B7H3-binding moiety.

In some embodiments, the linker sequence may comprise any one of the linker sequences selected from Table 2 disclosed herein, or an amino acid sequence having at least 80% identity thereof.

In some embodiments, the linker sequence may comprise a (G4S)3 linker (SEQ ID NO: 9), a β2M linker (SEQ ID NO: 12), a mutated IgG4 linker (SEQ ID NO: 14), or a GPcPcPc linker (SEQ ID NO: 16), or an amino acid sequence having at least 80% identity thereof.

In some embodiments, the linker sequence may be encoded by any one of SEQ ID NOs: 10, 11, 13, 15, or 17, or a nucleotide sequence having at least 80% sequence identity thereof.

In some embodiments, the linker sequence may comprise a (G4S)3 linker (SEQ ID NO: 9), or is encoded by SEQ ID NOs: 10 or 11.

In some embodiments, the linker sequence may comprise the amino acid sequence RSGVDSG (SEQ ID NO: 136) or is encoded by SEQ ID NO: 135.

In some embodiments of any of the polynucleotides disclosed herein, the extracellular antigen-binding domain may further comprise a leader sequence.

In some embodiments, the leader sequence may be derived from human immunoglobulin (IgG) heavy chain variable region of CD8a.

In some embodiments, the IgG-derived leader sequence may comprise the amino acid sequence of SEQ ID NO: 1, or an amino acid sequence having at least 80% sequence identity thereof.

In some embodiments, the nucleotide sequence encoding the IgG-derived leader sequence may comprise the sequence of SEQ ID NO: 2, or a nucleotide sequence having at least 80% sequence identity thereof.

In some embodiments of any of the polynucleotides disclosed herein, the CAR may further comprise a hinge domain between the extracellular target binding domain and the transmembrane domain.

In some embodiments, the hinge domain may be derived from CD8a, CD28, or an IgG.

In some embodiments, the hinge domain may be derived from CD28.

In some embodiments, the CD28 hinge domain may comprise the amino acid sequence of SEQ ID NO: 37, or an amino acid sequence having at least 80% sequence identity thereof.

In some embodiments, the nucleotide sequence encoding the CD28 hinge domain may comprise the sequence of SEQ ID NO: 38, or a nucleotide sequence having at least 80% sequence identity thereof.

In some embodiments, the hinge domain may be derived from CD8a.

In some embodiments, the CD8a hinge domain may comprise the amino acid sequence of SEQ ID NO: 137, or an amino acid sequence having at least 80% sequence identity thereof.

In some embodiments, the nucleotide sequence encoding the CD8a hinge domain may comprise the sequence of SEQ ID NO: 35, or a nucleotide sequence having at least 80% sequence identity thereof.

In some embodiments of any of the polynucleotides disclosed herein, the transmembrane domain may be derived from CD8a, CD28, CD8, CD4, CD3ζ, CD40, CD134 (OX-40), NKG2A/C/D/E or CD7.

In some embodiments of any of the polynucleotides disclosed herein, the transmembrane domain may be derived from CD28.

In some embodiments, the CD28 transmembrane domain may comprise the amino acid sequence SEQ ID NO: 53, or an amino acid sequence having at least 80% sequence identity thereof.

In some embodiments, the nucleotide sequence encoding the CD28 transmembrane domain may comprise the sequence SEQ ID NO: 52, or a nucleotide sequence having at least 80% sequence identity thereof.

In some embodiments of any of the polynucleotides disclosed herein, the transmembrane domain may be derived from CD8a.

In some embodiments, the CD8a transmembrane domain may comprise the amino acid sequence SEQ ID NO: 48, or an amino acid sequence having at least 80% sequence identity thereof.

In some embodiments, the nucleotide sequence encoding the CD8a transmembrane domain may comprise the sequence SEQ ID NO: 49, or a nucleotide sequence having at least 80% sequence identity thereof.

In some embodiments of any of the polynucleotides disclosed herein, the signaling domain may be derived from CD3ζ, DAP10, DAP12, Fc ε receptor I γ chain (FCER1G), CD3δ, CD3ε, CD3γ, CD226, NKG2D, or CD79A.

In some embodiments, the signaling domain may be derived from CD3ζ.

In some embodiments, the CD3ζ signaling domain may comprise the amino acid sequence SEQ ID NO: 69, or an amino acid sequence having at least 80% sequence identity thereof.

In some embodiments, the nucleotide sequence encoding the CD3ζ signaling domain may comprise the sequence SEQ ID NO: 70, 71, or 139, or a nucleotide sequence having at least 80% sequence identity thereof.

In some embodiments of any of the polynucleotides disclosed herein, the cytoplasmic domain may further comprise one or more costimulatory domains.

In some embodiments, the one or more costimulatory domains may be derived from CD28, CD27, CD40, CD134, CD226, CD79A, ICOS, 4-1BB, OX40 or MyD88, or any combination thereof.

In some embodiments, the cytoplasmic domain may comprise a CD28 costimulatory domain.

In some embodiments, the CD28 costimulatory domain may comprise the amino acid sequence of SEQ ID NO: 54, or an amino acid sequence having at least 80% sequence identity thereof.

In some embodiments, the nucleotide sequence encoding the CD28 costimulatory domain may comprise the sequence of SEQ ID NO: 55, 56, or 138, or a nucleotide sequence having at least 80% sequence identity thereof.

In some embodiments of any of the polynucleotides disclosed herein, the CAR may comprise the amino acid sequence of any one of SEQ ID NOs: 145 or 151, or an amino acid sequence having at least 80% sequence identity thereof.

In some embodiments, the nucleotide sequence encoding the CAR may comprise the sequence of any one of SEQ ID NOs: 144 or 150, or a nucleotide sequence having at least 80% sequence identity thereof.

In some embodiments of any of the polynucleotides disclosed herein, the polynucleotide further encodes at least one additional polypeptide.

In some embodiments, the at least one polypeptide may be a transduced host cell selection marker, an in vivo tracking marker, a cytokine, or a safety switch gene, dimerization moiety, or degradation moiety.

In some embodiments, the transduced host cell selection marker may be a truncated CD19 (tCD19) polypeptide.

In some embodiments, the tCD19 may comprise the amino acid sequence SEQ ID NO: 88, or an amino acid sequence having at least 80% sequence identity thereof.

In some embodiments, the nucleotide sequence encoding the tCD19 may comprise the nucleotide sequence SEQ ID NO: 89, or a nucleotide sequence having at least 80% sequence identity thereof.

In some embodiments of any of the polynucleotides disclosed herein, the sequence encoding the CAR may be operably linked to the sequence encoding at least an additional polypeptide sequence via a sequence encoding a self-cleaving peptide and/or an internal ribosomal entry site (IRES).

In some embodiments, the self-cleaving peptide may be a 2A peptide.

In some embodiments, the 2A peptide may be T2A, P2A, E2A, or F2A peptide.

In some embodiments, the 2A peptide may be a T2A peptide.

In some embodiments, the T2A peptide may comprise the amino acid sequence SEQ ID NO: 74, or an amino acid sequence having at least 80% sequence identity thereof.

In some embodiments, the sequence encoding the T2A peptide may comprise the nucleotide sequence SEQ ID NO: 75 or 76, or a nucleotide sequence having at least 80% sequence identity thereof.

In some embodiments, any of the polynucleotides disclosed herein may encode the amino acid sequence of any one of SEQ ID NOs: 145 or 151, or an amino acid sequence having at least 80% sequence identity thereof.

In some embodiments, any of the polynucleotides disclosed herein may comprise the nucleotide sequence of any one of SEQ ID NOs: 144 or 150, or a nucleotide sequence having at least 80% sequence identity thereof.

In some embodiments, any of the polynucleotides disclosed herein may be a DNA molecule.

In some embodiments, any of the polynucleotides disclosed herein may be an RNA molecule.

In some embodiments of any of the polynucleotides disclosed herein, the polynucleotide may be expressed in an inducible fashion, achieved with an inducible promoter, an inducible expression system, an artificial signaling circuit, and/or drug induced splicing.

In some embodiments, the promoter may be a T cell-specific promoter or an NK cell-specific promoter.

In another aspect, the present disclosure provides a chimeric antigen receptor (CAR) encoded by any of the polynucleotides disclosed herein.

In another aspect, the present disclosure provides a recombinant vector comprising any of the polynucleotides disclosed herein.

In some embodiments, the vector may be a viral vector.

In some embodiments, the viral vector may be a retroviral vector, a lentiviral vector, an adenoviral vector, an adeno-associated virus vector, an alphaviral vector, a herpes virus vector, a baculoviral vector, or a vaccinia virus vector.

In some embodiments, the viral vector may be a retroviral vector.

In some embodiments, the vector may be a non-viral vector.

In some embodiments, the non-viral vector may be a minicircle plasmid, a Sleeping Beauty transposon, a piggyBac transposon, or a single or double stranded DNA molecule that is used as a template for homology directed repair (HDR) based gene editing.

In another aspect, the present disclosure provides an isolated host cell comprising any of the polynucleotides disclosed herein or any of the recombinant vectors disclosed herein.

In another aspect, the present disclosure provides an isolated host cell comprising a chimeric antigen receptor (CAR) encoded by any of the polynucleotide disclosed herein.

In some embodiments, the host cell may be an immune cell.

In some embodiments of any of the isolated host cells disclosed herein, the host cell may be a T cell, a natural killer (NK) cell, a mesenchymal stem cell (MSC), or a macrophage.

In some embodiments, the host cell may be a T cell.

In some embodiments, the host cell may be a CD8+ T cell, a CD4+ T cell, a cytotoxic T cell, an αβ T cell receptor (TCR) T cell, an invariant natural killer T (iNKT) cell, a γδ T cell, a memory T cell, a memory stem T cell (TSCM), a naïve T cell, an effector T cell, a T-helper cell, or a regulatory T cell (Treg).

In some embodiments, the host cell may be a natural killer (NK) cell.

In some embodiments, the NK cell may be derived from peripheral, cord blood, IPSCs, and/or a cell line (e.g., NK-92 cells).

In some embodiments, the immune cell may be derived from an induced pluripotent stem (IPS) cell.

In some embodiments, any of the isolated host cells of the present disclosure may further genetically modified to enhance its function by expressing one or more additional genes (e.g., transcription factors (e.g., c-Jun) or cytokines (e.g., IL-15); or deleting one or more inhibitory genes (e.g., REGNASE-1, CISH, DNMT3A) with gene editing technologies (e.g., CRISPR-Cas9, base editors, or transcription activator-like effector nucleases (TALENs)).

In some embodiments, the host cell has been activated and/or expanded ex vivo.

In some embodiments, the host cell may be an allogeneic cell.

In some embodiments, the host cell may be an autologous cell.

In some embodiments, the host cell may be isolated from a subject having a cancer, wherein one or more cells of the cancer express GRP78 and/or B7H3.

In some embodiments, the cancer may be a hematologic malignancy.

In some embodiments, the hematologic malignancy is acute myeloid leukemia (AML), acute lymphoblastic leukemia (ALL), B-cell acute lymphoblastic leukemia (B-ALL), T cell acute lymphoblastic leukemia (T-ALL), a blastic plasmacytoid dendritic neoplasm (BPCDN), a hairy cell leukemia, or lymphoma.

In some embodiments, the host cell may be derived from a blood, marrow, tissue, or a tumor sample.

In another aspect, the present disclosure provides a pharmaceutical composition comprising any of the host cell disclosed herein and a pharmaceutically acceptable carrier and/or excipient.

In another aspect, the present disclosure provides a method of generating any of the isolated host cells disclosed herein, said method comprising genetically modifying the host cell with any of the polynucleotides disclosed herein or any of the recombinant vectors disclosed herein.

In some embodiments, the genetic modifying step may be conducted via viral gene delivery.

In some embodiments, the genetic modifying step may be conducted via non-viral gene delivery.

In some embodiments, the genetic modification may be conducted ex vivo.

In some embodiments of any of the methods disclosed herein, the method further may comprise activation and/or expansion of the host cell ex vivo before, after and/or during said genetic modification.

In another aspect, the present disclosure provides a method for killing a cancer cell expressing GRP78 and/or B7H3, said method comprising contacting said cell with any of the host cells disclosed herein or the pharmaceutical composition disclosed herein.

In another aspect, the present disclosure provides a method for treating a cancer in a subject in need thereof, wherein one or more cells of the tumor express GRP78 and/or B7H3, said method comprising administering to the subject a therapeutically effective amount of any of the host cells disclosed herein or the pharmaceutical composition disclosed herein.

In some embodiments, the cancer may be a hematologic malignancy.

In some embodiments, the hematologic malignancy may be acute myeloid leukemia (AML), acute lymphoblastic leukemia (ALL), B-cell acute lymphoblastic leukemia (B-ALL), T cell acute lymphoblastic leukemia (T-ALL), a blastic plasmacytoid dendritic neoplasm (BPCDN), a hairy cell leukemia, or lymphoma.

In some embodiments of any of the methods disclosed, the method may comprise:
a) isolating T cells, NK cells, mesenchymal stem cells or macrophages from the subject;
b) genetically modifying said T cells, NK cells, mesenchymal stem cells, or macrophages ex vivo with any of the polynucleotides disclosed herein or any of the vectors disclosed herein;
c) optionally, expanding and/or activating said T cells, NK cells, mesenchymal stem cells, or macrophages before, after or during step (b); and
d) introducing the genetically modified T cells, NK cells, mesenchymal stem cells, or macrophages into the subject.

In some embodiments of any of the methods disclosed herein, the subject may be human.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows a schematic of mono- and bispecific 78.123-CAR constructs. FIG. 1B is a graph depicting linker length and rigidity.

FIGS. 2A-2B illustrate transduction efficiency by CD19 detection and recombinant CD123 protein binding. Transduced T cells analyzed by flow cytometry for transduction efficiency by CD19 (FIG. 2A; GRP78 95.5±1.8%, β2M 92.3±1.4%, (G4S)3 96.7±1.9%, GPcPcPc 91.0±3.6%, mtIgG4 89.3±2.9%, N=4) and recombinant CD123 protein (FIG. 2B; CD123 74.48±4.5%, β2M 37.8±36%, (G4S)3 87.5±7.8%, GPcPcPc 83.6±9.2%, mtIgG4 41.0±32%).

FIGS. 3A-3D shows bispecific 78.123 CAR viability, expansion, and immunophenotype. Cell viability measured via trypan blue exclusion (FIG. 3A; day 2: p<0.001 NT vs GRP78, p<0.05 GRP78 vs mtIgG4; day 4 data ns, day 7 p<0.05 GRP78 vs (G4S)3, CD123 vs (G4S)3, CD123 vs GPcPcPc, β2M vs GPcPcPc; day 10:p<0.05 GRP78 vs (G4S)3, GRP78 vs GPcPcPc, GRP78 vs HER2, CD123 vs (G4S)3; two-way ANOVA mixed modeling). T cell expansion measured up until 10 days post transduction (FIG. 3B; data ns days 0-7; day 10 p<0.05 GRP78 vs HER2, CD123 vs HER2, β2M vs HER2, (G4S)3 vs mtIgG4, GPcPcPc vs HER2; two-way ANOVA mixed modeling). (Immunophenotype of CAR T cells on day 6-8 post transduction (FIGS. 3C-3D; CD4 vs CD8 data ns between all groups, EM: CCR7−, CD45RO+; CM: CCR7+, CD45RAO+; Naïve-like: CCR7+ CD45RO−; EMRA: CCR7−, CD45RO−, CD4 EM p<0.05 NT vs HER2, NT vs (G4S)3, (G4S)3 vs mtIgG4; CD8 EM p<0.01 NT vs (G4S)3, HER2 vs mtIgG4, p<0.05 HER2 vs (G4S)3).

FIGS. 4A-4I show (FIG. 4A) Western blot analysis of transduced 78.123 CAR T cells and probed for CD3z (left) and GAPDH (right) N=3, representative blots. Representative flow cytometry analysis of (FIG. 4B) GRP78-biotin-strepdavidin shown with (FIG. 4C) MFI and (FIG. 4D) CD123 with (FIG. 4E) MFI on target cell lines. Non transduced (NT), control CAR, single antigen CAR T cells (left), and bispecific CAR T cells (right) were cocultured with RPMI8402 (FIG. 4F) KG1a (FIG. 4G), MOLM13 (FIG. 4H), or recombinant protein (FIG. 4I; 1 μg/mL) at a 2:1 E:T ratio. Supernatants were harvested after 24 hours and analyzed for IL-2 by ELISA assay (N=4, One-Way ANOVA, ** p<0.01, *** p<0.001 ****p<0.0001).

FIGS. 5A-5H show IFNγ production by 78.123 CAR T cells and cytotoxicity assays of 78.123 CAR T cells. Non transduced (NT), control CAR, single antigen CAR T cells and bispecific 78.132 CAR T cells were cocultured with RPMI8402 (FIG. 5A) KG1a (FIG. 5B), MOLM13 (FIG. 5C), or recombinant protein (FIG. 5D, 1 μg/mL) at a 2:1 E:T ratio. Supernatants were harvested after 24 hours and analyzed for IFN-γ by ELISA assay (N=4, One-Way ANOVA, ** p<0.01, * ** p<0.001 ****p<0.0001). Flow cytometry-based cytotoxicity assay of CAR T cells single antigen (GRP78, CD123), bispecific (β2M, (G4S)3, GPcPcPc, mtIgG4), or control effector T cells (NT, HER2-CAR) against target cells of different antigen densities at 5 different E:T ratios (FIGS. 5E-5H) (RPMI8402 p<0.05 1:1 HER2 vs CD123, HER2 vs B2M, HER2 vs mIgG4, p<0.01 B2M vs G4S3, G4S2 vs mIgG4; 1:2 p<0.05 CD123 vs B2M, 1:4 p<0.05 HER2 vs CD123, HER2 vs B2M, B2M vs mIgG4, p<0.01 CD123 vs G4S3; MOLM13 and KG1a at 1:1 HER2 vs CARs p<0.01 all conditions, KG1a KO, HER2 vs GRP78, G4S3, B2M, mIgG4 p<0.01, data ns HER2 vs CD123 and HER2 vs GPcPcPc, Two-Way ANOVA Tukey's multiple comparisons).

FIGS. 6A-6C show serial stimulation assay with control and CAR T cells and CCRF target cells. Schematic of serial stimulation assay (FIG. 6A). Effector T cells and (FIG. 6B) KG1a or (FIG. 6C) KG1a KO cells were cocultured at a 1:1 E:T ratio. Fresh target cells were added every 72-hours if a luciferase-based cytotoxicity assay demonstrated greater than 50% killing (N=3). Double-gradient heat map (black 100% target cell lysis; white 0% target cell lysis).

FIGS. 7A-7C show evaluation of the anti-AML activity of mono- and bispecific CAR T cells in vivo using MOLM13 xenograft model. NSG mice were injected IV via tail vein with 5×10³ MOLM13.GFP.ffluc cells. On day 7, mice received a single 3×10⁶ dose of T cells. Mice were monitored via IVIS imaging and tracked for bioluminescence (FIG. 7A; Total flux photons/second) and survival of mice injected with MOLM13.GFP.ffluc cells (FIG. 7B; p<0.0001, Log-rank Mantel-Cox, CD123 vs G4S3 p<0.001).

FIGS. 8A-8D show evaluation of the anti-AML activity of mono- and bispecific CAR T cells in vivo using KG1a xenograft model (FIGS. 8A-8B) and a KG1a KO xenograft model (FIGS. 8C-8D)NSG mice were injected IV via tail vein with 1×10⁶ KG1a.GFP.ffluc cells. On day 7, mice received a single 3×10⁶ dose of T cells Mice were monitored via IVIS imaging and tracked for bioluminescence (FIG. 8A; Total flux photons/second) and survival (FIG. 8B; p<0.01, Log-rank Mantel-Cox). NSG mice were injected IV via tail vein with 1×10⁶ KG1aKO.GFP.ffluc cells. On day 7, mice received a single 3×10⁶ dose of T cells. Mice were monitored via IVIS imaging and tracked for bioluminescence (FIG. 8C; Total flux photons/second) and survival (FIG. 8D; p<0.01, Log-rank Mantel-Cox).

FIGS. 9A-9D show a serial stimulation assay with control and CAR T cells and CCRF target cells. Effector T cells and (FIG. 9A) RPMI8402, (FIG. 9B) MOLM13, (FIG. 9C) KG1a or (FIG. 9D) KG1a KO cells were cocultured at a 1:1 E:T ratio. Fresh target cells were added every 72-hours if cytotoxicity assay demonstrated greater than 50% killing. Number of stimulations were represented for each donor (N=4, One-Way ANOVA, ** p<0.01, *** p<0.001 ****p<0.0001).

FIGS. 10A-10D show tumor burden and T cell infiltrates from HER2, GRP78, CD123, or G4S3 mice. NSG mice were injected IV via tail vein with 5×10³ MOLM13.GFP.ffluc cells. On day 7, mice received a single 3×10⁶ dose of T cells. Bone Marrow and spleen were harvested from mice at endpoint for flow cytometry analysis. Flow cytometry analysis of CD3+ T cells (FIGS. 10A-10B) and tumor cells (FIGS. 10C-10D, CD45+ CD3−) in the (FIGS. 10A & 10C) bone marrow and spleen (FIGS. 10B & 10D, N=3-6, One-Way ANOVA, *p<0.05, ** p<0.01).

FIGS. 11A-11H show differences in checkpoint markers between bispecific CAR T cells and the CD123 monospecific CAR T cells. Flow cytometry analysis of HER2, GRP78, CD123, G4S3, and B2M T cells for expression of LAG3, PD1, and TIM3 on (FIGS. 11A-11C) CD4+ or (FIGS. 11D-11F) CD8+ effector T cells. Co-expression of LAG3, PD1, and TIM3 on (FIG. 11G) CD4+ or (FIG. 11H) CD8+ effector T cells on day 7 (CAR T cells are, left to right, NT, HER2, GRP78, CD123, (G4S)3, and B2M. One-Way ANOVA (A-F) or 2-Way ANOVA (FIGS. 11G-11H) * p<0.05, **p<0.01, *** p<0.001 ****p<0.0001.

FIGS. 12A-12H show the construction and testing of 78.B7H3-CAR T cells. (FIG. 12A) Schematic of mono and bispecific 78.B7H3 CAR constructs. Transduced T cells analyzed by flow cytometry for transduction efficiency by (Fab′)₂ (FIG. 12B, NT 2.54±2.7%, HER2 41.8±6.6%, GRP78 9.41±6.5%, B7H3 54.6±12.1%, CD28 86.7±1.9%, CD8 4.4±4.1%, One-Way ANOVA, ** p<0.01, *** p<0.001 ****p<0.0001 N=3) and CD19 (FIG. 12C, NT 1.84±1.0%, HER2 1.86±1.2%, GRP78 94.3±2.0%, B7H3 2.75±2.4%, CD28 92.6±1.1%, CD8 1.93±1.8%, One-Way ANOVA, ** p<0.01, *** p<0.001 ****p<0.0001, N=3). (FIG. 12D) Cell viability measured via trypan blue exclusion (day 7 p<0.05 NT vs GRP78 NT vs CD28, HER2 vs GRP78, HER2 vs CD28, p<0.01 GRP78 vs CD8 and day 10 P<0.05 NT vs GRP78, NT vs CD8, HER2 vs GRP78, HER2 vs CD28, GRP78 vs CD28, GRP78 vs CD8; two-way ANOVA mixed modeling). (FIG. 12E) T cell expansion measured up until 10 days post transduction (day 7 p<0.05 NT vs GRP78, HER2 vs GRP78, GRP78 vs B7H3, GRP78 vs CD28, B7H3 vs CD8, p<0.01 NT vs CD8, p<0.001 HER2 vs CD8, day 10 p<0.05 NT vs GRP78, NT vs CD8, HER2 vs GRP78, GRP78 vs CD28, B7H3 vs CD8, CD28 vs CD8, p<0.01 GRP78 vs B7H3; two-way ANOVA mixed modeling). (FIG. 12F) Immunophenotype of CAR T cells on day 6-8 post transduction (CD4: p<0.05 NT vs B7H3, B7H3 vs CD8, CD8: p<0.05 HER2 vs CD28 p<0.01 NT vs B7H3, GRP78 vs CD28, B7H3 vs CD8, p<0.001 NT vs CD28, CD28 vs CD8; EM: CCR7−, CD45RO+; CM: CCR7+, CD45RAO+; Naïve-like: CCR7+ CD45RO−; EMRA: CCR7−, CD45RO−, CD4 EM p<0.05 GRP78 vs B7H3, p<0.01 B7H3 vs CD28, CD4 Naïve-like p<0.05 B7H3 vs CD8; CD8 CM P<0.05 GRP78 vs CD8; 2-Way ANOVA mixed modeling). Cytotoxicity assay of CAR T cells single antigen against target cells THP-1 (FIG. 12G, GRP78+ B7H3+) and KG1a (FIG. 12H, GRP78± B7H3−) at 7 different E:T ratios (THP-1 1:1 p<0.05 HER2 vs GRP78, HER2 vs B7H3, HER2 vs CD28; KG1a 1:1 p<0.01 HER2 vs GRP78, HER2 v CD28; 2-Way ANOVA Tukey's multiple comparisons).

FIGS. 13A-13F show that 78.B7H3 CD28 bispecific CAR T cells elicit antigen specific cytotoxicity. (FIG. 13A) Transduced T cells analyzed by flow cytometry for transduction efficiency by G4S linker NT 0.25±0.2%, HER2 27.1±5.6%, GRP78 1.07±0.3%, CD123 64.4±15.8%, B7H3 86.2±7.9%, G4S3 79.9±12.6%, B2M 67.9±14.9%, GPcPcPc 69.0±12.9%, mtIgG4 76.9±12.8%, CD28 88.6±7.6%, CD8 43.4±11.0%, N=3. (FIG. 13B) Western blot analysis of T cells transduced CAR T cells and probed for CD3z (left) and GAPDH (right) N=3, representative blots shown. (FIG. 13C) Representative flow cytometry analysis of B7H3 (left) with MFI (right) on target cell lines. Non transduced (NT), control CAR, single antigen CAR T cells and bispecific CAR T cells were cocultured with KG1a (FIG. 13D), THP-1 (FIG. 13E), or recombinant B7H3 protein (FIG. 13F, 0.5 μg/mL) at a 2:1 E:T ratio. Supernatants were harvested after 24 hours and analyzed for IFN-g (left) and IL-2 (right) by ELISA assay (N=4, One-Way ANOVA, ** p<0.01, *** p<0.001 ****p<0.0001). One-Way ANOVA * p<0.05, **p<0.01, *** p<0.001 ****p<0.0001.

FIGS. 14A-14B show cell surface antigen expression on de novo, relapsed and primary AML. Antigen expression as determined by flow cytometry on (FIG. 14A) Primary samples new diagnosis (de novo N=1), relapsed (rAML; N=6) or therapy related (tAML; N=4) (Average expression of CD123 65.1±33.6% (left-hand columns), GRP78 46.9±20.7% (middle columns), and B7H3 69.9±18.9% (right-hand columns)) single and dual expression of each individual PDX lines antigen status and the comparison of (FIG. 14B) THP-1 (left-hand columns) vs PDX (right-hand columns) samples.

### DETAILED DESCRIPTION

The present invention provides, among other things, bispecific CARs targeting cell surface GRP78 and CD123 (also referred to as “78.123-CAR” herein), as well as bispecific CARs targeting cell surface GRP78 and B7H3 (also referred to as “78.B7H3-CAR” herein).

CD123 and GRP78 are two antigens widely present on the cell surface of AML. CD123 is highly expressed on myeloid leukemia blasts and leukemia stem cells and cell surface GRP78 expression is also limited to AML blasts. B7H3 is expressed on several malignancies, including solid tumors and leukemic blasts but not on normal hematopoietic stem cells (HSCs). As detailed in the Examples section below, an exemplary panel of CAR T cells were designed with a bispecific antigen binding domain combining the GRP78 peptide with the CD123 single chain variable fragment (scFv) for CAR T cell design (78.123-CAR). A second exemplary panel of CAR T cells were designed with a bispecific antigen binding domain combining the GRP78 peptide with the B7H3 single chain variable fragment (scFv) for CAR T cell design (78.B7H3-CAR).

The present invention discloses, in various aspects, ideal configurations for optimal bispecific 78.123-CAR designs and 78.B7H3-CAR designs that have enhanced antitumor activity and persistence.

The generated CAR can be expressed in various immune cells (for example but not limited to αβ T cells, γδ T cells, iNKT cells, NK cells, or macrophages). Further, the linkers between peptide and scFv for functional antigen binding domains can be broadly applied to other bispecific CAR T cell design. The data provided herein support that 78.123-CAR modified cells and 78.B7H3-CAR modified cells are effective in inhibiting growth of or killing a broad range of malignancies including as a non-limiting example hematological malignancies (e.g., AML).

### Definitions

The term “chimeric antigen receptor” or “CAR” as used herein is defined as a cell-surface receptor comprising an extracellular antigen-binding domain, a transmembrane domain and a cytoplasmic domain, comprising a lymphocyte activation domain and optionally at least one co-stimulatory signaling domain, all in a combination that is not naturally found together on a single protein. This particularly includes receptors wherein the extracellular domain and the cytoplasmic domain are not naturally found together on a single receptor protein. The chimeric antigen receptors of the present invention may be used with lymphocyte such as T cells and natural killer (NK) cells.

The terms “T cell” and “T lymphocyte” are interchangeable and used synonymously herein. As used herein, T cell includes thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+ CD8+ T cell, or any other subset of T cells. Other illustrative populations of T cells suitable for use in particular embodiments include naive T cells and memory T cells. Also included are “NKT cells”, which refer to a specialized population of T cells that express a semi-invariant αβ T cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T cells (yS T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation and were found to be an important source of IL-17 and to induce robust CD8+ cytotoxic T cell response. Also included are “regulatory T cells” or “Tregs” refers to T cells that suppress an abnormal or excessive immune response and play a role in immune tolerance. Tregs cells are typically transcription factor Foxp3-positive CD4+ T cells and can also include transcription factor Foxp3-negative regulatory T cells that are IL-10-producing CD4+ T cells.

The terms “natural killer cell” and “NK cell” are used interchangeably and used synonymously herein. As used herein, NK cell refers to a differentiated lymphocyte with a CD 16+ CD56+ and/or CD57+ TCR− phenotype. NKs are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

As used herein, the term “antigen” refers to any agent (e.g., protein, peptide, polysaccharide, glycoprotein, glycolipid, nucleic acid, portions thereof, or combinations thereof) molecule capable of being bound by a T cell receptor. An antigen is also able to provoke an immune response. An example of an immune response may involve, without limitation, antibody production, or the activation of specific immunologically competent cells, or both. A skilled artisan will understand that an antigen need not be encoded by a “gene” at all. It is readily apparent that an antigen can be generated synthesized or can be derived from a biological sample or might be a macromolecule besides a polypeptide. Such a biological sample can include, but is not limited to a tissue sample, a tumor sample, a cell or a fluid with other biological components, organisms, subunits of proteins/antigens, killed or inactivated whole cells or lysates.

The term “antigen-binding domain” or “antigen-binding moiety” refers to a target-specific binding element that may be any ligand that binds to the antigen of interest or a polypeptide or fragment thereof, wherein the ligand is either naturally derived or synthetic. Examples of antigen-binding domains include, but are not limited to, antibodies; polypeptides derived from antibodies, such as, for example, single chain variable fragments (scFv), Fab, Fab′, F(ab′)2, and Fv fragments; polypeptides derived from T cell receptors, such as, for example, TCR variable domains; secreted factors (e.g., cytokines, growth factors) that can be artificially fused to signaling domains (e.g., “zytokines”); and any ligand or receptor fragment (e.g., CD27, NKG2D) that binds to the antigen of interest. Combinatorial libraries could also be used to identify peptides binding with high affinity to the therapeutic target. In some embodiments, the CARs described herein comprises one or more peptides as an antigen-binding domain.

Terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen-specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Antibodies useful as a TCR-binding molecule include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgM1, IgM2, IgA1 and IgA2) or subclass.

The term “host cell” means any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5α, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, CHO, Saos, and PC12. In certain embodiments, the host cell is autologous. In certain embodiments, the host cell is allogenic.

Host cells of the present disclosure include immune cells (e.g., T cells and natural killer cells) that contain the DNA or RNA sequences encoding the CAR and express the CAR on the cell surface. Host cells may be used for enhancing immune cell activity (e.g., effector function), treatment of tumors, and treatment of autoimmune disease.

The terms “activation” or “stimulation” means to induce a change in their biologic state by which the cells (e.g., T cells and NK cells) express activation markers, produce cytokines, proliferate and/or become cytotoxic to target cells. All these changes can be produced by primary stimulatory signals. Co-stimulatory signals can amplify the magnitude of the primary signals and suppress cell death following initial stimulation resulting in a more durable activation state and thus a higher cytotoxic capacity. A “co-stimulatory signal” refers to a signal, which in combination with a primary signal, such as TCR/CD3 ligation, leads to T cell and/or NK cell proliferation and/or upregulation or downregulation of key molecules.

The terms “express” and “expression” mean allowing or causing the information in a gene or DNA sequence to become produced, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular, or transmembrane.

The term “tumor” refers to a benign or malignant abnormal growth of tissue. The term “tumor” includes cancer.

The term “effector function” refers to a specialized function of a cell. Effector function of a T cell, for example, may be cytolytic activity or helper activity including the secretion of cytokines.

As used herein, the term “safety switch” refers to any mechanism that is capable of removing or inhibiting the effect of CAR from a system (e.g., a culture or a subject).

The term “site-specific nuclease” as used herein refers to a nuclease capable of specifically recognizing and cleaving a nucleic acid (DNA or RNA) sequence.

The terms “genetically modified” or “genetically engineered” refers to the addition of extra genetic material in the form of DNA or RNA into a cell.

The term “tumor killing activity” as used herein refers to the ability of an immune cell to inhibit tumor growth and/or to kill the tumor cells (e.g., cancer cells).

The terms “expand” or “expansion” when used in relation to an immune cell refer to the ability of the immune cell to undergo cellular proliferation (i.e., to increase the number of cells). The terms used herein encompass both in vivo and in vitro immune cell expansion.

The terms “persist” or “persistence” when used in relation to an immune cell refer to the ability of the immune cell (and/or its progenies) to be maintained in a recipient (e.g., a subject) for a period of time. The terms used herein encompass both in vivo and in vitro immune cell persistence.

As used herein, the term “variant”, “derivative” or “derived from” in the context of proteins or polypeptides (e.g., CARs or domains thereof) refer to: (a) a polypeptide that has at least 40%, 45%, 50%, 55%, 60%, 65%, 70%, 75%, 80%, 85%, 90%, 95%, 98% or 99% sequence identity to the polypeptide it is a variant or derivative of, (b) a polypeptide encoded by a nucleotide sequence that has at least 40%, 45%, 50%, 55%, 60%, 65%, 70%, 75%, 80%, 85%, 90%, 95%, 98% or 99% sequence identity to a nucleotide sequence encoding the polypeptide it is a variant or derivative of; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to the polypeptide it is a variant or derivative of, (d) a polypeptide encoded by nucleic acids can hybridize under high, moderate or typical stringency hybridization conditions to nucleic acids encoding the polypeptide it is a variant or derivative of, (e) a polypeptide encoded by a nucleotide sequence that can hybridize under high, moderate or typical stringency hybridization conditions to a nucleotide sequence encoding a fragment of the polypeptide, it is a variant or derivative of, of at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, or at least 150 contiguous amino acids; or (f) a fragment of the polypeptide it is a variant or derivative of. The terms also encompass a fusion protein or polypeptide comprising the polypeptide it is a variant or derivative of. For example, a variant of a GRP78-binding peptide disclosed herein may include a fusion protein or polypeptide that comprises the GRP78-binding peptide and one or more additional polypeptides.

Percent sequence identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wisconsin). Information regarding hybridization conditions (e.g., high, moderate, and typical stringency conditions) have been described, see, e.g., U.S. Patent Application Publication No. US 2005/0048549 (e.g., paragraphs 72-73).

The variant or derivative of a protein or polypeptide may be a functional variant or derivative of the referenced protein or polypeptide. The term “functional variant” or “functional derivative” as used herein refers to a polypeptide or protein, or a polynucleotide encoding the polypeptide or protein, that retains at least one function of the referenced polypeptide or protein. The functional variant or derivative of a polypeptide or protein may retain one, two, three, four, five, or more functions of the referenced protein or polypeptide. For example, a functional variant or derivative of a GRP78-binding peptide may retain its ability to bind to GRP78.

The terms “vector”, “cloning vector,” “recombinant vector,” and “expression vector” mean the vehicle by which a DNA or RNA sequence (e.g., a foreign gene) can be introduced into a host cell, so as to genetically modify the host and promote expression (e.g., transcription and translation) of the introduced sequence. Vectors include plasmids, synthesized RNA and DNA molecules, phages, viruses, etc. In certain embodiments, the vector is a viral vector such as, but not limited to, viral vector is an adenoviral, adeno-associated, alphaviral, herpes, lentiviral, retroviral, or vaccinia vector.

As used herein, the term “operably linked,” or “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The terms “enhance” or “promote,” or “increase,” or “expand,” or “improve” refer generally to the ability of a composition contemplated herein to produce, elicit, or cause a greater physiological response (i.e., downstream effects) compared to the response caused by either vehicle or a control molecule/composition. A measurable physiological response may include an increase in immune cell expansion, activation, effector function, persistence, and/or an increase in tumor cell death killing ability, among others apparent from the understanding in the art and the description herein. In certain embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

The terms “decrease” or “lower,” or “lessen,” or “reduce,” or “abate” refer generally to the ability of composition contemplated herein to produce, elicit, or cause a lesser physiological response (i.e., downstream effects) compared to the response caused by either vehicle or a control molecule/composition. In certain embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The term “pharmaceutical composition,” as used herein, represents a composition comprising polynucleotides, vectors, peptides, compositions, or host cells described herein formulated for administration to a subject for treatment, abatement, or prevention of a disease.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “protein” is used herein encompasses all kinds of naturally occurring and synthetic proteins, including protein fragments of all lengths, fusion proteins, and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid, the term may also refer to the nucleic acid sequence including the portion coding for any amino acids added as an artifact of cloning, including any amino acids coded for by linkers.

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The term “carrier” refers to a diluent, adjuvant, excipient, or vehicle with which the compound is administered. Such pharmaceutical carriers can be sterile liquids, such as water and oils, including those of petroleum, animal, vegetable, or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art. If aspects of the disclosure are described as “comprising” a feature, or versions thereof (e.g., comprise), embodiments also are contemplated “consisting of” or “consisting essentially of” the feature.

The practice of the present disclosure employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437.

The technology illustratively described herein suitably may be practiced in the absence of any element(s) not specifically disclosed herein.

The terms and expressions which have been employed are used as terms of description and not of limitation, and use of such terms and expressions do not exclude any equivalents of the features shown and described or portions thereof, and various modifications are possible within the scope of the technology claimed.

### Chimeric Antigen Receptors

The present disclosure generally provides, among other things, chimeric antigen receptors (CARs), particularly bispecific CARs that specifically bind GRP78 and CD123 or to GRP78 and B7H3. Also provided are polypeptides of the CARs and other related molecules, polynucleotides, vectors, and cell compositions comprising the same. Pharmaceutical compositions comprising the polypeptides, polynucleotides, vectors, or cells of the present disclosure, and their uses in treating a cancer in a subject are also provided.

CARs are primarily comprised of 1) an extracellular domain comprising one or more antigen-binding moieties such as, but not limited to, an antigen binding peptide and/or a single-chain variable fragment (scFv), for example, an scFv derived from an antigen-specific monoclonal antibody, and 2) a signaling domain, such as the ζ-chain from the T cell receptor CD3. These two regions are fused together via a transmembrane domain. Without wishing to be bound by theory when two or more antigen binding moieties are present, one or more linkers may join the two antigen recognition moieties. Non-limiting examples of linkers include (G4S)3, mtIgG4, β2-microglobulin (β2M, or B2M), and GPcPcPc. A hinge domain is usually required to provide more flexibility and accessibility between the antigen-binding moiety and the transmembrane domain. Upon transduction, the lymphocyte expresses the CAR on its surface, and upon contact and ligation with the target antigen, it signals through the signaling domain (e.g., CD3ζ chain) inducing cytotoxicity and cellular activation.

CAR constructs with only the extracellular domain together with the signaling domain are termed first generation CARs. Second generation CARs usually comprise co-stimulatory polypeptides to boost the CAR-induced immune response. The most commonly used co-stimulating molecules include CD28 and 4-1BB, which may promote both T cell proliferation and cell survival. Third generation CARs typically include three signaling domains (e.g., CD3ζ, CD28, and 4-1BB), which may further improve lymphocyte cell survival and efficacy.

In some embodiments, the CAR is a first generation CAR. In certain embodiments, the CAR is a second generation CAR. In various embodiments, the CAR is a third generation CAR.

### Extracellular Domain

### Antigen-Binding Moiety

In certain aspects, CARs of the present disclosure comprise an extracellular antigen-binding domain, wherein the extracellular domain comprises one or more antigen-binding moieties.

The choice of antigen-binding moiety depends upon the type and number of antigens that define the surface of a target cell. For example, the antigen-binding moiety may be chosen to recognize an antigen that acts as a cell surface marker on target cells associated with a particular disease state. In certain embodiments, the CARs of the present disclosure can be genetically modified to target a tumor antigen of interest by way of engineering a desired antigen-binding moiety that specifically binds to an antigen (e.g., on a cancer cell). Non-limiting examples of cell surface markers that may act as targets for the antigen-binding moiety in the CAR of the invention include those associated with cancer cells.

In some embodiments, the extracellular domain of the present disclosure comprises one or more antigen-binding moieties specific for GRP78. GRP78 is also known as HSP70, binding immunoglobulin protein (BiP), heat shock 78 kDa protein 5 (HSPA5), or Byun1.

In some embodiments, the antigen-binding moiety recognizes CD123. CD123, also known as interleukin-3 receptor, is a molecule found on cells which helps transmit the signal of interleukin-3, a soluble cytokine important in the immune system. CD123 is expressed across acute myeloid leukemia (AML) subtypes, including leukemic stem cells. CD123 is also expressed at low levels in normal hematopoietic progenitor cells.

In some embodiments, the antigen-binding moiety recognizes B7H3. B7H3, also referred to as CD276, is a coreceptor belonging to the B7 family of immune checkpoint molecules and is an attractive target for immunotherapy as it is expressed on several malignancies, including solid tumors and leukemic blasts but not on normal hematopoietic stem cells (HSCs).

In some embodiments, the antigen-binding moiety comprises one or more antigen-binding polypeptides or functional variants thereof that binds to one or more antigen. In some embodiments, the antigen-binding polypeptide(s) is an antibody or an antibody fragment that binds to an antigen. Antigen-binding moieties may comprise antibodies and/or antibody fragments such as monoclonal antibodies, multispecific antibodies, chimeric antibodies, single-chain Fvs (scFv), single chain antibodies, Fab fragments, F(ab′) fragments, disulfide-linked Fvs (sdFv), intrabodies, minibodies, single domain antibody variable domains, nanobodies (VHHs), diabodies and anti-idiotypic (anti-Id) antibodies (including, e.g., anti-Id antibodies to antigen specific TCR), and epitope-binding fragments of any of the above. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

In certain embodiments, the antigen-binding moiety comprises a polypeptide or functional variant thereof that binds to GRP78. In certain embodiments, the antigen-binding moiety is a single chain variable fragment (scFv) that binds to GRP78. In some embodiments, the antigen-binding moiety is an anti-GRP78 single chain variable fragment (scFv). In certain embodiments, the GRP78-binding moiety is a GRP78-binding peptide.

In various embodiments, the target-binding domain comprises more than one GRP78-binding moiety. In certain embodiments, the target-binding domain comprises 1, 2, 3, 4, 5, 6, 7, 8, 9, or 10 GRP78-binding moieties. In various embodiments, the target-binding domain comprises one GRP78-binding moiety. In certain embodiment, the target-binding domain comprises two GRP78-binding moieties. In some embodiments, the target-binding domain comprises three GRP78-binding moieties.

In certain embodiments, when more than one GRP78-binding moiety is used in the CAR, each GRP78-binding moiety is operably linked via a linker sequence. In various embodiments, the linker sequence is 1, 2, 3, 4, 5, 6, 7, 8, 9, 10, 11, 12, 13, 14, 15, 16, 17, 18, 19, 20, or more than 20 amino acids in length. In various embodiments, GRP78-binding moieties may be separated by linkers of non-equal lengths such that the length of each linker is independently selected. In various embodiments, the linkers are of equal length. In some embodiments, the linkers each comprise independently selected sequences. In various embodiments, the linkers all comprise the same amino acid sequence. It is contemplated that any amino acid linkers can be used to link the GRP78-binding moieties. The GRP78-binding moieties are attached via linker sequences in a manner that does not interfere with each GRP78-binding moiety binding to GRP78. For example, each GRP78-binding moiety may be operably linked via a linker sequence of GGGGS (SEQ ID NO: 20).

In certain embodiments, the antigen-binding moiety comprises a polypeptide or functional variant thereof that binds to CD123. In certain embodiments, the antigen-binding moiety is a single chain variable fragment (scFv) that binds to a CD123. In some embodiments, the anti-CD123 scFv is derived from an mAb specific for CD123, or a functional variant thereof. For example, in some embodiments, the CD123-binding moiety is an anti-CD123 single chain variable fragment (scFv). In some embodiments, the anti-CD123 scFv is derived from antibody 26292 (scFV (292)). In some embodiments, the anti-CD123 scFv is derived from antibody 26716 (scFV (716)). The antibody 26292 and antibody 26716 are anti-IL3Ra antibodies described in U.S. Pat. No. 8,163,279, which is herein incorporated by reference in its entirety for all purposes.

In certain embodiments, the CD123-binding moiety comprises three heavy chain complementarity determining regions (CDRs) (HCDR1, HCDR2, and HCDR3) contained within a heavy chain variable region of the anti-CD123 scFv; and/or three light chain CDRs (LCDR1, LCDR2, and LCDR3) contained within a light chain variable region of the anti-CD123 scFv.

In certain embodiments, the antigen-binding moiety comprises a polypeptide or functional variant thereof that binds to B7H3. In certain embodiments, the antigen-binding moiety is a single chain variable fragment (scFv) that binds to B7H3. In some embodiments, the anti-B7H3 scFv is derived from an mAb specific for B7H3, or a functional variant thereof. For example, in some embodiments, the B7H3-binding moiety is an anti-B7H3 single chain variable fragment (scFv). In some embodiments, the anti-B7H3 scFv is derived from humanized B7H3-specific monoclonal antibody (mAb) MGA271. The antibody MGA271 is an anti-B7H3 mAb described in Loo et al., Development of an Fc-enhanced anti-B7-H3 monoclonal antibody with potent antitumor activity, Clin Cancer Res. 2012 Jul. 15; 18(14):3834-45, which is herein incorporated by reference in its entirety for all purposes.

In certain embodiments, the B7H3-binding moiety comprises three heavy chain complementarity determining regions (CDRs) (HCDR1, HCDR2, and HCDR3) contained within a heavy chain variable region of the anti-B7H3 scFv; and/or three light chain CDRs (LCDR1, LCDR2, and LCDR3) contained within a light chain variable region of the anti-B7H3 scFv.

In certain embodiments, the extracellular domain comprises a first antigen-binding moiety and a second antigen-binding moiety. In some embodiments, the first and/or second antigen-binding moiety may comprise a polypeptide or functional variant thereof that binds to GRP78. In some embodiments, the first and/or second antigen-binding moiety may comprise a polypeptide or functional variant thereof that binds to CD123. In some embodiments, the first and/or second antigen-binding moiety may comprise a polypeptide or functional variant thereof that binds to B7H3. In certain embodiments, the first and/or second antigen-binding moiety may comprise a single chain variable fragment (scFv) that binds to GRP78. In certain embodiments, the first and/or second antigen-binding moiety may comprise a single chain variable fragment (scFv) that binds to CD123. In certain embodiments, the first and/or second antigen-binding moiety may comprise a single chain variable fragment (scFv) that binds to B7H3. In some embodiments, the first and/or second antigen-binding moiety may comprise an anti-GRP78 single chain variable fragment (scFv). In some embodiments, the first and/or second antigen-binding moiety may comprise an anti-CD123 single chain variable fragment (scFv). In some embodiments, the first and/or second antigen-binding moiety may comprise an anti-B7H3 single chain variable fragment (scFv). In certain embodiments, the first and/or second antigen-binding moiety may comprise a GRP78-binding peptide. In certain embodiments, the first and/or second antigen-binding moiety may comprise a CD123-binding peptide. In certain embodiments, the first and/or second antigen-binding moiety may comprise a B7H3-binding peptide.

In some embodiments, the first antigen-binding moiety may comprise a GRP78-binding peptide and the second antigen-binding moiety may comprise an anti-CD123 single chain variable fragment (scFv). In some embodiments, the first antigen-binding moiety may comprise a GRP78-binding peptide and the second antigen-binding moiety may comprise an anti-B7H3 single chain variable fragment (scFv).

In some embodiments, the first and/or second antigen binding moiety may comprise a GRP78-binding peptide comprising the amino acid sequence set forth in SEQ ID NO: 92, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 92. In certain embodiments, the nucleotide sequence that encodes the GRP78-binding peptide comprises the nucleotide sequence that encodes the amino acid sequence of SEQ ID NO: 92, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 92. In certain embodiments, the nucleotide sequence that encodes the GRP78-binding peptide comprises the nucleotide sequence set forth in SEQ ID NO: 93, or a nucleotide sequence having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 93. In certain embodiments, the GRP78-binding peptide comprises the amino acid sequence set forth in SEQ ID NO: 92. In certain embodiments, the nucleotide sequence that encodes the GRP78-binding peptide comprises the nucleotide sequence set forth in SEQ ID NO: 93.

In some embodiments, the first and/or second antigen binding moiety may comprise an anti-CD123 scFv comprising a heavy chain variable domain (VH). In some embodiments, the VH of the anti-CD123 scFv may comprise the amino acid sequence set forth in SEQ ID NO: 94, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 94. In certain embodiments, the nucleotide sequence that encodes the VH of the anti-CD123 scFv comprises the nucleotide sequence that encodes the amino acid sequence of SEQ ID NO: 94, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 94. In certain embodiments, the nucleotide sequence that encodes the VH of the anti-CD123 scFv comprises the nucleotide sequence set forth in SEQ ID NO: 95, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 95. In certain embodiments, the VH of the anti-CD123 scFv comprises the amino acid sequence set forth in SEQ ID NO: 94. In certain embodiments, the nucleotide sequence that encodes the VH of the anti-CD123 scFv comprises the nucleotide sequence set forth in SEQ ID NO: 95.

In some embodiments, the first and/or second antigen binding moiety may comprise an anti-CD123 scFv comprising a light chain variable domain (VL). In some embodiments, the VL of the anti-CD123 scFv may comprise the amino acid sequence set forth in SEQ ID NO: 96, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 96. In certain embodiments, the nucleotide sequence that encodes the VL of anti-CD123 scFv comprises the nucleotide sequence that encodes the amino acid sequence of SEQ ID NO: 96, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 96. In certain embodiments, the nucleotide sequence that encodes the VL of the anti-CD123 scFv comprises the nucleotide sequence set forth in SEQ ID NO: 97, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 97. In certain embodiments, the VL of the anti-CD123 scFv comprises the amino acid sequence set forth in SEQ ID NO: 96. In certain embodiments, the nucleotide sequence that encodes the VL of the anti-CD123 scFv comprises the nucleotide sequence set forth in SEQ ID NO: 97.

In some embodiments, the first and/or second antigen binding moiety may comprise an anti-CD123 scFv comprising the amino acid sequence set forth in SEQ ID NO: 98, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 98. In certain embodiments, the nucleotide sequence that encodes the anti-CD123 scFv comprises the nucleotide sequence that encodes the amino acid sequence of SEQ ID NO: 98, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 98. In certain embodiments, the nucleotide sequence that encodes the anti-CD123 scFv comprises the nucleotide sequence set forth in SEQ ID NO: 99, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 99. In certain embodiments, the anti-CD123 scFv comprises the amino acid sequence set forth in SEQ ID NO: 98. In certain embodiments, the nucleotide sequence that encodes the anti-CD123 scFv comprises the nucleotide sequence set forth in SEQ ID NO: 99.

In some embodiments, the first and/or second antigen binding moiety may comprise an anti-CD123 scFv comprising the amino acid sequence set forth in SEQ ID NO: 127, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 127. In certain embodiments, the nucleotide sequence that encodes the anti-CD123 scFv comprises the nucleotide sequence that encodes the amino acid sequence of SEQ ID NO: 127, or a variant thereof having at least 50, at least 55, at least 60, at least 65, at least 70, at least 75, at least 80, at least 85, at least 90, at least 95, at least 98 or at least 99%, sequence identity with SEQ ID NO: 127. In certain embodiments, the nucleotide sequence that encodes the anti-CD123 scFv comprises the nucleotide sequence set forth in SEQ ID: 128, or a nucleotide sequence having at least 50, at least 55, at least 60, at least 65, at least 70, at least 75, at least 80, at least 85, at least 90, at least 95, at least 98 or at least 99%, sequence identity with SEQ ID NO: 128. In certain embodiments, the anti-CD123 scFv comprises the amino acid sequence set forth in SEQ ID NO: 127. In certain embodiments, the nucleotide sequence that encodes the anti-CD123 scFv comprises the nucleotide sequence set forth in SEQ ID: 128.

In certain embodiments, the CD123-binding moiety comprises three heavy chain complementarity determining regions (CDRs) (HCDR1, HCDR2, and HCDR3) contained within a heavy chain variable region (HCVR) sequence of SEQ ID NO: 94, or a variant thereof having at least 50, at least 55, at least 60, at least 65, at least 70, at least 75, at least 80, at least 85, at least 90, at least 95, at least 98 or at least 99%, sequence identity with SEQ ID NO: 94; and/or three light chain CDRs (LCDR1, LCDR2, and LCDR3) contained within a light chain variable region (LCVR) sequence of SEQ ID NO: 96, or a variant thereof having at least 50, at least 55, at least 60, at least 65, at least 70, at least 75, at least 80, at least 85, at least 90, at least 95, at least 98 or at least 99%, sequence identity with SEQ ID NO: 96.

In some embodiments, the first and/or second antigen binding moiety may comprise an anti-B7H3 scFv comprising a heavy chain variable domain (VH). In some embodiments, the VH of the anti-B7H3 scFv may comprise the amino acid sequence set forth in SEQ ID NO: 130, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 130. In certain embodiments, the nucleotide sequence that encodes the VH of the anti-B7H3 scFv comprises the nucleotide sequence that encodes the amino acid sequence of SEQ ID NO: 130, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 94. In certain embodiments, the nucleotide sequence that encodes the VH of the anti-B7H3 scFv comprises the nucleotide sequence set forth in SEQ ID NO: 129, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 129. In certain embodiments, the VH of the anti-B7H3 scFv comprises the amino acid sequence set forth in SEQ ID NO: 130. In certain embodiments, the nucleotide sequence that encodes the VH of the anti-B7H3 scFv comprises the nucleotide sequence set forth in SEQ ID NO: 129.

In some embodiments, the first and/or second antigen binding moiety may comprise an anti-B7H3 scFv comprising a light chain variable domain (VL). In some embodiments, the VL of the anti-B7H3 scFv may comprise the amino acid sequence set forth in SEQ ID NO: 132, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 132. In certain embodiments, the nucleotide sequence that encodes the VL of anti-B7H3 scFv comprises the nucleotide sequence that encodes the amino acid sequence of SEQ ID NO: 132, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 132. In certain embodiments, the nucleotide sequence that encodes the VL of the anti-B7H3 scFv comprises the nucleotide sequence set forth in SEQ ID NO: 131, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 131. In certain embodiments, the VL of the anti-B7H3 scFv comprises the amino acid sequence set forth in SEQ ID NO: 132. In certain embodiments, the nucleotide sequence that encodes the VL of the anti-B7H3 scFv comprises the nucleotide sequence set forth in SEQ ID NO: 131.

In some embodiments, the first and/or second antigen binding moiety may comprise an anti-B7H3 scFv comprising the amino acid sequence set forth in SEQ ID NO: 134, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 134. In certain embodiments, the nucleotide sequence that encodes the anti-B7H3 scFv comprises the nucleotide sequence that encodes the amino acid sequence of SEQ ID NO: 134, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 134. In certain embodiments, the nucleotide sequence that encodes the anti-B7H3 scFv comprises the nucleotide sequence set forth in SEQ ID NO: 133, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 133. In certain embodiments, the anti-B7H3 scFv comprises the amino acid sequence set forth in SEQ ID NO: 134. In certain embodiments, the nucleotide sequence that encodes the anti-B7H3 scFv comprises the nucleotide sequence set forth in SEQ ID NO: 133.

In certain embodiments, the B7H3-binding moiety comprises three heavy chain complementarity determining regions (CDRs) (HCDR1, HCDR2, and HCDR3) contained within a heavy chain variable region (HCVR) sequence of SEQ ID NO: 130, or a variant thereof having at least 50, at least 55, at least 60, at least 65, at least 70, at least 75, at least 80, at least 85, at least 90, at least 95, at least 98 or at least 99%, sequence identity with SEQ ID NO: 130; and/or three light chain CDRs (LCDR1, LCDR2, and LCDR3) contained within a light chain variable region (LCVR) sequence of SEQ ID NO: 132, or a variant thereof having at least 50, at least 55, at least 60, at least 65, at least 70, at least 75, at least 80, at least 85, at least 90, at least 95, at least 98 or at least 99%, sequence identity with SEQ ID NO: 132.

### Leader Sequence

In certain embodiments, the CARs of the present disclosure comprise a leader sequence. The leader sequence may be positioned amino-terminal to the extracellular antigen-binding domain. The leader sequence may be optionally cleaved from the antigen-binding moiety during cellular processing and localization of the CARs to the cellular membrane.

In some embodiments, the leader sequence may be derived from human immunoglobulin heavy chain variable region. In some embodiments, the leader sequence comprises the amino acid sequence set forth in SEQ ID NO: 1 or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 1. In certain embodiments, the nucleotide sequence encoding the leader sequence comprises the nucleotide sequence that encodes the amino acid sequence of SEQ ID NO: 1, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 1. In certain embodiments, the nucleotide sequence encoding the leader sequence comprises the sequence set forth in SEQ ID NO: 2, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 2. In certain embodiments, the leader sequence comprises the amino acid sequence of SEQ ID NO: 1. In certain embodiments, the nucleotide sequence encoding the leader sequence comprises the nucleotide sequence set forth in SEQ ID NO: 2.

In some embodiments, the leader sequence may be derived from CD8a. In some embodiments, the leader sequence comprises the amino acid sequence set forth in SEQ ID NO: 7 or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 7. In certain embodiments, the nucleotide sequence encoding the leader sequence comprises the nucleotide sequence that encodes the amino acid sequence of SEQ ID NO: 7, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 7. In certain embodiments, the nucleotide sequence encoding the leader sequence comprises the sequence set forth in SEQ ID NO: 8, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 8. In certain embodiments, the leader sequence comprises the amino acid sequence of SEQ ID NO: 7. In certain embodiments, the nucleotide sequence encoding the leader sequence comprises the nucleotide sequence set forth in SEQ ID NO: 8.

In various embodiments, the extracellular domain comprises an N-terminal leader sequence followed by one or more antigen binding moieties each separated by a linker.

In some embodiments, the extracellular domain comprises an N-terminal leader sequence followed by a first antigen binding moiety, e.g., a GRP78-binding moiety such as, but not limited to, a GRP78-binding peptide. In some embodiments, the extracellular antigen-binding domain comprises an N-terminal leader sequence followed by a first antigen-binding moiety and a second antigen-binding moiety each separated by a linker.

In some embodiments, the extracellular domain comprises the amino acid sequence set forth in SEQ ID NO: 100, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 100. In certain embodiments, the nucleotide sequence that encodes the extracellular antigen-binding domain comprises the nucleotide sequence that encodes the amino acid sequence of SEQ ID NO: 100, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 100. In certain embodiments, the nucleotide sequence that encodes the extracellular antigen-binding domain comprises the nucleotide sequence set forth in SEQ ID NO: 101, or a nucleotide sequence having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 101. In certain embodiments, the extracellular antigen-binding domain comprises the amino acid sequence set forth in SEQ ID NO: 100. In certain embodiments, the nucleotide sequence that encodes the extracellular antigen-binding domain comprises the nucleotide sequence set forth in SEQ ID NO: 101.

In some embodiments, the extracellular domain comprises the amino acid sequence set forth in SEQ ID NO: 102, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 102. In certain embodiments, the nucleotide sequence that encodes the extracellular antigen-binding domain comprises the nucleotide sequence that encodes the amino acid sequence of SEQ ID NO: 102, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 102. In certain embodiments, the nucleotide sequence that encodes the extracellular antigen-binding domain comprises the nucleotide sequence set forth in SEQ ID NO: 103, or a nucleotide sequence having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 103. In certain embodiments, the extracellular antigen-binding domain comprises the amino acid sequence set forth in SEQ ID NO: 102. In certain embodiments, the nucleotide sequence that encodes the extracellular antigen-binding domain comprises the nucleotide sequence set forth in SEQ ID NO: 103.

In some embodiments, the extracellular domain comprises the amino acid sequence set forth in SEQ ID NO: 104, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 104. In certain embodiments, the nucleotide sequence that encodes the extracellular antigen-binding domain comprises the nucleotide sequence that encodes the amino acid sequence of SEQ ID NO: 104, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 104. In certain embodiments, the nucleotide sequence that encodes the extracellular antigen-binding domain comprises the nucleotide sequence set forth in SEQ ID NO: 105, or a nucleotide sequence having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 105. In certain embodiments, the extracellular antigen-binding domain comprises the amino acid sequence set forth in SEQ ID NO: 104. In certain embodiments, the nucleotide sequence that encodes the extracellular antigen-binding domain comprises the nucleotide sequence set forth in SEQ ID NO: 105.

In some embodiments, the extracellular domain comprises the amino acid sequence set forth in SEQ ID NO: 106, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 106. In certain embodiments, the nucleotide sequence that encodes the extracellular antigen-binding domain comprises the nucleotide sequence that encodes the amino acid sequence of SEQ ID NO: 106, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 106. In certain embodiments, the nucleotide sequence that encodes the extracellular antigen-binding domain comprises the nucleotide sequence set forth in SEQ ID NO: 107, or a nucleotide sequence having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 107. In certain embodiments, the extracellular antigen-binding domain comprises the amino acid sequence set forth in SEQ ID NO: 106. In certain embodiments, the nucleotide sequence that encodes the extracellular antigen-binding domain comprises the nucleotide sequence set forth in SEQ ID NO: 107.

In some embodiments, the extracellular domain comprises the amino acid sequence set forth in SEQ ID NO: 141, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 141. In certain embodiments, the nucleotide sequence that encodes the extracellular antigen-binding domain comprises the nucleotide sequence that encodes the amino acid sequence of SEQ ID NO: 141, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 141. In certain embodiments, the nucleotide sequence that encodes the extracellular antigen-binding domain comprises the nucleotide sequence set forth in SEQ ID NO: 140, or a nucleotide sequence having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 140. In certain embodiments, the extracellular antigen-binding domain comprises the amino acid sequence set forth in SEQ ID NO: 141. In certain embodiments, the nucleotide sequence that encodes the extracellular antigen-binding domain comprises the nucleotide sequence set forth in SEQ ID NO: 140.

In some embodiments, the extracellular domain comprises the amino acid sequence set forth in SEQ ID NO: 147, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 147. In certain embodiments, the nucleotide sequence that encodes the extracellular antigen-binding domain comprises the nucleotide sequence that encodes the amino acid sequence of SEQ ID NO: 147, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 147. In certain embodiments, the nucleotide sequence that encodes the extracellular antigen-binding domain comprises the nucleotide sequence set forth in SEQ ID NO: 146, or a nucleotide sequence having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 146. In certain embodiments, the extracellular antigen-binding domain comprises the amino acid sequence set forth in SEQ ID NO: 147. In certain embodiments, the nucleotide sequence that encodes the extracellular antigen-binding domain comprises the nucleotide sequence set forth in SEQ ID NO: 146.

### Hinge Domain

In certain embodiments, the CARs may further comprise one or more hinge domains. In some embodiments, the CARs may further comprise a hinge domain between the extracellular antigen-binding domain, and the transmembrane domain, wherein the antigen-binding domain, hinge, and the transmembrane domain are in frame with each other.

A hinge domain can comprise any oligo- or polypeptide that functions to link the antigen-binding moiety to the transmembrane domain. A hinge domain can be used to provide more flexibility and accessibility for the antigen-binding moiety. A hinge domain may comprise up to 300 amino acids, preferably 10 to 100 amino acids and most preferably 25 to 50 amino acids. A hinge domain may be derived from all or part of naturally occurring molecules, such as from all or part of the extracellular region of CD8, CD4 or CD28, or from all or part of an antibody constant region. Alternatively, the hinge domain may be a synthetic sequence that corresponds to a naturally occurring linker region sequence, or may be an entirely synthetic linker region sequence. Non-limiting examples of hinge domains which may be used in accordance with the disclosure include a part of human CD8a chain, partial extracellular domain of CD28, FcγRIIIa receptor, IgG, IgM, IgA, IgD, IgE, an Ig hinge, or functional fragment thereof. In some embodiments, additional linking amino acids are added to the linker region to ensure that the antigen-binding moiety is an optimal distance from the transmembrane domain. In some embodiments, when the hinge domain is derived from an Ig, the linker may be mutated to prevent Fc receptor binding.

In some embodiments, the hinge domain may be derived from CD8a, CD28, or an immunoglobulin (IgG). For example, the IgG hinge may be from IgG1, IgG2, IgG3, IgG4, IgM1, IgM2, IgA1, IgA2, IgD, IgE, or a chimera thereof.

In certain embodiments, the hinge domain comprises an immunoglobulin IgG hinge or functional fragment thereof. In certain embodiments, the IgG hinge is from IgG1, IgG2, IgG3, IgG4, IgM1, IgM2, IgA1, IgA2, IgD, IgE, or a chimera thereof. In certain embodiments, the hinge domain comprises the CH1, CH2, CH3 and/or hinge region of the immunoglobulin. In certain embodiments, the hinge domain comprises the core hinge region of the immunoglobulin. The term “core hinge” can be used interchangeably with the term “short hinge” (a.k.a “SH”). Non-limiting examples of suitable hinge domains are the core immunoglobulin hinge regions listed in Table 1 (see also Wypych et al., JBC 2008 283(23): 16194-16205, which is incorporated herein by reference in its entirety for all purposes). In certain embodiments, the hinge domain is a fragment of the immunoglobulin hinge. In some embodiments, the hinge domain is a chimeric mouse IgG4/CD8a hinge, an IgG1-derived hinge-CH3 spacer, an IgG1-derived hinge-CH2-CH3 spacer, or an IgG1-derived modified hinge-CH2-CH3 spacer.

TABLE 1
Amino Acid Sequence of Short Hinge Regions
of IgG immunoglobulins

| IgG Subtype | Short Hinge Sequence | SEQ ID NO |
|---|---|---|
| IgG1 | EPKSCDKTHTCPPCP | SEQ ID NO: 39 |
| IgG1 | DLEPKSCDKTHTCPPCPDPK | SEQ ID NO: 31 |
| IgG2 | ERKCCVECPPCP | SEQ ID NO: 40 |
| IgG3 | ELKTPLGDTTHTCPRCP | SEQ ID NO: 41 |
| | (EPKSCDTPPPCPRCP)₃ | |
| IgG4 | ESKYGPPCPSCP | SEQ ID NO: 42 |

In certain embodiments, the hinge domain comprises an IgG1 hinge, or a variant thereof. In certain embodiments, the hinge domain comprises the short hinge structure of IgG1, IgG2, IgG3, or IgG4 or a variant thereof. In certain embodiments, hinge domain comprises a short hinge region and comprises the amino acid sequence set forth in SEQ ID NO: 31, 39, 40, 41, or 42, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 31, 39, 40, 41, or 42. In certain embodiments, the nucleotide sequence encoding the hinge comprising the short hinge region comprises the nucleotide sequence that encodes the amino acid sequence of SEQ ID NO: 31, 39, 40, 41, or 42, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 31, 39, 40, 41, or 42. In certain embodiments, hinge domain comprises a short hinge region and comprises the amino acid sequence set forth in SEQ ID NO: 31, 39, 40, 41, or 42.

In certain embodiments, the hinge domain comprises a short hinge region and comprises the amino acid sequence set forth in SEQ ID NO: 31, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 31. In certain embodiments, the nucleotide sequence encoding the hinge domain comprising the short hinge region comprises the nucleotide sequence that encodes the amino acid sequence of SEQ ID NO: 31, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 31. In certain embodiments, the hinge domain comprises a short hinge region and comprises the amino acid sequence set forth in SEQ ID NO: 31. In certain embodiments, the nucleotide sequence encoding the hinge comprising the short hinge region comprises the nucleotide sequence of SEQ ID NO: 32, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 32. In certain embodiments, the hinge domain comprising the short hinge region comprises the amino acid sequence of SEQ ID NO: 31. In certain embodiments, the nucleotide sequence encoding the hinge domain comprising the short hinge region comprises the nucleotide sequence of SEQ ID NO: 32.

In some embodiments, the hinge domain is derived from IgG4. In some embodiments, the hinge domain derived from IgG4 comprises the amino acid sequence set forth in SEQ ID NO: 33, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 33. In certain embodiments, the nucleotide sequence that encodes the IgG4 hinge domain comprises the nucleotide sequence that encodes the amino acid sequence of SEQ ID NO: 33, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 33. In certain embodiments, the nucleotide sequence that encodes the IgG4 hinge domain comprises the nucleotide sequence set forth in SEQ ID NO: 34, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 34. In certain embodiments, the IgG4 hinge domain comprises the amino acid sequence set forth in SEQ ID NO: 33. In certain embodiments, the nucleotide sequence that encodes the IgG4 hinge domain comprises the nucleotide sequence set forth in SEQ ID NO: 34.

In some embodiments, the hinge domain is derived from CD8a stalk or complete or partial sequences of the CD8a stalk, which are also called CD8a hinge. In some embodiments, the hinge domain derived from CD8a stalk comprises the amino acid sequence set forth in SEQ ID NO: 35, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 35. In certain embodiments, the nucleotide sequence that encodes the CD8a stalk hinge domain comprises the nucleotide sequence that encodes the amino acid sequence of SEQ ID NO: 35, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 35. In certain embodiments, the nucleotide sequence that encodes the CD8a stalk hinge domain comprises the nucleotide sequence set forth in SEQ ID NO: 36, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 36. In certain embodiments, the nucleotide sequence that encodes the CD8a stalk hinge domain comprises the nucleotide sequence set forth in SEQ ID NO: 137, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 137. In certain embodiments, the CD8a stalk hinge domain comprises the amino acid sequence set forth in SEQ ID NO: 35. In certain embodiments, the nucleotide sequence that encodes the CD8a stalk hinge domain comprises the nucleotide sequence set forth in SEQ ID NO: 36. In certain embodiments, the nucleotide sequence that encodes the CD8a stalk hinge domain comprises the nucleotide sequence set forth in SEQ ID NO: 137.

In some embodiments, the hinge domain is derived from CD28. In some embodiments, the hinge domain derived from CD28 hinge domain comprises the amino acid sequence set forth in SEQ ID NO: 37, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 37. In certain embodiments, the nucleotide sequence that encodes the CD28 hinge domain comprises the nucleotide sequence that encodes the amino acid sequence of SEQ ID NO: 37, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 37. In certain embodiments, the nucleotide sequence that encodes the CD28 hinge domain comprises the nucleotide sequence set forth in SEQ ID NO: 38, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 38. In certain embodiments, the CD28 hinge domain comprises the amino acid sequence set forth in SEQ ID NO: 37. In certain embodiments, the nucleotide sequence that encodes the CD28 hinge domain comprises the nucleotide sequence set forth in SEQ ID NO: 38.

In some embodiments, the hinge/transmembrane domain derived from CD28 hinge domain comprises the amino acid sequence set forth in SEQ ID NO: 52, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 52. In certain embodiments, the nucleotide sequence that encodes the CD28 hinge domain comprises the nucleotide sequence that encodes the amino acid sequence of SEQ ID NO: 52, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 52. In certain embodiments, the nucleotide sequence that encodes the CD28 hinge domain comprises the nucleotide sequence set forth in SEQ ID NO: 53, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 53. In certain embodiments, the CD28 hinge domain comprises the amino acid sequence set forth in SEQ ID NO: 52. In certain embodiments, the nucleotide sequence that encodes the CD28 hinge domain comprises the nucleotide sequence set forth in SEQ ID NO: 53.

In some embodiments, in addition to the sequences described above, the hinge domain may comprise any number of linker amino acids to allow for extra flexibility, rigidity, and/or accessibility.

### Linker Sequence

In certain embodiments, the CARs of the present disclosure comprise a linker sequence.

In some embodiments, the CARs may comprise a first antigen-binding moiety and a second antigen binding moiety, wherein the first and the second antigen-binding moieties may be joined by one or more linker sequences, and wherein the first antigen-binding moiety, linker sequence, and the second antigen-binding moiety are in frame with each other.

In some embodiments, the first and/or second antigen binding moieties may comprise an antigen-binding moiety comprising a single chain variable fragment (scFv), e.g., an anti-CD123 scFv or a B7H3 scFv. In some embodiments, the scFv may comprise a heavy chain variable domain (VH) and a light chain variable domain (VL), wherein the VH and the VL may be joined by one or more linker sequences disclosed herein.

In some embodiments, the linker may comprise any of the above-described sequences which may be used for the hinge domain, or functional variant thereof.

In some embodiments, the linker sequence may comprise any of the linker sequences listed in Table 2, or functional variant thereof.

TABLE 2
Amino Acid Sequence of Linkers

| Linker Sequence | SEQ ID NO |
|---|---|
| GGGGSGGGGSGGGGS | SEQ ID NO: 9 |
| IQRTPKIQVYSRHPAENGKSNFLNCYVSGFHPSDI | SEQ ID NO: 12 |
| EVDLLKNGERIEKVEHSDLSFSKDWSFYLLYYTEF | |
| TPTEKDEYACRVNHVTLSQPKIVKWDRD | |
| ESKYGPPCPSCPAPEFEGGPSVFLFPPKPKDTLMI | SEQ ID NO: 14 |
| SRTPEVTCVVVDVSQEDPEVQFNWYVDGVEVHNAK | |
| TKPREEQFQSTYRVVSVLTVLHQDWLNGKEYKCKV | |
| SNKGLPSSIEKTISKAKGQPREPQVYTLPPSQEEM | |
| TKNQVSLTCLVKGFYPSDIAVEWESNGQPENNYKT | |
| TPPVLDSDGSFFLYSRLTVDKSRWQEGNVFSCSVM | |
| HEALHNHYTQKSLSLSLGK | |
| AGSGGSGGSGGSPVPSTPPTNSSSTPPTPSPSPVP | SEQ ID NO: 16 |
| STPPTNSSSTPPTPSPSPVPSTPPTNSSSTPPTPS | |
| PSAS | |
| GGGGS | SEQ ID NO: 20 |
| GGGGSGGGGS | SEQ ID NO: 22 |
| GGGGSGGGGSGGGGSGGGGS | SEQ ID NO: 23 |
| KESGSVSSEQLAQFRSLD | SEQ ID NO: 24 |
| EGKSSGSGSESKST | SEQ ID NO: 25 |
| EGKSSGSGSESKSTQ | SEQ ID NO: 26 |
| GSTSGSGKSSEGKG | SEQ ID NO: 27 |
| SSADDAKKDDAKKDDAKKDDAKKDG | SEQ ID NO: 28 |
| EGKSSGSGSESKVD | SEQ ID NO: 29 |
| ESGSVSSEELAFRSLD | SEQ ID NO: 30 |

In some embodiments, linker sequences that may useful in the practice of the present invention include, without limitation, GGGGSGGGGSGGGGS ((G4S)3; SEQ ID NO: 9), GGGGS (SEQ ID NO: 20), GGGGSGGGGS ((G4S)2; SEQ ID NO: 22), GGGGSGGGGSGGGGSGGGGS ((G4S)4; SEQ ID NO: 23), KESGSVSSEQLAQFRSLD (SEQ ID NO: 24), EGKSSGSGSESKST (SEQ ID NO: 25), EGKSSGSGSESKSTQ (SEQ ID NO: 26), GSTSGSGKSSEGKG (SEQ ID NO: 27), SSADDAKKDDAKKDDAKKDDAKKDG (SEQ ID NO: 28), EGKSSGSGSESKVD (SEQ ID NO: 29), or ESGSVSSEELAFRSLD (SEQ ID NO: 30), or a functional variant thereof. Additional linkers include those described in, e.g., Whitlow and Filpula, Methods, Volume 2, Issue 2, April 1991, Pages 97-105, the content of which is incorporated herein by reference in its entirety.

According to certain embodiments, linkers which may be used in accordance with the invention may include a short linker (e.g., a (G4S)3 linker), a long linker (e.g., a mutated IgG4 linker), and/or a rigid linker(s) (e.g., a β2-microglobulin, β2M [B2M] and/or GPcPcPc linker). Without wishing to be bound by theory, the (G4S)3 linker is highly flexible due to its length (45 bp) and high glycine content, and is commonly used as a linker. A construct featuring a mutated IgG4 linker (mtIgG4), e.g., after the first antigen-binding moiety, may be included as a long linker (687 bp) that may generate a large gap between the first antigen-binding moiety (e.g., an GRP78-binding peptide) and the second antigen-binding moiety (e.g., an anti-CD123 scFv) that may be desirable. In some embodiments, two rigid linkers that may be used in accordance with the invention are a globular linker β2-microglobulin (294 bp) and a longer (222 bp), rigid proline-rich linker that adds three N-glycosylation sites between the antigen binding domains (GPcPcPc).

In some embodiments, the CARs of the present disclosure comprise a (G4S)3 linker. In some embodiments, the (G4S)3 linker comprises the amino acid sequence set forth in SEQ ID NO: 9, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 9. In certain embodiments, the nucleotide sequence that encodes the (G4S)3 linker comprises the nucleotide sequence that encodes the amino acid sequence of SEQ ID NO: 9, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 9. In certain embodiments, the nucleotide sequence that encodes the (G4S)3 linker comprises the nucleotide sequence set forth in SEQ ID NO: 10 or 11, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 10 or 11. In certain embodiments, the (G4S)3 linker comprises the amino acid sequence set forth in SEQ ID NO: 9. In certain embodiments, the nucleotide sequence that encodes the (G4S)3 linker comprises the nucleotide sequence set forth in SEQ ID NO: 10 or 11.

In some embodiments, the CARs of the present disclosure comprise a β2M linker. In some embodiments, the β2M linker comprises the amino acid sequence set forth in SEQ ID NO: 12, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 12. In certain embodiments, the nucleotide sequence that encodes the β2M linker comprises the nucleotide sequence that encodes the amino acid sequence of SEQ ID NO: 12, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 12. In certain embodiments, the nucleotide sequence that encodes the β2M linker comprises the nucleotide sequence set forth in SEQ ID NO: 13, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 13. In certain embodiments, the β2M linker comprises the amino acid sequence set forth in SEQ ID NO: 12. In certain embodiments, the nucleotide sequence that encodes the β2M comprises the nucleotide sequence set forth in SEQ ID NO: 13.

In some embodiments, the CARs of the present disclosure comprise a mtIgG4 linker. In some embodiments, the mtIgG4 linker comprises the amino acid sequence set forth in SEQ ID NO: 14, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 14. In certain embodiments, the nucleotide sequence that encodes the mtIgG4 linker comprises the nucleotide sequence that encodes the amino acid sequence of SEQ ID NO: 14, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 14. In certain embodiments, the nucleotide sequence that encodes the mtIgG4 linker comprises the nucleotide sequence set forth in SEQ ID NO: 15, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 15. In certain embodiments, the mtIgG4 linker comprises the amino acid sequence set forth in SEQ ID NO: 14. In certain embodiments, the nucleotide sequence that encodes the mtIgG4 comprises the nucleotide sequence set forth in SEQ ID NO: 15.

In some embodiments, the CARs of the present disclosure comprise a GPcPcPc linker. In some embodiments, the GPcPcPc linker comprises the amino acid sequence set forth in SEQ ID NO: 16, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 16. In certain embodiments, the nucleotide sequence that encodes the GPcPcPc linker comprises the nucleotide sequence that encodes the amino acid sequence of SEQ ID NO: 16, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 16. In certain embodiments, the nucleotide sequence that encodes the GPcPcPc linker comprises the nucleotide sequence set forth in SEQ ID NO: 17, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 17. In certain embodiments, the GPcPcPc linker comprises the amino acid sequence set forth in SEQ ID NO: 16. In certain embodiments, the nucleotide sequence that encodes the GPcPcPc comprises the nucleotide sequence set forth in SEQ ID NO: 17.

In some embodiments, the CARs of the present disclosure comprise spacers to provide restriction enzyme recognition sites. These spacers can be located between any of the domains described herein as well as upstream and/or downstream of linkers and between any of the regions (e.g., between the CD3 zeta region and the T2A region of the cytoplasmic domain). Non-limiting exemplary restriction enzymes include NotI, NcOI, BamHI, XhoI, SalI, and/or MluI. In an embodiment, the spacer comprises the amino acid sequence EF, AAA, or the amino acid sequence set forth in SEQ ID NO: 136. In certain embodiments, the nucleotide sequence that encodes the spacer comprises a nucleotide sequence that encodes the amino acid sequence EF, AAA, or the amino acid sequence set forth in SEQ ID NO: 136. In certain embodiments, the nucleotide sequence that encodes the spacer comprises the nucleotide sequence GAATTC, GCGGCCGCT, or the nucleotide sequence set forth in SEQ ID NO: 135. In certain embodiments, the spacer comprises the amino acid sequence EF, AAA, or the amino acid sequence set forth in SEQ ID NO: 136. In certain embodiments, the nucleotide sequence that encodes the spacer comprises the nucleotide sequence GAATTC, GCGGCCGCT, or the nucleotide sequence set forth in SEQ ID NO: 135.

### Transmembrane Domain

In certain aspects, the CARs of the present disclosure comprise a transmembrane domain, fused in frame between the extracellular antigen-binding domain and the cytoplasmic domain.

The transmembrane domain may be derived from the protein contributing to the extracellular antigen-binding domain, the protein contributing the signaling or co-signaling domain, or by a totally different protein. In some instances, the transmembrane domain can be selected or modified by amino acid substitution, deletions, or insertions to minimize interactions with other members of the CAR complex. In some instances, the transmembrane domain can be selected or modified by amino acid substitution, deletions, or insertions to avoid-binding of proteins naturally associated with the transmembrane domain. In certain embodiments, the transmembrane domain includes additional amino acids to allow for flexibility and/or optimal distance between the domains connected to the transmembrane domain.

The transmembrane domain may be derived either from a natural or from a synthetic source. Where the source is natural, the domain may be derived from any membrane-bound or transmembrane protein. Non-limiting examples of transmembrane domains of particular use in this disclosure may be derived from (i.e., comprise at least the transmembrane region(s) of) the α, β or ζ chain of the T-cell receptor, CD28, CD3 epsilon, CD45, CD4, CD5, CD8, CD8α, CD9, CD16, CD22, CD33, CD37, CD40, CD64, CD80, CD86, CD134, CD137, CD154. Alternatively, the transmembrane domain may be synthetic, in which case it will comprise predominantly hydrophobic residues such as leucine and valine. For example, a triplet of phenylalanine, tryptophan and/or valine can be found at each end of a synthetic transmembrane domain.

In some embodiments, the transmembrane domain may be derived from CD8α, CD28, CD8, CD4, CD3ζ, CD40, CD134 (OX-40), NKG2A/C/D/E, or CD7. In some embodiments, the transmembrane domain may be derived from CD28.

In certain embodiments, it will be desirable to utilize the transmembrane domain of the ζ, η or FcεR1γ chains which contain a cysteine residue capable of disulfide bonding, so that the resulting chimeric protein will be able to form disulfide linked dimers with itself, or with unmodified versions of the ζ, η or FcεR1γ chains or related proteins. In some instances, the transmembrane domain will be selected or modified by amino acid substitution to avoid-binding of such domains to the transmembrane domains of the same or different surface membrane proteins to minimize interactions with other members of the receptor complex. In other cases, it will be desirable to employ the transmembrane domain of ζ, η or FcεR1γ and −β. MB1 (Igα.), B29 or CD3-γ, ζ, or η, in order to retain physical association with other members of the receptor complex.

In certain embodiments, the transmembrane domain in the CARs of the disclosure is derived from the CD28 transmembrane domain. In certain embodiments, the CD28 transmembrane domain comprises the amino acid sequence set forth in SEQ ID NO: 43, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 43. In certain embodiments, the nucleotide sequence that encodes the CD28 transmembrane domain comprises the nucleotide sequence that encodes the amino acid sequence of SEQ ID NO: 43, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 43. In certain embodiments, the nucleotide sequence that encodes the CD28 transmembrane domain comprises the nucleotide sequence set forth in SEQ ID NO: 44 or 45, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 44 or 45. In certain embodiments, the CD28 transmembrane domain comprises the amino acid sequence set forth in SEQ ID NO: 43. In certain embodiments, the nucleotide sequence that encodes the CD28 transmembrane domain comprises the nucleotide sequence set forth in SEQ ID NO: 44 or 45.

In certain embodiments, the transmembrane domain in the CARs of the disclosure is derived from the CD28 transmembrane domain. In certain embodiments, the CD28 transmembrane domain comprises the amino acid sequence set forth in SEQ ID NO: 46, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 46. In certain embodiments, the nucleotide sequence that encodes the CD28 transmembrane domain comprises the nucleotide sequence that encodes the amino acid sequence of SEQ ID NO: 46, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 46. In certain embodiments, the nucleotide sequence that encodes the CD28 transmembrane domain comprises the nucleotide sequence set forth in SEQ ID NO: 47, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 47. In certain embodiments, the CD28 transmembrane domain comprises the amino acid sequence set forth in SEQ ID NO: 46. In certain embodiments, the nucleotide sequence that encodes the CD28 transmembrane domain comprises the nucleotide sequence set forth in SEQ ID NO: 47.

In certain embodiments, the hinge/transmembrane domain in the CARs of the disclosure is derived from the CD28 transmembrane domain. In certain embodiments, the CD28 transmembrane domain comprises the amino acid sequence set forth in SEQ ID NO: 52, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 52. In certain embodiments, the nucleotide sequence that encodes the CD28 transmembrane domain comprises the nucleotide sequence that encodes the amino acid sequence of SEQ ID NO: 52, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 52. In certain embodiments, the nucleotide sequence that encodes the CD28 transmembrane domain comprises the nucleotide sequence set forth in SEQ ID NO: 53, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 53. In certain embodiments, the CD28 transmembrane domain comprises the amino acid sequence set forth in SEQ ID NO: 52. In certain embodiments, the nucleotide sequence that encodes the CD28 transmembrane domain comprises the nucleotide sequence set forth in SEQ ID NO: 53.

In certain embodiments, the transmembrane domain in the CARs of the disclosure is derived from the CD8α transmembrane domain. In certain embodiments, the CD8α transmembrane domain comprises the amino acid sequence set forth in SEQ ID NO: 48, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 48. In certain embodiments, the nucleotide sequence that encodes the CD8α transmembrane domain comprises the nucleotide sequence that encodes the amino acid sequence of SEQ ID NO: 48, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 48. In certain embodiments, the nucleotide sequence that encodes the CD8α transmembrane domain comprises the nucleotide sequence set forth in SEQ ID NO: 49, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 49. In certain embodiments, the CD8α transmembrane domain comprises the amino acid sequence set forth in SEQ ID NO: 48. In certain embodiments, the nucleotide sequence that encodes the CD8α transmembrane domain comprises the nucleotide sequence set forth in SEQ ID NO: 49.

In certain embodiments, the transmembrane domain in the CARs of the disclosure is derived from the CD3ζ transmembrane domain. In certain embodiments, the CD3ζ transmembrane domain comprises the amino acid sequence set forth in SEQ ID NO: 50, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 50. In certain embodiments, the nucleotide sequence that encodes the CD3ζ transmembrane domain comprises the nucleotide sequence that encodes the amino acid sequence of SEQ ID NO: 50, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 50. In certain embodiments, the nucleotide sequence that encodes the CD3ζ transmembrane domain comprises the nucleotide sequence set forth in SEQ ID NO: 51, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 51. In certain embodiments, the CD3ζ transmembrane domain comprises the amino acid sequence set forth in SEQ ID NO: 50. In certain embodiments, the nucleotide sequence that encodes the CD3ζ transmembrane domain comprises the nucleotide sequence set forth in SEQ ID NO: 51.

### Cytoplasmic Domain

In certain aspects, CARs of the present disclosure comprise a cytoplasmic domain, which comprises one or more costimulatory domains and one or more signaling domains. The cytoplasmic domain, which comprises one or more costimulatory domains and one or more signaling domains, is responsible for activation of at least one of the normal effector functions of the lymphocyte in which the CARs have been placed in. The term “effector function” refers to a specialized function of a cell. Effector function of a T-cell, for example, may be cytolytic activity or helper activity including the secretion of cytokines. Thus, the term “signaling domain” refers to the portion of a protein which transduces the effector function signal and directs the cell to perform a specialized function. While usually the entire signaling domain is present, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the signaling domain sufficient to transduce the effector function signal.

Non-limiting examples of signaling domains which can be used in the CARs of the present disclosure include, e.g., signaling domains derived from DAP10, DAP12, Fc epsilon receptor I γ chain (FCER1G), FcR β, CD3δ, CD3ε, CD3γ, CD3ζ, CD5, CD22, CD226, CD66d, CD79A, and CD79B. In some embodiments, the CARs of the present disclosure comprise a signaling domain derived from CD3ζ.

In various embodiments, the CD3ζ signaling domain comprises the amino acid sequence set forth in SEQ ID NO: 69, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 69. In certain embodiments, the nucleotide sequence that encodes the CD3ζ signaling domain comprises the nucleotide sequence that encodes the amino acid sequence of SEQ ID NO: 69, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 69. In certain embodiments, the nucleotide sequence that encodes the CD3ζ signaling domain comprises the nucleotide sequence set forth in SEQ ID NO: 70, 71, or 139, or a nucleotide sequence having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 70, 71, or 139. In certain embodiments, the CD3ζ signaling domain comprises the amino acid sequence set forth in SEQ ID NO: 69. In certain embodiments, the nucleotide sequence that encodes the CD3ζ signaling domain comprises the nucleotide sequence set forth in SEQ ID NO: 70, 71, or 139.

In various embodiments the CD3ζ signaling domain comprises the amino acid sequence set forth in SEQ ID NO: 72, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 72. In certain embodiments, the nucleotide sequence that encodes the CD3ζ signaling domain comprises the nucleotide sequence that encodes the amino acid sequence of SEQ ID NO: 72, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 72. In certain embodiments, the nucleotide sequence that encodes the CD3ζ signaling domain comprises the nucleotide sequence set forth in SEQ ID NO: 73, or a nucleotide sequence having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 73. In certain embodiments, the CD3ζ signaling domain comprises the amino acid sequence set forth in SEQ ID NO: 72. In certain embodiments, the nucleotide sequence that encodes the CD3ζ signaling domain comprises the nucleotide sequence set forth in SEQ ID NO: 23.

In various embodiments, the cytoplasmic domain further comprises one or more costimulatory domains. Costimulatory domains can boost a CAR-induced immune response. Non-limiting examples of costimulatory domains include those derived from those derived from 4-1BB (CD137), CD28, CD40, ICOS, CD134 (OX-40), BTLA, CD27, CD30, GITR, CD226, CD79A, HVEM, MyD88, IL-2Rβ, STAT3-binding YXXQ, or any combination thereof. In some embodiments, the CAR of the present disclosure comprises one costimulatory domain. In certain embodiments, the cytoplasmic domain comprises a CD28 costimulatory domain. In various embodiments, the cytoplasmic domain comprises a 4-1BB costimulatory domain. In some embodiments, the cytoplasmic domain comprises an OX40 costimulatory domain.

In some embodiments, the CARs of the present disclosure comprise two or more costimulatory domains. In certain embodiments, the CARs of the present disclosure comprise two, three, four, five, six or more costimulatory domains. For example, the CARs of the present disclosure may comprise a costimulatory domain derived from 4-1BB and a costimulatory domain derived from CD28.

In certain embodiments, the CARs of the present disclosure comprise a cytoplasmic domain, which comprises a signaling domain, a MyD88 polypeptide or functional fragment thereof, and a CD40 cytoplasmic polypeptide region or a functional fragment thereof. In certain embodiments, the CAR lacks the CD40 transmembrane and/or CD40 extracellular domains. In certain embodiments, the CAR includes the CD40 transmembrane domain. In certain embodiments, the CAR includes the CD40 transmembrane domain and a portion of the CD40 extracellular domain, wherein the CD40 extracellular domain does not interact with natural or synthetic ligands of CD40.

In certain embodiments, the signaling domain is separated from the MyD88 polypeptide or functional fragment thereof and/or the CD40 cytoplasmic polypeptide region or a functional fragment thereof. In certain embodiments, the lymphocyte activation domain is separated from the MyD88 polypeptide or functional fragment thereof and/or the CD40 cytoplasmic polypeptide region or a functional fragment thereof by at least 1, 2, 3, 4, 5, 6, 7, 8, 9, 10, 11, 12, 13, 14, or 15 amino acids.

In some embodiments, the signaling domain(s) and costimulatory domain(s) can be in any order. In some embodiments, the signaling domain is upstream of the costimulatory domains. In some embodiments, the signaling domain is downstream from the costimulatory domains. In the cases where two or more costimulatory domains are included, the order of the costimulatory domains could be switched.

In various embodiments, the CD28 costimulatory domain comprises the amino acid sequence set forth in SEQ ID NO: 54, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 54. In certain embodiments, the nucleotide sequence that encodes the CD28 costimulatory domain comprises the nucleotide sequence that encodes the amino acid sequence of SEQ ID NO: 54, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 54. In certain embodiments, the nucleotide sequence that encodes the CD28 costimulatory domain comprises the nucleotide sequence set forth in SEQ ID NO: 55, 56, or 138, or a nucleotide sequence having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 55, 56, or 138. In certain embodiments, the CD28 costimulatory domain comprises the amino acid sequence set forth in SEQ ID NO: 54. In certain embodiments, the nucleotide sequence that encodes the CD28 costimulatory domain comprises the nucleotide sequence set forth in SEQ ID NO: 55, 56, or 138.

In various embodiments the 4-1BB costimulatory domain comprises the amino acid sequence set forth in SEQ ID NO: 57, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 57. In certain embodiments, the nucleotide sequence that encodes the 4-1BB costimulatory domain comprises the nucleotide sequence that encodes the amino acid sequence of SEQ ID NO: 57, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 57. In certain embodiments, the nucleotide sequence that encodes the 4-1BB costimulatory domain comprises the nucleotide sequence set forth in SEQ ID NO: 58, or a nucleotide sequence having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 58. In certain embodiments, the 4-1BB costimulatory domain comprises the amino acid sequence set forth in SEQ ID NO: 57. In certain embodiments, the nucleotide sequence that encodes the 4-1BB costimulatory domain comprises the nucleotide sequence set forth in SEQ ID NO: 58.

In various embodiments, the OX40 costimulatory domain comprises the amino acid sequence set forth in SEQ ID NO: 59, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 59. In certain embodiments, the nucleotide sequence that encodes the OX40 costimulatory domain comprises the nucleotide sequence that encodes the amino acid sequence of SEQ ID NO: 59, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 59. In certain embodiments, the nucleotide sequence that encodes the OX40 costimulatory domain comprises the nucleotide sequence set forth in SEQ ID NO: 60, or a nucleotide sequence having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 60. In certain embodiments, the OX40 costimulatory domain comprises the amino acid sequence set forth in SEQ ID NO: 59. In certain embodiments, the nucleotide sequence that encodes the OX40 costimulatory domain comprises the nucleotide sequence set forth in SEQ ID NO: 60.

In various embodiments, the cytoplasmic domain of the CAR comprises the amino acid sequence set forth in SEQ ID NO: 108, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 108. In certain embodiments, the nucleotide sequence that encodes the cytoplasmic domain of the CAR comprises the nucleotide sequence that encodes the amino acid sequence of SEQ ID NO: 108, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 108. In certain embodiments, the nucleotide sequence that encodes the cytoplasmic domain of the CAR comprises the nucleotide sequence set forth in SEQ ID NO: 109, or a nucleotide sequence having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 109. In certain embodiments, the cytoplasmic domain of the CAR comprises the amino acid sequence set forth in SEQ ID NO: 108. In certain embodiments, the nucleotide sequence that encodes the cytoplasmic domain of the CAR comprises the nucleotide sequence set forth in SEQ ID NO: 109.

In various embodiments, the cytoplasmic domain of the CAR comprises the amino acid sequence set forth in SEQ ID NO: 143, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 143. In certain embodiments, the nucleotide sequence that encodes the cytoplasmic domain of the CAR comprises the nucleotide sequence that encodes the amino acid sequence of SEQ ID NO: 143, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 143. In certain embodiments, the nucleotide sequence that encodes the cytoplasmic domain of the CAR comprises the nucleotide sequence set forth in SEQ ID NO: 142, or a nucleotide sequence having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 142. In certain embodiments, the cytoplasmic domain of the CAR comprises the amino acid sequence set forth in SEQ ID NO: 143. In certain embodiments, the nucleotide sequence that encodes the cytoplasmic domain of the CAR comprises the nucleotide sequence set forth in SEQ ID NO: 142.

In various embodiments, the cytoplasmic domain of the CAR comprises the amino acid sequence set forth in SEQ ID NO: 149, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 149. In certain embodiments, the nucleotide sequence that encodes the cytoplasmic domain of the CAR comprises the nucleotide sequence that encodes the amino acid sequence of SEQ ID NO: 149, or a variant thereof having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 149. In certain embodiments, the nucleotide sequence that encodes the cytoplasmic domain of the CAR comprises the nucleotide sequence set forth in SEQ ID NO: 148, or a nucleotide sequence having at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, or at least 99%, sequence identity with SEQ ID NO: 148. In certain embodiments, the cytoplasmic domain of the CAR comprises the amino acid sequence set forth in SEQ ID NO: 149. In certain embodiments, the nucleotide sequence that encodes the cytoplasmic domain of the CAR comprises the nucleotide sequence set forth in SEQ ID NO: 148.

### Additional Genes

In addition to the CAR construct, the CARs of the present disclosure may further comprise at least one additional gene that encodes an additional peptide. Examples of additional genes can include a transduced host cell selection marker, an in vivo tracking marker, cellular marker, epitope tag, a cytokine, a suicide gene, safety switch, or some other functional gene. In certain embodiments, the functional additional gene can induce the expression of another molecule. In certain embodiments, the functional additional gene can increase the safety of the CAR. For example, the CAR construct may comprise an additional gene which is truncated CD19 (tCD19). The tCD19 can be used as a tag. Expression of tCD19 may also help determine transduction efficiency.

Other examples of additional genes include genes that encode polypeptides with a biological function; examples include, but are not limited to, cytokines, chimeric cytokine receptors, dominant negative receptors, safety switches (CD20, truncated EGFR or HER2, inducible caspase 9 molecules). As another example, the CAR construct may comprise an additional gene which is a synNotch receptor. Once activated, the synNotch receptor can induce the expression of a target gene (e.g., a second CAR and/or bispecific molecule).

In some embodiments, the CARs of the present disclosure may comprise one or more additional nucleotide sequences encoding one or more additional polypeptide sequences. As a non-limiting example, the one or more additional polypeptide sequences may be selected from one or more cellular markers, epitope tags, cytokines, safety switches, dimerization moieties, or degradation moieties.

In certain embodiments, the CARs of the present disclosure comprise at least one additional gene (i.e., a second gene). In certain embodiments, the CARs of the present disclosure comprise one second gene. In other embodiments, the CARs of the present disclosure comprise two additional genes (i.e., a third gene). In yet another embodiment, the CARs of the present disclosure comprise three additional genes (i.e., a fourth gene). In certain embodiments, the additional genes are separated from each other and the CAR construct. For example, they may be separated by 2A sequences and/or an internal ribosomal entry sites (IRES). In certain examples, the CAR can be at any position of the polynucleotide chain (for example construct A: CAR, second gene, third gene, fourth gene; construct B: second gene, CAR, third gene, fourth gene; etc.)

Non-limiting examples of classes of additional genes that can be used to increase the effector function of CAR containing host cells, include (a) secretable cytokines (e.g., but not limited to, IL-7, IL-12, IL-15, IL-18), (b) membrane bound cytokines (e.g., but not limited to, IL-15), (c) chimeric cytokine receptors (e.g., but not limited to, IL-2/IL-7, IL-4/IL-7), (d) constitutive active cytokine receptors (e.g., but not limited to, C7R), (e) dominant negative receptors (DNR; e.g., but not limited to TGFRII DNR), (f) ligands of costimulatory molecules (e.g., but not limited to, CD80, 4-1BBL), (g) nuclear factor of activated T-cells (NFATs) (e.g., NFATc1, NFATc2, NFATc3, NFATc4, and NFAT5), (h) antibodies, including fragments thereof and bispecific antibodies (e.g., but not limited to, bispecific T-cell engagers (BiTEs)), or (i) a second CAR.

In some embodiments, the additional gene sequence may be derived from tCD19. In some embodiments, the tCD19 sequence comprises the amino acid sequence set forth in SEQ ID NO: 88 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 88. In certain embodiments, the nucleotide sequence encoding the tCD19 sequence comprises the nucleotide sequence that encodes the amino acid sequence of SEQ ID NO: 88, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 88. In certain embodiments, the nucleotide sequence encoding the tCD19 sequence comprises the sequence set forth in SEQ ID NO: 89, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 89. In certain embodiments, the tCD19 sequence comprises the amino acid sequence of SEQ ID NO: 88. In certain embodiments, the nucleotide sequence encoding the tCD19 sequence comprises the nucleotide sequence set forth in SEQ ID NO: 89.

In certain embodiments, the nucleotide sequence encoding the tCD19 sequence comprises the sequence set forth in SEQ ID NO: 90 or 91, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 90 or 91. In certain embodiments, the nucleotide sequence encoding the tCD19 sequence comprises the nucleotide sequence set forth in SEQ ID NO: 90 or 91.

In certain embodiments, the additional gene may be regulated by an NFAT dependent-promoter. Activation of the T-cell or other lymphocyte leads to activation of the transcription factor NFAT resulting in the induction of the expression of the protein encoded by the gene linked with the NFAT dependent promoter. One or more members of the NFAT family (i.e., NFATc1, NFATc2, NFATc3, NFATc4, and NFAT5) is expressed in most cells of the immune system. NFAT-dependent promoters and enhancers tend to have three to five NFAT binding sites.

In certain embodiments, the functional additional gene can be a suicide gene. A suicide gene is a recombinant gene that will cause the host cell that the gene is expressed in to undergo programmed cell death or antibody mediated clearance at a desired time. Suicide genes can function to increase the safety of the CAR. In another embodiment, the additional gene is an inducible suicide gene. Non-limiting examples of suicide genes include i) molecules that are expressed on the cell surface and can be targeted with a clinical grade monoclonal antibody including CD20, EGFR or a fragment thereof, HER2 or a fragment thereof, and ii) inducible suicide genes (e.g., but not limited to inducible caspase 9 (see Straathof et al. (2005) Blood. 105(11): 4247-4254; US Publ. No. 2011/0286980, each of which are incorporated herein by reference in their entirety for all purposes)).

In certain aspects, CARs of the present disclosure may be regulated by a safety switch. As used herein, the term “safety switch” refers to any mechanism that is capable of removing or inhibiting the effect of a CAR from a system (e.g., a culture or a subject). Safety switches can function to increase the safety of the CAR.

The function of the safety switch may be inducible. Non-limiting examples of safety switches include (a) molecules that are expressed on the cell surface and can be targeted with a clinical grade monoclonal antibody including CD20, EGFR or a fragment thereof, HER2 or a fragment thereof, and (b) inducible suicide genes (e.g., but not limited to herpes simplex virus thymidine kinase (HSV-TK) and inducible caspase 9 (see Straathof et al. (2005) Blood. 105(11): 4247-4254; US Publ. No. 2011/0286980, each of which are incorporated herein by reference in their entirety for all purposes).

In some embodiments, the safety switch is a CD20 polypeptide. Expression of human CD20 on the cell surface presents an attractive strategy for a safety switch. The inventors and others have shown that cells that express CD20 can be rapidly eliminated with the FDA approved monoclonal antibody rituximab through complement-mediated cytotoxicity and antibody-dependent cell-mediated cytotoxicity (see e.g., Griffioen, M., et al. Haematologica 94, 1316-1320 (2009), which is incorporated herein by reference in its entirety for all purposes). Rituximab is an anti-CD20 monoclonal antibody that has been FDA approved for Chronic Lymphocytic Leukemia (CLL) and Non-Hodgkin's Lymphoma (NHL), among others (Storz, U. MAbs 6, 820-837 (2014), which is incorporated herein by reference in its entirety for all purposes). The CD20 safety switch is non-immunogenic and can function as a reporter/selection marker in addition to a safety switch (Bonifant, C. L., et al. Mol Ther 24, 1615-1626 (2016); van Loenen, M. M., et al. Gene Ther 20, 861-867 (2013); each of which is incorporated herein by reference in its entirety for all purposes).

In some embodiments, the polynucleotide sequence(s) encoding the CARs of the present disclosure may be expressed in an inducible fashion, for example, as may be achieved with an inducible promoter, an inducible expression system, an artificial signaling circuits, and/or drug-induced splicing.

In some embodiments, the polynucleotide sequence(s) encoding the CARs of the present disclosure may be expressed in an inducible fashion, such as that which may be achieved with i) an inducible promoter, for example, but not limited to promotors that may be activated by T cell activation (e.g. NFAT, Nur66, IFNg) or hypoxia; ii) an inducible expression system, for example, but not limited to doxycycline- or tamoxifen-inducible expression system; iii) artificial signaling circuits including, but not limited to, SynNotch, and/or iv) drug-induced splicing. By way of a non-limiting example, drug-induced splicing methods and/or compositions useful in the practice of the present disclosure may be based those described in, for example, Monteys et al., 2021 [39], the contents of which is incorporated herein by reference in its entirety for all purposes.

In some embodiments, the polynucleotide sequence(s) encoding the CARs disclosed herein may be expressed as a ‘split molecule’ in which for example, transmembrane and intracellular signaling regions, or any other domains or regions of the CAR, may be assembled only in the presence of a heterodimerizing small molecule (e.g., small organic molecule, nucleic acid, polypeptide, or a fragment, isoform, variant, analog, or derivative thereof), as described in, for example, Wu et al., 2015³⁰, the contents of which is incorporated herein by reference in its entirety for all purposes.

In some embodiments, the polynucleotide sequence(s) encoding the CARs herein may further encode a moiety so that the stability of CAR may be regulated with a small molecule, including but not limited to, the “SWIFF” technology or an immunomodulatory drug (IMiD)-inducible degron as described, for example, in Juillerat et al., 2019³¹, Carbonneau et al., 2021³², and Jan et al., 202133, the contents of each of which is incorporated herein by reference in its entirety for all purposes.

In some embodiments, the sequence encoding an additional gene is operably linked to the sequence encoding CAR via a sequence encoding a self-cleaving peptide and/or an Internal Ribosome Entry Site (IRES) as disclosed herein.

Non-limiting examples of self-cleaving peptide sequences includes Thoseaasigna virus 2A (T2A; AEGRGSLLTCGDVEENPGP, SEQ ID NO: 77, EGRGSLLTCGDVEENPGP, SEQ ID NO: 74, or GSGEGRGSLLTCGDVEENPGP, SEQ ID NO: 78); the foot and mouth disease virus (FMDV) 2A sequence (F2A; GSGSRVTELLYRMKRAETYCPRPLLAIIIPTEARHKQKIVAPVKQLLNFDLLKLAGDVES NPGP, SEQ ID NO: 79), Sponge (Amphimedon queenslandica) 2A sequence (LLCFLLLLLSGDVELNPGP, SEQ ID NO: 80; or HHFMFLLLLLAGDIELNPGP, SEQ ID NO: 81); acorn worm 2A sequence (Saccoglossus kowalevskii) (WFLVLLSFILSGDIEVNPGP, SEQ ID NO: 82); amphioxus (Branchiostoma floridae) 2A sequence (KNCAMYMLLLSGDVETNPGP, SEQ ID NO: 83; or MVISQLMLKLAGDVEENPGP, SEQ ID NO: 84); porcine teschovirus-1 2A sequence (P2A; GSGATNFSLLKQAGDVEENPGP, SEQ ID NO: 85); and equine rhinitis A virus 2A sequence (E2A; GSGQCTNYALLKLAGDVESNPGP, SEQ ID NO: 86). In some embodiments, the separation sequence is a naturally occurring or synthetic sequence. In certain embodiments, the separation sequence includes the 2A consensus sequence D-X-E-X-NPGP (SEQ ID NO: 87), in which X is any amino acid residue.

Alternatively, an Internal Ribosome Entry Site (IRES) may be used to link the CAR and the additional gene. IRES is an RNA element that allows for translation initiation in a cap-independent manner. IRES can link two coding sequences in one bicistronic vector and allow the translation of both proteins in cells.

In some embodiments, the self-cleaving 2A peptide is a T2A peptide and comprises the amino acid sequence set forth in SEQ ID NO: 74. In some embodiments, the sequence encoding the T2A peptide comprises the nucleotide sequence SEQ ID NO: 75 or 76.

In some embodiments, the additional gene sequence may be derived from T2A. In some embodiments, the T2A sequence comprises the amino acid sequence set forth in SEQ ID NO: 74 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 74. In certain embodiments, the nucleotide sequence encoding the T2A sequence comprises the nucleotide sequence that encodes the amino acid sequence of SEQ ID NO: 74, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 74. In certain embodiments, the nucleotide sequence encoding the T2A sequence comprises the sequence set forth in SEQ ID NO: 75 or 76, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 75 or 76. In certain embodiments, the T2A sequence comprises the amino acid sequence of SEQ ID NO: 74. In certain embodiments, the nucleotide sequence encoding the T2A sequence comprises the nucleotide sequence set forth in SEQ ID NO: 75 or 76.

In certain embodiments, the host cells can be genetically modified to express not only CARs as disclosed herein but to also express fusion protein with signaling activity (e.g., costimulation, T-cell activation). These fusion proteins can improve host cell activation and/or responsiveness. In certain embodiments, the fusion protein can enhance the host cell's response to the target antigen. In certain embodiments, the fusion protein can impart resistance to suppression signals.

In certain embodiments, fusion proteins can comprise portions of CD4, CD8α, CD28, portions of a T-cell receptor, or an antigen-binding moiety (e.g., scFv) linked to a MyD88, CD40, and/or other signaling molecules.

In certain embodiments, the fusion protein comprises an extracellular antigen-binding domain (as disclosed above), a transmembrane domain (as described above) and a cytoplasmic domain, wherein the cytoplasmic domain comprises at least one co-stimulatory protein (as described above). In certain embodiments, the co-stimulatory fusion protein does not comprise a lymphocyte activation domain (e.g., CD3ζ). In certain embodiments, the at least one co-stimulatory protein can be a MyD88 polypeptide or functional fragment thereof, and/or a CD40 cytoplasmic polypeptide region or a functional fragment thereof.

In certain embodiments, the fusion protein comprises an extracellular domain (such as, but not limited to CD19, CD34), a transmembrane domain (as described above) and a cytoplasmic domain, wherein the cytoplasmic domain comprises at least one co-stimulatory protein (as described above). In certain embodiments, the fusion protein does not comprise a lymphocyte activation domain (e.g., CD3ζ). In certain embodiments, the at least one portion of the fusion protein can be a MyD88 polypeptide or functional fragment thereof, and/or a CD40 cytoplasmic polypeptide region or a functional fragment thereof.

Non-limiting examples of fusion proteins include, but are not limited to, the constructs in the publication of WO2019222579 and WO2016073875, which are incorporated herein by reference in their entirety for all purposes.

In certain embodiments, the fusion proteins are introduced into the host cell on a separate vector from the CAR. In certain embodiments, the fusion proteins are introduced into the host cell on the same vector as the CAR. In certain embodiments, the fusion proteins are introduced into the host cell on the same vector as the CAR but separated by a separation sequence such as 2A.

### Non-Limiting Examples of CARs

In certain embodiments, a 78.123-CAR of the disclosure comprises an extracellular 78.123-CAR domain comprising the amino acid sequence of SEQ ID NO: 100, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 100. In certain embodiments, the extracellular 78.123-CAR domain of a 78.123-CAR of the disclosure is encoded by a nucleotide sequence that encodes the amino acid sequence of SEQ ID NO: 100, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 100. In certain embodiments, the nucleotide sequence that encodes the extracellular 78.123-CAR domain of a 78.123-CAR of the disclosure comprises the nucleotide sequence set forth in SEQ ID NO: 101, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 101. In certain embodiments, a 78.123-CAR of the disclosure comprises an extracellular 78.123-CAR domain comprising the amino acid sequence set forth in SEQ ID NO: 100. In certain embodiments, the nucleotide sequence that encodes the extracellular 78.123-CAR domain of a 78.123-CAR of the disclosure comprises the nucleotide sequence set forth in SEQ ID NO: 101.

In certain embodiments, a 78.123-CAR of the disclosure comprises a cytoplasmic domain comprising the amino acid sequence of SEQ ID NO: 108, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 108. In certain embodiments, the cytoplasmic domain of a 78.123-CAR of the disclosure is encoded by a nucleotide sequence that encodes the amino acid sequence of SEQ ID NO: 108, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 108. In certain embodiments, the nucleotide sequence that encodes the cytoplasmic domain of a 78.123-CAR of the disclosure comprises the nucleotide sequence set forth in SEQ ID NO: 109, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 109. In certain embodiments, a 78.123-CAR of the disclosure comprises a cytoplasmic domain comprising the amino acid sequence set forth in SEQ ID NO: 108. In certain embodiments, the nucleotide sequence that encodes the cytoplasmic domain of a 78.123-CAR of the disclosure comprises the nucleotide sequence set forth in SEQ ID NO: 109.

In certain embodiments, a 78.123-CAR of the disclosure comprises the amino acid sequence of SEQ ID NO: 110, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 110. In certain embodiments, a 78.123-CAR of the disclosure is encoded by a nucleotide sequence that encodes the amino acid sequence of SEQ ID NO: 110, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 110. In certain embodiments, the nucleotide sequence that encodes a 78.123-CAR of the disclosure comprises the nucleotide sequence set forth in SEQ ID NO: 111, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 111. In certain embodiments, a 78.123-CAR of the disclosure comprises an amino acid sequence set forth in SEQ ID NO: 110. In certain embodiments, the nucleotide sequence that encodes a 78.123-CAR of the disclosure comprises the nucleotide sequence set forth in SEQ ID NO: 111.

In certain embodiments, a 78.123-CAR of the disclosure comprises an extracellular 78.123-CAR domain comprising the amino acid sequence of SEQ ID NO: 102, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 102. In certain embodiments, the extracellular 78.123-CAR domain of a 78.123-CAR of the disclosure is encoded by a nucleotide sequence that encodes the amino acid sequence of SEQ ID NO: 102, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 102. In certain embodiments, the nucleotide sequence that encodes the extracellular 78.123-CAR domain of a 78.123-CAR of the disclosure comprises the nucleotide sequence set forth in SEQ ID NO: 103, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 103. In certain embodiments, a 78.123-CAR of the disclosure comprises an extracellular 78.123-CAR domain comprising the amino acid sequence set forth in SEQ ID NO: 102. In certain embodiments, the nucleotide sequence that encodes the extracellular 78.123-CAR domain of a 78.123-CAR of the disclosure comprises the nucleotide sequence set forth in SEQ ID NO: 103.

In certain embodiments, a 78.123-CAR of the disclosure comprises a cytoplasmic domain comprising the amino acid sequence of SEQ ID NO: 108, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 108. In certain embodiments, the cytoplasmic domain of a 78.123-CAR of the disclosure is encoded by a nucleotide sequence that encodes the amino acid sequence of SEQ ID NO: 108, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 108. In certain embodiments, the nucleotide sequence that encodes the cytoplasmic domain of a 78.123-CAR of the disclosure comprises the nucleotide sequence set forth in SEQ ID NO: 109, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 109. In certain embodiments, a 78.123-CAR of the disclosure comprises a cytoplasmic domain comprising the amino acid sequence set forth in SEQ ID NO: 108. In certain embodiments, the nucleotide sequence that encodes the cytoplasmic domain of a 78.123-CAR of the disclosure comprises the nucleotide sequence set forth in SEQ ID NO: 109.

In certain embodiments, a 78.123-CAR of the disclosure comprises the amino acid sequence of SEQ ID NO: 112, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 112. In certain embodiments, a 78.123-CAR of the disclosure is encoded by a nucleotide sequence that encodes the amino acid sequence of SEQ ID NO: 112, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 112. In certain embodiments, the nucleotide sequence that encodes a 78.123-CAR of the disclosure comprises the nucleotide sequence set forth in SEQ ID NO: 113, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 113. In certain embodiments, a 78.123-CAR of the disclosure comprises an amino acid sequence set forth in SEQ ID NO: 112. In certain embodiments, the nucleotide sequence that encodes a 78.123-CAR of the disclosure comprises the nucleotide sequence set forth in SEQ ID NO: 113.

In certain embodiments, a 78.123-CAR of the disclosure comprises an extracellular 78.123-CAR domain comprising the amino acid sequence of SEQ ID NO: 104, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 104. In certain embodiments, the extracellular 78.123-CAR domain of a 78.123-CAR of the disclosure is encoded by a nucleotide sequence that encodes the amino acid sequence of SEQ ID NO: 104, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 104. In certain embodiments, the nucleotide sequence that encodes the extracellular 78.123-CAR domain of a 78.123-CAR of the disclosure comprises the nucleotide sequence set forth in SEQ ID NO: 105, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 105. In certain embodiments, a 78.123-CAR of the disclosure comprises an extracellular 78.123-CAR domain comprising the amino acid sequence set forth in SEQ ID NO: 104. In certain embodiments, the nucleotide sequence that encodes the extracellular 78.123-CAR domain of a 78.123-CAR of the disclosure comprises the nucleotide sequence set forth in SEQ ID NO: 105.

In certain embodiments, a 78.123-CAR of the disclosure comprises a cytoplasmic domain comprising the amino acid sequence of SEQ ID NO: 108, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 108. In certain embodiments, the cytoplasmic domain of a 78.123-CAR of the disclosure is encoded by a nucleotide sequence that encodes the amino acid sequence of SEQ ID NO: 108, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 108. In certain embodiments, the nucleotide sequence that encodes the cytoplasmic domain of a 78.123-CAR of the disclosure comprises the nucleotide sequence set forth in SEQ ID NO: 109, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 109. In certain embodiments, a 78.123-CAR of the disclosure comprises a cytoplasmic domain comprising the amino acid sequence set forth in SEQ ID NO: 108. In certain embodiments, the nucleotide sequence that encodes the cytoplasmic domain of a 78.123-CAR of the disclosure comprises the nucleotide sequence set forth in SEQ ID NO: 109.

In certain embodiments, a 78.123-CAR of the disclosure comprises the amino acid sequence of SEQ ID NO: 114, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 114. In certain embodiments, a 78.123-CAR of the disclosure is encoded by a nucleotide sequence that encodes the amino acid sequence of SEQ ID NO: 114, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 114. In certain embodiments, the nucleotide sequence that encodes a 78.123-CAR of the disclosure comprises the nucleotide sequence set forth in SEQ ID NO: 115, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 115. In certain embodiments, a 78.123-CAR of the disclosure comprises an amino acid sequence set forth in SEQ ID NO: 114. In certain embodiments, the nucleotide sequence that encodes a 78.123-CAR of the disclosure comprises the nucleotide sequence set forth in SEQ ID NO: 115.

In certain embodiments, a 78.123-CAR of the disclosure comprises an extracellular 78.123-CAR domain comprising the amino acid sequence of SEQ ID NO: 106, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 106. In certain embodiments, the extracellular 78.123-CAR domain of a 78.123-CAR of the disclosure is encoded by a nucleotide sequence that encodes the amino acid sequence of SEQ ID NO: 106, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 106. In certain embodiments, the nucleotide sequence that encodes the extracellular 78.123-CAR domain of a 78.123-CAR of the disclosure comprises the nucleotide sequence set forth in SEQ ID NO: 107, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 107. In certain embodiments, a 78.123-CAR of the disclosure comprises an extracellular 78.123-CAR domain comprising the amino acid sequence set forth in SEQ ID NO: 106. In certain embodiments, the nucleotide sequence that encodes the extracellular 78.123-CAR domain of a 78.123-CAR of the disclosure comprises the nucleotide sequence set forth in SEQ ID NO: 107

In certain embodiments, a 78.123-CAR of the disclosure comprises a cytoplasmic domain comprising the amino acid sequence of SEQ ID NO: 108, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 108. In certain embodiments, the cytoplasmic domain of a 78.123-CAR of the disclosure is encoded by a nucleotide sequence that encodes the amino acid sequence of SEQ ID NO: 108, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 108. In certain embodiments, the nucleotide sequence that encodes the cytoplasmic domain of a 78.123-CAR of the disclosure comprises the nucleotide sequence set forth in SEQ ID NO: 109, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 109. In certain embodiments, a 78.123-CAR of the disclosure comprises a cytoplasmic domain comprising the amino acid sequence set forth in SEQ ID NO: 108. In certain embodiments, the nucleotide sequence that encodes the cytoplasmic domain of a 78.123-CAR of the disclosure comprises the nucleotide sequence set forth in SEQ ID NO: 109.

In certain embodiments, a 78.123-CAR of the disclosure comprises the amino acid sequence of SEQ ID NO: 116, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 116. In certain embodiments, a 78.123-CAR of the disclosure is encoded by a nucleotide sequence that encodes the amino acid sequence of SEQ ID NO: 116, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 116. In certain embodiments, the nucleotide sequence that encodes a 78.123-CAR of the disclosure comprises the nucleotide sequence set forth in SEQ ID NO: 117, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 117. In certain embodiments, a 78.123-CAR of the disclosure comprises an amino acid sequence set forth in SEQ ID NO: 116. In certain embodiments, the nucleotide sequence that encodes a 78.123-CAR of the disclosure comprises the nucleotide sequence set forth in SEQ ID NO: 117.

In certain embodiments, a 78.B7H3-CAR of the disclosure comprises an extracellular 78.B7H3-CAR domain comprising the amino acid sequence of SEQ ID NO: 141, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 141. In certain embodiments, the extracellular 78.B7H3-CAR domain of a 78.B7H3-CAR of the disclosure is encoded by a nucleotide sequence that encodes the amino acid sequence of SEQ ID NO: 141, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 141. In certain embodiments, the nucleotide sequence that encodes the extracellular 78.B7H3-CAR domain of a 78.B7H3-CAR of the disclosure comprises the nucleotide sequence set forth in SEQ ID NO: 140, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 140. In certain embodiments, a 78.B7H3-CAR of the disclosure comprises an extracellular 78.B7H3-CAR domain comprising the amino acid sequence set forth in SEQ ID NO: 141. In certain embodiments, the nucleotide sequence that encodes the extracellular 78.B7H3-CAR domain of a 78.B7H3-CAR of the disclosure comprises the nucleotide sequence set forth in SEQ ID NO: 140.

In certain embodiments, a 78.B7H3-CAR of the disclosure comprises a cytoplasmic domain comprising the amino acid sequence of SEQ ID NO: 143, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 143. In certain embodiments, the cytoplasmic domain of a 78.B7H3-CAR of the disclosure is encoded by a nucleotide sequence that encodes the amino acid sequence of SEQ ID NO: 143, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 143. In certain embodiments, the nucleotide sequence that encodes the cytoplasmic domain of a 78.B7H3-CAR of the disclosure comprises the nucleotide sequence set forth in SEQ ID NO: 142, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 142. In certain embodiments, a 78.B7H3-CAR of the disclosure comprises a cytoplasmic domain comprising the amino acid sequence set forth in SEQ ID NO: 143. In certain embodiments, the nucleotide sequence that encodes the cytoplasmic domain of a 78.B7H3-CAR of the disclosure comprises the nucleotide sequence set forth in SEQ ID NO: 142.

In certain embodiments, a 78.B7H3-CAR of the disclosure comprises the amino acid sequence of SEQ ID NO: 145, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 145. In certain embodiments, a 78.B7H3-CAR of the disclosure is encoded by a nucleotide sequence that encodes the amino acid sequence of SEQ ID NO: 145, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 145. In certain embodiments, the nucleotide sequence that encodes a 78.B7H3-CAR of the disclosure comprises the nucleotide sequence set forth in SEQ ID NO: 144, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 144. In certain embodiments, a 78.B7H3-CAR of the disclosure comprises an amino acid sequence set forth in SEQ ID NO: 145. In certain embodiments, the nucleotide sequence that encodes a 78.B7H3-CAR of the disclosure comprises the nucleotide sequence set forth in SEQ ID NO: 144.

In certain embodiments, a 78.B7H3-CAR of the disclosure comprises an extracellular 78.B7H3-CAR domain comprising the amino acid sequence of SEQ ID NO: 147, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 147. In certain embodiments, the extracellular 78.B7H3-CAR domain of a 78.B7H3-CAR of the disclosure is encoded by a nucleotide sequence that encodes the amino acid sequence of SEQ ID NO: 147, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 147. In certain embodiments, the nucleotide sequence that encodes the extracellular 78.B7H3-CAR domain of a 78.B7H3-CAR of the disclosure comprises the nucleotide sequence set forth in SEQ ID NO: 146, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 146. In certain embodiments, a 78.B7H3-CAR of the disclosure comprises an extracellular 78.B7H3-CAR domain comprising the amino acid sequence set forth in SEQ ID NO: 147. In certain embodiments, the nucleotide sequence that encodes the extracellular 78.B7H3-CAR domain of a 78.B7H3-CAR of the disclosure comprises the nucleotide sequence set forth in SEQ ID NO: 146.

In certain embodiments, a 78.B7H3-CAR of the disclosure comprises a cytoplasmic domain comprising the amino acid sequence of SEQ ID NO: 149, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 149. In certain embodiments, the cytoplasmic domain of a 78.B7H3-CAR of the disclosure is encoded by a nucleotide sequence that encodes the amino acid sequence of SEQ ID NO: 149, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 149. In certain embodiments, the nucleotide sequence that encodes the cytoplasmic domain of a 78.B7H3-CAR of the disclosure comprises the nucleotide sequence set forth in SEQ ID NO: 148, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 148. In certain embodiments, a 78.B7H3-CAR of the disclosure comprises a cytoplasmic domain comprising the amino acid sequence set forth in SEQ ID NO: 149. In certain embodiments, the nucleotide sequence that encodes the cytoplasmic domain of a 78.B7H3-CAR of the disclosure comprises the nucleotide sequence set forth in SEQ ID NO: 148.

In certain embodiments, a 78.B7H3-CAR of the disclosure comprises the amino acid sequence of SEQ ID NO: 151, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 151. In certain embodiments, a 78.B7H3-CAR of the disclosure is encoded by a nucleotide sequence that encodes the amino acid sequence of SEQ ID NO: 151, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 151. In certain embodiments, the nucleotide sequence that encodes a 78.B7H3-CAR of the disclosure comprises the nucleotide sequence set forth in SEQ ID NO: 150, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 150. In certain embodiments, a 78.B7H3-CAR of the disclosure comprises an amino acid sequence set forth in SEQ ID NO: 151. In certain embodiments, the nucleotide sequence that encodes a 78.B7H3-CAR of the disclosure comprises the nucleotide sequence set forth in SEQ ID NO: 150.

In various embodiments, the polynucleotide encoding the CAR is a DNA molecule. In various embodiments, the polynucleotide encoding the CAR is an RNA molecule.

In one aspect, the present disclosure provides CAR polypeptides encoded by a polynucleotide described above.

### Vectors

The present disclosure provides recombinant vectors comprising a polynucleotide encoding a CAR comprising polynucleotides encoding the proteins disclosed above. In certain embodiments, the polynucleotide is operatively linked to at least one regulatory element for expression of the chimeric antigen receptor.

In certain embodiments, recombinant vectors of the disclosure comprise the nucleotide sequence of SEQ ID NO: 119, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 119. In certain embodiments, recombinant vectors comprise a nucleotide sequence that encodes the amino acid sequence of SEQ ID NO: 118, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 118.

In certain embodiments, recombinant vectors of the disclosure comprise the nucleotide sequence of SEQ ID NO: 121, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 121. In certain embodiments, recombinant vectors comprise a nucleotide sequence that encodes the amino acid sequence of SEQ ID NO: 120, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 120.

In certain embodiments, recombinant vectors of the disclosure comprise the nucleotide sequence of SEQ ID NO: 123, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 123. In certain embodiments, recombinant vectors comprise a nucleotide sequence that encodes the amino acid sequence of SEQ ID NO: 122, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 122.

In certain embodiments, recombinant vectors of the disclosure comprise the nucleotide sequence of SEQ ID NO: 125, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 125. In certain embodiments, recombinant vectors comprise a nucleotide sequence that encodes the amino acid sequence of SEQ ID NO: 124, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 124.

In certain embodiments, recombinant vectors of the disclosure comprise the nucleotide sequence of SEQ ID NO: 144, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 144. In certain embodiments, recombinant vectors comprise a nucleotide sequence that encodes the amino acid sequence of SEQ ID NO: 145, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 145.

In certain embodiments, recombinant vectors of the disclosure comprise the nucleotide sequence of SEQ ID NO: 150, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 150. In certain embodiments, recombinant vectors comprise a nucleotide sequence that encodes the amino acid sequence of SEQ ID NO: 151, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 151.

In certain embodiments, the recombinant vector comprises a polynucleotide encoding a CAR, wherein the polynucleotide is operatively linked to at least one additional gene. In some embodiments, the additional gene is a tCD19. In some embodiments, the additional gene is 2A. In some embodiments, the polynucleotide is operatively linked to at least two additional genes comprising a tCD19 and 2A.

In certain embodiments, the vector is a viral vector. In certain embodiments, the viral vector can be, but is not limited to, a retroviral vector, an adenoviral vector, an adeno-associated virus vector, an alphaviral vector, a herpes virus vector, and a vaccinia virus vector. In some embodiments, the viral vector is a lentiviral vector.

In some embodiments, the vector is a non-viral vector. The viral vector may be a plasmid or a transposon (such as a PiggyBac- or a Sleeping Beauty transposon).

In certain embodiments, the polynucleotide encoding the CAR is operably linked to at least a regulatory element. The regulatory element can be capable of mediating expression of the CAR in the host cell. Regulatory elements include, but are not limited to, promoters, enhancers, initiation sites, polyadenylation (polyA) tails, IRES elements, response elements, and termination signals. In certain embodiments, the regulatory element regulates CAR expression. In certain embodiments, the regulatory element increased the expression of the CAR. In certain embodiments, the regulatory element increased the expression of the CAR once the host cell is activated. In certain embodiments, the regulatory element decreases expression of the CAR. In certain embodiments, the regulatory element decreases expression of the CAR once the host cell is activated.

### Isolated Host Cells

In one aspect, provided herein is an isolated host cell comprising a polynucleotide described above or a recombinant vector described above. In a further aspect, provided herein is an isolated host cell comprising a CAR encoded by a polynucleotide described above.

In certain embodiments, the host cell is an immune cell. In some embodiments, the host cell is a T cell, a natural killer (NK) cell, a mesenchymal stem cell (MSC), or a macrophage.

In various embodiments, the host cell is a T cell. T cells may include, but are not limited to, thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+ CD8+ T cell, or any other subset of T cells. Other illustrative populations of T cells suitable for use in particular embodiments include naive T cells memory T cells, NKT cells, and iNKT cells.

In some embodiments, the T cell is selected from a CD8+ T cell, a CD4+ T cell, a cytotoxic T cell, an αβ T cell receptor (TCR) T cell, a natural killer T (NKT) cell, an invariant natural killer T (iNKT) cell, a T6 T cell, a memory T cell, a memory stem T cell (TSCM, a naïve T cell, an effector T cell, a T-helper cell, and a regulatory T cell (Treg).

In various embodiments, the host cell is a natural killer (NK) cell. NK cell refers to a differentiated lymphocyte with a CD3− CD16+, CD3− CD56+, CD16+ CD56+ and/or CD57+ TCR− phenotype.

In various embodiments, other host immune cells are selected, for example, but not limited to, macrophages. In various embodiments, the host immune cell is a dendritic cell, a Langerhans cell, or a B cell. In various embodiments, the host immune cell is a professional antigen-presenting cell (APC). In various embodiments, the host immune cell is a Non-professional APC.

In various embodiments, the host cell has been activated and/or expanded ex vivo.

In various embodiments, the host cell is an allogeneic cell. In various embodiments, the host cell is an autologous cell.

In certain embodiments, the host cell is isolated from a subject having a cancer. In certain embodiments, one or more cells of the cancer express GRP78 and/or CD123 and/or B7H3. In some embodiments, the host cell is isolated from a subject having a tumor. In various embodiments, the cancer is a solid tumor, a brain tumor, or a hematologic malignancy. In certain embodiments, the hematologic malignancy is acute myeloid leukemia (AML), acute lymphoblastic leukemia (ALL), B-cell acute lymphoblastic leukemia (B-ALL), T cell acute lymphoblastic leukemia (T-ALL), or lymphoma.

In some embodiments, the tumor can be found within, but not limited to, breast tissue, prostate tissue, bladder tissue, oral and/or dental tissue, head and/or neck tissue, stomach tissue, liver tissue, colorectal tissue, lung tissue, brain tissue, ovary, cervix, esophagus, skin, lymph nodes, and/or bone. In some embodiments, the tumor is a cancer. In some embodiments, the cancer can be, but not limited to, osteosarcoma, rhabdomyosarcoma, Ewing sarcoma and other Ewing sarcoma family of tumors, neuroblastoma, ganglioneuroblastoma, desmoplastic small round cell tumor, malignant peripheral nerve sheath tumor, synovial sarcoma, undifferentiated sarcoma, adrenocortical carcinoma, hepatoblastoma, Wilms tumor, rhabdoid tumor, high grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, acute lymphoblastic leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma and other liver cancers, head and neck cancers, leiomyosarcoma, and melanoma. In some embodiments, the tumor is a solid tumor. In various embodiments, the solid tumor is Ewings sarcoma, lung adenocarcinoma, osteosarcoma, breast cancer, or prostate cancer. In certain embodiments, the brain tumor is glioblastoma or neuroblastoma.

In certain embodiments, the host cell is isolated from a subject having a tumor, wherein one or more cells of the tumor cells express GRP78 and/or CD123 and/or B7H3. Non-limiting examples of tumors or cancer cells that express GRP78 and/or CD123 and/or B7H3 include any of the above listed tumors or cancers.

In some embodiments, the host cell is derived from a blood, marrow, tissue, or a tumor sample.

In certain aspects, the present disclosure provides a method of generating an isolated host cell described herein. The method includes genetically modifying the host cell with the polynucleotide described herein or the recombinant vector described herein. In some embodiments, the genetic modifying step is conducted via viral gene delivery. In some embodiments, the genetic modifying step is conducted via non-viral gene delivery. In some embodiments, the genetically modifying step is conducted ex vivo. In some embodiments, the method further comprises activation and/or expansion of the host cell ex vivo before, after and/or during said genetic modification.

### Isolation Enrichment

The host cells may be autologous/autogeneic (“self”) or non-autologous (“non-self,” e.g., allogeneic, syngeneic or xenogeneic). In certain embodiments, the host cells are obtained from a mammalian subject. In other embodiments, the host cells are obtained from a primate subject. In certain embodiments, the host cells are obtained from a human subject.

Lymphocytes can be obtained from sources such as, but not limited to, peripheral blood mononuclear cells, bone marrow, lymph nodes tissue, cord blood, thymus issue, tissue from a site of infection, ascites, pleural effusion, spleen tissue, and tumors. Lymphocytes may also be generated by differentiation of stem cells. In certain embodiments, lymphocytes can be obtained from blood collected from a subject using techniques generally known to the skilled person, such as sedimentation, e.g., FICOLL™ separation.

In certain embodiments, cells from the circulating blood of a subject are obtained by apheresis. An apheresis device typically contains lymphocytes, including T cells, monocytes, granulocytes, B cells, other nucleated white blood cells, red blood cells, and platelets. In certain embodiments, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. A washing step may be accomplished by methods known to those in the art, such as, but not limited to, using a semiautomated flowthrough centrifuge (e.g., Cobe 2991 cell processor, or the Baxter CytoMate). After washing, the cells may be resuspended in a variety of biocompatible buffers, cell culture medias, or other saline solution with or without buffer.

In certain embodiments, host cells can be isolated from peripheral blood mononuclear cells (PBMCs) by lysing the red blood cells and depleting the monocytes. As an example, the cells can be sorted by centrifugation through a PERCOLL™ gradient. In certain embodiments, after isolation of PBMC, both cytotoxic and helper T lymphocytes can be sorted into naive, memory, and effector T cell subpopulations either before or after activation, expansion, and/or genetic modification.

In certain embodiments, T lymphocytes can be enriched. For example, a specific subpopulation of T lymphocytes, expressing one or more markers such as, but not limited to, CD3, CD4, CD8, CD14, CD15, CD16, CD19, CD27, CD28, CD34, CD36, CD45RA, CD45RO, CD56, CD62, CD62L, CD122, CD123, CD127, CD235a, CCR7, HLA-DR or a combination thereof using either positive or negative selection techniques. In certain embodiments, the T lymphocytes for use in the compositions of the disclosure do not express or do not substantially express one or more of the following markers: CD57, CD244, CD160, PD-1, CTLA4, TIM3, and LAG3.

In certain embodiments, NK cells can be enriched. For example, a specific subpopulation of T lymphocytes, expressing one or more markers such as, but not limited to, CD2, CD16, CD56, CD57, CD94, CD122 or a combination thereof using either positive or negative selection techniques.

### Stimulation Activation

In order to reach sufficient therapeutic doses of host cell compositions, host cells are often subjected to one or more rounds of stimulation/activation. In certain embodiments, a method of producing host cells for administration to a subject comprises stimulating the host cells to become activated in the presence of one or more stimulatory signals or agents (e.g., compound, small molecule, e.g., small organic molecule, nucleic acid, polypeptide, or a fragment, isoform, variant, analog, or derivative thereof). In certain embodiments, a method of producing host cells for administration to a subject comprises stimulating the host cells to become activated and to proliferate in the presence of one or more stimulatory signals or agents.

Host cells (e.g., T lymphocytes and NK cells) can be activated by inducing a change in their biologic state by which the cells express activation markers, produce cytokines, proliferate and/or become cytotoxic to target cells. All these changes can be produced by primary stimulatory signals. Co-stimulatory signals amplify the magnitude of the primary signals and suppress cell death following initial stimulation resulting in a more durable activation state and thus a higher cytotoxic capacity.

T cells can be activated generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety for all purposes.

In certain embodiments, the T cell based host cells can be activated by binding to an agent that activates CD3ζ.

In other embodiments, a CD2-binding agent may be used to provide a primary stimulation signal to the T cells. For example, and not by limitation, CD2 agents include, but are not limited to, CD2 ligands and anti-CD2 antibodies, e.g., the Tl 1.3 antibody in combination with the Tl 1.1 or Tl 1.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986) J. Immunol. 137:1097-1100). Other antibodies which bind to the same epitopes as any of the above described antibodies can also be used.

In certain embodiments, the host cells are activated by administering phorbol myristate acetate (PMA) and ionomycine. In certain embodiments, the host cells are activated by administering an appropriate antigen that induces activation and then expansion. In certain embodiments, PMA, ionomycin, and/or appropriate antigen are administered with CD3 induce activation and/or expansion.

In general, the activating agents used in the present disclosure includes, but is not limited to, an antibody, a fragment thereof and a proteinaceous binding molecule with antibody-like functions. Examples of (recombinant) antibody fragments are Fab fragments, Fv fragments, single-chain Fv fragments (scFv), a divalent antibody fragment such as an (Fab)2′-fragment, diabodies, triabodies (Iliades, P., et al., FEBS Lett (1997) 409, 437-441), decabodies (Stone, E., et al., Journal of Immunological Methods (2007) 318, 88-94) and other domain antibodies (Holt, L. J., et al., Trends Biotechnol. (2003), 21, 11, 484-490). The divalent antibody fragment may be an (Fab)2′-fragment, or a divalent single-chain Fv fragment while the monovalent antibody fragment may be selected from the group consisting of a Fab fragment, a Fv fragment, and a single-chain Fv fragment (scFv).

In certain embodiments, one or more binding sites of the CD3ζ agents may be a bivalent proteinaceous artificial binding molecule such as a dimeric lipocalin mutein (i.e., duocalin). In certain embodiments the receptor binding reagent may have a single second binding site, (i.e., monovalent). Examples of monovalent agents include, but are not limited to, a monovalent antibody fragment, a proteinaceous binding molecule with antibody-like binding properties or an MHC molecule. Examples of monovalent antibody fragments include, but are not limited to a Fab fragment, a Fv fragment, and a single-chain Fv fragment (scFv), including a divalent single-chain Fv fragment.

The agent that specifically binds CD3 includes, but is not limited to, an anti-CD3− antibody, a divalent antibody fragment of an anti-CD3 antibody, a monovalent antibody fragment of an anti-CD3-antibody, and a proteinaceous CD3-binding molecule with antibody-like binding properties. A proteinaceous CD3-binding molecule with antibody-like binding properties can be an aptamer, a mutein based on a polypeptide of the lipocalin family, a glubody, a protein based on the ankyrin scaffold, a protein based on the crystalline scaffold, an adnectin, and an avimer. It also can be coupled to a bead.

In certain embodiments, the activating agent (e.g., CD3-binding agents) can be present in a concentration of about 0.1 to about 10 μg/ml. In certain embodiments, the activating agent (e.g., CD3-binding agents) can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the activating agent (e.g., CD3-binding agents) is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In certain embodiments, the CD3-binding agents can be present in a concentration of 1 μg/ml.

NK cells can be activated generally using methods as described, for example, in U.S. Pat. Nos. 7,803,376, 6,949,520, 6,693,086, 8,834,900, 9,404,083, 9,464,274, 7,435,596, 8,026,097, 8,877,182; U.S. Patent Applications US2004/0058445, US2007/0160578, US2013/0011376, US2015/0118207, US2015/0037887; and PCT Patent Application WO2016/122147, each of which is incorporated herein by reference in its entirety for all purposes.

In certain embodiments, the NK based host cells can be activated by, for example and not limitation, inhibition of inhibitory receptors on NK cells (e.g., KIR2DL1, KIR2DL2/3, KIR2DL4, KIR2DL5A, KIR2DL5B, KIR3DL1, KIR3DL2, KIR3DL3, LILRB1, NKG2A, NKG2C, NKG2E or LTLRB5 receptor).

In certain embodiments, the NK based host cells can be activated by, for example and not limitation, feeder cells (e.g., native K562 cells or K562 cells that are genetically modified to express 4-1BBL and cytokines such as IL15 or IL21).

In other embodiments, interferons or macrophage-derived cytokines can be used to activate NK cells. For example and not limitation, such interferons include but are not limited to interferon alpha and interferon gamma, and such cytokines include but are not limited to IL-15, IL-2, IL-21.

In certain embodiments, the NK activating agent can be present in a concentration of about 0.1 to about 10 μg/ml. In certain embodiments, the NK activating agent can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the NK activating agent is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μg/ml, about 0.9 μg/ml, about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, or about 10 μg/ml. In certain embodiments, the NK activating agent can be present in a concentration of 1 μg/ml.

In certain embodiments, the activating agent is attached to a solid support such as, but not limited to, a bead, an absorbent polymer present in culture plate or well or other matrices such as, but not limited to, Sepharose or glass; may be expressed (such as in native or recombinant forms) on cell surface of natural or recombinant cell line by means known to those skilled in the art.

### Polynucleotide Transfer

The host cells can be genetically modified after stimulation/activation. In certain embodiments, the host cells are modified within 12 hours, 16 hours, 24 hours, 36 hours, or 48 hours of stimulation/activation. In certain embodiments, the cells are modified within 16 to 24 hours after stimulation/activation. In certain embodiments, the host cells are modified within 24 hours.

In order to genetically modify the host cell to express the CAR or other related molecule (e.g., TCR or bispecific antibody), the polynucleotide construct must be transferred into the host cell. Polynucleotide transfer may be via viral or non-viral gene methods. Suitable methods for polynucleotide delivery for use with the current methods include any method known by those of skill in the art, by which a polynucleotide can be introduced into an organelle, cell, tissue, or organism.

In some embodiments, polynucleotides are transferred to the cell in a non-viral vector. Non-viral vectors suitable for use in this invention include but are not limited to minicircle plasmids, transposon systems (e.g., Sleeping Beauty, piggyBac), or single or double stranded DNA molecules that are used as templates for homology directed repair (HDR) based gene editing.

Nucleic acid vaccines can be used to transfer polynucleotides into the host cells. Such vaccines include, but are not limited to non-viral polynucleotide vectors, “naked” DNA and RNA, and viral vectors. Methods of genetically modifying cells with these vaccines, and for optimizing the expression of genes included in these vaccines are known to those of skill in the art.

In certain embodiments, the host cells can be genetically modified by methods ordinarily used by one of skill in the art. In certain embodiments, the host cells can be transduced via retroviral transduction. References describing retroviral transduction of genes are Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993), each of which is incorporated herein by reference in its entirety for all purposes.

One method of genetic modification includes ex vivo modification. Various methods are available for transfecting cells and tissues removed from a subject via ex vivo modification. For example, retroviral gene transfer in vitro can be used to genetically modified cells removed from the subject and the cell transferred back into the subject. See e.g., Wilson et al., Science, 244:1344-1346, 1989 and Nabel et al., Science, 244(4910):1342-1344, 1989, both of which are incorporated herein by reference in their entity for all purposes. In certain embodiments, the host cells may be removed from the subject and transfected ex vivo using the polynucleotides (e.g., expression vectors) of the disclosure. In certain embodiments, the host cells obtained from the subject can be transfected or transduced with the polynucleotides (e.g., expression vectors) of the disclosure and then administered back to the subject.

Another method of gene transfer includes injection. In certain embodiments, a cell or a polynucleotide or viral vector may be delivered to a cell, tissue, or organism via one or more injections (e.g., a needle injection). Non-limiting methods of injection include injection of a composition (e.g., a saline based composition). Polynucleotides can also be introduced by direct microinjection. Non-limiting sites of injection include, subcutaneous, intradermal, intramuscular, intranodal (allows for direct delivery of antigen to lymphoid tissues). intravenous, intraprotatic, intratumor, intralymphatic (allows direct administration of DCs) and intraperitoneal. It is understood that proper site of injection preparation is necessary (e.g., shaving of the site of injection to observe proper needle placement).

Electroporation is another method of polynucleotide delivery. See e.g., Potter et al., (1984) Proc. Nat'l Acad. Sci. USA, 81, 7161-7165 and Tur-Kaspa et al., (1986) Mol. Cell Biol., 6, 716-718, both of which are incorporated herein in their entirety for all purposes. Electroporation involves the exposure of a suspension of cells and DNA to a high-voltage electric discharge. In certain embodiments, cell wall-degrading enzymes, such as pectin-degrading enzymes, can be employed to render the host cells more susceptible to genetic modification by electroporation than untreated cells. See e.g., U.S. Pat. No. 5,384,253, incorporated herein by reference in its entirety for all purposes.

In vivo electroporation involves a basic injection technique in which a vector is injected intradermally in a subject. Electrodes then apply electrical pulses to the intradermal site causing the cells localized there (e.g., resident dermal dendritic cells), to take up the vector. These tumor antigen-expressing dendritic cells activated by local inflammation can then migrate to lymph-nodes.

Methods of electroporation for use with this invention include, for example, Sardesai, N. Y., and Weiner, D. B., Current Opinion in Immunotherapy 23:421-9 (2011) and Ferraro, B. et al., Human Vaccines 7:120-127 (2011), both of which are hereby incorporated by reference herein in their entirety for all purposes.

Additional methods of polynucleotide transfer include liposome-mediated transfection (e.g., polynucleotide entrapped in a lipid complex suspended in an excess of aqueous solution. See e.g., Ghosh and Bachhawat, (1991) In: Liver Diseases, Targeted Diagnosis and Therapy Using Specific Receptors and Ligands. pp. 87-104). Also contemplated is a polynucleotide complexed with Lipofectamine, or Superfect); DEAE-dextran (e.g., a polynucleotide is delivered into a cell using DEAE-dextran followed by polyethylene glycol. See e.g., Gopal, T. V., Mol Cell Biol. 1985 May; 5(5):1188-90); calcium phosphate (e.g., polynucleotide is introduced to the cells using calcium phosphate precipitation. See e.g., Graham and van der Eb, (1973) Virology, 52, 456-467; Chen and Okayama, Mol. Cell Biol., 7(8):2745-2752, 1987), and Rippe et al., Mol. Cell Biol., 10:689-695, 1990); sonication loading (introduction of a polynucleotide by direct sonic loading. See e.g., Fechheimer et al., (1987) Proc. Nat'l Acad Sci. USA, 84, 8463-8467); microprojectile bombardment (e.g., one or more particles may be coated with at least one polynucleotide and delivered into cells by a propelling force. See e.g., U.S. Pat. Nos. 5,550,318; 5,538,880; 5,610,042; and PCT Application WO 94/09699; Klein et al., (1987) Nature, 327, 70-73, Yang et al., (1990) Proc. Nat'l Acad Sci. USA, 87, 9568-9572); and receptor-mediated transfection (e.g., selective uptake of macromolecules by receptor-mediated endocytosis that will be occurring in a target cell using cell type-specific distribution of various receptors. See e.g., Wu and Wu, (1987) J. Biol. Chem., 262, 4429-4432; Wagner et al., Proc. Natl. Acad Sci. USA, 87(9):3410-3414, 1990; Perales et al., Proc. Natl. Acad Sci. USA, 91:4086-4090, 1994; Myers, EPO 0273085; Wu and Wu, Adv. Drug Delivery Rev., 12:159-167, 1993; Nicolau et al., (1987) Methods Enzymol., 149, 157-176), each reference cited here is incorporated by reference in their entirety for all purposes.

In further embodiments, host cells are genetically modified using gene editing with homology-directed repair (HDR). Homology-directed repair (HDR) is a mechanism used by cells to repair double strand DNA breaks. In HDR, a donor polynucleotide with homology to the site of the double strand DNA break is used as a template to repair the cleaved DNA sequence, resulting in the transfer of genetic information from the donor polynucleotide to the DNA. As such, new nucleic acid material may be inserted or copied into a target DNA cleavage site. Double strand DNA breaks in host cells may be induced by a site-specific nuclease. Suitable site-specific nucleases for use in the present invention include, but are not limited to, RNA-guided endonuclease (e.g., CRISPR-associated (Cas) proteins), zinc finger nuclease, a TALEN nuclease, or mega-TALEN nuclease. For example, a site-specific nuclease (e.g., a Cas9+guide RNA) capable of inducing a double strand break in a target DNA sequence is introduced to a host cell, along with a donor polynucleotide encoding a CAR of the present disclosure and optionally an additional protein (e.g., TCR or bispecific antibody).

### Expansion Proliferation

After the host cells are activated and transduced, the cells are cultured to proliferate. T cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of T cells can include interleukins, such as IL-2, IL-7, IL-15, or IL-21 (see for example Cornish et al. 2006, Blood. 108(2):600-8, Bazdar and Sieg, 2007, Journal of Virology, 2007, 81(22):12670-12674, Battalia et al, 2013, Immunology, 139(1):109-120, each of which is incorporated herein by reference in its entirety for all purposes). Other illustrative examples for agents that may be used for the expansion of T cells are agents that bind to CD8, CD45 or CD90, such as αCD8, αCD45 or αCD90 antibodies. Illustrative examples of T cell population including antigen-specific T cells, T helper cells, cytotoxic T cells, memory T cell (an illustrative example of memory T cells are CD62L+ CD8+ specific central memory T cells) or regulatory T cells (an illustrative example of Treg are CD4+CD25+CD45RA+ Treg cells).

Additional agents that can be used to expand T lymphocytes includes methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety for all purposes.

In certain embodiments, the agent(s) used for expansion (e.g., IL-2) are administered at about 20 units/ml to about 200 units/ml. In certain embodiments, the agent(s) used for expansion (e.g., IL-2) are administered at about 25 units/ml to about 190 units/ml, about 30 units/ml to about 180 units/ml, about 35 units/ml to about 170 units/ml, about 40 units/ml to about 160 units/ml, about 45 units/ml to about 150 units/ml, about 50 units/ml to about 140 units/ml, about 55 units/ml to about 130 units/ml, about 60 units/ml to about 120 units/ml, about 65 units/ml to about 110 units/ml, about 70 units/ml to about 100 units/ml, about 75 units/ml to about 95 units/ml, or about 80 units/ml to about 90 units/ml. In certain embodiments, the agent(s) used for expansion (e.g., IL-2) are administered at about 20 units/ml, about 25 units/ml, about 30 units/ml, 35 units/ml, 40 units/ml, 45 units/ml, about 50 units/ml, about 55 units/ml, about 60 units/ml, about 65 units/ml, about 70 units/ml, about 75 units/ml, about 80 units/ml, about 85 units/ml, about 90 units/ml, about 95 units/ml, about 100 units/ml, about 105 units/ml, about 110 units/ml, about 115 units/ml, about 120 units/ml, about 125 units/ml, about 130 units/ml, about 135 units/ml, about 140 units/ml, about 145 units/ml, about 150 units/ml, about 155 units/ml, about 160 units/ml, about 165 units/ml, about 170 units/ml, about 175 units/ml, about 180 units/ml, about 185 units/ml, about 190 units/ml, about 195 units/ml, or about 200 units/ml. In certain embodiments, the agent(s) used for expansion (e.g., IL-2) are administered at about 5 mg/ml to about 10 ng/ml. In certain embodiments, the agent(s) used for expansion (e.g., IL-2) are administered at about 5.5 ng/ml to about 9.5 ng/ml, about 6 ng/ml to about 9 ng/ml, about 6.5 ng/ml to about 8.5 ng/ml, or about 7 ng/ml to about 8 ng/ml. In certain embodiments, the agent(s) used for expansion (e.g., IL-2) are administered at about 5 ng/ml, 6 ng/ml, 7 ng/ml, 8 ng/ml, 9, ng/ml, or 10 ng/ml.

After the host cells are activated and transduced, the cells are cultured to proliferate. NK cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of natural killer cells can include agents that bind to CD16 or CD56, such as for example αCD16 or αCD56 antibodies. In certain embodiments, the binding agent includes antibodies (see for example Hoshino et al, Blood. 1991 Dec. 15; 78(12):3232-40, which is incorporated herein by reference in its entirety for all purposes). Other agents that may be used for expansion of NK cells may be IL-15 (see for example Vitale et al. 2002. The Anatomical Record. 266:87-92, which is hereby incorporated by reference in its entirety for all purposes).

Conditions appropriate for T cell culture include appropriate media. Non-limiting examples of appropriate media include Minimal Essential Media (MEM), RPMI Media 1640, Lonza RPMI 1640, Advanced RPMI, Clicks, AIM-V, DMEM, a-MEM, F-12, TexMACS, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion.

Examples of other additives for host cell expansion include, but are not limited to, surfactant, plasmanate, pH buffers such as HEPES, and reducing agents such as N-acetyl-cysteine and 2-mercaptoethanol, Antibiotics (e.g., penicillin and streptomycin), are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In certain embodiments, host cells of the present disclosure may be modified such that the expression of an endogenous TCR, MHC molecule, or other immunogenic molecule is decreased or eliminated. When allogeneic cells are used, rejection of the therapeutic cells may be a concern as it may cause serious complications such as the graft-versus-host disease (GvHD). Although not wishing to be bound by theory, immunogenic molecules (e.g., endogenous TCRs and/or MHC molecules) are typically expressed on the cell surface and are involved in self vs non-self discrimination. Decreasing or eliminating the expression of such molecules may reduce or eliminate the ability of the therapeutic cells to cause GvHD.

In certain embodiments, expression of an endogenous TCR in the host cells is decreased or eliminated. In a particular embodiment, expression of an endogenous TCR (e.g., αβ TCR) in the host cells is decreased or eliminated. Expression of the endogenous TCR may be decreased or eliminated by disrupting the TRAC locus, TCR beta constant locus, and/or CD3 locus. In certain embodiments, expression of an endogenous TCR may be decreased or eliminated by disrupting one or more of the TRAC, TRBC1, TRBC2, CD3E, CD3G, and/or CD3D locus.

In certain embodiments, expression of one or more endogenous MHC molecules in the host cells is decreased or eliminated. Modified MHC molecule may be an MHC class I or class II molecule. In certain embodiments, expression of an endogenous MHC molecule may be decreased or eliminated by disrupting one or more of the MHC, β2M, TAP1, TAP2, CIITA, RFX5, RFXAP and/or RFXANK locus.

Expression of an endogenous TCR, an MHC molecule, and/or any other immunogenic molecule in the host cell can be disrupted using genome editing techniques such as Clustered regularly interspaced short palindromic repeats (CRISPR)/Cas, zinc finger nucleases (ZFNs), transcription activator-like effector nucleases (TALENs), and Meganucleases. These genome editing methods may disrupt a target gene by entirely knocking out all of its output or partially knocking down its expression. In a particular embodiment, expression of the endogenous TCR, an MHC molecule and/or any other immunogenic molecule in the host cell is disrupted using the CRISPR/Cas technique.

### Pharmaceutical Compositions

In another aspect, the present disclosure provides for pharmaceutical compositions comprising the isolated host cells described above. Compositions of the present disclosure include, but are not limited to, pharmaceutical compositions.

In one aspect, the present disclosure provides a pharmaceutical composition comprising a polynucleotide or a recombinant vector encoding a CAR described herein, and a pharmaceutically accepted carrier and/or excipient.

In another aspect, the present disclosure provides pharmaceutical composition comprising the modified host cells comprising a CAR described herein and a pharmaceutically acceptable carrier and/or excipient.

Excipients included in the pharmaceutical composition will have different purposes depending, for example, on host cells used, the polynucleotide or recombinant vector used, the CAR(s) used, and the mode of administration. Examples of generally used excipients include, without limitation: saline, buffered saline, dextrose, water, glycerol, ethanol, and combinations thereof, stabilizing agents, solubilizing agents and surfactants, buffers and preservatives, tonicity agents, bulking agents, and lubricating agents. Pharmaceutical compositions comprising isolated host cells will typically have been prepared and cultured in the absence of any non-human components, such as animal serum (e.g., bovine serum albumin).

Examples of pharmaceutical carriers include but are not limited to sterile liquids, such as water and oils, including those of petroleum, animal, vegetable, or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Compositions comprising modified host cells disclosed herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions comprising modified host cells disclosed herein may comprise one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the compositions are formulated to be introduced into the subject by parenteral administration, e.g., intravascular (intravenous or intraarterial), intraperitoneal, intratumoral, intraventricular, intrapleural or intramuscular administration. The parenteral preparation can be enclosed in ampoules, disposable syringes or multiple dose vials made of glass or plastic. An injectable pharmaceutical composition is preferably sterile. In some embodiments, the composition is reconstituted from a lyophilized preparation prior to administration.

In some embodiments, the modified host cells may be mixed with substances that adhere to or penetrate the host cells prior to administration of the host cells. A non-limiting example of the substances is nanoparticles.

### Therapeutic Methods

In one aspect, the present disclosure provides a method for killing a tumor or cancer cell expressing GRP78 and/or CD123 and/or B7H3 comprising contacting the cell with the host cell(s), or the pharmaceutical composition(s) described herein.

In one aspect, the present disclosure provides a method for treating a tumor in a subject in need thereof. One or more cells of the tumor expresses GRP78 and/or CD123 and/or B7H3. The method comprises administering to the subject a therapeutically effective amount of the modified host cell(s) comprising a CAR described herein or the pharmaceutical composition.

In various embodiments, the cancer is a solid tumor, a brain tumor, or a hematologic malignancy. In certain embodiments, the hematologic malignancy is AML, ALL, B-ALL, T-ALL, or lymphoma. Examples of tumors are, but not limited to, the soft tissue tumors (e.g., lymphomas), and tumors of the blood and blood-forming organs (e.g., leukemias), and solid tumors, which is one that grows in an anatomical site outside the bloodstream (e.g., carcinomas). Examples of cancer include, but are not limited to, carcinoma, lymphoma, blastoma, sarcoma (e.g., Ewing sarcoma and other Ewing sarcoma family of tumors, osteosarcoma or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung), cancer of the peritoneum, hepatocellular cancer, gastric or stomach cancer (e.g., including gastrointestinal cancer, pancreatic cancer), cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, primary or metastatic melanoma, multiple myeloma and B-cell lymphoma, non-Hodgkin's lymphoma, Hodgkin's lymphoma, brain (e.g., high grade glioma, diffuse pontine glioma, ependymoma, neuroblastoma, or glioblastoma), as well as head and neck cancer, and associated metastases. Additional examples of tumors can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

In various embodiments, the tumor is selected from osteosarcoma, rhabdomyosarcoma, Ewing sarcoma and other Ewing sarcoma family of tumors, neuroblastoma, ganglioneuroblastoma, desmoplastic small round cell tumor, malignant peripheral nerve sheath tumor, synovial sarcoma, undifferentiated sarcoma, adrenocortical carcinoma, hepatoblastoma, Wilms tumor, rhabdoid tumor, high grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma and other liver cancers, head and neck cancers, leiomyosarcoma, and melanoma. In various embodiments, the tumor is a solid tumor. In various embodiments, the solid tumor is Ewings sarcoma, lung adenocarcinoma, osteosarcoma, breast cancer, or prostate cancer. In certain embodiments, the tumor is a brain tumor. In some embodiments, the brain tumor is glioblastoma or neuroblastoma.

In some embodiments, the modified host cell(s) comprising a CAR described herein or the pharmaceutical composition may be used to target the immune microenvironment, for example, immune cells which may comprise the environment around a tumor. For example, the modified host cell(s) comprising a CAR described herein or the pharmaceutical composition may target an immune cell including, without limitation, a macrophage, a granulogcyte, or a mast cell in the immune microenvironment. In some embodiments, the one or more tumor cells surrounded by the immune microenvironment may express GRP78 and/or CD123 and/or B7H3.

In some embodiments, the therapeutic method of the present disclosure includes one or more of the following steps: a) isolating immune cells (e.g., T cells, iNKT cells, mesenchymal stem cells, macrophages, or NK cells) from the subject or donor; b) genetically modifying the immune cells (e.g., T cells, iNKT cells, mesenchymal stem cells, macrophages, or NK cells) ex vivo with the polynucleotide or the recombinant vector encoding a CAR described herein; c) optionally, expanding and/or activating the modified the immune cells (e.g., T cells, iNKT cells, mesenchymal stem cells, macrophages, or NK cells) before, after and/or during step b); and d) introducing a therapeutically effective amount of the modified immune cells (e.g., T cells, iNKT cells, mesenchymal stem cells, macrophages, or NK cells) into the subject. In some embodiments, the immune cell is an αβ TCR T cell, a T6 T cell, a macrophage, a mesenchymal stem cell, a NK cell, or an iNKT cell.

In some embodiments, the modified host cell is an autologous cell. In some embodiments, the modified host cell is an allogeneic cell. In cases where the host cell is isolated from a donor, the method may further include a method to prevent graft vs. host disease (GVHD) and host cell rejection.

In some embodiments, the modified host cells may also express a CD20 polypeptide as a safety switch. Accordingly, the method may further include administering an anti-CD20 antibody to the subject for removal of the isolated host cells. The anti-CD20 antibody is administered in an amount effective for sufficient removal of the isolated host cells from the subject. In some embodiments, the anti-CD20 antibody is administered in an amount effective for removal of more than 50% of the isolated host cells from the subject. For example, the anti-CD20 antibody may be administered in an amount effective for removal of more than 55%, more than 60%, more than 65%, more than 70%, more than 75%, more than 80%, more than 85%, more than 90%, more than 95%, more than 98%, more than 99%, or about 100% of the isolated host cells from the subject. The anti-CD20 antibody may be administered in an amount effective for removal of about 50% to about 70%, about 60% to about 80%, about 70% to about 90%, or about 80% to about 100% of the isolated host cells from the subject.

Non-limiting examples of anti-CD20 antibodies that can be used for removal of the isolated host cells include Rituximab, Ibritumomab tiuxetan, Tositumomab, Ofatumumab, Ocrelizumab, TRU-015, Veltuzumab, AME-133v, PRO131921, and Obinutuzumab. In some embodiments, the anti-CD20 antibody is Rituximab.

In some embodiments of any of the therapeutic methods described above, the composition is administered in a therapeutically effective amount. The dosages of the composition administered in the methods of the invention will vary widely, depending upon the subject's physical parameters, the frequency of administration, the manner of administration, the clearance rate, and the like. The initial dose may be larger and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve in vivo persistence of modified host cells. It is also contemplated that a variety of doses will be effective to improve in vivo effector function of modified host cells.

In some embodiments, composition comprising the modified host cells manufactured by the methods described herein may be administered at a dosage of 10² to 10¹⁰ cells/kg body weight, 10⁵ to 10⁹ cells/kg body weight, 10⁵ to 10⁸ cells/kg body weight, 10⁵ to 10⁷ cells/kg body weight, 10⁷ to 10⁹ cells/kg body weight, or 10⁷ to 10⁸ cells/kg body weight, including all integer values within those ranges. The number of modified host cells will depend on the therapeutic use for which the composition is intended for.

Modified host cells may be administered multiple times at dosages listed above. The modified host cells may be allogeneic, syngeneic, xenogeneic, or autologous to the patient undergoing therapy.

The compositions and methods described in the present disclosure may be utilized in conjunction with other types of therapy for tumors, such as chemotherapy, surgery, radiation, gene therapy, and so forth.

It is also contemplated that when used to treat various diseases/disorders, the compositions and methods of the present disclosure can be utilized with other therapeutic methods/agents suitable for the same or similar diseases/disorders. Such other therapeutic methods/agents can be co-administered (simultaneously or sequentially) to generate additive or synergistic effects. Suitable therapeutically effective dosages for each agent may be lowered due to the additive action or synergy.

In some embodiments of any of the above therapeutic methods, the method further comprises administering to the subject one or more additional compounds selected from the group consisting of immuno-suppressives, biologicals, probiotics, prebiotics, and cytokines (e.g., GM-CSF, IFN or IL-2).

In some embodiments, the method described herein further comprises providing exogenous GM-CSF, in addition to the GM-CSF produced by the immune cells, to enhance the function of immune cells expressing a CAR of the present disclosure. Exogenous GM-CSF may be provided by, for example and not limitation, i) injection of the FDA-approved GM-CSF drug Sargramostin (Leukine™) or ii) the use of nonviral or viral vectors to express GM-CSF (e.g., FDA-approved GM-CSF expressing oncolytic virus talimogene laherparepvec [TVEC, Imlygic™]). These drugs could be given before, with, or after the administration (e.g., infusion) of the immune cells expressing a CAR of the present disclosure to patients.

As a non-limiting example, the invention can be combined with other therapies that block inflammation (e.g., via blockage of 11, INFα/β, IL6, TNF, IL23, etc.).

The methods and compositions of the invention can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 4-1BB, OX40, etc.). The methods of the invention can be also combined with other treatments that possess the ability to modulate NKT function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e). The methods of the invention can also be combined with other treatments such as midostaurin, enasidenib, or a combination thereof.

Therapeutic methods of the invention can be combined with additional immunotherapies and therapies. For example, when used for treating tumors, the compositions of the invention can be used in combination with conventional therapies, such as, e.g., surgery, radiotherapy, chemotherapy or combinations thereof, depending on type of the tumor, patient condition, other health issues, and a variety of factors. In certain aspects, other therapeutic agents useful for combination tumor therapy with the inhibitors of the invention include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In one embodiment, the modified host cells of the invention can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments of the present disclosure include, for example, aminoglutethimide, amsacrine, anastrozole, asparaginase, azacitidine, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, decitabine, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-tumor agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In various embodiments of the methods described herein, the subject is a human. The subject may be a juvenile, a pediatric subject, or an adult, of any age or sex. In some embodiments, the subject is under the age of 18. In various embodiments, the subject is less than about 3 months, about 6 months, about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, or about 18 years of age. In some embodiments, the subject is about 19 years, about 20 years, about 25 years, about 30 years, about 35 years, about 40 years, abo 45 years, about 50 years, about 55 years, about 60 years, about 65 years, about 70 years, about 75 years, about 80 years, about 85 years, about 90 years, about 95 years, or about 100 years old.

In accordance with the present disclosure there may be numerous tools and techniques within the skill of the art, such as those commonly used in molecular biology, pharmacology, and microbiology. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ.

### EXAMPLES

The following examples are provided to further describe some of the embodiments disclosed herein. The examples are intended to illustrate, not to limit, the disclosed embodiments.

### Example 1. Generation and Characterization of 78.123-CAR T Cells

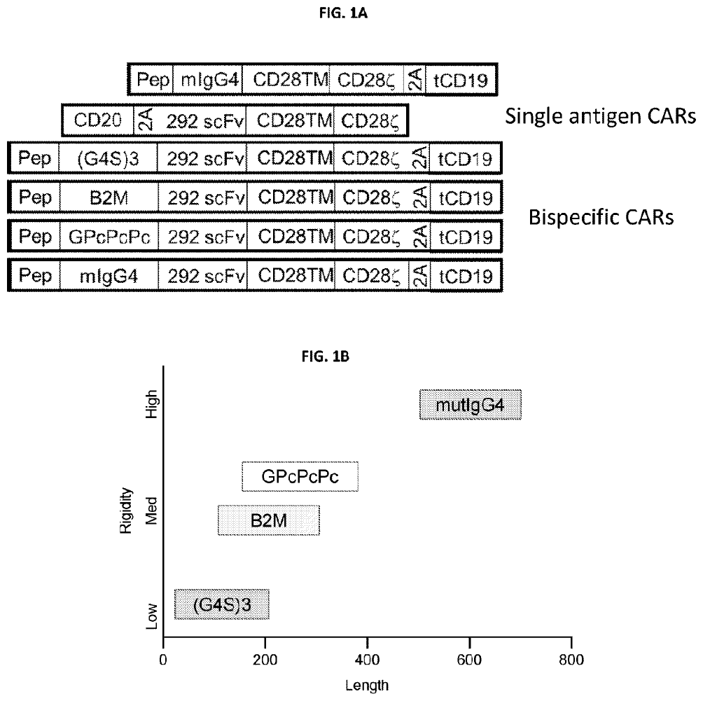

To target cell surface GRP78 and CD123 positive acute myeloid leukemia (AML) cells, a panel of four bispecific-CARs with a common backbone including a CD28 transmembrane domain, a CD28 costimulatory domain, and a CD3ζ activation domain, were designed. Bispecific CARs were subcloned into a retroviral vector upstream of a T2A sequence and truncated CD19 (tCD19) tag (FIG. 1A). The four bispecific CARs had four different linkers with different flexibilities joining the two antigen recognition domains (FIG. 1B). The selected linkers included a short ((G4S)3), a long (mtIgG4), and two rigid linkers (β2-microglobulin, β2M [B2M] and GPcPcPc). The (G4S)3 linker is highly flexible due to its length (45 bp) and high glycine content and commonly used as a linker. A construct featuring a mutated IgG4 after the GRP78 antigen recognition domain was included to test a long linker (687 bp) that generates a large gap between both binding domains. The two rigid linkers included a globular linker β2-microglobulin (294 bp) and a longer (222 bp), rigid proline-rich linker that adds three N-glycosylation sites between the antigen binding domains (GPcPcPc).

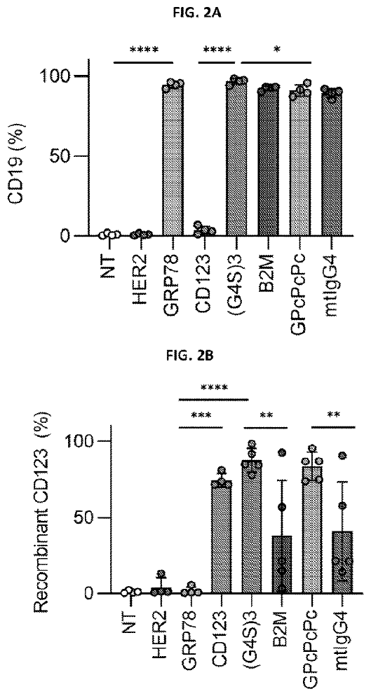

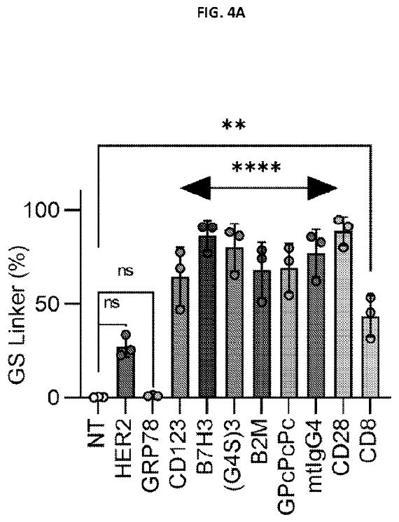

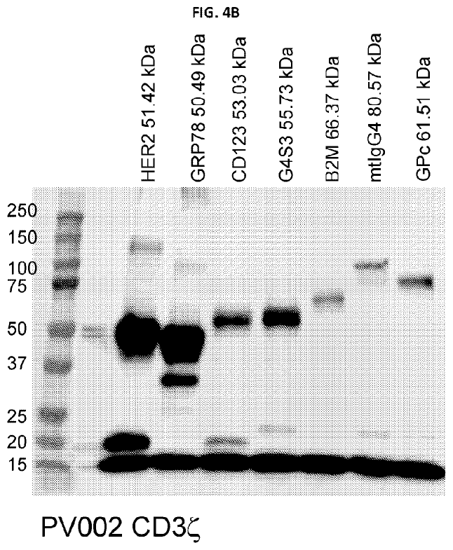

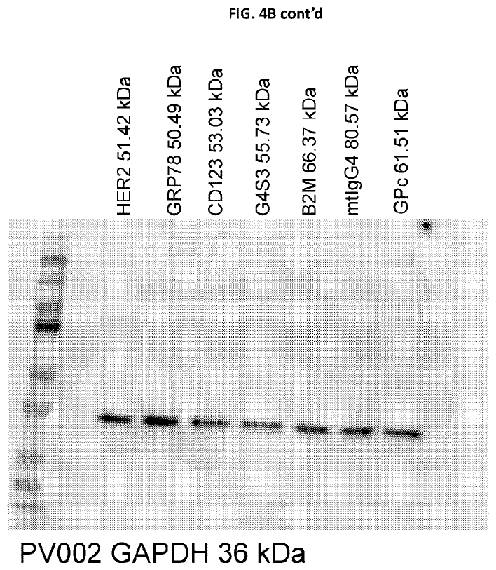

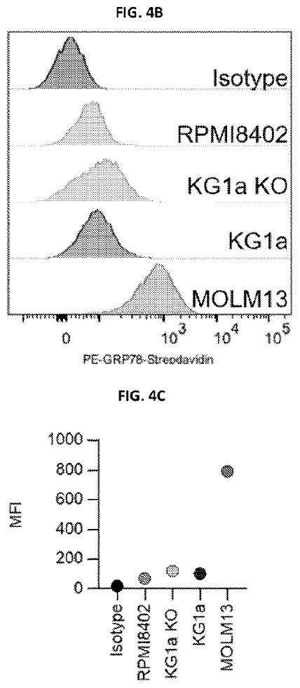

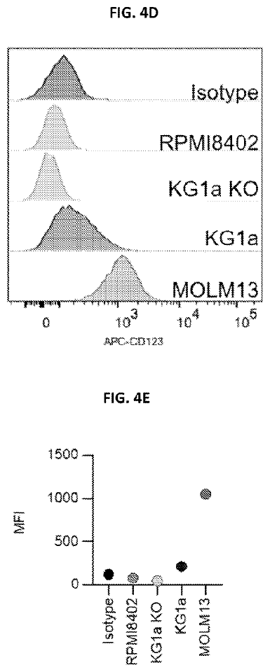

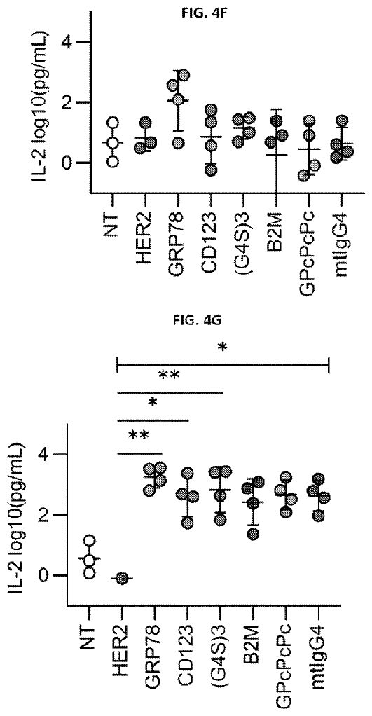

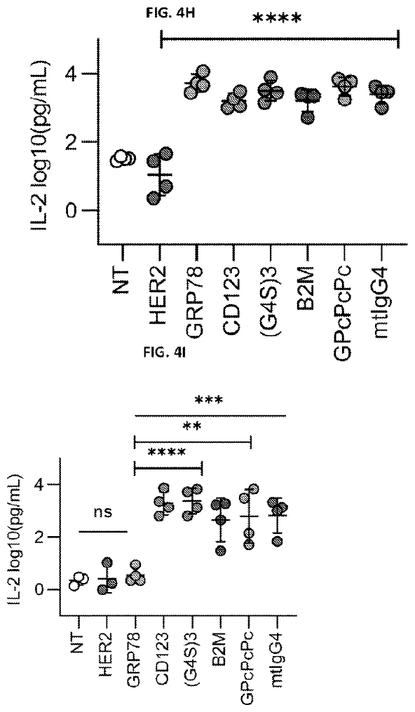

Transduction efficiency measured by tCD19 and recombinant CD123 protein binding showed no statistically significant difference in tCD19 expression between CARs (FIG. 2A). However, not all bispecific CARs bound recombinant CD123 protein with the same efficiency as CD123-CAR T cells (FIG. 2B). Western blot analysis confirmed protein expression of each bispecific CAR (FIG. 4A).

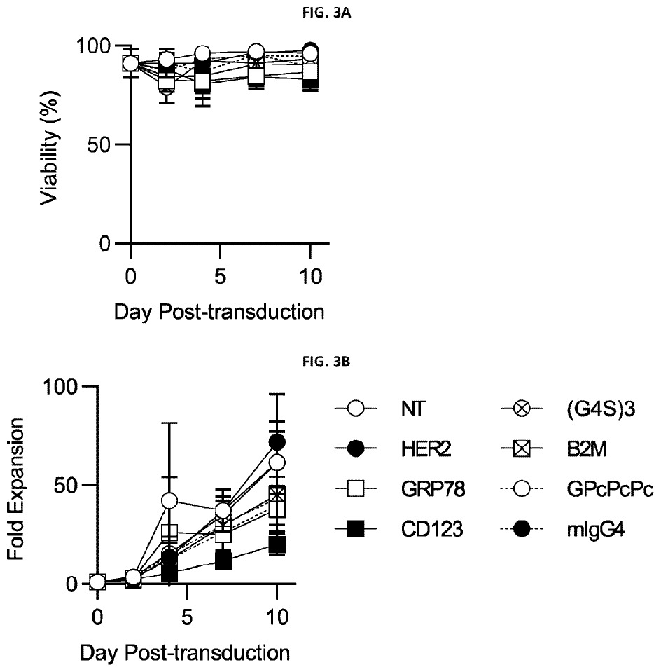

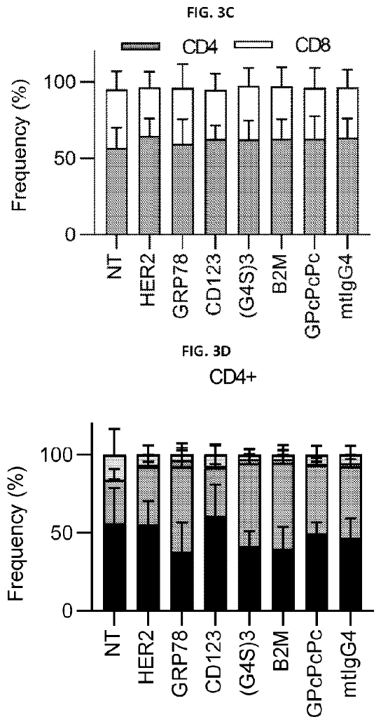

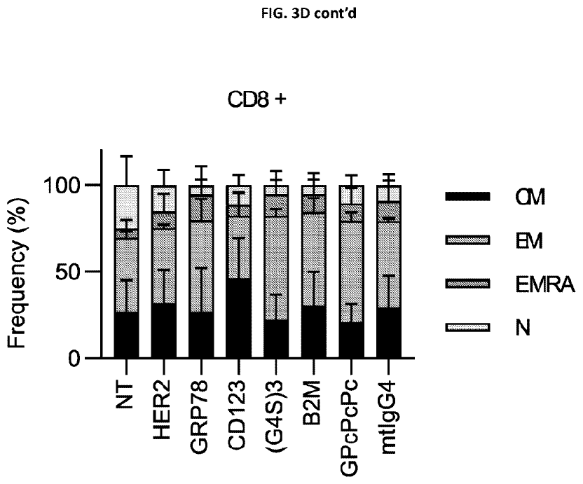

While GRP78-CAR and CD123-CAR T cells had a decrease in viability and expansion when compared to NT T cells, differences in expansion and viability in bispecific CAR T cells were not observed (FIGS. 3A-3B). Immunophenotype determination via flow cytometry demonstrated no significant difference in CD4:CD8 ratio among effector T cells (FIG. 3C). However, when effector memory (EM: CCR7−, CD45RO+), central memory (CM: CCR7+, CD45RAO+), Naïve-like (N: CCR7+ CD45RO−), and effector memory RA (EMRA: CCR7−, CD45RO−) were evaluated, 78.(G4S)3.123 bispecific CARs had significantly more CD4 and CD8 EM T cells compared to NT T cells (FIG. 3D).

### Example 2. 78.123-CAR T Cells Recognize AML Cells Expressing Cell Surface GRP78 and/or CD123 In Vitro

To determine 78.123-CAR T cell effector function, an array of AML cell lines was analyzed for surface GRP78 and CD123 expression. While no cell line was completely negative for GRP78, a KG1a knockout (KO) GRP78⁺/CD123⁻, KG1a GRP78⁺/CD123⁺, and MOLM13 GRP78⁺/CD123⁺ were used (FIGS. 4B-4E). MOLM13 had the highest antigen expression and mean fluorescence intensity (MFI).

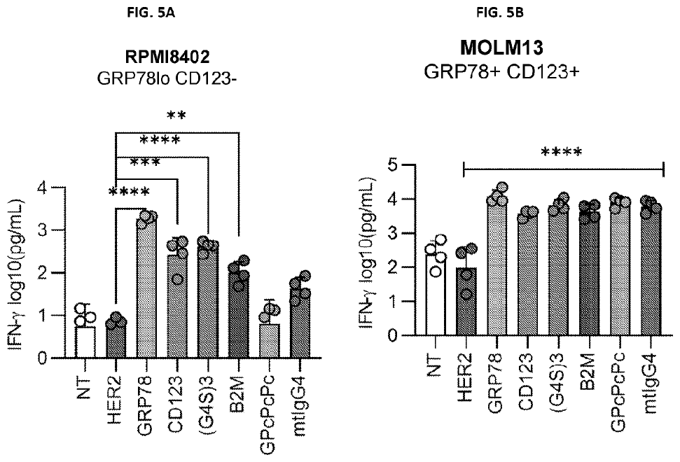

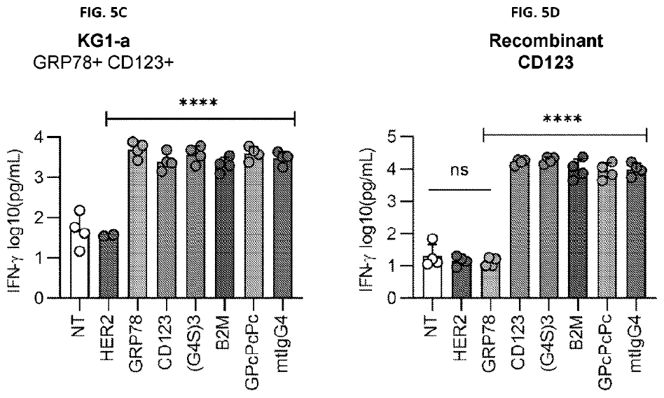

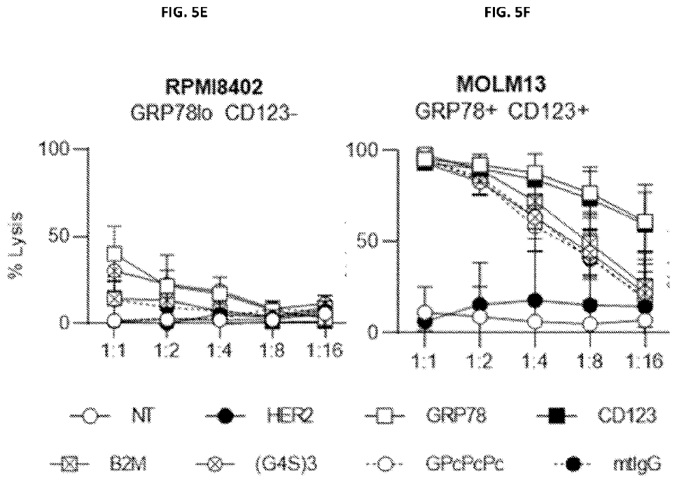

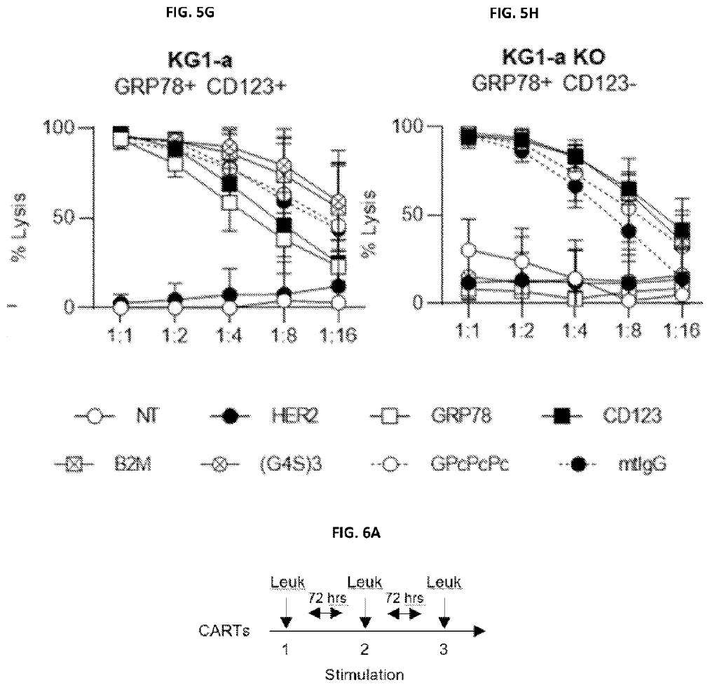

Cytokine secretion was determined to confirm antigen specificity and co-cultured GRP78−, CD123−, β2M−, (G4S)3−, GPcPcPc−, mtIgG4− CAR T cells with KG1a, MOLM13, or recombinant CD123 protein at an effector:target (E:T) ratio of 2:1. Non-transduced (NT) and HER2-CAR T cells served as negative controls. IFN-γ or IL-2 concentrations in culture media were measured by enzyme-linked immunosorbent assay (ELISA) after 24 hours in culture. When T cells were cocultured with KG1a or MOLM13 cells (GRP78+/CD123+), monospecific and bispecific CAR T cells secreted significantly more IFN-γ and IL-2 compared to controls, with no significant differences between the mono- vs bispecific (FIGS. 4F-4H, 5A-5C). When CAR T cells were cocultured in the presence of recombinant CD123 protein, GRP78 CAR T cells did not secrete IFN-γ or IL-2 similar to NT and HER2 CAR T cells. There was no significant difference in cytokine secretion between CD123-CAR T cells and 78.123-bispecific CAR T cells but there was significant secretion compared to controls (FIGS. 4I, 5D).

To confirm the above findings, anti-tumor activity was measured after a 24-hour coculture assay at E:T ratios of 1:1, 1:2, 1:4, 1:8, and 1:16. In the presence of GRP78 and absent CD123 antigen expression in RPMI8402 cells, CAR T cells were not active against target cells (FIG. 5E). When both antigens were present at high levels, MOLM13, and low levels, KG1a, all CARs had statistically significant antitumor activity compared to control NT or HER2 CAR T cells (FIG. 5F-5G). However, in the absence of CD123 antigen in the KG1a KO target cells, GPcPcPc bispecific CAR did not have antitumor activity, suggesting inability to recognize GRP78 antigen. (FIG. 5H).

### Example 3. 78.123-CAR T Cells Sequentially Kill Leukemia Cells

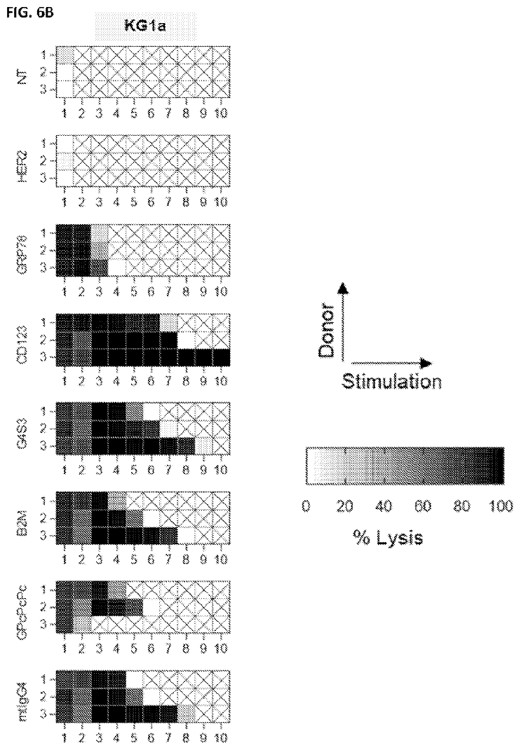

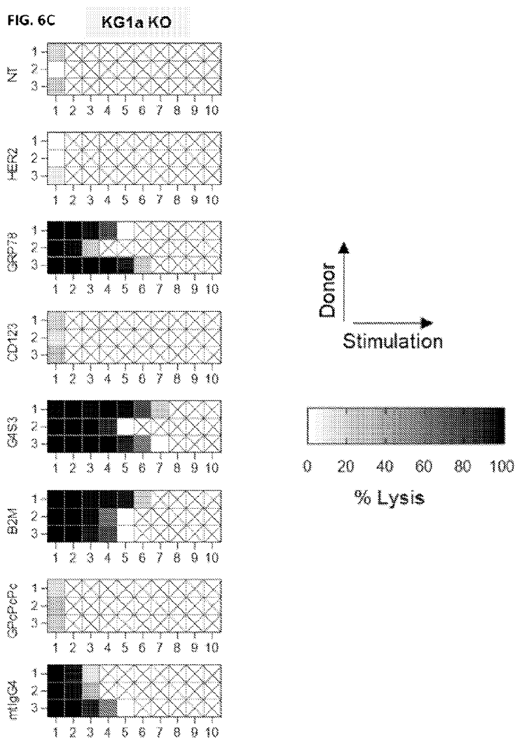

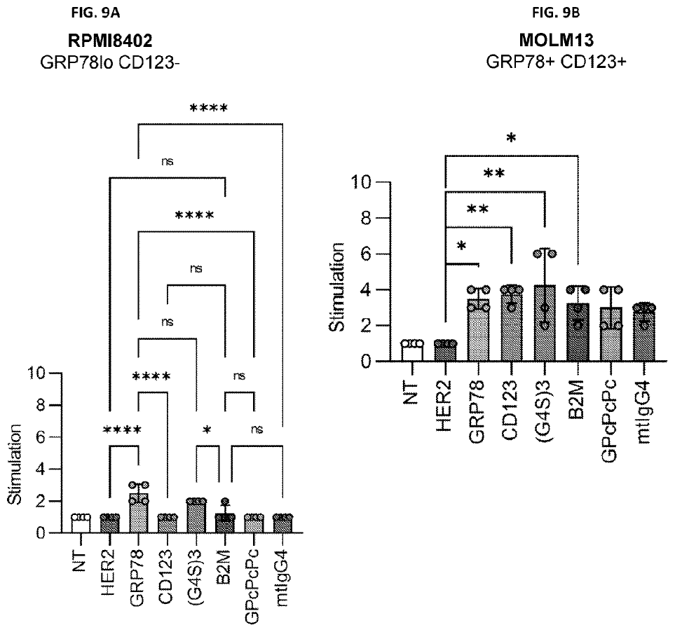

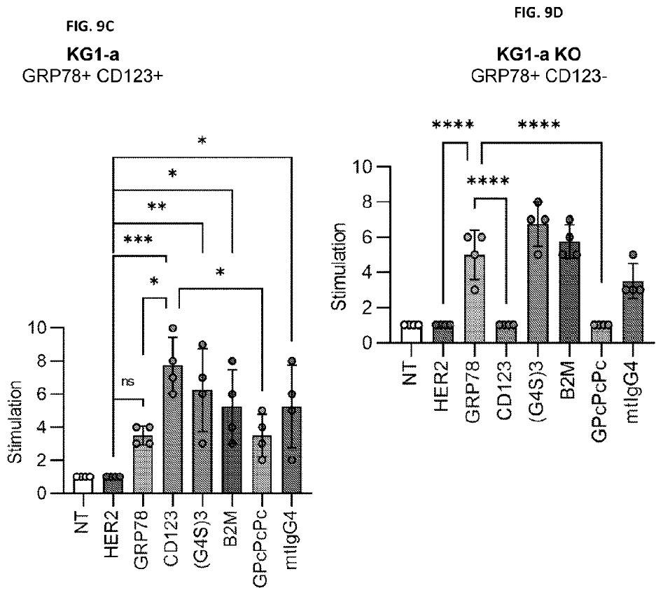

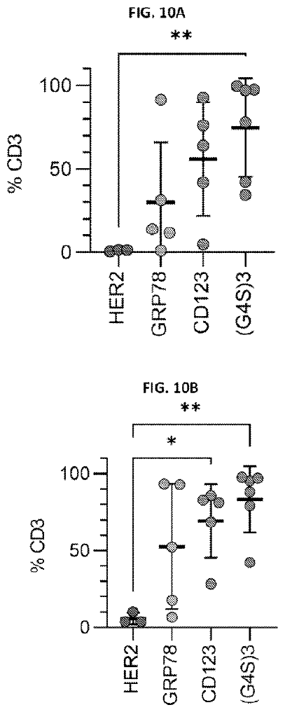

To determine the capability of bispecific CAR T cells to repeatedly kill tumor cells in a sequential stimulation assay that mimics chronic antigen exposure, a serial stimulation assay was used. Every 72 hours, CAR T cells were challenged at a 1:1 E:T ratio (FIG. 6A, FIG. 9A-9D). In repeat stimulations against the KG1a (CD123+, GRP78+) target cell line, the CD123 CAR outperformed the GRP78 CAR up to 6 stimulations (FIG. 6B, 9C). mtIgG4, B2M and G4S3 bispecific CARs were able to repeatedly kill between 3-9 times against the KG1a cell line. The KG1a KO cell line, the bispecific CAR with the GPcPcPc linker, did not respond to the GRP78 antigen. The mtIgG4 did not respond as well to the KG1a KO cell line compared to the GRP78 CAR, or bispecific B2M or G4S3 CARs (FIG. 6C, 9D). When CAR T cells were repeatedly exposed to the MOLM13 cell line (CD123+, GRP78+, high antigen expression), the monospecific and bispecific CARs only sequentially killed between 4-6 times, whereas against the KG1a cell line, CAR sequentially killed 4-10 times (FIG. 9B). Of note, in a setting mimicking antigen escape and where CD123 antigen expression was lacking (KG1aKO), bispecific CARs (G4S2, B2M, mIgG4) outperformed single specificity CD123-CAR, with B2M and G4S3 also lasting for more stimulations than single specificity GRP78-CARs (FIG. 6B-6C, 9D). In repeat stimulations against the KG1a KO cell line, the bispecific CAR with the GPcPcPc linker did not respond to the GRP78 antigen. In KG1a target cell line repeat stimulation assays the CD123 CAR outperformed the GRP78 CAR up to 6 stimulations (FIG. 6B-6C). mtIgG4, β2M and (G4S)3 bispecific CARs were able to repeatedly kill between 3-9 times against the KG1a cell line. When CAR T cells were repeatedly exposed to the MOLM13 cell line (high antigen expression), the monospecific and bispecific CARs only sequentially kill between 4-6 times whereas against the KG1a cell line, the CARs sequentially killed 4-10 times (FIG. 6B-6C).

### Example 4. 78.123-CAR T Cells have Potent Anti-AML Activity In Vivo

The anti-AML activity of mono- and bispecific CAR T cells was evaluated in vivo using an established MOLM13.GFP.ffluc xenograft model. Due to previous in vitro data suggesting the GPcPcPc did not respond to GRP78, the HER2−, GRP78−, CD123− CARs and bispecific β2M, (G4S)3, and mtIgG4 CAR T cells were tested. Mice received 5×10³ MOLM13.GFP.ffluc cells intravenously (i.v.) and 7 days later received a single infusion of 3×10⁶ CAR T cells.

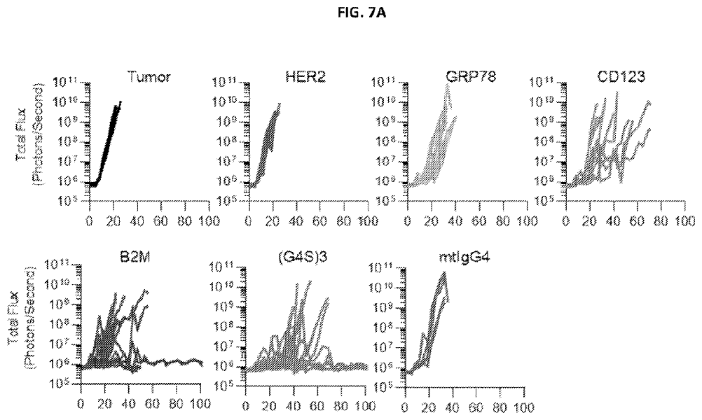

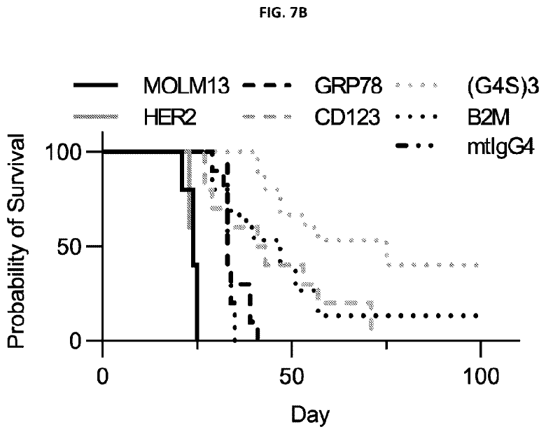

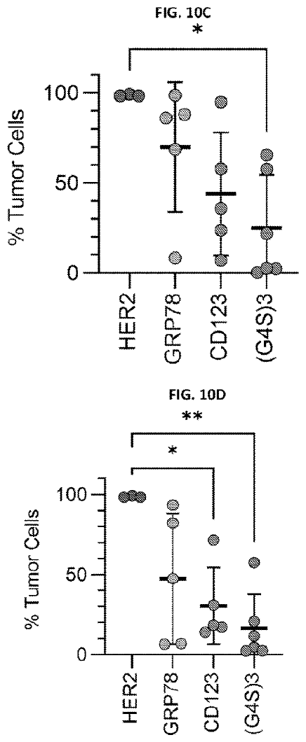

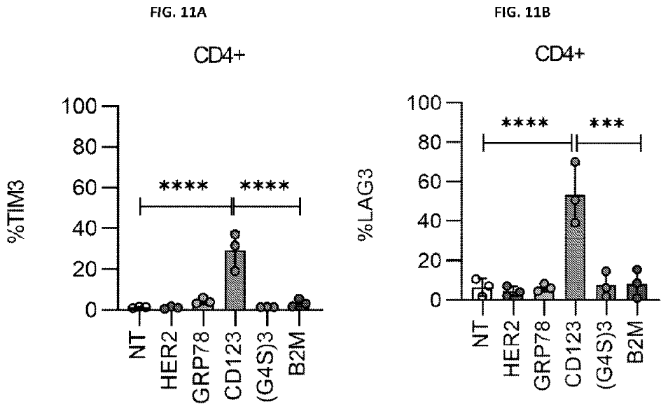

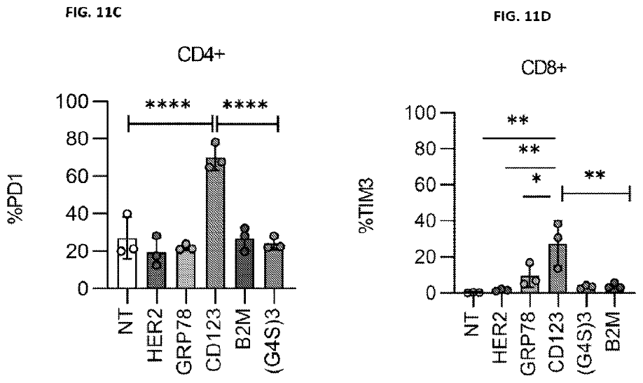

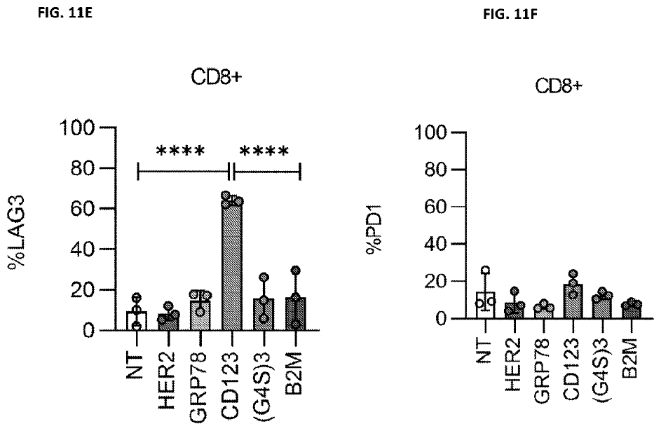

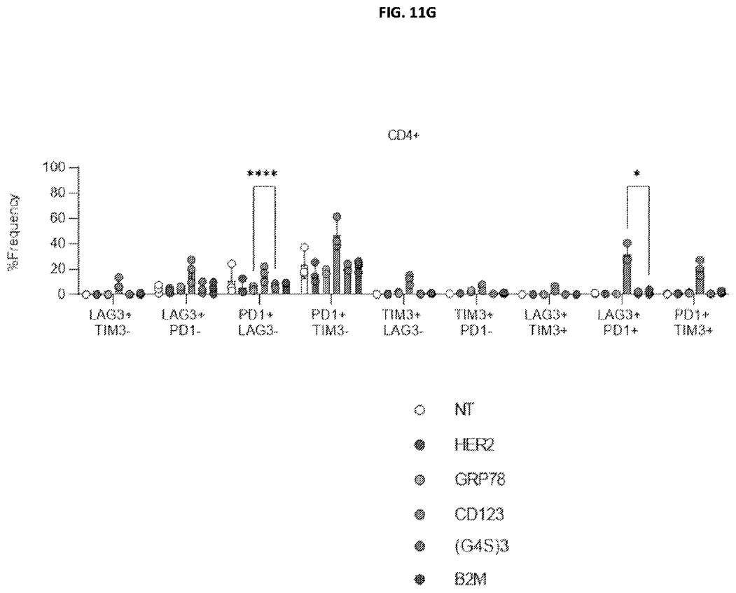

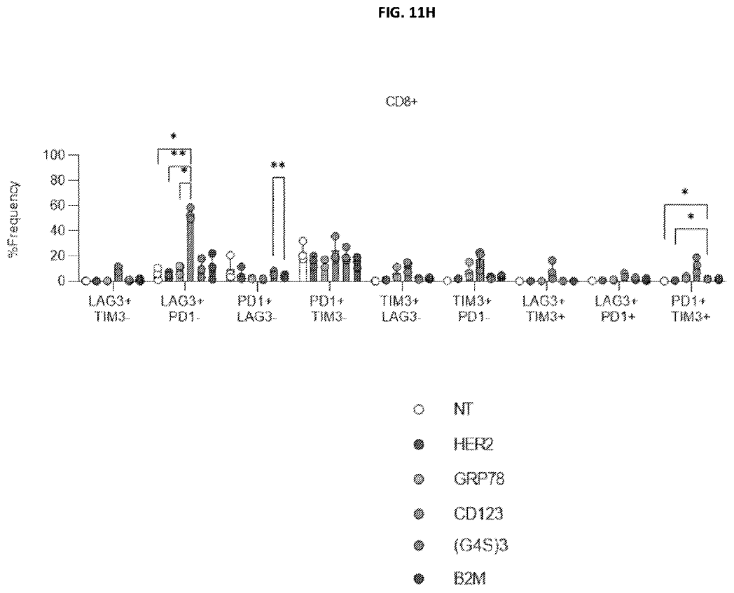

Compared to tumor only control or HER2 treated mice, all animals had a significant reduction in tumor burden and survival advantage (FIGS. 7A-7B, p<0.001, N=5-15). Leukemia recurred in all mice treated with monospecific-CAR T cells, but 7/15 (G4S)3- and 6/15 β2M-sustained complete responses. Graft-versus-host disease (GvHD) was observed in 5/15 mice treated with β2M and 2/15 treated with (G4S)3 CAR T cells. Mice treated with GRP78 (N=3), CD123 (N=8), (G4S)3 (N=5), or β2M (N=6) recurred with extramedullary chloromas between days 25-57. These data demonstrate that in presence of both antigens at higher densities, the bispecific 78.123 CAR performs better than the GRP78− or CD123− only CAR. At the endpoint, bone marrow and spleen were harvested from HER2, GRP78, CD123, or G4S3 mice to measure tumor burden and T cell infiltrates (FIG. 10A-10D). Mice treated with G4S3 bispecific CAR T cells had significantly less tumor and more T cells at endpoint compared to HER2 controls. To better understand the phenotype of the 78.123 CAR T cells, the inventors measured TIM3+, PD1+, and LAG3+ expression, and found significant differences in checkpoint markers between bispecific CAR T cells and the CD123 monospecific CAR T cells (FIG. 11A-11H).

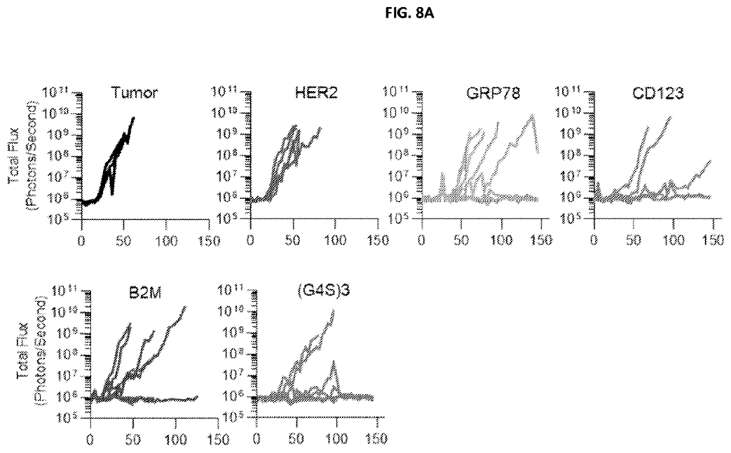

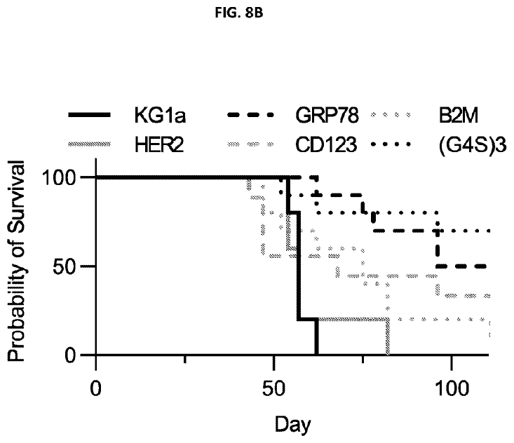

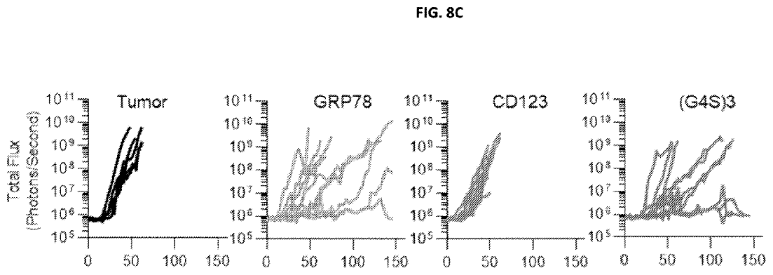

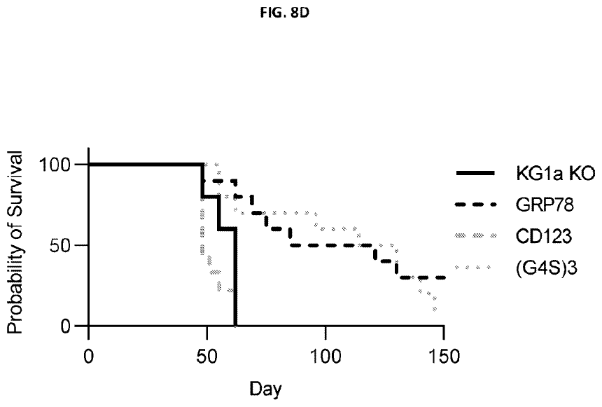

The β2M and (G4S)3 in a KG1a model were next tested to see how the bispecific 78.123 CAR T cells would respond to low antigen expression, particularly CD123. Mice received 1×10⁶ KG1a.GFP.ffluc cells intravenously, and 7 days later received a single infusion of 3×10⁶ CAR T cells. Compared to tumor only and HER2 treated-mice, GRP78 CAR and (G4S)3 tumor control resulted in survival advantage (FIGS. 8A-8B, N=5-10). This result demonstrates that the bispecific 78.123 CAR outperforms the CD123-CAR.

To confirm the single antigen specificity of β2M and (G4S)3 bispecific CAR T cells in vivo, both were tested in a KG1a KO.GFP.ffluc xenograft model. Mice received 3×10⁶ KG1a.KO.GFP.ffluc cells intravenously and 7 days later received a single infusion of 3×10⁶ CAR T cells. Mice treated with β2M had transient disease control but 60% succumbed to non-tumor related morbidities such as GvHD. Mice treated with GRP78 and (G4S)3 CAR T cells have demonstrated antitumor activity thus far (FIGS. 8C-8D).

### Example 5. Generating 78.B7H3 Bispecific CAR T Cells with Different Hinge Domains

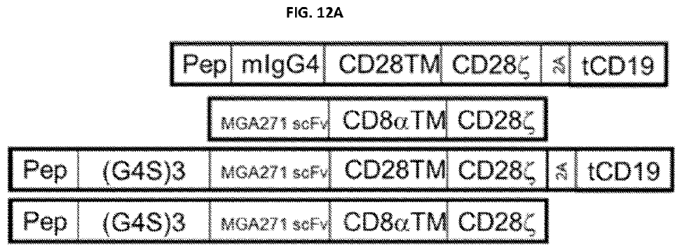

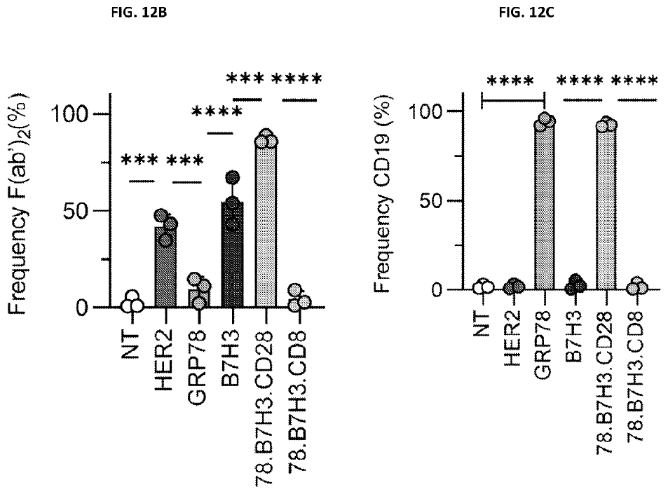

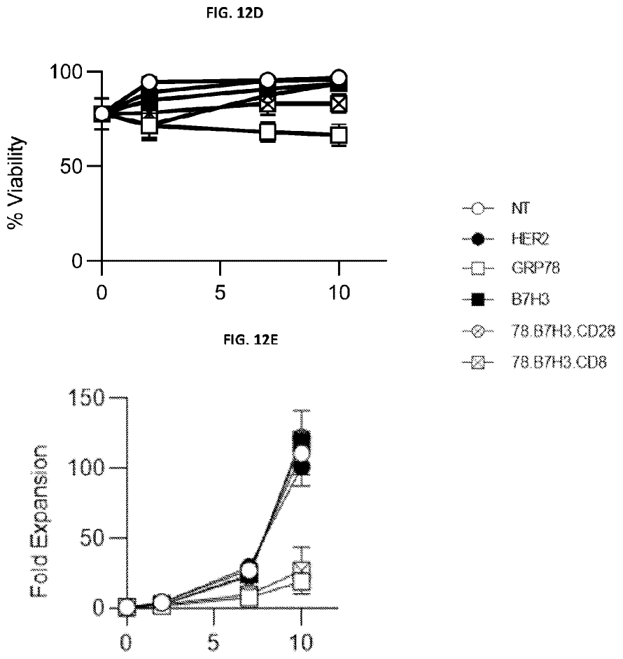

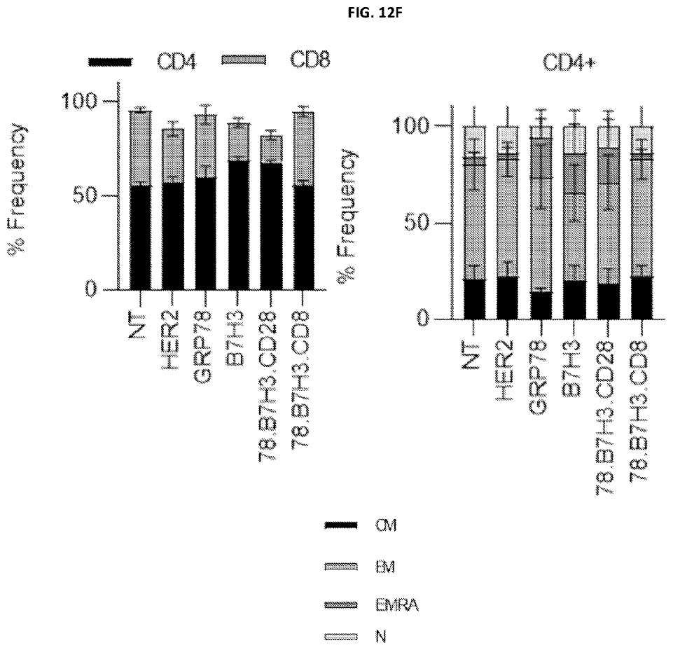

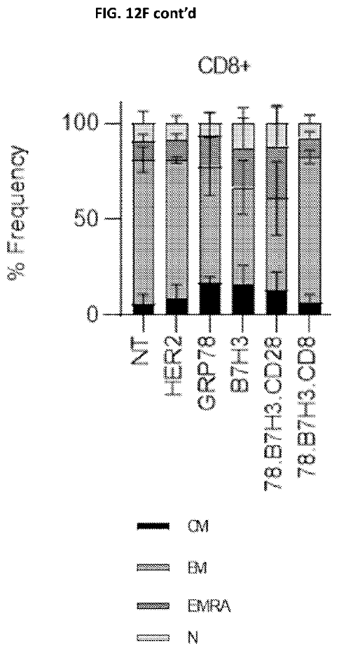

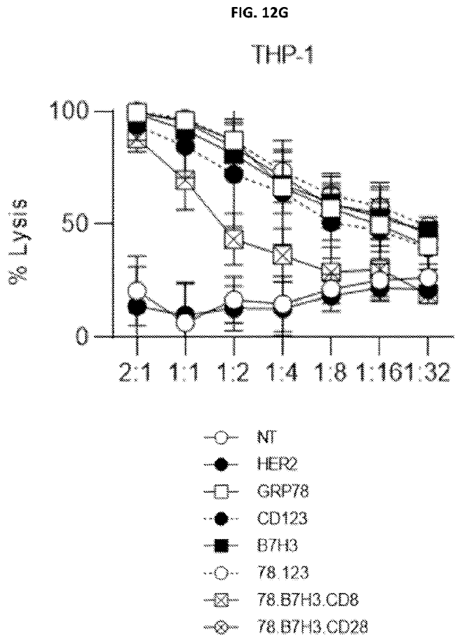

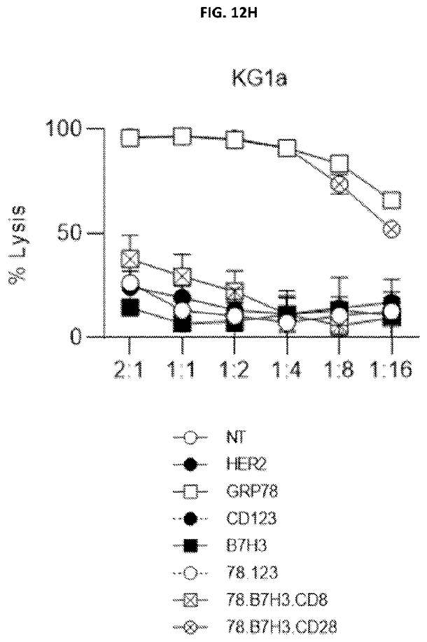

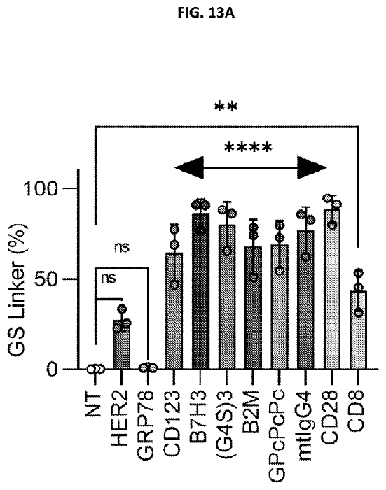

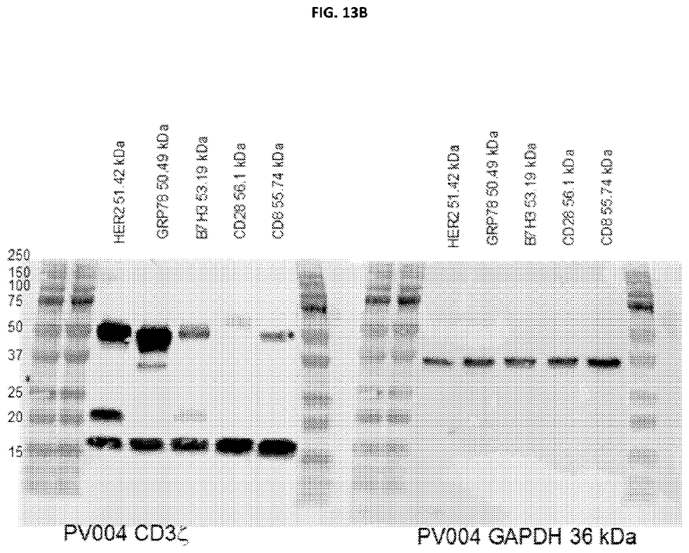

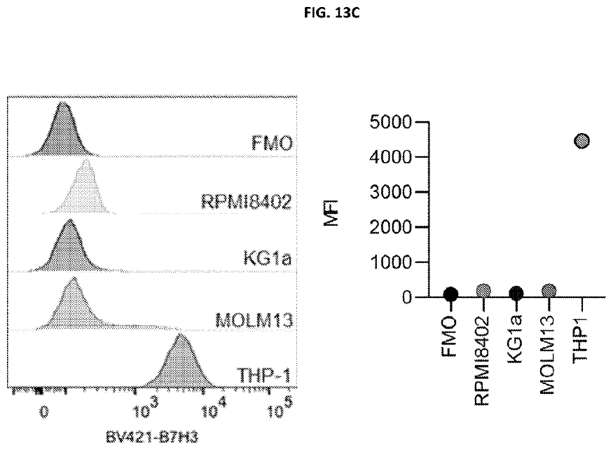

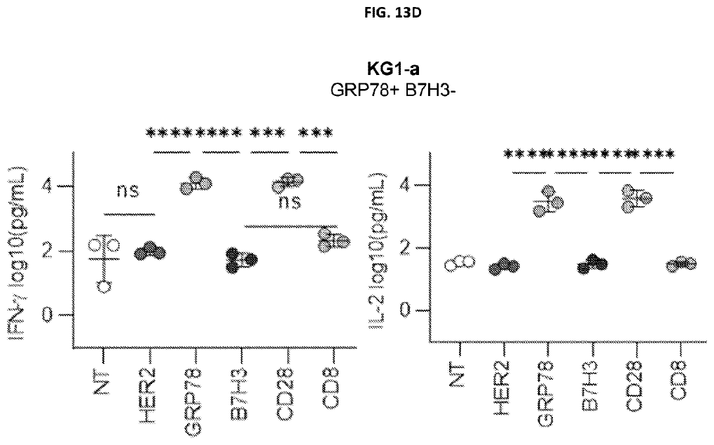

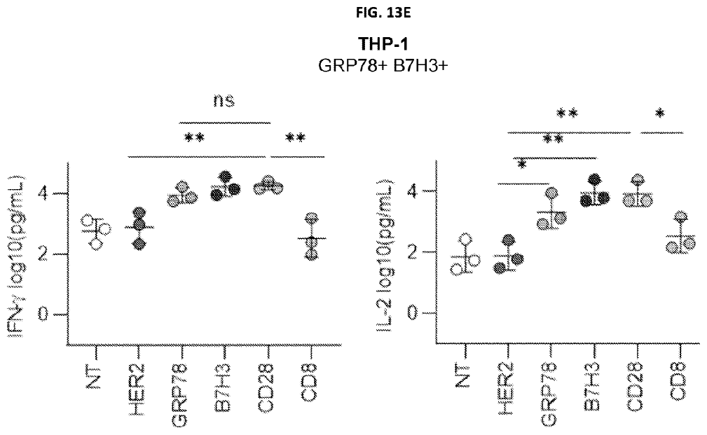

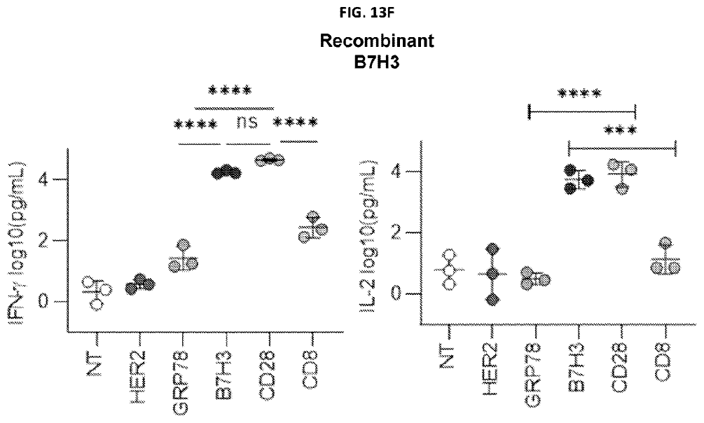

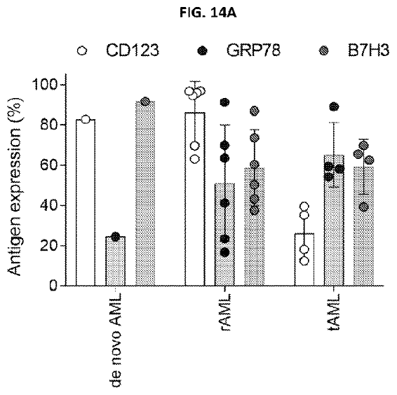

To test whether this bispecific approach could be extrapolated to other scFvs, the inventors designed a CAR consisting of a GRP78-specific peptide and B7H3-scFv binding domain (78.B7H3), using a G4S linker between peptide-scFv. Two different hinge/transmembrane domains, CD28 or CD8α H/TM, were tested (CD28, CD8 respectively, FIG. 12A). Transduction efficiency was measured by F(ab)₂, CD19, and GS linker detection (FIG. 12B-12C, FIG. 13A). Bispecific CAR constructs with the CD8 hinge were not expressed at the cell surface as well as the CD28 hinge constructs. However, the CAR protein was being efficiently made as indicated by Western blot analysis (FIG. 13B).

While GRP78-CAR had a similar decrease in viability and expansion when compared to CD8 hinge CAR T cells, the inventors did not see as pronounced differences in the CD28 construct compared to controls (FIGS. 12D-12E). The immunophenotype of the T cells suggests that there are more CD4+ T cells in B7H3 monospecific and CD28 bispecific CAR T cells compared to controls (FIG. 12F). However, when effector memory (EM: CCR7−, CD45RO+), central memory, Naïve-like, and effector memory RA were evaluated, significant differences in the CD4+EM between GRP78 and B7H3 as well as B7H3 and CD28 were found (FIG. 12F). The CD8 bispecific CAR demonstrated differences in CD4 Naïve-like and CD8+ central memory T cells compared to monospecific CAR T cells.

### Example 6. 78.B7H3 CD28 Bispecific CAR T Cells Elicit Antigen Specific Cytotoxicity

AML cell lines were analyzed for surface B7H3 expression to measure effector function by the 78.B7H3 CAR T cells. THP-1 expressed both antigens by percentage and mean fluorescence intensity (MFI) (FIG. 13C-13D). KG1a cells (GRP78+, B7H3−) were used as a B7H3 negative control.

To ensure antigen specificity, IFN-γ and IL-2 secretion were measured by ELISA when NT, HER2, GRP78, B7H3, CD28, or CD8 CAR T cells were cocultured with KG1a (GRP78+/B7H3−), THP-1 (GRP78+/B7H3+) or recombinant B7H3 protein (FIG. 13D-13F). The bispecific CD28 CAR elicited robust secretion of IFN-γ and IL-2 at an effector:target (E:T) ratio of 2:1 in the presence of one or both antigens, whereas the CD8 bispecific CAR did not result in a strong cytokine response.

Next, the cytotoxicity of the target cell lines was measured after a 24-hour coculture assay at E:T ratios of 2:1 1:1, 1:2, 1:4, 1:8, 1:16, and 1:32. In the presence of both antigens, GRP78, B7H3, and CD28 CAR T cells resulted in a statistically significant difference in tumor lysis (FIG. 12G). The CD8 bispecific CAR did not have anti-leukemia activity when cultured in the presence of both antigens that was statistically significant. In the presence of GRP78 only, the single specificity B7-H3 CAR did not recognize the tumor cells, while the bispecific CD28 CAR T cells maintained antitumor activity, suggesting that the bispecific CD28 CAR T cells were able to engage with both antigens (FIG. 12H). The CD28 hinge was the only design that resulted in robust cytolytic activity when stimulated with either GRP78, B7H3, or both. When the 78.B7H3 CAR T cells were cocultured with THP-1 cells, there was significant secretion of IFN-γ and IL2 and target cell death in comparison to controls.

### Example 7. GRP78, CD123, and B7H3 are Expressed on AML Primary and PDX Samples

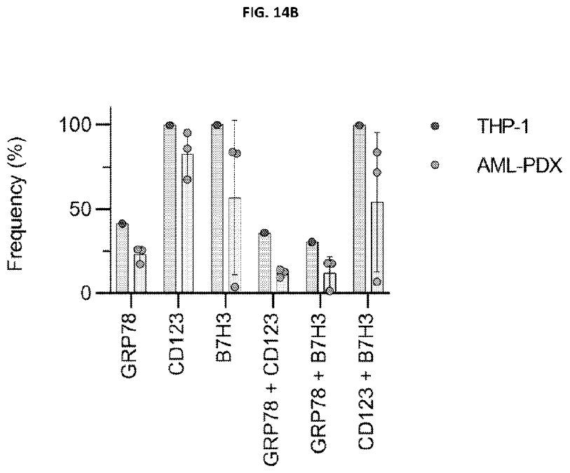

To ensure the feasibility of targeting GRP78, CD123, and B7H3 antigens in a bispecific approach for AML, cell surface antigen expression was measured on de novo, relapsed and primary AML (FIG. 14A). All three antigens are expressed in primary samples to variable degrees with expression ranging from 12.5-96.9% for CD123, 16.7-91.3% GRP78, and 37.5-91.7% in B7H3, highly suggestive of a heterogeneous disease with multiple targeted interventions necessary. Next, single and double antigen expression was measured on 3 PDX-derived cell lines compared to antigen density on the THP-1 cell line. The inventors found that expression was also highly variable but consistent with the tumor cell line (FIG. 14B). Taken together, these data highlight the need for dual-antigen targeting and the use of GRP78, CD123, and B7H3 bispecific CAR T cells.

The above-described Examples explored bispecific targeting of CD123, a target with expression on leukemic blasts and leukemia stem cells,²⁵ GRP78, a target that has the potential for broad applicability and limited on-target off-tumor side effects, and B7H3, a coreceptor belonging to the B7 family of immune checkpoint molecules expressed on several malignancies, including solid tumors and leukemic blasts but not on normal hematopoietic stem cells (HSCs). Two novel bispecific CAR structures were established by combining a peptide and an scFv, simultaneously targeting two antigens (either GRP78 and CD123, or GRP78 and B7H3) widely expressed on AML blasts, and thereby providing a strategy to circumvent immune escape.

CAR configuration is essential to the functioning and anti-tumor activity. Each domain of a CAR has a distinct purpose and must provide sufficient stability, flexibility, and function. Previously described scFv-scFv approaches have shown that certain configurations can impact antigen accessibility to both binding domains rendering these tandem CARs less effective in targeting more than one antigen.²⁶

The in vitro and in vivo data of the present disclosure supports that the linker length and complexity contributed to optimal bispecific CAR functioning. This analysis was achieved by pursuing a peptide-scFv based approach to dual antigen targeting. The Examples herein demonstrated the ability of the bispecific CARs described herein to actively engage both GRP78 antigen and CD123 antigen, as well as GRP78 antigen and B7H3 antigen, with the bispecific antigen binding domains disclosed herein, allowing for dual specificity. While most scFvs separate the variable and light chains by a glycine-serine linker, it has also been the most widely used linker for bispecific CARs combining two scFvs. The systematic approach applied in the present Examples verified the advantage of using a flexible linker for engagement of both a 13-mer peptide and scFv. In addition, utilizing a peptide and an scFv in tandem to achieve dual antigen specificity may bypass structural and spatial interferences encountered when using two different scFvs. Previous work by Qin et al., showed that when combining the CD19 and CD22 scFvs for B cell leukemia treatment, not all configurations gave access to both binding domains via recombinant protein, rendering these tandem CARs ineffective.²⁶ These data highlight the importance of CAR design, and the complicated nature of bispecific antigen binding domains.

There was no significant difference observed in vitro between bispecific CAR engagement between GRP78 or CD123, or GRP78 and B7H3, and the bispecific CARs functioned in an OR gate style. OR gate CARs can provide a promising strategy for dual targeting. The ability to recognize both antigens may be important to overcome AML heterogeneity and tumor immune escape, which is what the approach disclosed in the present Examples was designed to overcome, while mitigating toxicity due to the antigens common to leukemic blasts and normal tissues. There is an ongoing clinical trial analyzing a compound CAR against AML antigens CD33 and CLL-1 (NCT03795779) that has documented cases of minimal residual disease negative remission.²⁷ In addition, a bispecific and split CAR (BissCAR) targeting CD13 and TIM3 has shown promising preclinical results for anti-AML activity.²⁸ These data support pursuit of a dual-targeted CAR therapy approach for AML.

The above-described Examples demonstrated that targeting two different antigens using a peptide and a scFV antigen recognition domain is feasible. A (G4S)3-linker in between antigen recognition domains preserved specificity to GRP78 and CD123.

Below are the methods used in the Examples described above.

Cell lines and culture methods. The following cell lines were procured from American Type Culture Collection (ATCC, Manassas, VA): 293T, KG1a, and THP-1 cell lines. The RPMI8402 and MOLM13 cell line was purchased from Leibniz Institute (DSMZ, German Collection of Microorganisms and Cell Cultures, Braunschweig, Germany). All guide RNAs for CD123 knockout were designed and validated by the Center for Advanced Genome Engineering (CAGE) at St. Jude Children's Research Hospital. sgRNAs were designed to target unique sites within the genome with at least 3 base pairs (bp) of mismatch between the target site and any other site in the genome whenever possible, and common single-nucleotide polymorphisms were avoided. Cells were subsequently sorted and expanded to generate a KG1a KO cell line. MOLM13, KG1a, and KG1a KO expressing an enhanced green fluorescence protein/firefly luciferase fusion protein (GFP.ffluc) were generated as previously reported.²³ The LM7 and LM7 B7H3 KO osteosarcoma cell lines were kindly provided by the DeRenzo lab.³⁴ Cell lines were cultured in RPMI 1640 (ThermoFisher Scientific) or DMEM (GE Life Sciences) and grown in humidified incubators at 37° C. and 5% COR₂R. All culture media was supplemented with 10% Fetal Bovine Serum (Thermo Scientific) and GlutaMAX (2 mmol/L; Invitrogen, Carlsbad, CA). Cell lines were authenticated using the ATCC's human STR profiling cell authentication service and routinely checked for Mycoplasma by the MycoAlert Mycoplasma Detection Kit (Lonza). Three patient-derived xenograft (PDX) cell lines were established by intravenous (i.v.) injection of pediatric primary AML samples into NSG-S mice and subsequently propagated in vivo.³⁵ These cells were used for flow cytometry to evaluate antigen status.

Generation of retroviral vectors. The GRP78 CAR synthesized cDNAs (GeneArt, ThermoFisher Scientific, Waltham, MA) encoding the IgG heavy chain leader sequence and one copy of the GRP78-specific peptide CTVALPGGYVRVC (SEQ ID NO: 92).²⁴ These were subcloned into a pSFG retroviral vector that encoded a mutant IgG4 hinge, a CD28 transmembrane domain, a CD28.CD3z signaling domain, a T2A ribosomal skip sequence and truncated CD19 (tCD19) to enable detection of transduced cells. The pSFG retroviral CD123 CAR was generated by Takara's infusion cloning of the previously described CD20 T2A CD123 CAR in lentiviral backbone. Linker sequences with the CD123 scFv were synthesized via IDT. Each construct was engineered using infusion cloning to be inserted after the GRP78 peptide. The generation of control-CARs (HER2-CAR.CD28.CD3ζ) have been previously reported.^{36,37} The B7H3.CD8α.CD28ζ CAR has been previously reported in a lentiviral backbone and since subcloned into a pSFG retroviral vector.³⁴ The sequence of all cloned constructs was confirmed by sequencing performed by Hartwell Center DNA Sequencing Core at St. Jude Children's Research Hospital with Big Dye® Terminator (v3.1) Chemistry on Applied Biosystems 3730XL DNA Analyzers (Thermo Fisher Scientific, Waltham). RD114-pseudotyped retroviral particles were generated as previously described. Retroviral transduction was performed as previously described.

Generation of CAR T cells. All methods involving human subjects were carried out in accordance with the Declaration of Helsinki. Human peripheral blood mononuclear cells (PBMCs) from healthy donors were obtained under a St. Jude Children's Research Hospital (St. Jude) IRB approved protocol, after acquiring informed consent. PBMCs were stimulated on CD3 (1 μg/mL, Miltenyi Biotec, Bergisch Gladbach, Germany) and CD28 (1 μg/mL, Miltenyi Biotec, Germany) antibody-coated, non-tissue culture treated 24-well plates (Corning, Corning, NY). Human interleukin (IL) 7 (10 ng/mL, Peprotech, Rocky Hill, NJ) and IL-15 (5 ng/mL, Peprotech) were added to cultures on day 2. On day 3, T cells were transduced with retroviral particles on RetroNectin (Takara Bio USA, Mountainview CA) coated plates in the presence IL-7 (10 ng/mL) and IL-15 (5 ng/mL). T cells were subsequently expanded with IL-7 and IL-15. Non-transduced (NT) T cells were activated with CD3/CD28 antibodies and expanded in parallel with IL-7 and IL-15. Following expansion for 5-7 days the transduced cells were analyzed for CAR expression using flow cytometry and subsequently used for functional assays.

Flow cytometric analysis. Cells were stained with fluorochrome-conjugated primary antibodies for 30 min at room temperature and washed with FACS buffer (5% FBS in 1×PBS) prior to analysis. Cell surface GRP78 was detected by a GRP78-specific peptide with an N-terminal Biotin tag (Biotin-Ahx-CTVALPGGYVRVC; SEQ ID NO: 126) was obtained from Genscript (Piscataway, NJ) in combination with Streptavidin PE (BioLegend, San Diego, CA. CCAT #405204) using a 2-step staining protocol. Cells were incubated with GRP78 peptide for 30 minutes at room temperature, protected from light. Cells were washed twice prior to staining with additional antibodies. Recombinant CD123 Recombinant Human IL3RA protein conjugated to APC (Creative BioMart, CAT #IL3RA-3248HA) was used to detect CD123 scFv binding. The following antibodies were purchased from BD Biosciences: CCR7 Pacific Blue (BD Pharmigen, Clone G043H7 CAT #353210), CD45RO PerCP-Cy5.5 (BD Pharmigen, Clone UCHL1, CAT #560607), CD123 APC (BD Pharmigen, Clone 7G3, CAT #560087), CD3 BV421 (BD Horizon, Clone SK7, CAT #563797), CD3 BV786 (BD Horizon, Clone SK7, CAT #563800), CD19 PE-Cy7 (BD Pharmigen, Clone HIB19, CAT #560728), CD20 BV650 (BD Horizon, Clone 2H7, CAT #563780). Other antibodies included: DAPI, Live/Dead Fixable Aqua Dead Cell Stain Kit (Invitrogen, Cat #L34957); eFluor780 (Thermo Fischer, CAT #65-0865-15), CD8 APC-H7 (Biolegend, Clone SK1, CAT #560179), CD4 Alexa Fluor 700 (Biolegend, Clone SK3, CAT #344622), CD45 APC-Cy7 (Biolegend, Clone 2D1, 368516), CD19 PE (Beckman Coulter, CAT #IM1285U). Cells were washed and filtered after staining with 1×PBS+5% FBS. All samples were acquired on FACS Canto II, Lyric instruments, or LSRFortessa (BD Biosciences). The analysis was performed using FlowJo 10.5.3 software (BD Biosciences).

Cytotoxicity assays. To determine the cytotoxic potential of the CAR T cells flow cytometry-based and luciferase-based cytotoxicity assays were used. In flow cytometry-based cytotoxicity assays, target cells RPI8402, KG1a, and KG1a KO were stained with CFSE per manufacturer protocol (Cayman Chemical, Ann Arbour, MI) for 30 minutes and washed in complete media. In 96-well round bottom plates, 5×10⁴ target cells were plated in each well. NT or CAR T cells were co-cultured at effector to target ratios of 2:1, 1:1, 1:2, 1:4, 1:8, and 1:16. Cells were incubated for 24 hours in a cell culture incubator. After 24 hours, plates were centrifuged at 2000 g for 1 minute and liquid was removed. Cells were resuspended in 200 uL from a stock of PBS+5% FBS+15 μL of Count Bright™ Absolute Counting Beads per mL (Invitrogen, Walthman, MA, CAT #C36950). 100 bead events were collected per well and cytotoxicity was determined by the absolute count of remaining CFSE+ target cells compared to controls. For luciferase-based cytotoxicity assays, NT or CAR T cells were co-cultured with 5×10⁴ GFP.ffluc MOLM13 target cells or GFP.ffluc THP-1 target cells at the same E:T ratios in a 96-well tissue culture plates overnight. After 24 hours, plates were centrifuged at 2000 g for 1 minute and liquid was removed. In the luciferase-based assay, 100 μL of MOLM13.GFPffluc cells were incubated with D-Luciferin. Luminescence was measured on a Tecan Infinite® 200 (Life Sciences-Tecan, Mannedorf, Switzerland) and analyzed using Magellan Software (Life Sciences-Tecan).

Cytokine ELISA. RPMI8402, KG1a, MOLM13, and recombinant CD123 protein (1 ug/well, R&D, Minneapolis, MN) were co-cultured with effector cells at a 2:1 E:T ratio. NT, HER2-CAR, GRP78-CAR, CD123, CAR and bispecific 78.123 CARs or 78.B7H3 CARs were used as effector T cells. T cells were incubated with antigen for 24 hours, supernatants were collected, and IFN-γ and IL-2 levels were determined using ELISAs (R&D Systems) as per the manufacturer's protocols.

Repeat Stimulation. 5×10⁵ effector T cells were plated at a 1:1 effector to target (E:T) ratio with RPMI8402, KG1a, KG1a KO, MOLM13, or THP-1 target cells. All target cells expressed GFP.ffluc. Three days later, antitumor activity was determined by flow-based cytotoxicity assay or luciferase-based assay. For flow-based cytotoxicity assay, a similar protocol was followed to the basic 24-hour cytotoxicity assay however, prior to the addition of Counting Beads, cells were stained for 30 minutes using eFluor780 (1:1000 dilution, Thermo Fischer, CAT #65-0865-15) and CD3 BV421 (BD Horizon, Clone SK7, CAT #563797). Cells were washed, and counting beads were added for analysis. In conditions where there was greater than 50% tumor lysis, fresh 5×10⁵ tumor cells were added.

In vivo studies. All in vivo studies were carried out following protocols approved by the Institutional Animal Care and Use Committee in accordance with the American Association for Laboratory Animal Science at St. Jude. The studies were performed using NSG (NOD.Cg-Prkdcscid/Il2rgtm1Wjl/SzJ) mice obtained from St. Jude's in-house breeding colony. 5×10³ MOLM13.GFP.ffluc cells, 1×10⁶ KG1a.GFP.ffluc, or 3×10⁶ KG1aKO.GFP.ffluc, were injected intravenously (i.v.) by tail vein injection. 3×10⁶ total T cells were injected 7 days later. Tumor growth was monitored by twice weekly bioluminescence imaging using an IVIS®-200 imaging system (IVIS, Xenogen Corp., Alameda, CA) as previously described. Mice were euthanized at predefined endpoints or when they met euthanasia criteria in accordance with St. Jude's Animal Resource Center.

Western Blot. Cells were lysed using RIPA buffer and Halt Protease and Phosphatase Inhibitor Cocktail (CAT #78440, Thermo Scientific). Cell lysates were centrifuged at 2000 g for 10 minutes at 4C and supernatant was stored at −80C. Whole cell lysates were quantified using Pierce BCA Protein Assay Kit (CAT #23227, Thermo Scientific). The samples were boiled for 10 minutes in 4× Laemmli Sample buffer (CAT #1610747, Bio-Rad Laboratories) containing B-mercaptoethanol. SDS Page was performed using Mini-PROTEAN® TGX™ Precast Gels and a Mini-PROTEAN Tetra Cell system (Bio-Rad laboratories). The proteins were transferred to a PVDF membrane (Millipore) and probed with primary antibodies at 1:1000 dilution (CD3z Clone-6B10.2: CAT #sc-1239; GAPDH Clone 6C5: Cat. No. sc-32233, Santa Cruz Biotechnology). The blots were developed using Clarity Western ECL Blotting Substrate (CAT #1705060, Bio-Rad Laboratories) and imaged on the Odyssey® Fc Imaging System from LI-COR Biosciences and LI-COR Image Studio™ software version 5.2.

Statistical analysis. Descriptive statistics were calculated for all outcomes. The one- or two-factor ANOVA test was used to examine overall differences in outcomes between multiple constructs. (Comparisons across multiple groups were performed by one- or two-factor ANOVA when appropriate.) The overall test was followed by pairwise comparisons using t-test when appropriate (i.e., overall test p<0.05). Generalized linear model was used to access the overall difference in outcomes with repeated measurements to account for intra subject correlation in each subject/donor. Log rank test was used to test difference between constructs of all survival outcomes. Statistical analyses were conducted with GraphPad Prism.

### REFERENCES

1 Bouchkouj, N. et al. FDA Approval Summary: Axicabtagene Ciloleucel for Relapsed or Refractory Large B-cell Lymphoma. Clin Cancer Res 25, 1702-1708, doi:10.1158/1078-0432.CCR-18-2743 (2019).
2 O'Leary, M. C. et al. FDA Approval Summary: Tisagenlecleucel for Treatment of Patients with Relapsed or Refractory B-cell Precursor Acute Lymphoblastic Leukemia. Clin Cancer Res 25, 1142-1146, doi:10.1158/1078-0432.CCR-18-2035 (2019).
3 Jen, E. Y. et al. FDA Approval: Blinatumomab for Patients with B-cell Precursor Acute Lymphoblastic Leukemia in Morphologic Remission with Minimal Residual Disease. Clin Cancer Res 25, 473-477, doi:10.1158/1078-0432.CCR-18-2337 (2019).
4 Ding, L. et al. Clonal evolution in relapsed acute myeloid leukaemia revealed by whole-genome sequencing. Nature 481, 506-510, doi:10.1038/nature10738 (2012).
Haubner, S. et al. Coexpression profile of leukemic stem cell markers for combinatorial targeted therapy in AML. Leukemia 33, 64-74, doi:10.1038/s41375-018-0180-3 (2019).
6 Vetrie, D., Helgason, G. V. & Copland, M. The leukaemia stem cell: similarities, differences and clinical prospects in CML and AML. Nature Reviews Cancer 20, 158-173, doi:10.1038/s41568-019-0230-9 (2020).
7 Shlush, L. I. et al. Tracing the origins of relapse in acute myeloid leukaemia to stem cells. Nature 547, 104-108, doi:10.1038/nature22993 (2017).
8 Yilmaz, M. et al. Late relapse in acute myeloid leukemia (AML): clonal evolution or therapy-related leukemia?Blood Cancer J 9, 7, doi:10.1038/s41408-019-0170-3 (2019).
9 Riddell, S. R., Jensen, M. C. & June, C. H. Chimeric antigen receptor-modified T cells: clinical translation in stem cell transplantation and beyond. Biol Blood Marrow Transplant 19, S2-5, doi:10.1016/j.bbmt.2012.10.021 (2013).
10 Sadelain, M., Brentjens, R. & Riviere, I. The basic principles of chimeric antigen receptor design. Cancer Discov 3, 388-398, doi:10.1158/2159-8290.Cd-12-0548 (2013).
11 Perna, F. et al. Integrating Proteomics and Transcriptomics for Systematic Combinatorial Chimeric Antigen Receptor Therapy of AML. Cancer Cell 32, 506-519 e505, doi:10.1016/j.ccell.2017.09.004 (2017).
12 Epperly, R., Gottschalk, S. & Velasquez, M. P. Harnessing T Cells to Target Pediatric Acute Myeloid Leukemia: CARs, BiTEs, and Beyond. Children (Basel) 7, doi:10.3390/children7020014 (2020).
13 Moretti, S. et al. CD123 (interleukin 3 receptor alpha chain). J Biol Regul Homeost Agents 15, 98-100 (2001).
14 Lee, D. W. et al. T cells expressing CD19 chimeric antigen receptors for acute lymphoblastic leukaemia in children and young adults: a phase 1 dose-escalation trial. Lancet 385, 517-528, doi:10.1016/s0140-6736(14)61403-3 (2015).
Maude, S. L. et al. Chimeric Antigen Receptor T Cells for Sustained Remissions in Leukemia. New England Journal of Medicine 371, 1507-1517, doi:10.1056/NEJMoa1407222 (2014).
16 Jacoby, E. et al. CD19 CAR immune pressure induces B-precursor acute lymphoblastic leukaemia lineage switch exposing inherent leukaemic plasticity. Nature Communications 7, 12320, doi:10.1038/ncomms12320 (2016).
17 Orlando, E. J. et al. Genetic mechanisms of target antigen loss in CAR19 therapy of acute lymphoblastic leukemia. Nat Med 24, 1504-1506, doi:10.1038/s41591-018-0146-z (2018).
18 Sotillo, E. et al. Convergence of Acquired Mutations and Alternative Splicing of CD19 Enables Resistance to CART-19 Immunotherapy. Cancer Discovery 5, 1282-1295, doi:10.1158/2159-8290.Cd-15-1020 (2015).
19 Dai, H. et al. Bispecific CAR-T cells targeting both CD19 and CD22 for therapy of adults with relapsed or refractory B cell acute lymphoblastic leukemia. Journal of Hematology & Oncology 13, 30, doi:10.1186/s13045-020-00856-8 (2020).
20 Zah, E., Lin, M. Y., Silva-Benedict, A., Jensen, M. C. & Chen, Y. Y. T Cells Expressing CD19/CD20 Bispecific Chimeric Antigen Receptors Prevent Antigen Escape by Malignant B Cells. Cancer Immunol Res 4, 498-508, doi:10.1158/2326-6066.Cir-15-0231 (2016).
21 Zah, E. et al. Systematically optimized BCMA/CS1 bispecific CAR-T cells robustly control heterogeneous multiple myeloma. Nature Communications 11, 2283, doi:10.1038/s41467-020-16160-5 (2020).
22 Klein, J. S., Jiang, S., Galimidi, R. P., Keeffe, J. R. & Bjorkman, P. J. Design and characterization of structured protein linkers with differing flexibilities. Protein Eng Des Sel 27, 325-330, doi:10.1093/protein/gzu043 (2014).
23 Bonifant, C. L. et al. CD123-Engager T Cells as a Novel Immunotherapeutic for Acute Myeloid Leukemia. Mol Ther 24, 1615-1626, doi:10.1038/mt.2016.116 (2016).
24 Kim, Y. et al. Targeting heat shock proteins on cancer cells: selection, characterization, and cell-penetrating properties of a peptidic GRP78 ligand. Biochemistry 45, 9434-9444, doi:10.1021/bi060264j (2006).
25 Jordan, C. T. et al. The interleukin-3 receptor alpha chain is a unique marker for human acute myelogenous leukemia stem cells. Leukemia 14, 1777-1784, doi:10.1038/sj.leu.2401903 (2000).
26 Qin, H. et al. Preclinical Development of Bivalent Chimeric Antigen Receptors Targeting Both CD19 and CD22. Mol Ther Oncolytics 11, 127-137, doi:10.1016/j.omto.2018.10.006 (2018).
27 Liu, F. et al. First-in-Human CLL1-CD33 Compound CAR T Cell Therapy Induces Complete Remission in Patients with Refractory Acute Myeloid Leukemia: Update on Phase 1 Clinical Trial. Blood 132, 901-901, doi:10.1182/blood-2018-99-110579 (2018).
28 He, X. et al. Bispecific and split CAR T cells targeting CD13 and TIM3 eradicate acute myeloid leukemia. Blood 135, 713-723, doi:10.1182/blood.2019002779 (2020).
29 N Hebbar, R Epperly, A Vaidya, U Thanekar, SE Moore, M Umeda, J Ma, S Patil, D Langfitt, S Huang, C Cheng, JM Klco, S Gottschalk, MP Velasquez CAR T cells redirected to cell surface GRP78 display robust anti-AML activity and do not target HPCs. Under Review at Nature Communications.
30. Wu C-Y, Roybal K T, Puchner E M, Onuffer J, Lim W A. Remote control of therapeutic T cells through a small molecule-gated chimeric receptor. Science. 2015 Oct. 16; 350(6258):aab4077. doi: 10.1126/science.aab4077. Epub 2015 Sep. 24.
31. Juillerat A, Tkach F, Busser B W, Temburni S, Valton J, Duclert A, Poirot L, Depil S, and Duchateau P. Modulation of chimeric antigen receptor surface expression by a small molecule switch. BMC Biotechnol. 2019; 19: 44. Published online 2019 Jul. 3. doi: 10.1186/s12896-019-0537-3.
32. Carbonneau S, Sharma S, Peng L, Rajan V, Hainzl D, Henault M, Yang C, Hale J, Shulok J, Tallarico J, Porter J, Brogdon J L, Dranoff G, Bradner J E, Hild M, and Guimaraes C P. An IMiD-inducible degron provides reversible regulation for chimeric antigen receptor expression and activity. Cell Chemical Biology. Volume 28, Issue 6, 17 Jun. 2021, Pages 802-812.e6
33. Jan M, Scarfò I, Larson R C, Walker A, Schmidts A, Guirguis A A, Gasser J A, Slabicki M, Bouffard A B, Castano A P, Kann M C, Cabral M L, Tepper A, Grinshpun D E, Sperling A S, Kyung T, Sievers Q L, Birnbaum M E, Maus M V, Ebert B L. Reversible ON- and OFF-switch chimeric antigen receptors controlled by lenalidomide. Sci Transl Med. 2021 Jan. 6; 13(575):eabb6295. doi: 10.1126/scitranslmed.abb6295.
34. Nguyen, P. et al. Route of 41BB/41BBL Costimulation Determines Effector Function of B7-H3-CAR.CD28ζ T Cells. Mol Ther Oncolytics 18, 202-214, doi:10.1016/j.omto.2020.06.018 (2020).
35. Klco, J. M. et al. Functional heterogeneity of genetically defined subclones in acute myeloid leukemia. Cancer Cell 25, 379-392, doi:10.1016/j.ccr.2014.01.031 (2014).
36. Ahmed, N. et al. Immunotherapy for osteosarcoma: genetic modification of T cells overcomes low levels of tumor antigen expression. Mol Ther 17, 1779-1787, doi:10.1038/mt.2009.133 (2009).
37. Diaconu, I. et al. Inducible Caspase-9 Selectively Modulates the Toxicities of CD19-Specific Chimeric Antigen Receptor-Modified T Cells. Mol Ther 25, 580-592, doi:10.1016/j.ymthe.2017.01.011 (2017).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

List of Sequences

SEQ ID NO: 1 IgG Heavy Chain Signal Peptide
MDWIWRILFLVGAATGAHS
SEQ ID NO: 2 IgG Heavy Chain Signal Peptide
ATGGACTGGATTTGGCGGATCCTGTTTCTCGTGGGAGCCGCCACAGGCGCCCATTCT
SEQ ID NO: 3 Leader
MDWIWRILFLVGAATGAHS
SEQ ID NO: 4 Leader
ATGGACTGGATCTGGCGGATTCTGTTCCTCGTGGGAGCCGCCACAGGCGCTCACTCA
SEQ ID NO: 5 Leader
ATGGACTGGATCTGGCGCATCCTCTTCCTCGTCGGCGCTGCTACCGGCGCTCATTCT
SEQ ID NO: 6 Leader
ATGGACTGGATCTGGCGCATCCTGTTTCTTGTGGGAGCCGCCACAGGCGCCCATTCT
SEQ ID NO: 7 CD8α Leader
MALPVTALLLPLALLLHAARP
SEQ ID NO: 8 CD8α Leader
ATGGCCTTACCAGTGACCGCCTTGCTCCTGCCGCTGGCCTTGCTGCTCCACGCCGCC
AGGCCG
SEQ ID NO: 9 G4S3 linker
GGGGSGGGGSGGGGS
SEQ ID NO: 10 G4S3 linker
GGAGGCGGAGGATCAGGCGGCGGAGGAAGTGGGGGAGGCGGATCT
SEQ ID NO: 11 (G4S)3 linker
GGAGGCGGAGGAAGTGGAGGGGGAGGATCAGGCGGCGGAGGCAGC
SEQ ID NO: 12 B2M linker
IQRTPKIQVYSRHPAENGKSNFLNCYVSGFHPSDIEVDLLKNGERIEKVEHSDLSFSKDW
SFYLLYYTEFTPTEKDEYACRVNHVTLSQPKIVKWDRD
SEQ ID NO: 13 B2M linker
ATACAACGCACGCCTAAAATCCAAGTCTATAGTCGGCACCCGGCGGAAAATGGGAA
ATCTAATTTCCTTAACTGCTATGTGTCCGGTTTCCACCCATCCGACATTGAGGTAGAC
CTGCTGAAAAACGGGGAACGGATAGAAAAGGTCGAACACAGTGATCTGTCCTTTAG
TAAGGATTGGTCTTTTTATCTGCTCTACTACACAGAGTTTACTCCGACAGAAAAAGA
CGAGTACGCGTGCCGCGTAAACCACGTAACACTGAGCCAACCGAAGATTGTGAAGT
GGGATAGAGAT
SEQ ID NO: 14 mtIgG4 linker
ESKYGPPCPSCPAPEFEGGPSVFLFPPKPKDTLMISRTPEVTCVVVDVSQEDPEVQFNWY
VDGVEVHNAKTKPREEQFQSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGK
SEQ ID NO: 15 mtIgG4 linker
GAGTCTAAGTACGGCCCTCCTTGTCCTAGCTGCCCCGCTCCTGAATTTGAAGGCGGC
CCTTCCGTGTTCCTGTTTCCTCCAAAGCCTAAGGACACCCTGATGATCAGCAGAACC
CCTGAAGTGACCTGCGTGGTGGTGGACGTGTCCCAAGAGGATCCTGAGGTGCAGTTC
AATTGGTACGTGGACGGCGTGGAAGTGCACAACGCCAAGACCAAGCCTAGAGAGGA
ACAGTTCCAGAGCACCTACAGAGTGGTGTCCGTGCTGACAGTGCTGCACCAGGATTG
GCTGAACGGCAAAGAGTACAAGTGCAAGGTGTCCAACAAGGGCCTGCCTAGCAGCA
TCGAGAAAACCATCAGCAAGGCCAAGGGCCAGCCAAGAGAACCCCAGGTGTACAC
ACTGCCTCCAAGCCAAGAGGAAATGACCAAGAACCAGGTGTCCCTGACCTGCCTGG
TCAAGGGCTTCTACCCTTCCGATATCGCCGTGGAATGGGAGAGCAATGGCCAGCCTG
AGAACAACTACAAGACCACACCTCCTGTGCTGGACAGCGACGGCTCATTCTTCCTGT
ACAGCAGACTGACCGTGGACAAGAGCAGATGGCAAGAGGGCAACGTGTTCAGCTGC
AGCGTGATGCACGAGGCCCTGCACAACCACTACACCCAGAAGTCTCTGAGCCTGAG
CCTCGGCAAG
SEQ ID NO: 16 GPcPcPc linker
AGSGGSGGSGGSPVPSTPPTNSSSTPPTPSPSPVPSTPPTNSSSTPPTPSPSPVPSTPPTNSSS
TPPTPSPSAS
SEQ ID NO: 17 GPcPcPc linker
GCAGGCAGTGGTGGAAGTGGTGGGTCAGGCGGTTCCCCGGTTCCATCAACACCACC
AACCAATAGCTCATCTACCCCGCCAACACCAAGTCCCTCTCCCGTCCCCAGCACACC
CCCGACGAACTCATCCTCCACACCGCCAACCCCGTCTCCTTCCCCGGTTCCCTCTACA
CCTCCGACAAACTCTAGTTCAACGCCTCCCACTCCATCACCTTCCGCATCA
SEQ ID NO: 18 Linker
GGCGGCGGCGGATCAGGCGGCGGAGGAAGCGGAGGTGGGGGGTCC
SEQ ID NO: 19 Linker
GGAGGCGGAGGTTCAGGTGGCGGAGGAAGTGGCGGCGGAGGATCA
SEQ ID NO: 20 Linker
GGGGS
SEQ ID NO: 21 Linker
GGAGGCGGAGGTTCA
SEQ ID NO: 22 (G4S)2 linker
GGGGSGGGGS
SEQ ID NO: 23 (G4S)4 linker
GGGGSGGGGSGGGGSGGGGS
SEQ ID NO: 24 Linker
KESGSVSSEQLAQFRSLD
SEQ ID NO: 25 Linker
EGKSSGSGSESKST
SEQ ID NO: 26 Linker
EGKSSGSGSESKSTQ
SEQ ID NO: 27 Linker
GSTSGSGKSSEGKG
SEQ ID NO: 28 Linker
SSADDAKKDDAKKDDAKKDDAKKDG
SEQ ID NO: 29 Linker
EGKSSGSGSESKVD
SEQ ID NO: 30 Linker
ESGSVSSEELAFRSLD
SEQ ID NO: 31 Short Hinge
DLEPKSCDKTHTCPPCPDPK
SEQ ID NO: 32 Short Hinge
GATCTCGAGCCCAAATCTTGTGACAAAACTCACACATGCCCACCGTGCCCGGATCCC
AAG
SEQ ID NO: 33 Hinge
ESKYGPPCPSCPAPEFEGGPSVFLFPPKPKDTLMISRTPEVTCVVVDVSQEDPEVQFNWY
VDGVEVHNAKTKPREEQFQSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGKGS
SEQ ID NO: 34 Hinge
GAGTCTAAGTACGGCCCTCCTTGTCCTAGCTGCCCCGCTCCTGAATTTGAAGGCGGC
CCTTCCGTGTTCCTGTTTCCTCCAAAGCCTAAGGACACCCTGATGATCAGCAGAACC
CCTGAAGTGACCTGCGTGGTGGTGGACGTGTCCCAAGAGGATCCTGAGGTGCAGTTC
AATTGGTACGTGGACGGCGTGGAAGTGCACAACGCCAAGACCAAGCCTAGAGAGGA
ACAGTTCCAGAGCACCTACAGAGTGGTGTCCGTGCTGACAGTGCTGCACCAGGATTG
GCTGAACGGCAAAGAGTACAAGTGCAAGGTGTCCAACAAGGGCCTGCCTAGCAGCA
TCGAGAAAACCATCAGCAAGGCCAAGGGCCAGCCAAGAGAACCCCAGGTGTACAC
ACTGCCTCCAAGCCAAGAGGAAATGACCAAGAACCAGGTGTCCCTGACCTGCCTGG
TCAAGGGCTTCTACCCTTCCGATATCGCCGTGGAATGGGAGAGCAATGGCCAGCCTG
AGAACAACTACAAGACCACACCTCCTGTGCTGGACAGCGACGGCTCATTCTTCCTGT
ACAGCAGACTGACCGTGGACAAGAGCAGATGGCAAGAGGGCAACGTGTTCAGCTGC
AGCGTGATGCACGAGGCCCTGCACAACCACTACACCCAGAAGTCTCTGAGCCTGAG
CCTCGGCAAGGGCTCC
SEQ ID NO: 35 Hinge
TTTPAPRPPTPAPTIASQPLSLRPEACRPAAGGAVHTRGLDFACD
SEQ ID NO: 36 Hinge
ACCACCACACCAGCTCCTCGGCCTCCAACTCCTGCTCCTACAATAGCCAGCCAGCCT
CTGTCTCTGAGGCCCGAAGCATGCAGACCTGCTGCTGGCGGAGCCGTGCATACAAG
AGGACTGGATTTCGCCTGCGAC
SEQ ID NO: 37 CD28 hinge
IEVMYPPPYLDNEKSNGTIIHVKGKHLCPSPLFPGPSKP
SEQ ID NO: 38 CD28 hinge
ATCGAAGTGATGTACCCGCCTCCTTACCTGGACAACGAGAAGTCCAACGGCACCAT
CATCCACGTGAAGGGAAAGCACCTGTGTCCTTCTCCACTGTTCCCCGGACCTAGCAA
GCC
SEQ ID NO: 39 IgG1 hinge
EPKSCDKTHTCPPCP
SEQ ID NO: 40 IgG2 hinge
ERKCCVECPPCP
SEQ ID NO: 41 IgG3 hinge
ELKTPLGDTTHTCPRCP(EPKSCDTPPPCPRCP)3
SEQ ID NO: 42 IgG4 hinge
ESKYGPPCPSCP
SEQ ID NO: 43 CD28 transmembrane
FWVLVVVGGVLACYSLLVTVAFIIFWV
SEQ ID NO: 44 CD28 transmembrane
TTTCTGGGTGCTCGTTGTTGTTGGCGGCGTGCTGGCCTGTTACAGCCTGCTGGTTACC
GTGGCCTTCATCATCTTTTGGGTC
SEQ ID NO: 45 CD28 Transmembrane
TTCTGGGTGCTGGTGGTCGTGGGCGGAGTGCTGGCCTGTTACAGCCTGCTCGTGACC
GTGGCCTTCATCATCTTTTGGGTG
SEQ ID NO: 46 CD28 Transmembrane
FWVLVVVGGVLACYSLLVTVAFIIFWVRSKRSRLLH
SEQ ID NO: 47 CD28 Transmembrane
TTCTGGGTGCTGGTGGTCGTGGGCGGAGTGCTGGCCTGTTACAGCCTGCTCGTGACC
GTGGCCTTCATCATCTTTTGGGTGCGCAGCAAGCGGAGCCGGCTGCTGCAC
SEQ ID NO: 48 CD8α transmembrane domain
IYIWAPLAGTCGVLLLSLVITLYC
SEQ ID NO: 49 CD8α transmembrane domain
ATCTACATCTGGGCCCCTCTGGCTGGAACATGTGGCGTTCTGCTGCTGAGCCTGGTC
ATCACCCTGTACTGC
SEQ ID NO: 50 CD3ζ transmembrane domain
LCYLLDGILFIYGVILTALFL
SEQ ID NO: 51 CD3ζ transmembrane domain
CTGTGCTACCTGCTGGACGGCATCCTGTTCATCTACGGCGTGATCCTGACCGCCCTG
TTCCTG
SEQ ID NO: 52 CD28 hinge/transmembrane
IEVMYPPPYLDNEKSNGTIIHVKGKHLCPSPLFPGPSKPFWVLVVVGGVLACYSLLVTVA
FIIFWV
SEQ ID NO: 53 CD28 hinge/transmembrane
ATCGAAGTGATGTACCCGCCTCCTTACCTGGACAACGAGAAGTCCAACGGCACCAT
CATCCACGTGAAGGGAAAGCACCTGTGTCCTTCTCCACTGTTCCCCGGACCTAGCAA
GCCTTTCTGGGTGCTCGTTGTTGTTGGCGGCGTGCTGGCCTGTTACAGCCTGCTGGTT
ACCGTGGCCTTCATCATCTTTTGGGTC
SEQ ID NO: 54 CD28 costimulatory
RSKRSRLLHSDYMNMTPRRPGPTRKHYQPYAPPRDFAAYRS
SEQ ID NO: 55 CD28 costimulatory
CGAAGCAAGCGGAGCCGGCTGCTGCACAGCGACTACATGAACATGACCCCTAGACG
GCCCGGACCAACCAGAAAGCACTACCAGCCTTACGCTCCTCCTAGAGATTTCGCCGC
CTACCGGTCC
SEQ ID NO: 56 CD28 Costimulatory
CGCAGCAAGCGGAGCCGGCTGCTGCACAGCGACTACATGAACATGACCCCCAGACG
GCCTGGCCCCACCAGAAAGCACTACCAGCCTTACGCCCCTCCCAGAGACTTCGCCGC
CTACCGGTCC
SEQ ID NO: 57 4-1BB costimulatory domain
KRGRKKLLYIFKQPFMRPVQTTQEEDGCSCRFPEEEEGGCEL
SEQ ID NO: 58 4-1BB costimulatory domain
AAACGGGGCAGAAAGAAACTCCTGTATATATTCAAACAACCATTTATGAGACCAGT
ACAAACTACTCAAGAGGAAGATGGCTGTAGCTGCCGATTTCCAGAAGAAGAAGAAG
GAGGATGTGAACTG
SEQ ID NO: 59 OX40 costimulatory domain
RDQRLPPDAHKPPGGGSFRTPIQEEQADAHSTLAKI
SEQ ID NO: 60 OX40 costimulatory domain
AGGGACCAGAGGCTGCCCCCCGATGCCCACAAGCCCCCTGGGGGAGGCAGTTTCCG
GACCCCCATCCAAGAGGAGCAGGCCGACGCCCACTCCACCCTGGCCAAGATC
SEQ ID NO: 61 MyD88 fragment 1 costimulatory domain
AAGGPGAGSAAPVSSTSSLPLAALNMRVRRRLSLFLNVRTQVAADWTALAEEMDFEYL
EIRQLETQADPTGRLLDAWQGRPGASVGRLLELLTKLGRDDVLLELGPSIEEDCQKYILK
QQQEEAEKPLQVAAVDSSVPRTAELAGITTLDDPLGHMPERFDAFICYCPSDI
SEQ ID NO: 62 MyD88 fragment 1 costimulatory domain
GCTGCTGGCGGACCTGGCGCCGGATCTGCTGCTCCTGTGTCTAGCACAAGCAGCCTG
CCTCTGGCCGCCCTGAACATGAGAGTGCGGAGAAGGCTGAGCCTGTTCCTGAACGT
GCGGACACAGGTGGCCGCCGATTGGACAGCCCTGGCCGAGGAAATGGACTTCGAGT
ACCTGGAAATCCGGCAGCTGGAAACCCAGGCCGACCCTACAGGCAGACTGCTGGAT
GCTTGGCAGGGCAGACCAGGCGCTTCTGTGGGAAGGCTGCTGGAACTGCTGACCAA
GCTGGGCAGGGACGACGTGCTGCTGGAACTGGGCCCTAGCATCGAAGAGGACTGCC
AGAAGTACATCCTGAAGCAGCAGCAGGAAGAGGCCGAGAAGCCTCTGCAGGTGGC
AGCCGTGGATAGCAGCGTGCCAAGAACAGCCGAGCTGGCCGGCATCACCACCCTGG
ATGATCCTCTGGGCCACATGCCCGAGAGATTCGACGCCTTCATCTGCTACTGCCCCA
GCGACATC
SEQ ID NO: 63 MyD88 fragment 2 costimulatory domain
MAAGGPGAGSAAPVSSTSSLPLAALNMRVRRRLSLFLNVRTQVAADWTALAEEMDFE
YLEIRQLETQADPTGRLLDAWQGRPGASVGRLLDLLTKLGRDDVLLELGPSIEEDCQKYI
LKQQQEEAEKPLQVAAVDSSVPRTAELAGITTLDDPLGHMPERFDAFICYCPSDI
SEQ ID NO: 64 MyD88 fragment 2 costimulatory domain
ATGGCCGCTGGGGGCCCAGGCGCCGGATCAGCTGCTCCCGTATCTTCTACTTCTTCT
TTGCCGCTGGCTGCTCTGAACATGCGCGTGAGAAGACGCCTCTCCCTGTTCCTTAAC
GTTCGCACACAAGTCGCTGCCGATTGGACCGCCCTTGCCGAAGAAATGGACTTTGAA
TACCTGGAAATTAGACAACTTGAAACACAGGCCGACCCCACTGGCAGACTCCTGGA
CGCATGGCAGGGAAGACCTGGTGCAAGCGTTGGACGGCTCCTGGATCTCCTGACAA
AACTGGGACGCGACGACGTACTGCTTGAACTCGGACCTAGCATTGAAGAAGACTGC
CAAAAATATATCCTGAAACAACAACAAGAAGAAGCCGAAAAACCTCTCCAAGTCGC
AGCAGTGGACTCATCAGTACCCCGAACAGCTGAGCTTGCTGGGATTACTACACTCGA
CGACCCACTCGGACATATGCCTGAAAGATTCGACGCTTTCATTTGCTATTGCCCCTCT
GACATA
SEQ ID NO: 65 MyD88 fragment 3 costimulatory domain
AAGGPGAGSAAPVSSTSSLPLAALNMRVRRRLSLFLNVRTQVAADWTALAEEMDFEYL
EIRQLETQADPTGRLLDAWQGRPGASVGRLLDLLTKLGRDDVLLELGPSIEEDCQKYILK
QQQEEAEKPLQVAAVDSSVPRTAELAGITTLDDPLGHMPERFDAFICYCPSDI
SEQ ID NO: 66 MyD88 fragment 3 costimulatory domain
GCCGCTGGGGGCCCAGGCGCCGGATCAGCTGCTCCCGTATCTTCTACTTCTTCTTTGC
CGCTGGCTGCTCTGAACATGCGCGTGAGAAGACGCCTCTCCCTGTTCCTTAACGTTC
GCACACAAGTCGCTGCCGATTGGACCGCCCTTGCCGAAGAAATGGACTTTGAATAC
CTGGAAATTAGACAACTTGAAACACAGGCCGACCCCACTGGCAGACTCCTGGACGC
ATGGCAGGGAAGACCTGGTGCAAGCGTTGGACGGCTCCTGGATCTCCTGACAAAAC
TGGGACGCGACGACGTACTGCTTGAACTCGGACCTAGCATTGAAGAAGACTGCCAA
AAATATATCCTGAAACAACAACAAGAAGAAGCCGAAAAACCTCTCCAAGTCGCAGC
AGTGGACTCATCAGTACCCCGAACAGCTGAGCTTGCTGGGATTACTACACTCGACGA
CCCACTCGGACATATGCCTGAAAGATTCGACGCTTTCATTTGCTATTGCCCCTCTGAC
ATA
SEQ ID NO: 67 CD40 costimulatory domain
KKVAKKPTNKAPHPKQEPQEINFPDDLPGSNTAAPVQETLHGCQPVTQEDGKESRISVQ
ERQ
SEQ ID NO: 68 CD40 costimulatory domain
AAGAAGGTGGCCAAGAAGCCCACCAACAAGGCCCCCCACCCCAAGCAGGAACCCC
AGGAAATCAACTTCCCCGACGACCTGCCCGGCAGCAATACTGCTGCACCCGTGCAG
GAAACCCTGCACGGCTGTCAGCCTGTGACCCAGGAAGATGGCAAAGAAAGCCGGAT
CTCTGTGCAGGAACGCCAG
SEQ ID NO: 69 CD3z
RVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
SEQ ID NO: 70 CD3z
AGAGTGAAGTTCAGCAGGAGCGCAGACGCCCCCGCGTACCAGCAGGGCCAGAACC
AGCTCTATAACGAGCTCAATCTAGGACGAAGAGAGGAGTACGATGTTTTGGACAAG
AGACGTGGCCGGGACCCTGAGATGGGGGGAAAGCCGAGAAGGAAGAACCCTCAGG
AAGGCCTGTACAATGAACTGCAGAAAGATAAGATGGCGGAGGCCTACAGTGAGATT
GGGATGAAAGGCGAGCGCCGGAGGGGCAAGGGGCACGATGGCCTTTACCAGGGTCT
CAGTACAGCCACCAAGGACACCTACGACGCCCTTCACATGCAGGCCCTGCCCCCTCG
C
SEQ ID NO: 71 CD3 zeta activation
AGAGTGAAGTTCAGCAGAAGCGCCGACGCCCCTGCCTATCAGCAGGGCCAGAACCA
GCTGTACAACGAGCTGAACCTGGGCAGACGGGAAGAGTACGACGTGCTGGACAAGC
GGAGAGGCAGGGACCCTGAGATGGGCGGCAAGCCCAGAAGAAAGAACCCCCAGGA
AGGCCTGTATAACGAACTGCAGAAAGACAAGATGGCCGAGGCCTACAGCGAGATCG
GCATGAAGGGCGAGCGGAGAAGAGGCAAGGGCCACGATGGCCTGTACCAGGGACT
GAGCACCGCCACCAAGGACACCTACGACGCCCTGCACATGCAGGCCCTGCCTCCAA
GA
SEQ ID NO: 72 Zeta
RVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPRAS
RA
SEQ ID NO: 73 Zeta
AGAGTGAAGTTCAGCAGAAGCGCCGACGCCCCTGCCTATCAGCAGGGCCAGAACCA
GCTGTACAACGAGCTGAACCTGGGCAGACGGGAAGAGTACGACGTGCTGGACAAGC
GGAGAGGCAGGGACCCTGAGATGGGCGGCAAGCCCAGAAGAAAGAACCCCCAGGA
AGGCCTGTATAACGAACTGCAGAAAGACAAGATGGCCGAGGCCTACAGCGAGATCG
GCATGAAGGGCGAGCGGAGAAGAGGCAAGGGCCACGATGGCCTGTACCAGGGACT
GAGCACCGCCACCAAGGACACCTACGACGCCCTGCACATGCAGGCCCTGCCTCCAA
GAGCCTCTAGAGCC
SEQ ID NO: 74 T2A ribosomal skipping
EGRGSLLTCGDVEENPGP
SEQ ID NO: 75 T2A ribosomal skipping
GAAGGCAGAGGCTCTCTGCTGACATGTGGCGACGTGGAAGAGAATCCTGGACCT
SEQ ID NO: 76 T2A
GAGGGCAGAGGCAGCCTGCTGACATGTGGCGACGTGGAAGAGAACCCAGGCCCC
SEQ ID NO: 77 Thoseaasigna virus 2A
AEGRGSLLTCGDVEENPGP
SEQ ID NO: 78 Thoseaasigna virus 2A
GSGEGRGSLLTCGDVEENPGP
SEQ ID NO: 79 FMDV2A
GSGSRVTELLYRMKRAETYCPRPLLAIHPTEARHKQKIVAPVKQLLNFDLLKLAGDVES
NPGP
SEQ ID NO: 80 Sponge 2A
LLCFLLLLLSGDVELNPGP
SEQ ID NO: 81 Sponge 2A
HHFMFLLLLLAGDIELNPGP
SEQ ID NO: 82 Acorn Worm 2A
WFLVLLSFILSGDIEVNPGP

SEQ ID NO: 83 Amphioxus 2A
KNCAMYMLLLSGDVETNPGP
SEQ ID NO: 84 Amphioxus 2A
MVISQLMLKLAGDVEENPGP
SEQ ID NO: 85 Porcine Teschovirus-1 2A
GSGATNFSLLKQAGDVEENPGP
SEQ ID NO: 86 Equine Rhinitis A Virus 2A
GSGQCTNYALLKLAGDVESNPGP
SEQ ID NO: 87 2A consensus
D-X-E-X-NPGP
SEQ ID NO: 88 Truncated CD19

MPPPRLLFFLLFLTPMEVRPEEPLVVKVEEGDNAVLQCLKGTSDGPTQQLTWSRESPLKP
FLKLSLGLPGLGIHMRPLAIWLFIFNVSQQMGGFYLCQPGPPSEKAWQPGWTVNVEGSG
ELFRWNVSDLGGLGCGLKNRSSEGPSSPSGKLMSPKLYVWAKDRPEIWEGEPPCLPPRD
SLNQSLSQDLTMAPGSTLWLSCGVPPDSVSRGPLSWTHVHPKGPKSLLSLELKDDRPAR
DMWVMETGLLLPRATAQDAGKYYCHRGNLTMSFHLEITARPVLWHWLLRTGGWKVS
AVTLAYLIFCLCSLVGILHLQRALVLRRKRKRMTDPTRRF
SEQ ID NO: 89 Truncated CD19
ATGCCTCCCCCCAGACTGCTGTTCTTCCTGCTGTTCCTGACCCCTATGGAAGTGCGGC
CCGAGGAACCCCTGGTCGTGAAAGTGGAAGAGGGCGACAACGCCGTGCTGCAGTGT
CTGAAGGGCACCTCCGATGGCCCTACCCAGCAGCTGACCTGGTCCAGAGAGAGCCC
CCTGAAGCCCTTCCTGAAGCTGTCTCTGGGCCTGCCTGGCCTGGGCATCCATATGAG
GCCACTGGCCATCTGGCTGTTCATCTTCAACGTGTCCCAGCAGATGGGAGGCTTCTA
CCTGTGCCAGCCTGGCCCACCTTCTGAGAAGGCTTGGCAGCCTGGCTGGACCGTGAA
CGTGGAAGGATCTGGCGAGCTGTTCCGGTGGAACGTGTCCGATCTGGGCGGCCTGG
GATGCGGCCTGAAGAACAGATCTAGCGAGGGCCCCAGCAGCCCCAGCGGCAAACTG
ATGAGCCCCAAGCTGTACGTGTGGGCCAAGGACAGACCCGAGATTTGGGAGGGCGA
GCCCCCTTGCCTGCCCCCTAGAGATAGCCTGAACCAGAGCCTGAGCCAGGACCTGA
CAATGGCCCCTGGCAGCACACTGTGGCTGAGCTGTGGCGTGCCACCCGACTCTGTGT
CTAGAGGCCCTCTGAGCTGGACCCACGTGCACCCTAAGGGCCCTAAGAGCCTGCTGT
CCCTGGAACTGAAGGACGACAGGCCCGCCAGAGATATGTGGGTCATGGAAACCGGC
CTGCTGCTGCCTAGAGCCACAGCCCAGGATGCCGGCAAGTACTACTGCCACAGAGG
CAACCTGACCATGAGCTTCCACCTGGAAATCACCGCCAGACCCGTGCTGTGGCACTG
GCTGCTGAGAACCGGCGGATGGAAAGTGTCCGCCGTGACTCTGGCCTACCTGATCTT
CTGCCTGTGCTCCCTCGTGGGCATCCTGCATCTGCAGAGGGCTCTGGTGCTGCGGCG
GAAGCGGAAGAGAATGACCGACCCTACCCGGCGGTTC
SEQ ID NO: 90 tCD19
ATGCCTCCCCCCAGACTGCTGTTCTTCCTGCTGTTCCTGACCCCTATGGAAGTGCGGC
CCGAGGAACCCCTGGTCGTGAAAGTGGAAGAGGGCGACAACGCCGTGCTGCAGTGT
CTGAAGGGCACCTCCGATGGCCCTACCCAGCAGCTGACCTGGTCCAGAGAGAGCCC
GCCACTGGCCATCTGGCTGTTCATCTTCAACGTGTCCCAGCAGATGGGAGGCTTCTA
CCTGTGCCAGCCTGGCCCACCTTCTGAGAAGGCTTGGCAGCCTGGCTGGACCGTGAA
CGTGGAAGGATCTGGCGAGCTGTTCCGGTGGAACGTGTCCGATCTGGGCGGCCTGG
GATGCGGCCTGAAGAACAGATCTAGCGAGGGCCCCAGCAGCCCCAGCGGCAAACTG
ATGAGCCCCAAGCTGTACGTGTGGGCCAAGGACAGACCCGAGATTTGGGAGGGCGA
GCCCCCTTGCCTGCCCCCTAGAGATAGCCTGAACCAGAGCCTGAGCCAGGACCTGA
CAATGGCCCCTGGCAGCACACTGTGGCTGAGCTGTGGCGTGCCACCCGACTCTGTGT
CTAGAGGCCCTCTGAGCTGGACCCACGTGCACCCTAAGGGCCCTAAGAGCCTGCTGT
CCCTGGAACTGAAGGACGACAGGCCCGCCAGAGATATGTGGGTCATGGAAACCGGC
CTGCTGCTGCCTAGAGCCACAGCCCAGGATGCCGGCAAGTACTACTGCCACAGAGG
CAACCTGACCATGAGCTTCCACCTGGAAATCACCGCCAGACCCGTGCTGTGGCACTG
GCTGCTGAGAACCGGCGGATGGAAAGTGTCCGCCGTGACTCTGGCCTACCTGATCTT
CTGCCTGTGCTCCCTCGTGGGCATCCTGCATCTGCAGAGGGCTCTGGTGCTGCGGCG
GAAGCGGAAGAGAATGACCGACCCTACCCGGCGGTTC
SEQ ID NO: 91 tCD19
ATGCCTCCCCCCAGACTGCTGTTCTTCCTGCTGTTCCTGACCCCTATGGAAGTGCGGC
CCGAGGAACCCCTGGTCGTGAAAGTGGAAGAGGGCGACAACGCCGTGCTGCAGTGT
CTGAAGGGCACCTCCGATGGCCCTACCCAGCAGCTGACCTGGTCCAGAGAGAGCCC
CCTGAAGCCCTTCCTGAAGCTGTCTCTGGGCCTGCCTGGCCTGGGCATCCATATGAG
GCCACTGGCCATCTGGCTGTTCATCTTCAACGTGTCCCAGCAGATGGGAGGCTTCTA
CCTGTGCCAGCCTGGCCCACCTTCTGAGAAGGCTTGGCAGCCTGGCTGGACCGTGAA
CGTGGAAGGATCTGGCGAGCTGTTCCGGTGGAACGTGTCCGATCTGGGCGGCCTGG
GATGCGGCCTGAAGAACAGATCTAGCGAGGGCCCCAGCAGCCCCAGCGGCAAACTG
ATGAGCCCCAAGCTGTACGTGTGGGCCAAGGACAGACCCGAGATTTGGGAGGGCGA
GCCCCCTTGCCTGCCCCCTAGAGATAGCCTGAACCAGAGCCTGAGCCAGGACCTGA
CAATGGCCCCTGGCAGCACACTGTGGCTGAGCTGTGGCGTGCCACCCGACTCTGTGT
CTAGAGGCCCTCTGAGCTGGACCCACGTGCACCCTAAGGGCCCTAAGAGCCTGCTGT
CCCTGGAACTGAAGGACGACAGGCCCGCCAGAGATATGTGGGTCATGGAAACCGGC
CTGCTGCTGCCTAGAGCCACAGCCCAGGATGCCGGCAAGTACTACTGCCACAGAGG
CAACCTGACCATGAGCTTCCACCTGGAAATCACCGCCAGACCCGTGCTGTGGCACTG
GCTGCTGAGAACCGGCGGATGGAAAGTGTCCGCCGTGACTCTGGCCTACCTGATCTT
CTGCCTGTGCTCCCTCGTGGGCATCCTGCATCTGCAGAGGGCTCTGGTGCTGCGGCG
GAAGCGGAAGAGAATGACCGACCCTACCCGGCGGTTCTAA
SEQ ID NO: 92 GRP78 peptide
CTVALPGGYVRVC
SEQ ID NO: 93 GRP78 peptide
TGTACTGTGGCCCTTCCTGGTGGATACGTTAGAGTGTGC
SEQ ID NO: 94 CD123 Variable Heavy Chain
QVQLQQPGAELVRPGASVKLSCKASGYTFTSYWMNWVKQRPDQGLEWIGRIDPYDSET
HYNQKFKDKAILTVDKSSSTAYMQLSSLTSEDSAVYYCARGNWDDYWGQGTTLTVSS
SEQ ID NO: 95 CD123 Variable Heavy Chain
CAGGTGCAGCTGCAGCAGCCTGGCGCTGAACTCGTGCGGCCAGGCGCTTCTGTGAA
GCTGAGCTGTAAAGCCAGCGGCTACACCTTCACCAGCTACTGGATGAACTGGGTCA
AGCAGCGGCCCGACCAGGGCCTGGAATGGATCGGAAGAATCGACCCCTACGACAGC
GAGACACACTACAACCAGAAGTTCAAGGACAAGGCCATCCTGACCGTGGACAAGAG
CAGCAGCACCGCCTACATGCAGCTGTCCAGCCTGACCAGCGAGGACAGCGCCGTGT
ACTACTGCGCCAGAGGCAACTGGGACGACTACTGGGGCCAGGGCACAACCCTGACA
GTGTCTAGC
SEQ ID NO: 96 CD123 Variable Light Chain
DVQITQSPSYLAASPGETITINCRASKSISKDLAWYQEKPGKTNKLLIYSGSTLQSGIPSRF
SGSGSGTDFTLTISSLEPEDFAMYYCQQHNKYPYTFGGGTKLEIKS
SEQ ID NO: 97 CD123 Variable Light Chain
GATGTGCAGATTACCCAGTCCCCCAGCTACCTGGCCGCCTCTCCTGGCGAGACAATC
ACCATCAACTGCCGGGCCAGCAAGAGCATCTCCAAGGACCTGGCCTGGTATCAGGA
AAAGCCCGGCAAGACCAACAAGCTGCTGATCTACAGCGGCTCCACCCTGCAGTCCG
GCATCCCCAGCAGATTTTCCGGCAGCGGCTCTGGCACCGACTTCACCCTGACCATCA
GCTCCCTGGAACCCGAGGACTTTGCCATGTACTATTGCCAGCAGCACAACAAGTACC
CTTACACCTTCGGCGGAGGCACCAAGCTGGAAATCAAGAGC
SEQ ID NO: 98 CD123 scFv
QVQLQQPGAELVRPGASVKLSCKASGYTFTSYWMNWVKQRPDQGLEWIGRIDPYDSET
HYNQKFKDKAILTVDKSSSTAYMQLSSLTSEDSAVYYCARGNWDDYWGQGTTLTVSS
GGGGSGGGGSGGGGSDVQITQSPSYLAASPGETITINCRASKSISKDLAWYQEKPGKTN
KLLIYSGSTLQSGIPSRFSGSGSGTDFTLTISSLEPEDFAMYYCQQHNKYPYTFGGGTKLEI
KS
SEQ ID NO: 99 CD123 scFv
CAGGTGCAGCTGCAGCAGCCTGGCGCTGAACTCGTGCGGCCAGGCGCTTCTGTGAA
GCTGAGCTGTAAAGCCAGCGGCTACACCTTCACCAGCTACTGGATGAACTGGGTCA
AGCAGCGGCCCGACCAGGGCCTGGAATGGATCGGAAGAATCGACCCCTACGACAGC
GAGACACACTACAACCAGAAGTTCAAGGACAAGGCCATCCTGACCGTGGACAAGAG
CAGCAGCACCGCCTACATGCAGCTGTCCAGCCTGACCAGCGAGGACAGCGCCGTGT
ACTACTGCGCCAGAGGCAACTGGGACGACTACTGGGGCCAGGGCACAACCCTGACA
GTGTCTAGCGGAGGCGGAGGATCAGGCGGCGGAGGAAGTGGGGGAGGCGGATCTG
ATGTGCAGATTACCCAGTCCCCCAGCTACCTGGCCGCCTCTCCTGGCGAGACAATCA
CCATCAACTGCCGGGCCAGCAAGAGCATCTCCAAGGACCTGGCCTGGTATCAGGAA
AAGCCCGGCAAGACCAACAAGCTGCTGATCTACAGCGGCTCCACCCTGCAGTCCGG
CATCCCCAGCAGATTTTCCGGCAGCGGCTCTGGCACCGACTTCACCCTGACCATCAG
CTCCCTGGAACCCGAGGACTTTGCCATGTACTATTGCCAGCAGCACAACAAGTACCC
TTACACCTTCGGCGGAGGCACCAAGCTGGAAATCAAGAGC
SEQ ID NO: 100 GRP78-G4S3-CD123-CAR_Extracellular domain
MDWIWRILFLVGAATGAHSCTVALPGGYVRVCEFGGGGSGGGGSGGGGSAAAQVQLQ
QPGAELVRPGASVKLSCKASGYTFTSYWMNWVKQRPDQGLEWIGRIDPYDSETHYNQ
KFKDKAILTVDKSSSTAYMQLSSLTSEDSAVYYCARGNWDDYWGQGTTLTVSSGGGGS
GGGGSGGGGSDVQITQSPSYLAASPGETITINCRASKSISKDLAWYQEKPGKTNKLLIYS
GSTLQSGIPSRFSGSGSGTDFTLTISSLEPEDFAMYYCQQHNKYPYTFGGGTKLEIKSIEV
MYPPPYLDNEKSNGTIIHVKGKHLCPSPLFPGPSKP
SEQ ID NO: 101 GRP78-G4S3-CD123-CAR_Extracellular domain
ATGGACTGGATTTGGCGGATCCTGTTTCTCGTGGGAGCCGCCACAGGCGCCCATTCT
TGTACTGTGGCCCTTCCTGGTGGATACGTTAGAGTGTGCGAATTCGGAGGCGGAGGA
TCAGGCGGCGGAGGAAGTGGGGGAGGCGGATCTGCGGCCGCTCAGGTGCAGCTGCA
GCAGCCTGGCGCTGAACTCGTGCGGCCAGGCGCTTCTGTGAAGCTGAGCTGTAAAG
CCAGCGGCTACACCTTCACCAGCTACTGGATGAACTGGGTCAAGCAGCGGCCCGAC
CAGGGCCTGGAATGGATCGGAAGAATCGACCCCTACGACAGCGAGACACACTACAA
CCAGAAGTTCAAGGACAAGGCCATCCTGACCGTGGACAAGAGCAGCAGCACCGCCT
ACATGCAGCTGTCCAGCCTGACCAGCGAGGACAGCGCCGTGTACTACTGCGCCAGA
GGCAACTGGGACGACTACTGGGGCCAGGGCACAACCCTGACAGTGTCTAGCGGAGG
CGGAGGATCAGGCGGCGGAGGAAGTGGGGGAGGCGGATCTGATGTGCAGATTACCC
AGTCCCCCAGCTACCTGGCCGCCTCTCCTGGCGAGACAATCACCATCAACTGCCGGG
CCAGCAAGAGCATCTCCAAGGACCTGGCCTGGTATCAGGAAAAGCCCGGCAAGACC
AACAAGCTGCTGATCTACAGCGGCTCCACCCTGCAGTCCGGCATCCCCAGCAGATTT
TCCGGCAGCGGCTCTGGCACCGACTTCACCCTGACCATCAGCTCCCTGGAACCCGAG
GACTTTGCCATGTACTATTGCCAGCAGCACAACAAGTACCCTTACACCTTCGGCGGA
GGCACCAAGCTGGAAATCAAGAGCATCGAAGTGATGTACCCGCCTCCTTACCTGGA
CAACGAGAAGTCCAACGGCACCATCATCCACGTGAAGGGAAAGCACCTGTGTCCTT
CTCCACTGTTCCCCGGACCTAGCAAGCC
SEQ ID NO: 102 GRP78-B2M-CD123-CAR_Extracellular domain
MDWIWRILFLVGAATGAHSCTVALPGGYVRVCEFIQRTPKIQVYSRHPAENGKSNFLNC
YVSGFHPSDIEVDLLKNGERIEKVEHSDLSFSKDWSFYLLYYTEFTPTEKDEYACRVNHV
TLSQPKIVKWDRDAAAQVQLQQPGAELVRPGASVKLSCKASGYTFTSYWMNWVKQRP
DQGLEWIGRIDPYDSETHYNQKFKDKAILTVDKSSSTAYMQLSSLTSEDSAVYYCARGN
WDDYWGQGTTLTVSSGGGGSGGGGSGGGGSDVQITQSPSYLAASPGETITINCRASKSIS
KDLAWYQEKPGKTNKLLIYSGSTLQSGIPSRFSGSGSGTDFTLTISSLEPEDFAMYYCQQ
HNKYPYTFGGGTKLEIKSIEVMYPPPYLDNEKSNGTIIHVKGKHLCPSPLFPGPSKP
SEQ ID NO: 103 GRP78-B2M-CD123-CA_Extracellular domain
ATGGACTGGATTTGGCGGATCCTGTTTCTCGTGGGAGCCGCCACAGGCGCCCATTCT
TGTACTGTGGCCCTTCCTGGTGGATACGTTAGAGTGTGCGAATTCATACAACGCACG
CCTAAAATCCAAGTCTATAGTCGGCACCCGGCGGAAAATGGGAAATCTAATTTCCTT
AACTGCTATGTGTCCGGTTTCCACCCATCCGACATTGAGGTAGACCTGCTGAAAAAC
GGGGAACGGATAGAAAAGGTCGAACACAGTGATCTGTCCTTTAGTAAGGATTGGTC
TTTTTATCTGCTCTACTACACAGAGTTTACTCCGACAGAAAAAGACGAGTACGCGTG
CCGCGTAAACCACGTAACACTGAGCCAACCGAAGATTGTGAAGTGGGATAGAGATG
CGGCCGCTCAGGTGCAGCTGCAGCAGCCTGGCGCTGAACTCGTGCGGCCAGGCGCT
TCTGTGAAGCTGAGCTGTAAAGCCAGCGGCTACACCTTCACCAGCTACTGGATGAAC
TGGGTCAAGCAGCGGCCCGACCAGGGCCTGGAATGGATCGGAAGAATCGACCCCTA
CGACAGCGAGACACACTACAACCAGAAGTTCAAGGACAAGGCCATCCTGACCGTGG
ACAAGAGCAGCAGCACCGCCTACATGCAGCTGTCCAGCCTGACCAGCGAGGACAGC
GCCGTGTACTACTGCGCCAGAGGCAACTGGGACGACTACTGGGGCCAGGGCACAAC
CCTGACAGTGTCTAGCGGAGGCGGAGGATCAGGCGGCGGAGGAAGTGGGGGAGGC
GGATCTGATGTGCAGATTACCCAGTCCCCCAGCTACCTGGCCGCCTCTCCTGGCGAG
ACAATCACCATCAACTGCCGGGCCAGCAAGAGCATCTCCAAGGACCTGGCCTGGTA
TCAGGAAAAGCCCGGCAAGACCAACAAGCTGCTGATCTACAGCGGCTCCACCCTGC
AGTCCGGCATCCCCAGCAGATTTTCCGGCAGCGGCTCTGGCACCGACTTCACCCTGA
CCATCAGCTCCCTGGAACCCGAGGACTTTGCCATGTACTATTGCCAGCAGCACAACA
AGTACCCTTACACCTTCGGCGGAGGCACCAAGCTGGAAATCAAGAGCATCGAAGTG
ATGTACCCGCCTCCTTACCTGGACAACGAGAAGTCCAACGGCACCATCATCCACGTG
AAGGGAAAGCACCTGTGTCCTTCTCCACTGTTCCCCGGACCTAGCAAGCC
SEQ ID NO: 104 GRP78-mtIgG4-CD123-CAR_Extracellular domain
MDWIWRILFLVGAATGAHSCTVALPGGYVRVCEFESKYGPPCPSCPAPEFEGGPSVFLFP
PKPKDTLMISRTPEVTCVVVDVSQEDPEVQFNWYVDGVEVHNAKTKPREEQFQSTYRV
VSVLTVLHQDWLNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSRLTVDKSRWQEG
NVFSCSVMHEALHNHYTQKSLSLSLGKAAAQVQLQQPGAELVRPGASVKLSCKASGYT
FTSYWMNWVKQRPDQGLEWIGRIDPYDSETHYNQKFKDKAILTVDKSSSTAYMQLSSL
TSEDSAVYYCARGNWDDYWGQGTTLTVSSGGGGSGGGGSGGGGSDVQITQSPSYLAA
SPGETITINCRASKSISKDLAWYQEKPGKTNKLLIYSGSTLQSGIPSRFSGSGSGTDFTLTIS
SLEPEDFAMYYCQQHNKYPYTFGGGTKLEIKSIEVMYPPPYLDNEKSNGTIIHVKGKHL
CPSPLFPGPSKP
SEQ ID NO: 105 GRP78-mtIgG4-CD123-CAR_Extracellular domain
ATGGACTGGATTTGGCGGATCCTGTTTCTCGTGGGAGCCGCCACAGGCGCCCATTCT
TGTACTGTGGCCCTTCCTGGTGGATACGTTAGAGTGTGCGAATTCGAGTCTAAGTAC
GGCCCTCCTTGTCCTAGCTGCCCCGCTCCTGAATTTGAAGGCGGCCCTTCCGTGTTCC
TGTTTCCTCCAAAGCCTAAGGACACCCTGATGATCAGCAGAACCCCTGAAGTGACCT
GCGTGGTGGTGGACGTGTCCCAAGAGGATCCTGAGGTGCAGTTCAATTGGTACGTG
GACGGCGTGGAAGTGCACAACGCCAAGACCAAGCCTAGAGAGGAACAGTTCCAGA
GCACCTACAGAGTGGTGTCCGTGCTGACAGTGCTGCACCAGGATTGGCTGAACGGC
AAAGAGTACAAGTGCAAGGTGTCCAACAAGGGCCTGCCTAGCAGCATCGAGAAAAC
CATCAGCAAGGCCAAGGGCCAGCCAAGAGAACCCCAGGTGTACACACTGCCTCCAA
GCCAAGAGGAAATGACCAAGAACCAGGTGTCCCTGACCTGCCTGGTCAAGGGCTTC
TACCCTTCCGATATCGCCGTGGAATGGGAGAGCAATGGCCAGCCTGAGAACAACTA
CAAGACCACACCTCCTGTGCTGGACAGCGACGGCTCATTCTTCCTGTACAGCAGACT
GACCGTGGACAAGAGCAGATGGCAAGAGGGCAACGTGTTCAGCTGCAGCGTGATGC
ACGAGGCCCTGCACAACCACTACACCCAGAAGTCTCTGAGCCTGAGCCTCGGCAAG
GCGGCCGCTCAGGTGCAGCTGCAGCAGCCTGGCGCTGAACTCGTGCGGCCAGGCGC
TTCTGTGAAGCTGAGCTGTAAAGCCAGCGGCTACACCTTCACCAGCTACTGGATGAA
CTGGGTCAAGCAGCGGCCCGACCAGGGCCTGGAATGGATCGGAAGAATCGACCCCT
ACGACAGCGAGACACACTACAACCAGAAGTTCAAGGACAAGGCCATCCTGACCGTG
GACAAGAGCAGCAGCACCGCCTACATGCAGCTGTCCAGCCTGACCAGCGAGGACAG
CGCCGTGTACTACTGCGCCAGAGGCAACTGGGACGACTACTGGGGCCAGGGCACAA
CCCTGACAGTGTCTAGCGGAGGCGGAGGATCAGGCGGCGGAGGAAGTGGGGGAGG
CGGATCTGATGTGCAGATTACCCAGTCCCCCAGCTACCTGGCCGCCTCTCCTGGCGA
GACAATCACCATCAACTGCCGGGCCAGCAAGAGCATCTCCAAGGACCTGGCCTGGT
ATCAGGAAAAGCCCGGCAAGACCAACAAGCTGCTGATCTACAGCGGCTCCACCCTG
CAGTCCGGCATCCCCAGCAGATTTTCCGGCAGCGGCTCTGGCACCGACTTCACCCTG
ACCATCAGCTCCCTGGAACCCGAGGACTTTGCCATGTACTATTGCCAGCAGCACAAC
AAGTACCCTTACACCTTCGGCGGAGGCACCAAGCTGGAAATCAAGAGCATCGAAGT
GATGTACCCGCCTCCTTACCTGGACAACGAGAAGTCCAACGGCACCATCATCCACGT
GAAGGGAAAGCACCTGTGTCCTTCTCCACTGTTCCCCGGACCTAGCAAGCC
SEQ ID NO: 106 GRP78-GPcPcPc-CD123-CAR_Extracellular domain
MDWIWRILFLVGAATGAHSCTVALPGGYVRVCEFAGSGGSGGSGGSPVPSTPPTNSSST
PPTPSPSPVPSTPPTNSSSTPPTPSPSPVPSTPPTNSSSTPPTPSPSASAAAQVQLQQPGAELV
RPGASVKLSCKASGYTFTSYWMNWVKQRPDQGLEWIGRIDPYDSETHYNQKFKDKAIL
TVDKSSSTAYMQLSSLTSEDSAVYYCARGNWDDYWGQGTTLTVSSGGGGSGGGGSGG
GGSDVQITQSPSYLAASPGETITINCRASKSISKDLAWYQEKPGKTNKLLIYSGSTLQSGIP
SRFSGSGSGTDFTLTISSLEPEDFAMYYCQQHNKYPYTFGGGTKLEIKSIEVMYPPPYLD
NEKSNGTIIHVKGKHLCPSPLFPGPSKP
SEQ ID NO: 107 GRP78-GPcPcPc-CD123-CAR_Extracellular domain
ATGGACTGGATTTGGCGGATCCTGTTTCTCGTGGGAGCCGCCACAGGCGCCCATTCT
TGTACTGTGGCCCTTCCTGGTGGATACGTTAGAGTGTGCGAATTCGCAGGCAGTGGT
GGAAGTGGTGGGTCAGGCGGTTCCCCGGTTCCATCAACACCACCAACCAATAGCTC
ATCTACCCCGCCAACACCAAGTCCCTCTCCCGTCCCCAGCACACCCCCGACGAACTC
ATCCTCCACACCGCCAACCCCGTCTCCTTCCCCGGTTCCCTCTACACCTCCGACAAAC
TCTAGTTCAACGCCTCCCACTCCATCACCTTCCGCATCAGCGGCCGCTCAGGTGCAG
CTGCAGCAGCCTGGCGCTGAACTCGTGCGGCCAGGCGCTTCTGTGAAGCTGAGCTGT
AAAGCCAGCGGCTACACCTTCACCAGCTACTGGATGAACTGGGTCAAGCAGCGGCC
CGACCAGGGCCTGGAATGGATCGGAAGAATCGACCCCTACGACAGCGAGACACACT
ACAACCAGAAGTTCAAGGACAAGGCCATCCTGACCGTGGACAAGAGCAGCAGCACC
GCCTACATGCAGCTGTCCAGCCTGACCAGCGAGGACAGCGCCGTGTACTACTGCGC
CAGAGGCAACTGGGACGACTACTGGGGCCAGGGCACAACCCTGACAGTGTCTAGCG
GAGGCGGAGGATCAGGCGGCGGAGGAAGTGGGGGAGGCGGATCTGATGTGCAGAT
TACCCAGTCCCCCAGCTACCTGGCCGCCTCTCCTGGCGAGACAATCACCATCAACTG
CCGGGCCAGCAAGAGCATCTCCAAGGACCTGGCCTGGTATCAGGAAAAGCCCGGCA
AGACCAACAAGCTGCTGATCTACAGCGGCTCCACCCTGCAGTCCGGCATCCCCAGC
AGATTTTCCGGCAGCGGCTCTGGCACCGACTTCACCCTGACCATCAGCTCCCTGGAA
CCCGAGGACTTTGCCATGTACTATTGCCAGCAGCACAACAAGTACCCTTACACCTTC
GGCGGAGGCACCAAGCTGGAAATCAAGAGCATCGAAGTGATGTACCCGCCTCCTTA
CCTGGACAACGAGAAGTCCAACGGCACCATCATCCACGTGAAGGGAAAGCACCTGT
GTCCTTCTCCACTGTTCCCCGGACCTAGCAAGCC
SEQ ID NO: 108 CAR_Cytoplasmic domain
RSKRSRLLHSDYMNMTPRRPGPTRKHYQPYAPPRDFAAYRSRVKFSRSADAPAYQQGQ
NQLYNELNLGRREEYDVLDKRRGRDPEMGGKPRRKNPQEGLYNELQKDKMAEAYSEI
GMKGERRRGKGHDGLYQGLSTATKDTYDALHMQALPPR
SEQ ID NO: 109 CAR_Cytoplasmic domain
CGAAGCAAGCGGAGCCGGCTGCTGCACAGCGACTACATGAACATGACCCCTAGACG
GCCCGGACCAACCAGAAAGCACTACCAGCCTTACGCTCCTCCTAGAGATTTCGCCGC
CTACCGGTCCAGAGTGAAGTTCAGCAGGAGCGCAGACGCCCCCGCGTACCAGCAGG
GCCAGAACCAGCTCTATAACGAGCTCAATCTAGGACGAAGAGAGGAGTACGATGTT
TTGGACAAGAGACGTGGCCGGGACCCTGAGATGGGGGGAAAGCCGAGAAGGAAGA
ACCCTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCCTTT
ACCAGGGTCTCAGTACAGCCACCAAGGACACCTACGACGCCCTTCACATGCAGGCC
CTGCCCCCTCGC
SEQ ID NO: 110 GRP78-G4S3-CD123-CAR
MDWIWRILFLVGAATGAHSCTVALPGGYVRVCEFGGGGSGGGGGGGGSAAAQVQLQ
QPGAELVRPGASVKLSCKASGYTFTSYWMNWVKQRPDQGLEWIGRIDPYDSETHYNQ
KFKDKAILTVDKSSSTAYMQLSSLTSEDSAVYYCARGNWDDYWGQGTTLTVSSGGGGS
GGGGSGGGGSDVQITQSPSYLAASPGETITINCRASKSISKDLAWYQEKPGKTNKLLIYS
GSTLQSGIPSRFSGSGSGTDFTLTISSLEPEDFAMYYCQQHNKYPYTFGGGTKLEIKSIEV
MYPPPYLDNEKSNGTIIHVKGKHLCPSPLFPGPSKPFWVLVVVGGVLACYSLLVTVAFII
FWVRSKRSRLLHSDYMNMTPRRPGPTRKHYQPYAPPRDFAAYRSRVKFSRSADAPAYQ
QGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
SEQ ID NO: 111 GRP78-G4S3-CD123-CAR
ATGGACTGGATTTGGCGGATCCTGTTTCTCGTGGGAGCCGCCACAGGCGCCCATTCT
TGTACTGTGGCCCTTCCTGGTGGATACGTTAGAGTGTGCGAATTCGGAGGCGGAGGA
TCAGGCGGCGGAGGAAGTGGGGGAGGCGGATCTGCGGCCGCTCAGGTGCAGCTGCA
GCAGCCTGGCGCTGAACTCGTGCGGCCAGGCGCTTCTGTGAAGCTGAGCTGTAAAG
CCAGCGGCTACACCTTCACCAGCTACTGGATGAACTGGGTCAAGCAGCGGCCCGAC
CAGGGCCTGGAATGGATCGGAAGAATCGACCCCTACGACAGCGAGACACACTACAA
CCAGAAGTTCAAGGACAAGGCCATCCTGACCGTGGACAAGAGCAGCAGCACCGCCT
ACATGCAGCTGTCCAGCCTGACCAGCGAGGACAGCGCCGTGTACTACTGCGCCAGA
GGCAACTGGGACGACTACTGGGGCCAGGGCACAACCCTGACAGTGTCTAGCGGAGG
CGGAGGATCAGGCGGCGGAGGAAGTGGGGGAGGCGGATCTGATGTGCAGATTACCC
AGTCCCCCAGCTACCTGGCCGCCTCTCCTGGCGAGACAATCACCATCAACTGCCGGG
CCAGCAAGAGCATCTCCAAGGACCTGGCCTGGTATCAGGAAAAGCCCGGCAAGACC
AACAAGCTGCTGATCTACAGCGGCTCCACCCTGCAGTCCGGCATCCCCAGCAGATTT
TCCGGCAGCGGCTCTGGCACCGACTTCACCCTGACCATCAGCTCCCTGGAACCCGAG
GACTTTGCCATGTACTATTGCCAGCAGCACAACAAGTACCCTTACACCTTCGGCGGA
GGCACCAAGCTGGAAATCAAGAGCATCGAAGTGATGTACCCGCCTCCTTACCTGGA
CAACGAGAAGTCCAACGGCACCATCATCCACGTGAAGGGAAAGCACCTGTGTCCTT
CTCCACTGTTCCCCGGACCTAGCAAGCCTTTCTGGGTGCTCGTTGTTGTTGGCGGCGT
GCTGGCCTGTTACAGCCTGCTGGTTACCGTGGCCTTCATCATCTTTTGGGTCCGAAGC
AAGCGGAGCCGGCTGCTGCACAGCGACTACATGAACATGACCCCTAGACGGCCCGG
ACCAACCAGAAAGCACTACCAGCCTTACGCTCCTCCTAGAGATTTCGCCGCCTACCG
GTCCAGAGTGAAGTTCAGCAGGAGCGCAGACGCCCCCGCGTACCAGCAGGGCCAGA
ACCAGCTCTATAACGAGCTCAATCTAGGACGAAGAGAGGAGTACGATGTTTTGGAC
AAGAGACGTGGCCGGGACCCTGAGATGGGGGGAAAGCCGAGAAGGAAGAACCCTC
AGGAAGGCCTGTACAATGAACTGCAGAAAGATAAGATGGCGGAGGCCTACAGTGA
GATTGGGATGAAAGGCGAGCGCCGGAGGGGCAAGGGGCACGATGGCCTTTACCAG
GGTCTCAGTACAGCCACCAAGGACACCTACGACGCCCTTCACATGCAGGCCCTGCCC
CCTCGC
SEQ ID NO: 112 GRP78-B2M-CD123-CAR
MDWIWRILFLVGAATGAHSCTVALPGGYVRVCEFIQRTPKIQVYSRHPAENGKSNFLNC
YVSGFHPSDIEVDLLKNGERIEKVEHSDLSFSKDWSFYLLYYTEFTPTEKDEYACRVNHV
TLSQPKIVKWDRDAAAQVQLQQPGAELVRPGASVKLSCKASGYTFTSYWMNWVKQRP
DQGLEWIGRIDPYDSETHYNQKFKDKAILTVDKSSSTAYMQLSSLTSEDSAVYYCARGN
WDDYWGQGTTLTVSSGGGGSGGGGSGGGGSDVQITQSPSYLAASPGETITINCRASKSIS
KDLAWYQEKPGKTNKLLIYSGSTLQSGIPSRFSGSGSGTDFTLTISSLEPEDFAMYYCQQ
HNKYPYTFGGGTKLEIKSIEVMYPPPYLDNEKSNGTIIHVKGKHLCPSPLFPGPSKPFWVL
VVVGGVLACYSLLVTVAFIIFWVRSKRSRLLHSDYMNMTPRRPGPTRKHYQPYAPPRDF
AAYRSRVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHMQA
LPPR
SEQ ID NO: 113 GRP78-B2M-CD123-CAR
ATGGACTGGATTTGGCGGATCCTGTTTCTCGTGGGAGCCGCCACAGGCGCCCATTCT
TGTACTGTGGCCCTTCCTGGTGGATACGTTAGAGTGTGCGAATTCATACAACGCACG
CCTAAAATCCAAGTCTATAGTCGGCACCCGGCGGAAAATGGGAAATCTAATTTCCTT
AACTGCTATGTGTCCGGTTTCCACCCATCCGACATTGAGGTAGACCTGCTGAAAAAC
GGGGAACGGATAGAAAAGGTCGAACACAGTGATCTGTCCTTTAGTAAGGATTGGTC
TTTTTATCTGCTCTACTACACAGAGTTTACTCCGACAGAAAAAGACGAGTACGCGTG
CCGCGTAAACCACGTAACACTGAGCCAACCGAAGATTGTGAAGTGGGATAGAGATG
CGGCCGCTCAGGTGCAGCTGCAGCAGCCTGGCGCTGAACTCGTGCGGCCAGGCGCT
TCTGTGAAGCTGAGCTGTAAAGCCAGCGGCTACACCTTCACCAGCTACTGGATGAAC
TGGGTCAAGCAGCGGCCCGACCAGGGCCTGGAATGGATCGGAAGAATCGACCCCTA
CGACAGCGAGACACACTACAACCAGAAGTTCAAGGACAAGGCCATCCTGACCGTGG
ACAAGAGCAGCAGCACCGCCTACATGCAGCTGTCCAGCCTGACCAGCGAGGACAGC
GCCGTGTACTACTGCGCCAGAGGCAACTGGGACGACTACTGGGGCCAGGGCACAAC
CCTGACAGTGTCTAGCGGAGGCGGAGGATCAGGCGGCGGAGGAAGTGGGGGAGGC
GGATCTGATGTGCAGATTACCCAGTCCCCCAGCTACCTGGCCGCCTCTCCTGGCGAG
ACAATCACCATCAACTGCCGGGCCAGCAAGAGCATCTCCAAGGACCTGGCCTGGTA
TCAGGAAAAGCCCGGCAAGACCAACAAGCTGCTGATCTACAGCGGCTCCACCCTGC
AGTCCGGCATCCCCAGCAGATTTTCCGGCAGCGGCTCTGGCACCGACTTCACCCTGA
CCATCAGCTCCCTGGAACCCGAGGACTTTGCCATGTACTATTGCCAGCAGCACAACA
AGTACCCTTACACCTTCGGCGGAGGCACCAAGCTGGAAATCAAGAGCATCGAAGTG
ATGTACCCGCCTCCTTACCTGGACAACGAGAAGTCCAACGGCACCATCATCCACGTG
AAGGGAAAGCACCTGTGTCCTTCTCCACTGTTCCCCGGACCTAGCAAGCCTTTCTGG
GTGCTCGTTGTTGTTGGCGGCGTGCTGGCCTGTTACAGCCTGCTGGTTACCGTGGCCT
TCATCATCTTTTGGGTCCGAAGCAAGCGGAGCCGGCTGCTGCACAGCGACTACATGA
ACATGACCCCTAGACGGCCCGGACCAACCAGAAAGCACTACCAGCCTTACGCTCCT
CCTAGAGATTTCGCCGCCTACCGGTCCAGAGTGAAGTTCAGCAGGAGCGCAGACGC
CCCCGCGTACCAGCAGGGCCAGAACCAGCTCTATAACGAGCTCAATCTAGGACGAA
GAGAGGAGTACGATGTTTTGGACAAGAGACGTGGCCGGGACCCTGAGATGGGGGGA
AAGCCGAGAAGGAAGAACCCTCAGGAAGGCCTGTACAATGAACTGCAGAAAGATA
AGATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGACG
CCCTTCACATGCAGGCCCTGCCCCCTCGC
SEQ ID NO: 114 GRP78-mtIgG4-CD123-CAR
MDWIWRILFLVGAATGAHSCTVALPGGYVRVCEFESKYGPPCPSCPAPEFEGGPSVFLFP
PKPKDTLMISRTPEVTCVVVDVSQEDPEVQFNWYVDGVEVHNAKTKPREEQFQSTYRV
VSVLTVLHQDWLNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSRLTVDKSRWQEG
NVFSCSVMHEALHNHYTQKSLSLSLGKAAAQVQLQQPGAELVRPGASVKLSCKASGYT
FTSYWMNWVKQRPDQGLEWIGRIDPYDSETHYNQKFKDKAILTVDKSSSTAYMQLSSL
TSEDSAVYYCARGNWDDYWGQGTTLTVSSGGGGSGGGGSGGGGSDVQITQSPSYLAA
SPGETITINCRASKSISKDLAWYQEKPGKTNKLLIYSGSTLQSGIPSRFSGSGSGTDFTLTIS
SLEPEDFAMYYCQQHNKYPYTFGGGTKLEIKSIEVMYPPPYLDNEKSNGTIIHVKGKHL
CPSPLFPGPSKPFWVLVVVGGVLACYSLLVTVAFIIFWVRSKRSRLLHSDYMNMTPRRP
GPTRKHYQPYAPPRDFAAYRSRVKFSRSADAPAYQQGQNQLYNELNLGRREEYDVLDK
RRGRDPEMGGKPRRKNPQEGLYNELQKDKMAEAYSEIGMKGERRRGKGHDGLYQGLS
TATKDTYDALHMQALPPR
SEQ ID NO: 115 GRP78-mtIgG4-CD123-CAR
ATGGACTGGATTTGGCGGATCCTGTTTCTCGTGGGAGCCGCCACAGGCGCCCATTCT
TGTACTGTGGCCCTTCCTGGTGGATACGTTAGAGTGTGCGAATTCGAGTCTAAGTAC
GGCCCTCCTTGTCCTAGCTGCCCCGCTCCTGAATTTGAAGGCGGCCCTTCCGTGTTCC
TGTTTCCTCCAAAGCCTAAGGACACCCTGATGATCAGCAGAACCCCTGAAGTGACCT
GCGTGGTGGTGGACGTGTCCCAAGAGGATCCTGAGGTGCAGTTCAATTGGTACGTG
GACGGCGTGGAAGTGCACAACGCCAAGACCAAGCCTAGAGAGGAACAGTTCCAGA
GCACCTACAGAGTGGTGTCCGTGCTGACAGTGCTGCACCAGGATTGGCTGAACGGC
AAAGAGTACAAGTGCAAGGTGTCCAACAAGGGCCTGCCTAGCAGCATCGAGAAAAC
CATCAGCAAGGCCAAGGGCCAGCCAAGAGAACCCCAGGTGTACACACTGCCTCCAA
GCCAAGAGGAAATGACCAAGAACCAGGTGTCCCTGACCTGCCTGGTCAAGGGCTTC
TACCCTTCCGATATCGCCGTGGAATGGGAGAGCAATGGCCAGCCTGAGAACAACTA
CAAGACCACACCTCCTGTGCTGGACAGCGACGGCTCATTCTTCCTGTACAGCAGACT
GACCGTGGACAAGAGCAGATGGCAAGAGGGCAACGTGTTCAGCTGCAGCGTGATGC
ACGAGGCCCTGCACAACCACTACACCCAGAAGTCTCTGAGCCTGAGCCTCGGCAAG
GCGGCCGCTCAGGTGCAGCTGCAGCAGCCTGGCGCTGAACTCGTGCGGCCAGGCGC
TTCTGTGAAGCTGAGCTGTAAAGCCAGCGGCTACACCTTCACCAGCTACTGGATGAA
CTGGGTCAAGCAGCGGCCCGACCAGGGCCTGGAATGGATCGGAAGAATCGACCCCT
ACGACAGCGAGACACACTACAACCAGAAGTTCAAGGACAAGGCCATCCTGACCGTG
GACAAGAGCAGCAGCACCGCCTACATGCAGCTGTCCAGCCTGACCAGCGAGGACAG
CGCCGTGTACTACTGCGCCAGAGGCAACTGGGACGACTACTGGGGCCAGGGCACAA
CCCTGACAGTGTCTAGCGGAGGCGGAGGATCAGGCGGCGGAGGAAGTGGGGGAGG
CGGATCTGATGTGCAGATTACCCAGTCCCCCAGCTACCTGGCCGCCTCTCCTGGCGA
GACAATCACCATCAACTGCCGGGCCAGCAAGAGCATCTCCAAGGACCTGGCCTGGT
ATCAGGAAAAGCCCGGCAAGACCAACAAGCTGCTGATCTACAGCGGCTCCACCCTG
CAGTCCGGCATCCCCAGCAGATTTTCCGGCAGCGGCTCTGGCACCGACTTCACCCTG
ACCATCAGCTCCCTGGAACCCGAGGACTTTGCCATGTACTATTGCCAGCAGCACAAC
AAGTACCCTTACACCTTCGGCGGAGGCACCAAGCTGGAAATCAAGAGCATCGAAGT
GATGTACCCGCCTCCTTACCTGGACAACGAGAAGTCCAACGGCACCATCATCCACGT
GAAGGGAAAGCACCTGTGTCCTTCTCCACTGTTCCCCGGACCTAGCAAGCCTTTCTG
GGTGCTCGTTGTTGTTGGCGGCGTGCTGGCCTGTTACAGCCTGCTGGTTACCGTGGC
CTTCATCATCTTTTGGGTCCGAAGCAAGCGGAGCCGGCTGCTGCACAGCGACTACAT
GAACATGACCCCTAGACGGCCCGGACCAACCAGAAAGCACTACCAGCCTTACGCTC
CTCCTAGAGATTTCGCCGCCTACCGGTCCAGAGTGAAGTTCAGCAGGAGCGCAGAC
GCCCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGGGG
GAAAGCCGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGGC
AAGGGGCACGATGGCCTTTACCAGGGTCTCAGTACAGCCACCAAGGACACCTACGA
CGCCCTTCACATGCAGGCCCTGCCCCCTCGC
SEQ ID NO: 116 GRP78-GPcPcPc-CD123-CAR
MDWIWRILFLVGAATGAHSCTVALPGGYVRVCEFAGSGGSGGSGGSPVPSTPPTNSSST
PPTPSPSPVPSTPPTNSSSTPPTPSPSPVPSTPPTNSSSTPPTPSPSASAAAQVQLQQPGAELV
RPGASVKLSCKASGYTFTSYWMNWVKQRPDQGLEWIGRIDPYDSETHYNQKFKDKAIL
TVDKSSSTAYMQLSSLTSEDSAVYYCARGNWDDYWGQGTTLTVSSGGGGSGGGGSGG
GGSDVQITQSPSYLAASPGETITINCRASKSISKDLAWYQEKPGKTNKLLIYSGSTLQSGIP
SRFSGSGSGTDFTLTISSLEPEDFAMYYCQQHNKYPYTFGGGTKLEIKSIEVMYPPPYLD
NEKSNGTIIHVKGKHLCPSPLFPGPSKPFWVLVVVGGVLACYSLLVTVAFIIFWVRSKRS
RLLHSDYMNMTPRRPGPTRKHYQPYAPPRDFAAYRSRVKFSRSADAPAYQQGQNQLY
NELNLGRREEYDVLDKRRGRDPEMGGKPRRKNPQEGLYNELQKDKMAEAYSEIGMKG
ERRRGKGHDGLYQGLSTATKDTYDALHMQALPPR
SEQ ID NO: 117 GRP78-GPcPcPc-CD123-CAR
ATGGACTGGATTTGGCGGATCCTGTTTCTCGTGGGAGCCGCCACAGGCGCCCATTCT
TGTACTGTGGCCCTTCCTGGTGGATACGTTAGAGTGTGCGAATTCGCAGGCAGTGGT
GGAAGTGGTGGGTCAGGCGGTTCCCCGGTTCCATCAACACCACCAACCAATAGCTC
ATCTACCCCGCCAACACCAAGTCCCTCTCCCGTCCCCAGCACACCCCCGACGAACTC
ATCCTCCACACCGCCAACCCCGTCTCCTTCCCCGGTTCCCTCTACACCTCCGACAAAC
TCTAGTTCAACGCCTCCCACTCCATCACCTTCCGCATCAGCGGCCGCTCAGGTGCAG
CTGCAGCAGCCTGGCGCTGAACTCGTGCGGCCAGGCGCTTCTGTGAAGCTGAGCTGT
AAAGCCAGCGGCTACACCTTCACCAGCTACTGGATGAACTGGGTCAAGCAGCGGCC
CGACCAGGGCCTGGAATGGATCGGAAGAATCGACCCCTACGACAGCGAGACACACT
ACAACCAGAAGTTCAAGGACAAGGCCATCCTGACCGTGGACAAGAGCAGCAGCACC
GCCTACATGCAGCTGTCCAGCCTGACCAGCGAGGACAGCGCCGTGTACTACTGCGC
CAGAGGCAACTGGGACGACTACTGGGGCCAGGGCACAACCCTGACAGTGTCTAGCG
GAGGCGGAGGATCAGGCGGCGGAGGAAGTGGGGGAGGCGGATCTGATGTGCAGAT
TACCCAGTCCCCCAGCTACCTGGCCGCCTCTCCTGGCGAGACAATCACCATCAACTG
CCGGGCCAGCAAGAGCATCTCCAAGGACCTGGCCTGGTATCAGGAAAAGCCCGGCA
AGACCAACAAGCTGCTGATCTACAGCGGCTCCACCCTGCAGTCCGGCATCCCCAGC
AGATTTTCCGGCAGCGGCTCTGGCACCGACTTCACCCTGACCATCAGCTCCCTGGAA
CCCGAGGACTTTGCCATGTACTATTGCCAGCAGCACAACAAGTACCCTTACACCTTC
GGCGGAGGCACCAAGCTGGAAATCAAGAGCATCGAAGTGATGTACCCGCCTCCTTA
CCTGGACAACGAGAAGTCCAACGGCACCATCATCCACGTGAAGGGAAAGCACCTGT
GTCCTTCTCCACTGTTCCCCGGACCTAGCAAGCCTTTCTGGGTGCTCGTTGTTGTTGG
CGGCGTGCTGGCCTGTTACAGCCTGCTGGTTACCGTGGCCTTCATCATCTTTTGGGTC
CGAAGCAAGCGGAGCCGGCTGCTGCACAGCGACTACATGAACATGACCCCTAGACG
GCCCGGACCAACCAGAAAGCACTACCAGCCTTACGCTCCTCCTAGAGATTTCGCCGC
CTACCGGTCCAGAGTGAAGTTCAGCAGGAGCGCAGACGCCCCCGCGTACCAGCAGG
GCCAGAACCAGCTCTATAACGAGCTCAATCTAGGACGAAGAGAGGAGTACGATGTT
TTGGACAAGAGACGTGGCCGGGACCCTGAGATGGGGGGAAAGCCGAGAAGGAAGA
ACCCTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCCTTT
ACCAGGGTCTCAGTACAGCCACCAAGGACACCTACGACGCCCTTCACATGCAGGCC
CTGCCCCCTCGC
SEQ ID NO: 118 GRP78-G4S3-CD123-CAR_full-length
MDWIWRILFLVGAATGAHSCTVALPGGYVRVCEFGGGGSGGGGSGGGGSAAAQVQLQ
QPGAELVRPGASVKLSCKASGYTFTSYWMNWVKQRPDQGLEWIGRIDPYDSETHYNQ
KFKDKAILTVDKSSSTAYMQLSSLTSEDSAVYYCARGNWDDYWGQGTTLTVSSGGGGS
GGGGSGGGGSDVQITQSPSYLAASPGETITINCRASKSISKDLAWYQEKPGKTNKLLIYS
GSTLQSGIPSRFSGSGSGTDFTLTISSLEPEDFAMYYCQQHNKYPYTFGGGTKLEIKSIEV
MYPPPYLDNEKSNGTIIHVKGKHLCPSPLFPGPSKPFWVLVVVGGVLACYSLLVTVAFII
FWVRSKRSRLLHSDYMNMTPRRPGPTRKHYQPYAPPRDFAAYRSRVKFSRSADAPAYQ
QGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPRRSGVDSGEGRGSLLTC
GDVEENPGPMPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPGPPSEKAWQPG
WTVNVEGSGELFRWNVSDLGGLGCGLKNRSSEGPSSPSGKLMSPKLYVWAKDRPEIWE
GEPPCLPPRDSLNQSLSQDLTMAPGSTLWLSCGVPPDSVSRGPLSWTHVHPKGPKSLLSL
ELKDDRPARDMWVMETGLLLPRATAQDAGKYYCHRGNLTMSFHLEITARPVLWHWLL
RTGGWKVSAVTLAYLIFCLCSLVGILHLQRALVLRRKRKRMTDPTRRF*
SEQ ID NO: 119 GRP78-G4S3-CD123-CAR_full-length
ATGGACTGGATTTGGCGGATCCTGTTTCTCGTGGGAGCCGCCACAGGCGCCCATTCT
TGTACTGTGGCCCTTCCTGGTGGATACGTTAGAGTGTGCGAATTCGGAGGCGGAGGA
TCAGGCGGCGGAGGAAGTGGGGGAGGCGGATCTGCGGCCGCTCAGGTGCAGCTGCA
GCAGCCTGGCGCTGAACTCGTGCGGCCAGGCGCTTCTGTGAAGCTGAGCTGTAAAG
CCAGCGGCTACACCTTCACCAGCTACTGGATGAACTGGGTCAAGCAGCGGCCCGAC
CAGGGCCTGGAATGGATCGGAAGAATCGACCCCTACGACAGCGAGACACACTACAA
CCAGAAGTTCAAGGACAAGGCCATCCTGACCGTGGACAAGAGCAGCAGCACCGCCT
ACATGCAGCTGTCCAGCCTGACCAGCGAGGACAGCGCCGTGTACTACTGCGCCAGA
GGCAACTGGGACGACTACTGGGGCCAGGGCACAACCCTGACAGTGTCTAGCGGAGG
CGGAGGATCAGGCGGCGGAGGAAGTGGGGGAGGCGGATCTGATGTGCAGATTACCC
AGTCCCCCAGCTACCTGGCCGCCTCTCCTGGCGAGACAATCACCATCAACTGCCGGG
CCAGCAAGAGCATCTCCAAGGACCTGGCCTGGTATCAGGAAAAGCCCGGCAAGACC
AACAAGCTGCTGATCTACAGCGGCTCCACCCTGCAGTCCGGCATCCCCAGCAGATTT
TCCGGCAGCGGCTCTGGCACCGACTTCACCCTGACCATCAGCTCCCTGGAACCCGAG
GACTTTGCCATGTACTATTGCCAGCAGCACAACAAGTACCCTTACACCTTCGGCGGA
GGCACCAAGCTGGAAATCAAGAGCATCGAAGTGATGTACCCGCCTCCTTACCTGGA
CAACGAGAAGTCCAACGGCACCATCATCCACGTGAAGGGAAAGCACCTGTGTCCTT
CTCCACTGTTCCCCGGACCTAGCAAGCCTTTCTGGGTGCTCGTTGTTGTTGGCGGCGT
GCTGGCCTGTTACAGCCTGCTGGTTACCGTGGCCTTCATCATCTTTTGGGTCCGAAGC
AAGCGGAGCCGGCTGCTGCACAGCGACTACATGAACATGACCCCTAGACGGCCCGG
ACCAACCAGAAAGCACTACCAGCCTTACGCTCCTCCTAGAGATTTCGCCGCCTACCG
GTCCAGAGTGAAGTTCAGCAGGAGCGCAGACGCCCCCGCGTACCAGCAGGGCCAGA
ACCAGCTCTATAACGAGCTCAATCTAGGACGAAGAGAGGAGTACGATGTTTTGGAC
AAGAGACGTGGCCGGGACCCTGAGATGGGGGGAAAGCCGAGAAGGAAGAACCCTC
AGGAAGGCCTGTACAATGAACTGCAGAAAGATAAGATGGCGGAGGCCTACAGTGA
GATTGGGATGAAAGGCGAGCGCCGGAGGGGCAAGGGGCACGATGGCCTTTACCAG
GGTCTCAGTACAGCCACCAAGGACACCTACGACGCCCTTCACATGCAGGCCCTGCCC
CCTCGCAGATCTGGCGTCGACTCTGGCGAAGGCAGAGGCTCTCTGCTGACATGTGGC
GACGTGGAAGAGAATCCTGGACCTATGCCTCCCCCCAGACTGCTGTTCTTCCTGCTG
TTCCTGACCCCTATGGAAGTGCGGCCCGAGGAACCCCTGGTCGTGAAAGTGGAAGA
GGGCGACAACGCCGTGCTGCAGTGTCTGAAGGGCACCTCCGATGGCCCTACCCAGC
AGCTGACCTGGTCCAGAGAGAGCCCCCTGAAGCCCTTCCTGAAGCTGTCTCTGGGCC
TGCCTGGCCTGGGCATCCATATGAGGCCACTGGCCATCTGGCTGTTCATCTTCAACG
TGTCCCAGCAGATGGGAGGCTTCTACCTGTGCCAGCCTGGCCCACCTTCTGAGAAGG
CTTGGCAGCCTGGCTGGACCGTGAACGTGGAAGGATCTGGCGAGCTGTTCCGGTGG
AACGTGTCCGATCTGGGCGGCCTGGGATGCGGCCTGAAGAACAGATCTAGCGAGGG
CCCCAGCAGCCCCAGCGGCAAACTGATGAGCCCCAAGCTGTACGTGTGGGCCAAGG
ACAGACCCGAGATTTGGGAGGGCGAGCCCCCTTGCCTGCCCCCTAGAGATAGCCTG
AACCAGAGCCTGAGCCAGGACCTGACAATGGCCCCTGGCAGCACACTGTGGCTGAG
CTGTGGCGTGCCACCCGACTCTGTGTCTAGAGGCCCTCTGAGCTGGACCCACGTGCA
CCCTAAGGGCCCTAAGAGCCTGCTGTCCCTGGAACTGAAGGACGACAGGCCCGCCA
GAGATATGTGGGTCATGGAAACCGGCCTGCTGCTGCCTAGAGCCACAGCCCAGGAT
GCCGGCAAGTACTACTGCCACAGAGGCAACCTGACCATGAGCTTCCACCTGGAAAT
CACCGCCAGACCCGTGCTGTGGCACTGGCTGCTGAGAACCGGCGGATGGAAAGTGT
CCGCCGTGACTCTGGCCTACCTGATCTTCTGCCTGTGCTCCCTCGTGGGCATCCTGCA
TCTGCAGAGGGCTCTGGTGCTGCGGCGGAAGCGGAAGAGAATGACCGACCCTACCC
GGCGGTTCTAA
SEQ ID NO: 120 GRP78-B2M-CD123-CAR_full-length
MDWIWRILFLVGAATGAHSCTVALPGGYVRVCEFIQRTPKIQVYSRHPAENGKSNFLNC
YVSGFHPSDIEVDLLKNGERIEKVEHSDLSFSKDWSFYLLYYTEFTPTEKDEYACRVNHV
TLSQPKIVKWDRDAAAQVQLQQPGAELVRPGASVKLSCKASGYTFTSYWMNWVKQRP
DQGLEWIGRIDPYDSETHYNQKFKDKAILTVDKSSSTAYMQLSSLTSEDSAVYYCARGN
WDDYWGQGTTLTVSSGGGGSGGGGSGGGGSDVQITQSPSYLAASPGETITINCRASKSIS
KDLAWYQEKPGKTNKLLIYSGSTLQSGIPSRFSGSGSGTDFTLTISSLEPEDFAMYYCQQ
HNKYPYTFGGGTKLEIKSIEVMYPPPYLDNEKSNGTIIHVKGKHLCPSPLFPGPSKPFWVL
VVVGGVLACYSLLVTVAFIIFWVRSKRSRLLHSDYMNMTPRRPGPTRKHYQPYAPPRDF
AAYRSRVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHMQA
LPPRRSGVDSGEGRGSLLTCGDVEENPGPMPPPRLLFFLLFLTPMEVRPEEPLVVKVEEG
DNAVLQCLKGTSDGPTQQLTWSRESPLKPFLKLSLGLPGLGIHMRPLAIWLFIFNVSQQM
GGFYLCQPGPPSEKAWQPGWTVNVEGSGELFRWNVSDLGGLGCGLKNRSSEGPSSPSG
KLMSPKLYVWAKDRPEIWEGEPPCLPPRDSLNQSLSQDLTMAPGSTLWLSCGVPPDSVS
RGPLSWTHVHPKGPKSLLSLELKDDRPARDMWVMETGLLLPRATAQDAGKYYCHRGN
LTMSFHLEITARPVLWHWLLRTGGWKVSAVTLAYLIFCLCSLVGILHLQRALVLRRKRK
RMTDPTRRF*
SEQ ID NO: 121 GRP78-B2M-CD123-CAR_full-length
ATGGACTGGATTTGGCGGATCCTGTTTCTCGTGGGAGCCGCCACAGGCGCCCATTCT
TGTACTGTGGCCCTTCCTGGTGGATACGTTAGAGTGTGCGAATTCATACAACGCACG
CCTAAAATCCAAGTCTATAGTCGGCACCCGGCGGAAAATGGGAAATCTAATTTCCTT
AACTGCTATGTGTCCGGTTTCCACCCATCCGACATTGAGGTAGACCTGCTGAAAAAC
GGGGAACGGATAGAAAAGGTCGAACACAGTGATCTGTCCTTTAGTAAGGATTGGTC
TTTTTATCTGCTCTACTACACAGAGTTTACTCCGACAGAAAAAGACGAGTACGCGTG
CCGCGTAAACCACGTAACACTGAGCCAACCGAAGATTGTGAAGTGGGATAGAGATG
CGGCCGCTCAGGTGCAGCTGCAGCAGCCTGGCGCTGAACTCGTGCGGCCAGGCGCT
TCTGTGAAGCTGAGCTGTAAAGCCAGCGGCTACACCTTCACCAGCTACTGGATGAAC
TGGGTCAAGCAGCGGCCCGACCAGGGCCTGGAATGGATCGGAAGAATCGACCCCTA
CGACAGCGAGACACACTACAACCAGAAGTTCAAGGACAAGGCCATCCTGACCGTGG
ACAAGAGCAGCAGCACCGCCTACATGCAGCTGTCCAGCCTGACCAGCGAGGACAGC
GCCGTGTACTACTGCGCCAGAGGCAACTGGGACGACTACTGGGGCCAGGGCACAAC
CCTGACAGTGTCTAGCGGAGGCGGAGGATCAGGCGGCGGAGGAAGTGGGGGAGGC
GGATCTGATGTGCAGATTACCCAGTCCCCCAGCTACCTGGCCGCCTCTCCTGGCGAG
ACAATCACCATCAACTGCCGGGCCAGCAAGAGCATCTCCAAGGACCTGGCCTGGTA
TCAGGAAAAGCCCGGCAAGACCAACAAGCTGCTGATCTACAGCGGCTCCACCCTGC
AGTCCGGCATCCCCAGCAGATTTTCCGGCAGCGGCTCTGGCACCGACTTCACCCTGA
CCATCAGCTCCCTGGAACCCGAGGACTTTGCCATGTACTATTGCCAGCAGCACAACA
AGTACCCTTACACCTTCGGCGGAGGCACCAAGCTGGAAATCAAGAGCATCGAAGTG
ATGTACCCGCCTCCTTACCTGGACAACGAGAAGTCCAACGGCACCATCATCCACGTG
AAGGGAAAGCACCTGTGTCCTTCTCCACTGTTCCCCGGACCTAGCAAGCCTTTCTGG
GTGCTCGTTGTTGTTGGCGGCGTGCTGGCCTGTTACAGCCTGCTGGTTACCGTGGCCT
TCATCATCTTTTGGGTCCGAAGCAAGCGGAGCCGGCTGCTGCACAGCGACTACATGA
ACATGACCCCTAGACGGCCCGGACCAACCAGAAAGCACTACCAGCCTTACGCTCCT
CCTAGAGATTTCGCCGCCTACCGGTCCAGAGTGAAGTTCAGCAGGAGCGCAGACGC
CCCCGCGTACCAGCAGGGCCAGAACCAGCTCTATAACGAGCTCAATCTAGGACGAA
GAGAGGAGTACGATGTTTTGGACAAGAGACGTGGCCGGGACCCTGAGATGGGGGGA
AAGCCGAGAAGGAAGAACCCTCAGGAAGGCCTGTACAATGAACTGCAGAAAGATA
AGATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGACG
CCCTTCACATGCAGGCCCTGCCCCCTCGCAGATCTGGCGTCGACTCTGGCGAAGGCA
GAGGCTCTCTGCTGACATGTGGCGACGTGGAAGAGAATCCTGGACCTATGCCTCCCC
CCAGACTGCTGTTCTTCCTGCTGTTCCTGACCCCTATGGAAGTGCGGCCCGAGGAAC
CCCTGGTCGTGAAAGTGGAAGAGGGCGACAACGCCGTGCTGCAGTGTCTGAAGGGC
ACCTCCGATGGCCCTACCCAGCAGCTGACCTGGTCCAGAGAGAGCCCCCTGAAGCC
CTTCCTGAAGCTGTCTCTGGGCCTGCCTGGCCTGGGCATCCATATGAGGCCACTGGC
CATCTGGCTGTTCATCTTCAACGTGTCCCAGCAGATGGGAGGCTTCTACCTGTGCCA
GCCTGGCCCACCTTCTGAGAAGGCTTGGCAGCCTGGCTGGACCGTGAACGTGGAAG
GATCTGGCGAGCTGTTCCGGTGGAACGTGTCCGATCTGGGCGGCCTGGGATGCGGCC
TGAAGAACAGATCTAGCGAGGGCCCCAGCAGCCCCAGCGGCAAACTGATGAGCCCC
AAGCTGTACGTGTGGGCCAAGGACAGACCCGAGATTTGGGAGGGCGAGCCCCCTTG
CCTGCCCCCTAGAGATAGCCTGAACCAGAGCCTGAGCCAGGACCTGACAATGGCCC
CTGGCAGCACACTGTGGCTGAGCTGTGGCGTGCCACCCGACTCTGTGTCTAGAGGCC
CTCTGAGCTGGACCCACGTGCACCCTAAGGGCCCTAAGAGCCTGCTGTCCCTGGAAC
TGAAGGACGACAGGCCCGCCAGAGATATGTGGGTCATGGAAACCGGCCTGCTGCTG
CCTAGAGCCACAGCCCAGGATGCCGGCAAGTACTACTGCCACAGAGGCAACCTGAC
CATGAGCTTCCACCTGGAAATCACCGCCAGACCCGTGCTGTGGCACTGGCTGCTGAG
AACCGGCGGATGGAAAGTGTCCGCCGTGACTCTGGCCTACCTGATCTTCTGCCTGTG
CTCCCTCGTGGGCATCCTGCATCTGCAGAGGGCTCTGGTGCTGCGGCGGAAGCGGA
AGAGAATGACCGACCCTACCCGGCGGTTCTAA
SEQ ID NO: 122 GRP78-mtIgG4-CD123-CAR_full-length
MDWIWRILFLVGAATGAHSCTVALPGGYVRVCEFESKYGPPCPSCPAPEFEGGPSVFLFP
PKPKDTLMISRTPEVTCVVVDVSQEDPEVQFNWYVDGVEVHNAKTKPREEQFQSTYRV
VSVLTVLHQDWLNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSRLTVDKSRWQEG
NVFSCSVMHEALHNHYTQKSLSLSLGKAAAQVQLQQPGAELVRPGASVKLSCKASGYT
FTSYWMNWVKQRPDQGLEWIGRIDPYDSETHYNQKFKDKAILTVDKSSSTAYMQLSSL
TSEDSAVYYCARGNWDDYWGQGTTLTVSSGGGGSGGGGSGGGGSDVQITQSPSYLAA
SPGETITINCRASKSISKDLAWYQEKPGKTNKLLIYSGSTLQSGIPSRFSGSGSGTDFTLTIS
SLEPEDFAMYYCQQHNKYPYTFGGGTKLEIKSIEVMYPPPYLDNEKSNGTIIHVKGKHL
CPSPLFPGPSKPFWVLVVVGGVLACYSLLVTVAFIIFWVRSKRSRLLHSDYMNMTPRRP
GPTRKHYQPYAPPRDFAAYRSRVKFSRSADAPAYQQGQNQLYNELNLGRREEYDVLDK
RRGRDPEMGGKPRRKNPQEGLYNELQKDKMAEAYSEIGMKGERRRGKGHDGLYQGLS
TATKDTYDALHMQALPPRRSGVDSGEGRGSLLTCGDVEENPGPMPPPRLLFFLLFLTPM
EVRPEEPLVVKVEEGDNAVLQCLKGTSDGPTQQLTWSRESPLKPFLKLSLGLPGLGIHM
RPLAIWLFIFNVSQQMGGFYLCQPGPPSEKAWQPGWTVNVEGSGELFRWNVSDLGGLG
CGLKNRSSEGPSSPSGKLMSPKLYVWAKDRPEIWEGEPPCLPPRDSLNQSLSQDLTMAP
GSTLWLSCGVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYLIFCLCSLV
GILHLQRALVLRRKRKRMTDPTRRF*
SEQ ID NO: 123 GRP78-mtIgG4-CD123-CAR_full-length
ATGGACTGGATTTGGCGGATCCTGTTTCTCGTGGGAGCCGCCACAGGCGCCCATTCT
TGTACTGTGGCCCTTCCTGGTGGATACGTTAGAGTGTGCGAATTCGAGTCTAAGTAC
GGCCCTCCTTGTCCTAGCTGCCCCGCTCCTGAATTTGAAGGCGGCCCTTCCGTGTTCC
TGTTTCCTCCAAAGCCTAAGGACACCCTGATGATCAGCAGAACCCCTGAAGTGACCT
GCGTGGTGGTGGACGTGTCCCAAGAGGATCCTGAGGTGCAGTTCAATTGGTACGTG
GACGGCGTGGAAGTGCACAACGCCAAGACCAAGCCTAGAGAGGAACAGTTCCAGA
GCACCTACAGAGTGGTGTCCGTGCTGACAGTGCTGCACCAGGATTGGCTGAACGGC
AAAGAGTACAAGTGCAAGGTGTCCAACAAGGGCCTGCCTAGCAGCATCGAGAAAAC
CATCAGCAAGGCCAAGGGCCAGCCAAGAGAACCCCAGGTGTACACACTGCCTCCAA
GCCAAGAGGAAATGACCAAGAACCAGGTGTCCCTGACCTGCCTGGTCAAGGGCTTC
TACCCTTCCGATATCGCCGTGGAATGGGAGAGCAATGGCCAGCCTGAGAACAACTA
CAAGACCACACCTCCTGTGCTGGACAGCGACGGCTCATTCTTCCTGTACAGCAGACT
GACCGTGGACAAGAGCAGATGGCAAGAGGGCAACGTGTTCAGCTGCAGCGTGATGC
ACGAGGCCCTGCACAACCACTACACCCAGAAGTCTCTGAGCCTGAGCCTCGGCAAG
GCGGCCGCTCAGGTGCAGCTGCAGCAGCCTGGCGCTGAACTCGTGCGGCCAGGCGC
TTCTGTGAAGCTGAGCTGTAAAGCCAGCGGCTACACCTTCACCAGCTACTGGATGAA
CTGGGTCAAGCAGCGGCCCGACCAGGGCCTGGAATGGATCGGAAGAATCGACCCCT
ACGACAGCGAGACACACTACAACCAGAAGTTCAAGGACAAGGCCATCCTGACCGTG
GACAAGAGCAGCAGCACCGCCTACATGCAGCTGTCCAGCCTGACCAGCGAGGACAG
CGCCGTGTACTACTGCGCCAGAGGCAACTGGGACGACTACTGGGGCCAGGGCACAA
CCCTGACAGTGTCTAGCGGAGGCGGAGGATCAGGCGGCGGAGGAAGTGGGGGAGG
CGGATCTGATGTGCAGATTACCCAGTCCCCCAGCTACCTGGCCGCCTCTCCTGGCGA
GACAATCACCATCAACTGCCGGGCCAGCAAGAGCATCTCCAAGGACCTGGCCTGGT
ATCAGGAAAAGCCCGGCAAGACCAACAAGCTGCTGATCTACAGCGGCTCCACCCTG
CAGTCCGGCATCCCCAGCAGATTTTCCGGCAGCGGCTCTGGCACCGACTTCACCCTG
ACCATCAGCTCCCTGGAACCCGAGGACTTTGCCATGTACTATTGCCAGCAGCACAAC
AAGTACCCTTACACCTTCGGCGGAGGCACCAAGCTGGAAATCAAGAGCATCGAAGT
GATGTACCCGCCTCCTTACCTGGACAACGAGAAGTCCAACGGCACCATCATCCACGT
GAAGGGAAAGCACCTGTGTCCTTCTCCACTGTTCCCCGGACCTAGCAAGCCTTTCTG
GGTGCTCGTTGTTGTTGGCGGCGTGCTGGCCTGTTACAGCCTGCTGGTTACCGTGGC
CTTCATCATCTTTTGGGTCCGAAGCAAGCGGAGCCGGCTGCTGCACAGCGACTACAT
GAACATGACCCCTAGACGGCCCGGACCAACCAGAAAGCACTACCAGCCTTACGCTC
CTCCTAGAGATTTCGCCGCCTACCGGTCCAGAGTGAAGTTCAGCAGGAGCGCAGAC
GCCCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGGGG
GAAAGCCGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGGC
AAGGGGCACGATGGCCTTTACCAGGGTCTCAGTACAGCCACCAAGGACACCTACGA
CGCCCTTCACATGCAGGCCCTGCCCCCTCGCAGATCTGGCGTCGACTCTGGCGAAGG
CAGAGGCTCTCTGCTGACATGTGGCGACGTGGAAGAGAATCCTGGACCTATGCCTCC
CCCCAGACTGCTGTTCTTCCTGCTGTTCCTGACCCCTATGGAAGTGCGGCCCGAGGA
ACCCCTGGTCGTGAAAGTGGAAGAGGGCGACAACGCCGTGCTGCAGTGTCTGAAGG
GCACCTCCGATGGCCCTACCCAGCAGCTGACCTGGTCCAGAGAGAGCCCCCTGAAG
CCCTTCCTGAAGCTGTCTCTGGGCCTGCCTGGCCTGGGCATCCATATGAGGCCACTG
GCCATCTGGCTGTTCATCTTCAACGTGTCCCAGCAGATGGGAGGCTTCTACCTGTGC
CAGCCTGGCCCACCTTCTGAGAAGGCTTGGCAGCCTGGCTGGACCGTGAACGTGGA
AGGATCTGGCGAGCTGTTCCGGTGGAACGTGTCCGATCTGGGCGGCCTGGGATGCG
GCCTGAAGAACAGATCTAGCGAGGGCCCCAGCAGCCCCAGCGGCAAACTGATGAGC
CCCAAGCTGTACGTGTGGGCCAAGGACAGACCCGAGATTTGGGAGGGCGAGCCCCC
TTGCCTGCCCCCTAGAGATAGCCTGAACCAGAGCCTGAGCCAGGACCTGACAATGG
CCCCTGGCAGCACACTGTGGCTGAGCTGTGGCGTGCCACCCGACTCTGTGTCTAGAG
GCCCTCTGAGCTGGACCCACGTGCACCCTAAGGGCCCTAAGAGCCTGCTGTCCCTGG
AACTGAAGGACGACAGGCCCGCCAGAGATATGTGGGTCATGGAAACCGGCCTGCTG
CTGCCTAGAGCCACAGCCCAGGATGCCGGCAAGTACTACTGCCACAGAGGCAACCT
GACCATGAGCTTCCACCTGGAAATCACCGCCAGACCCGTGCTGTGGCACTGGCTGCT
GAGAACCGGCGGATGGAAAGTGTCCGCCGTGACTCTGGCCTACCTGATCTTCTGCCT
GTGCTCCCTCGTGGGCATCCTGCATCTGCAGAGGGCTCTGGTGCTGCGGCGGAAGCG
GAAGAGAATGACCGACCCTACCCGGCGGTTCTAA
SEQ ID NO: 124 GRP78-GPcPcPc-CD123-CAR_full-length
MDWIWRILFLVGAATGAHSCTVALPGGYVRVCEFAGSGGSGGSGGSPVPSTPPTNSSST
PPTPSPSPVPSTPPTNSSSTPPTPSPSPVPSTPPTNSSSTPPTPSPSASAAAQVQLQQPGAELV
RPGASVKLSCKASGYTFTSYWMNWVKQRPDQGLEWIGRIDPYDSETHYNQKFKDKAIL
TVDKSSSTAYMQLSSLTSEDSAVYYCARGNWDDYWGQGTTLTVSSGGGGSGGGGSGG
GGSDVQITQSPSYLAASPGETITINCRASKSISKDLAWYQEKPGKTNKLLIYSGSTLQSGIP
SRFSGSGSGTDFTLTISSLEPEDFAMYYCQQHNKYPYTFGGGTKLEIKSIEVMYPPPYLD
NEKSNGTIIHVKGKHLCPSPLFPGPSKPFWVLVVVGGVLACYSLLVTVAFIIFWVRSKRS
RLLHSDYMNMTPRRPGPTRKHYQPYAPPRDFAAYRSRVKFSRSADAPAYQQGQNQLY
NELNLGRREEYDVLDKRRGRDPEMGGKPRRKNPQEGLYNELQKDKMAEAYSEIGMKG
ERRRGKGHDGLYQGLSTATKDTYDALHMQALPPRRSGVDSGEGRGSLLTCGDVEENPG
PMPPPRLLFFLLFLTPMEVRPEEPLVVKVEEGDNAVLQCLKGTSDGPTQQLTWSRESPLK
PFLKLSLGLPGLGIHMRPLAIWLFIFNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPR
DSLNQSLSQDLTMAPGSTLWLSCGVPPDSVSRGPLSWTHVHPKGPKSLLSLELKDDRPA
RDMWVMETGLLLPRATAQDAGKYYCHRGNLTMSFHLEITARPVLWHWLLRTGGWKV
SAVTLAYLIFCLCSLVGILHLQRALVLRRKRKRMTDPTRRF*
SEQ ID NO: 125 GRP78-GPcPcPc-CD123-CAR_full-length
ATGGACTGGATTTGGCGGATCCTGTTTCTCGTGGGAGCCGCCACAGGCGCCCATTCT
TGTACTGTGGCCCTTCCTGGTGGATACGTTAGAGTGTGCGAATTCGCAGGCAGTGGT
GGAAGTGGTGGGTCAGGCGGTTCCCCGGTTCCATCAACACCACCAACCAATAGCTC
ATCTACCCCGCCAACACCAAGTCCCTCTCCCGTCCCCAGCACACCCCCGACGAACTC
ATCCTCCACACCGCCAACCCCGTCTCCTTCCCCGGTTCCCTCTACACCTCCGACAAAC
TCTAGTTCAACGCCTCCCACTCCATCACCTTCCGCATCAGCGGCCGCTCAGGTGCAG
CTGCAGCAGCCTGGCGCTGAACTCGTGCGGCCAGGCGCTTCTGTGAAGCTGAGCTGT
AAAGCCAGCGGCTACACCTTCACCAGCTACTGGATGAACTGGGTCAAGCAGCGGCC
CGACCAGGGCCTGGAATGGATCGGAAGAATCGACCCCTACGACAGCGAGACACACT
ACAACCAGAAGTTCAAGGACAAGGCCATCCTGACCGTGGACAAGAGCAGCAGCACC
GCCTACATGCAGCTGTCCAGCCTGACCAGCGAGGACAGCGCCGTGTACTACTGCGC
CAGAGGCAACTGGGACGACTACTGGGGCCAGGGCACAACCCTGACAGTGTCTAGCG
GAGGCGGAGGATCAGGCGGCGGAGGAAGTGGGGGAGGCGGATCTGATGTGCAGAT
TACCCAGTCCCCCAGCTACCTGGCCGCCTCTCCTGGCGAGACAATCACCATCAACTG
CCGGGCCAGCAAGAGCATCTCCAAGGACCTGGCCTGGTATCAGGAAAAGCCCGGCA
AGACCAACAAGCTGCTGATCTACAGCGGCTCCACCCTGCAGTCCGGCATCCCCAGC
AGATTTTCCGGCAGCGGCTCTGGCACCGACTTCACCCTGACCATCAGCTCCCTGGAA
CCCGAGGACTTTGCCATGTACTATTGCCAGCAGCACAACAAGTACCCTTACACCTTC
GGCGGAGGCACCAAGCTGGAAATCAAGAGCATCGAAGTGATGTACCCGCCTCCTTA
CCTGGACAACGAGAAGTCCAACGGCACCATCATCCACGTGAAGGGAAAGCACCTGT
GTCCTTCTCCACTGTTCCCCGGACCTAGCAAGCCTTTCTGGGTGCTCGTTGTTGTTGG
CGGCGTGCTGGCCTGTTACAGCCTGCTGGTTACCGTGGCCTTCATCATCTTTTGGGTC
CGAAGCAAGCGGAGCCGGCTGCTGCACAGCGACTACATGAACATGACCCCTAGACG
GCCCGGACCAACCAGAAAGCACTACCAGCCTTACGCTCCTCCTAGAGATTTCGCCGC
CTACCGGTCCAGAGTGAAGTTCAGCAGGAGCGCAGACGCCCCCGCGTACCAGCAGG
GCCAGAACCAGCTCTATAACGAGCTCAATCTAGGACGAAGAGAGGAGTACGATGTT
TTGGACAAGAGACGTGGCCGGGACCCTGAGATGGGGGGAAAGCCGAGAAGGAAGA
ACCCTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCCTTT
ACCAGGGTCTCAGTACAGCCACCAAGGACACCTACGACGCCCTTCACATGCAGGCC
CTGCCCCCTCGCAGATCTGGCGTCGACTCTGGCGAAGGCAGAGGCTCTCTGCTGACA
TGTGGCGACGTGGAAGAGAATCCTGGACCTATGCCTCCCCCCAGACTGCTGTTCTTC
CTGCTGTTCCTGACCCCTATGGAAGTGCGGCCCGAGGAACCCCTGGTCGTGAAAGTG
GAAGAGGGCGACAACGCCGTGCTGCAGTGTCTGAAGGGCACCTCCGATGGCCCTAC
CCAGCAGCTGACCTGGTCCAGAGAGAGCCCCCTGAAGCCCTTCCTGAAGCTGTCTCT
GGGCCTGCCTGGCCTGGGCATCCATATGAGGCCACTGGCCATCTGGCTGTTCATCTT
CAACGTGTCCCAGCAGATGGGAGGCTTCTACCTGTGCCAGCCTGGCCCACCTTCTGA
GAAGGCTTGGCAGCCTGGCTGGACCGTGAACGTGGAAGGATCTGGCGAGCTGTTCC
GGTGGAACGTGTCCGATCTGGGCGGCCTGGGATGCGGCCTGAAGAACAGATCTAGC
GAGGGCCCCAGCAGCCCCAGCGGCAAACTGATGAGCCCCAAGCTGTACGTGTGGGC
CAAGGACAGACCCGAGATTTGGGAGGGCGAGCCCCCTTGCCTGCCCCCTAGAGATA
GCCTGAACCAGAGCCTGAGCCAGGACCTGACAATGGCCCCTGGCAGCACACTGTGG
CTGAGCTGTGGCGTGCCACCCGACTCTGTGTCTAGAGGCCCTCTGAGCTGGACCCAC
GTGCACCCTAAGGGCCCTAAGAGCCTGCTGTCCCTGGAACTGAAGGACGACAGGCC
CGCCAGAGATATGTGGGTCATGGAAACCGGCCTGCTGCTGCCTAGAGCCACAGCCC
AGGATGCCGGCAAGTACTACTGCCACAGAGGCAACCTGACCATGAGCTTCCACCTG
GAAATCACCGCCAGACCCGTGCTGTGGCACTGGCTGCTGAGAACCGGCGGATGGAA
AGTGTCCGCCGTGACTCTGGCCTACCTGATCTTCTGCCTGTGCTCCCTCGTGGGCATC
CTGCATCTGCAGAGGGCTCTGGTGCTGCGGCGGAAGCGGAAGAGAATGACCGACCC
TACCCGGCGGTTCTAA
SEQ ID NO: 126 GRP78-specific peptide with an N-terminal Biotin Tag
Biotin-Ahx-CTVALPGGYVRVC
SEQ ID NO: 127 scFv (716) amino acid sequence
QIQLVQSGPELKKPGETVKISCKASGYIFTNYGMNWVKQAPGKSFKWMGWINTYTGES
TYSADFKGRFAFSLETSASTAYLHINDLKNEDTATYFCARSGGYDPMDYWGQGTSVTV
SSGGGGSGGGGSGGGGSDIVLTQSPASLAVSLGQRATISCRASESVDNYGNTFMHWYQ
QKPGQPPKLLIYRASNLESGIPARFSGSGSRTDFTLTINPVEADDVATYYCQQSNEDPPTF
GAGTKLELK
SEQ ID NO: 128 scFv (716) DNA sequence
CAGATTCAGCTGGTGCAGTCTGGCCCCGAGCTGAAGAAACCCGGCGAGACAGTGAA
GATCAGCTGCAAGGCCAGCGGCTACATCTTCACCAACTACGGCATGAACTGGGTCA
AGCAGGCCCCTGGCAAGAGCTTCAAGTGGATGGGCTGGATCAACACCTACACCGGC
GAGAGCACCTACAGCGCCGACTTCAAGGGCAGATTCGCCTTCAGCCTGGAAACCAG
CGCCAGCACCGCCTACCTGCACATCAACGACCTGAAGAACGAGGACACCGCCACCT
ACTTTTGCGCCAGAAGCGGCGGCTACGACCCTATGGATTATTGGGGCCAGGGCACC
AGCGTGACCGTGTCTAGCGGAGGCGGAGGAAGTGGCGGCGGAGGATCTGGGGGAG
GCGGATCTGATATCGTGCTGACCCAGAGCCCTGCCAGCCTGGCTGTGTCTCTGGGAC
AGAGAGCCACCATCAGCTGTCGGGCCAGCGAGAGCGTGGACAATTACGGCAACACC
TTCATGCACTGGTATCAGCAGAAGCCCGGCCAGCCCCCCAAGCTGCTGATCTACAGA
GCCAGCAACCTGGAAAGCGGCATCCCCGCCAGATTTTCCGGCAGCGGCAGCAGAAC
CGACTTCACCCTGACCATCAACCCCGTGGAAGCCGACGACGTGGCCACCTATTACTG
CCAGCAGAGCAACGAGGACCCCCCTACCTTTGGAGCCGGCACCAAGCTGGAACTGA
AG
SEQ ID NO: 129 B7H3 (MGA271) heavy chain
GAAGTTCAGCTGGTTGAGTCTGGCGGCGGACTGGTTCAACCAGGCGGATCTCTGAG
ACTGAGCTGTGCCGCCAGCGGCTTCACCTTTAGCAGCTTTGGCATGCACTGGGTCCG
ACAGGCCCCTGGCAAAGGACTTGAGTGGGTCGCCTACATCAGCAGCGATAGCAGCG
CCATCTACTACGCCGACACCGTGAAGGGCAGATTCACCATCAGCCGGGACAACGCC
AAGAACAGCCTGTACCTGCAGATGAACTCCCTGCGCGACGAGGATACCGCCGTGTA
CTATTGTGGCAGAGGCAGAGAGAACATCTATTACGGCAGCAGACTGGACTACTGGG
GCCAGGGAACAACCGTGACAGTCTCT
SEQ ID NO: 130 B7H3 (MGA271) heavy chain
EVQLVESGGGLVQPGGSLRLSCAASGFTFSSFGMHWVRQAPGKGLEWVAYISSDSSAIY
YADTVKGRFTISRDNAKNSLYLQMNSLRDEDTAVYYCGRGRENIYYGSRLDYWGQGT
TVTVS
SEQ ID NO: 131 B7H3 (MGA271) light chain
GATATTCAGCTGACTCAGAGCCCCAGCTTCCTGAGCGCCTCTGTGGGAGACAGAGTG
ACCATCACATGCAAGGCCAGCCAGAACGTGGACACCAACGTGGCCTGGTATCAGCA
GAAGCCTGGCAAGGCTCCCAAGGCTCTGATCTACAGCGCCAGCTACAGATACAGCG
GCGTGCCCAGCAGATTTTCTGGCAGCGGCTCTGGCACCGACTTCACCCTGACCATAT
CTAGCCTGCAGCCTGAGGACTTCGCCACCTACTACTGCCAGCAGTACAACAACTACC
CCTTCACCTTCGGCCAGGGCACCAAGCTGGAAATCAAG
SEQ ID NO: 132 B7H3 (MGA271) light chain
DIQLTQSPSFLSASVGDRVTITCKASQNVDTNVAWYQQKPGKAPKALIYSASYRYSGVP
SRFSGSGSGTDFTLTISSLQPEDFATYYCQQYNNYPFTFGQGTKLEIK
SEQ ID NO: 133 B7H3 (MGA271) scFv
GAAGTTCAGCTGGTTGAGTCTGGCGGCGGACTGGTTCAACCAGGCGGATCTCTGAG
ACTGAGCTGTGCCGCCAGCGGCTTCACCTTTAGCAGCTTTGGCATGCACTGGGTCCG
ACAGGCCCCTGGCAAAGGACTTGAGTGGGTCGCCTACATCAGCAGCGATAGCAGCG
CCATCTACTACGCCGACACCGTGAAGGGCAGATTCACCATCAGCCGGGACAACGCC
AAGAACAGCCTGTACCTGCAGATGAACTCCCTGCGCGACGAGGATACCGCCGTGTA
CTATTGTGGCAGAGGCAGAGAGAACATCTATTACGGCAGCAGACTGGACTACTGGG
GCCAGGGAACAACCGTGACAGTCTCTTCTGGTGGCGGAGGAAGCGGAGGCGGAGGT
TCAGGCGGCGGAGGATCTGATATTCAGCTGACTCAGAGCCCCAGCTTCCTGAGCGCC
TCTGTGGGAGACAGAGTGACCATCACATGCAAGGCCAGCCAGAACGTGGACACCAA
CGTGGCCTGGTATCAGCAGAAGCCTGGCAAGGCTCCCAAGGCTCTGATCTACAGCG
CCAGCTACAGATACAGCGGCGTGCCCAGCAGATTTTCTGGCAGCGGCTCTGGCACC
GACTTCACCCTGACCATATCTAGCCTGCAGCCTGAGGACTTCGCCACCTACTACTGC
CAGCAGTACAACAACTACCCCTTCACCTTCGGCCAGGGCACCAAGCTGGAAATCAA
G
SEQ ID NO: 133 B7H3 (MGA271) scFv
EVQLVESGGGLVQPGGSLRLSCAASGFTFSSFGMHWVRQAPGKGLEWVAYISSDSSAIY
YADTVKGRFTISRDNAKNSLYLQMNSLRDEDTAVYYCGRGRENIYYGSRLDYWGQGT
TVTVSSGGGGSGGGGSGGGGSDIQLTQSPSFLSASVGDRVTITCKASQNVDINVAWYQ
QKPGKAPKALIYSASYRYSGVPSRFSGSGSGTDFTLTISSLQPEDFATYYCQQYNNYPFTF
GQGTKLEIK
SEQ ID NO: 135 Spacer
AGATCTGGCGTCGACTCTGGC
SEQ ID NO: 136 Spacer
RSGVDSG
SEQ ID NO: 137 CD8α hinge/stalk/transmembrane
ACCACCACACCAGCTCCTCGGCCTCCAACTCCTGCTCCTACAATTGCCAGCCAGCCT
CTGTCTCTGAGGCCCGAAGCTTGTAGACCTGCTGCTGGCGGAGCCGTGCATACAAGA
GGACTGGATTTCGCCTGCGACATCTACATCTGGGCCCCTCTGGCTGGAACATGTGGC
GTTCTGCTGCTGAGCCTGGTCATCACCCTGTACTGC
SEQ ID NO: 138 CD28 costimulatory domain
CGGTCCAAGAGAAGCAGACTGCTGCACAGCGACTACATGAACATGACCCCTAGACG
GCCCGGACCTACCAGAAAGCACTACCAGCCTTACGCTCCTCCTAGAGATTTCGCCGC
CTACCGGTCC
SEQ ID NO: 139 CD3 zeta
AGAGTGAAGTTCTCCAGATCCGCCGATGCTCCCGCCTATCAGCAGGGACAGAACCA
GCTGTACAACGAGCTGAACCTGGGGAGAAGAGAAGAGTACGACGTGCTGGACAAG
CGGAGAGGCAGGGATCCTGAGATGGGCGGCAAGCCCAGACGGAAGAATCCTCAAG
AGGGCCTGTATAATGAGCTGCAGAAAGACAAGATGGCCGAGGCCTACAGCGAGATC
GGAATGAAGGGCGAGCGCAGAAGAGGCAAGGGACACGATGGACTGTACCAGGGCC
TGAGCACCGCCACCAAGGATACCTATGATGCCCTGCACATGCAGGCCCTGCCTCCAA
GA
SEQ ID NO: 140 GRP78-G4S3-B7H3-CD28-CAR (78.B7H3-CAR) extracellular domain
ATGGACTGGATTTGGCGGATCCTGTTTCTCGTGGGAGCCGCCACAGGCGCCCATTCT
TGTACTGTGGCCCTTCCTGGTGGATACGTTAGAGTGTGCGAATTCGGAGGCGGAGGA
TCAGGCGGCGGAGGAAGTGGGGGAGGCGGATCTGCGGCCGCTGAAGTTCAGCTGGT
TGAGTCTGGCGGCGGACTGGTTCAACCAGGCGGATCTCTGAGACTGAGCTGTGCCGC
CAGCGGCTTCACCTTTAGCAGCTTTGGCATGCACTGGGTCCGACAGGCCCCTGGCAA
AGGACTTGAGTGGGTCGCCTACATCAGCAGCGATAGCAGCGCCATCTACTACGCCG
ACACCGTGAAGGGCAGATTCACCATCAGCCGGGACAACGCCAAGAACAGCCTGTAC
CTGCAGATGAACTCCCTGCGCGACGAGGATACCGCCGTGTACTATTGTGGCAGAGG
CAGAGAGAACATCTATTACGGCAGCAGACTGGACTACTGGGGCCAGGGAACAACCG
TGACAGTCTCTTCTGGTGGCGGAGGAAGCGGAGGCGGAGGTTCAGGCGGCGGAGGA
TCTGATATTCAGCTGACTCAGAGCCCCAGCTTCCTGAGCGCCTCTGTGGGAGACAGA
GTGACCATCACATGCAAGGCCAGCCAGAACGTGGACACCAACGTGGCCTGGTATCA
GCAGAAGCCTGGCAAGGCTCCCAAGGCTCTGATCTACAGCGCCAGCTACAGATACA
GCGGCGTGCCCAGCAGATTTTCTGGCAGCGGCTCTGGCACCGACTTCACCCTGACCA
TATCTAGCCTGCAGCCTGAGGACTTCGCCACCTACTACTGCCAGCAGTACAACAACT
ACCCCTTCACCTTCGGCCAGGGCACCAAGCTGGAAATCAAG
SEQ ID NO: 141 GRP78-G4S3-B7H3-CD28-CAR (78.B7H3-CAR) extracellular domain
MDWIWRILFLVGAATGAHSCTVALPGGYVRVCEFGGGGSGGGGSGGGGSAAA
EVQLVESGGGLVQPGGSLRLSCAASGFTFSSFGMHWVRQAPGKGLEWVAYISSDSSAIY
YADTVKGRFTISRDNAKNSLYLQMNSLRDEDTAVYYCGRGRENIYYGSRLDYWGQGT
TVTVSSGGGGSGGGGSGGGGSDIQLTQSPSFLSASVGDRVTITCKASQNVDTNVAWYQ
QKPGKAPKALIYSASYRYSGVPSRFSGSGSGTDFTLTISSLQPEDFATYYCQQYNNYPFTF
GQGTKLEIK
SEQ ID NO: 142 GRP78-G4S3-B7H3-CD28-CAR (78.B7H3-CAR) cytoplasmic domain
CGAAGCAAGCGGAGCCGGCTGCTGCACAGCGACTACATGAACATGACCCCTAGACG
GCCCGGACCAACCAGAAAGCACTACCAGCCTTACGCTCCTCCTAGAGATTTCGCCGC
CTACCGGTCCAGAGTGAAGTTCAGCAGGAGCGCAGACGCCCCCGCGTACCAGCAGG
GCCAGAACCAGCTCTATAACGAGCTCAATCTAGGACGAAGAGAGGAGTACGATGTT
TTGGACAAGAGACGTGGCCGGGACCCTGAGATGGGGGGAAAGCCGAGAAGGAAGA
ACCCTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCCTTT
ACCAGGGTCTCAGTACAGCCACCAAGGACACCTACGACGCCCTTCACATGCAGGCC
CTGCCCCCTCGCAGATCTGGCGTCGACTCTGGCGAAGGCAGAGGCTCTCTGCTGACA
TGTGGCGACGTGGAAGAGAATCCTGGACCTATGCCTCCCCCCAGACTGCTGTTCTTC
CTGCTGTTCCTGACCCCTATGGAAGTGCGGCCCGAGGAACCCCTGGTCGTGAAAGTG
GAAGAGGGCGACAACGCCGTGCTGCAGTGTCTGAAGGGCACCTCCGATGGCCCTAC
CCAGCAGCTGACCTGGTCCAGAGAGAGCCCCCTGAAGCCCTTCCTGAAGCTGTCTCT
GGGCCTGCCTGGCCTGGGCATCCATATGAGGCCACTGGCCATCTGGCTGTTCATCTT
CAACGTGTCCCAGCAGATGGGAGGCTTCTACCTGTGCCAGCCTGGCCCACCTTCTGA
GAAGGCTTGGCAGCCTGGCTGGACCGTGAACGTGGAAGGATCTGGCGAGCTGTTCC
GGTGGAACGTGTCCGATCTGGGCGGCCTGGGATGCGGCCTGAAGAACAGATCTAGC
GAGGGCCCCAGCAGCCCCAGCGGCAAACTGATGAGCCCCAAGCTGTACGTGTGGGC
CAAGGACAGACCCGAGATTTGGGAGGGCGAGCCCCCTTGCCTGCCCCCTAGAGATA
GCCTGAACCAGAGCCTGAGCCAGGACCTGACAATGGCCCCTGGCAGCACACTGTGG
CTGAGCTGTGGCGTGCCACCCGACTCTGTGTCTAGAGGCCCTCTGAGCTGGACCCAC
GTGCACCCTAAGGGCCCTAAGAGCCTGCTGTCCCTGGAACTGAAGGACGACAGGCC
CGCCAGAGATATGTGGGTCATGGAAACCGGCCTGCTGCTGCCTAGAGCCACAGCCC
AGGATGCCGGCAAGTACTACTGCCACAGAGGCAACCTGACCATGAGCTTCCACCTG
GAAATCACCGCCAGACCCGTGCTGTGGCACTGGCTGCTGAGAACCGGCGGATGGAA
AGTGTCCGCCGTGACTCTGGCCTACCTGATCTTCTGCCTGTGCTCCCTCGTGGGCATC
CTGCATCTGCAGAGGGCTCTGGTGCTGCGGCGGAAGCGGAAGAGAATGACCGACCC
TACCCGGCGGTTCTAA
SEQ ID NO: 143 GRP78-G4S3-B7H3-CD28-CAR (78.B7H3-CAR) cytoplasmic domain
RSKRSRLLHSDYMNMTPRRPGPTRKHYQPYAPPRDFAAYRSRVKFSRSADAPAYQQGQ
NQLYNELNLGRREEYDVLDKRRGRDPEMGGKPRRKNPQEGLYNELQKDKMAEAYSEI
GMKGERRRGKGHDGLYQGLSTATKDTYDALHMQALPPRRSGVDSGEGRGSLLTCGDV
EENPGPMPPPRLLFFLLFLTPMEVRPEEPLVVKVEEGDNAVLQCLKGTSDGPTQQLTWS
RESPLKPFLKLSLGLPGLGIHMRPLAIWLFIFNVSQQMGGFYLCQPGPPSEKAWQPGWT
VNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLEL
KDDRPARDMWVMETGLLLPRATAQDAGKYYCHRGNLTMSFHLEITARPVLWHWLLR
TGGWKVSAVTLAYLIFCLCSLVGILHLQRALVLRRKRKRMTDPTRRF
SEQ ID NO: 144 GRP78-G4S3-B7H3-CD28-CAR (78.B7H3-CAR) full length
ATGGACTGGATTTGGCGGATCCTGTTTCTCGTGGGAGCCGCCACAGGCGCCCATTCT
TGTACTGTGGCCCTTCCTGGTGGATACGTTAGAGTGTGCGAATTCGGAGGCGGAGGA
TCAGGCGGCGGAGGAAGTGGGGGAGGCGGATCTGCGGCCGCTGAAGTTCAGCTGGT
TGAGTCTGGCGGCGGACTGGTTCAACCAGGCGGATCTCTGAGACTGAGCTGTGCCGC
CAGCGGCTTCACCTTTAGCAGCTTTGGCATGCACTGGGTCCGACAGGCCCCTGGCAA
AGGACTTGAGTGGGTCGCCTACATCAGCAGCGATAGCAGCGCCATCTACTACGCCG
ACACCGTGAAGGGCAGATTCACCATCAGCCGGGACAACGCCAAGAACAGCCTGTAC
CTGCAGATGAACTCCCTGCGCGACGAGGATACCGCCGTGTACTATTGTGGCAGAGG
CAGAGAGAACATCTATTACGGCAGCAGACTGGACTACTGGGGCCAGGGAACAACCG
TGACAGTCTCTTCTGGTGGCGGAGGAAGCGGAGGCGGAGGTTCAGGCGGCGGAGGA
TCTGATATTCAGCTGACTCAGAGCCCCAGCTTCCTGAGCGCCTCTGTGGGAGACAGA
GTGACCATCACATGCAAGGCCAGCCAGAACGTGGACACCAACGTGGCCTGGTATCA
GCAGAAGCCTGGCAAGGCTCCCAAGGCTCTGATCTACAGCGCCAGCTACAGATACA
GCGGCGTGCCCAGCAGATTTTCTGGCAGCGGCTCTGGCACCGACTTCACCCTGACCA
TATCTAGCCTGCAGCCTGAGGACTTCGCCACCTACTACTGCCAGCAGTACAACAACT
ACCCCTTCACCTTCGGCCAGGGCACCAAGCTGGAAATCAAGATCGAAGTGATGTAC
CCGCCTCCTTACCTGGACAACGAGAAGTCCAACGGCACCATCATCCACGTGAAGGG
AAAGCACCTGTGTCCTTCTCCACTGTTCCCCGGACCTAGCAAGCCTTTCTGGGTGCTC
GTTGTTGTTGGCGGCGTGCTGGCCTGTTACAGCCTGCTGGTTACCGTGGCCTTCATCA
TCTTTTGGGTCCGAAGCAAGCGGAGCCGGCTGCTGCACAGCGACTACATGAACATG
ACCCCTAGACGGCCCGGACCAACCAGAAAGCACTACCAGCCTTACGCTCCTCCTAG
AGATTTCGCCGCCTACCGGTCCAGAGTGAAGTTCAGCAGGAGCGCAGACGCCCCCG
CGTACCAGCAGGGCCAGAACCAGCTCTATAACGAGCTCAATCTAGGACGAAGAGAG
GAGTACGATGTTTTGGACAAGAGACGTGGCCGGGACCCTGAGATGGGGGGAAAGCC
GAGAAGGAAGAACCCTCAGGAAGGCCTGTACAATGAACTGCAGAAAGATAAGATG
GCGGAGGCCTACAGTGAGATTGGGATGAAAGGCGAGCGCCGGAGGGGCAAGGGGC
ACGATGGCCTTTACCAGGGTCTCAGTACAGCCACCAAGGACACCTACGACGCCCTTC
ACATGCAGGCCCTGCCCCCTCGCAGATCTGGCGTCGACTCTGGCGAAGGCAGAGGC
TCTCTGCTGACATGTGGCGACGTGGAAGAGAATCCTGGACCTATGCCTCCCCCCAGA
CTGCTGTTCTTCCTGCTGTTCCTGACCCCTATGGAAGTGCGGCCCGAGGAACCCCTG
GTCGTGAAAGTGGAAGAGGGCGACAACGCCGTGCTGCAGTGTCTGAAGGGCACCTC
CGATGGCCCTACCCAGCAGCTGACCTGGTCCAGAGAGAGCCCCCTGAAGCCCTTCCT
GAAGCTGTCTCTGGGCCTGCCTGGCCTGGGCATCCATATGAGGCCACTGGCCATCTG
GCTGTTCATCTTCAACGTGTCCCAGCAGATGGGAGGCTTCTACCTGTGCCAGCCTGG
CCCACCTTCTGAGAAGGCTTGGCAGCCTGGCTGGACCGTGAACGTGGAAGGATCTG
GCGAGCTGTTCCGGTGGAACGTGTCCGATCTGGGCGGCCTGGGATGCGGCCTGAAG
AACAGATCTAGCGAGGGCCCCAGCAGCCCCAGCGGCAAACTGATGAGCCCCAAGCT
GTACGTGTGGGCCAAGGACAGACCCGAGATTTGGGAGGGCGAGCCCCCTTGCCTGC
CCCCTAGAGATAGCCTGAACCAGAGCCTGAGCCAGGACCTGACAATGGCCCCTGGC
AGCACACTGTGGCTGAGCTGTGGCGTGCCACCCGACTCTGTGTCTAGAGGCCCTCTG
AGCTGGACCCACGTGCACCCTAAGGGCCCTAAGAGCCTGCTGTCCCTGGAACTGAA
GGACGACAGGCCCGCCAGAGATATGTGGGTCATGGAAACCGGCCTGCTGCTGCCTA
GAGCCACAGCCCAGGATGCCGGCAAGTACTACTGCCACAGAGGCAACCTGACCATG
AGCTTCCACCTGGAAATCACCGCCAGACCCGTGCTGTGGCACTGGCTGCTGAGAACC
GGCGGATGGAAAGTGTCCGCCGTGACTCTGGCCTACCTGATCTTCTGCCTGTGCTCC
CTCGTGGGCATCCTGCATCTGCAGAGGGCTCTGGTGCTGCGGCGGAAGCGGAAGAG
AATGACCGACCCTACCCGGCGGTTCTAA
SEQ ID NO: 145 GRP78-G4S3-B7H3-CD28-CAR (78.B7H3-CAR) full length
MDWIWRILFLVGAATGAHSCTVALPGGYVRVCEFGGGGSGGGGSGGGGSAAA
EVQLVESGGGLVQPGGSLRLSCAASGFTFSSFGMHWVRQAPGKGLEWVAYISSDSSAIY
YADTVKGRFTISRDNAKNSLYLQMNSLRDEDTAVYYCGRGRENIYYGSRLDYWGQGT
TVTVSSGGGGSGGGGSGGGGSDIQLTQSPSFLSASVGDRVTITCKASQNVDTNVAWYQ
QKPGKAPKALIYSASYRYSGVPSRFSGSGSGTDFTLTISSLQPEDFATYYCQQYNNYPFTF
GQGTKLEIKIEVMYPPPYLDNEKSNGTIIHVKGKHLCPSPLFPGPSKPFWVLVVVGGVLA
CYSLLVTVAFIIFWVRSKRSRLLHSDYMNMTPRRPGPTRKHYQPYAPPRDFAAYRSRVK
FSRSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPRRSGVD
SGEGRGSLLTCGDVEENPGPMPPPRLLFFLLFLTPMEVRPEEPLVVKVEEGDNAVLQCL
KGTSDGPTQQLTWSRESPLKPFLKLSLGLPGLGIHMRPLAIWLFIFNVSQQMGGFYLCQP
GPPSEKAWQPGWTVNVEGSGELFRWNVSDLGGLGCGLKNRSSEGPSSPSGKLMSPKLY
VWAKDRPEIWEGEPPCLPPRDSLNQSLSQDLTMAPGSTLWLSCGVPPDSVSRGPLSWTH
VHPKGPKSLLSLELKDDRPARDMWVMETGLLLPRATAQDAGKYYCHRGNLTMSFHLEI
TARPVLWHWLLRTGGWKVSAVTLAYLIFCLCSLVGILHLQRALVLRRKRKRMTDPTRR
F
SEQ ID NO: 146 GRP78-G4S3-B7H3-CD8α-CAR (78.B7H3-CAR) extracellular domain
ATGGACTGGATTTGGCGGATCCTGTTTCTCGTGGGAGCCGCCACAGGCGCCCATTCT
TGTACTGTGGCCCTTCCTGGTGGATACGTTAGAGTGTGCGAATTCGGAGGCGGAGGA
TCAGGCGGCGGAGGAAGTGGGGGAGGCGGATCTGAAGTTCAGCTGGTTGAGTCTGG
CGGCGGACTGGTTCAACCAGGCGGATCTCTGAGACTGAGCTGTGCCGCCAGCGGCT
TCACCTTTAGCAGCTTTGGCATGCACTGGGTCCGACAGGCCCCTGGCAAAGGACTTG
AGTGGGTCGCCTACATCAGCAGCGATAGCAGCGCCATCTACTACGCCGACACCGTG
AAGGGCAGATTCACCATCAGCCGGGACAACGCCAAGAACAGCCTGTACCTGCAGAT
GAACTCCCTGCGCGACGAGGATACCGCCGTGTACTATTGTGGCAGAGGCAGAGAGA
ACATCTATTACGGCAGCAGACTGGACTACTGGGGCCAGGGAACAACCGTGACAGTC
TCTTCTGGTGGCGGAGGAAGCGGAGGCGGAGGTTCAGGCGGCGGAGGATCTGATAT
TCAGCTGACTCAGAGCCCCAGCTTCCTGAGCGCCTCTGTGGGAGACAGAGTGACCAT
CACATGCAAGGCCAGCCAGAACGTGGACACCAACGTGGCCTGGTATCAGCAGAAGC
CTGGCAAGGCTCCCAAGGCTCTGATCTACAGCGCCAGCTACAGATACAGCGGCGTG
CCCAGCAGATTTTCTGGCAGCGGCTCTGGCACCGACTTCACCCTGACCATATCTAGC
CTGCAGCCTGAGGACTTCGCCACCTACTACTGCCAGCAGTACAACAACTACCCCTTC
ACCTTCGGCCAGGGCACCAAGCTGGAAATCAAG
SEQ ID NO: 147 GRP78-G4S3-B7H3-CD8α-CAR (78.B7H3-CAR) extracellular domain
MDWIWRILFLVGAATGAHSCTVALPGGYVRVCEFGGGGSGGGGSGGGGSEVQLVESG
GGLVQPGGSLRLSCAASGFTFSSFGMHWVRQAPGKGLEWVAYISSDSSAIYYADTVKG
RFTISRDNAKNSLYLQMNSLRDEDTAVYYCGRGRENIYYGSRLDYWGQGTTVTVSSGG
GGSGGGGSGGGGSDIQLTQSPSFLSASVGDRVTITCKASQNVDTNVAWYQQKPGKAPK
ALIYSASYRYSGVPSRFSGSGSGTDFTLTISSLQPEDFATYYCQQYNNYPFTFGQGTKLEI
K
SEQ ID NO: 148 GRP78-G4S3-B7H3-CD8α-CAR (78.B7H3-CAR) cytoplasmic domain
CGGTCCAAGAGAAGCAGACTGCTGCACAGCGACTACATGAACATGACCCCTAGACG
GCCCGGACCTACCAGAAAGCACTACCAGCCTTACGCTCCTCCTAGAGATTTCGCCGC
CTACCGGTCCAGAGTGAAGTTCTCCAGATCCGCCGATGCTCCCGCCTATCAGCAGGG
ACAGAACCAGCTGTACAACGAGCTGAACCTGGGGAGAAGAGAAGAGTACGACGTG
CTGGACAAGCGGAGAGGCAGGGATCCTGAGATGGGCGGCAAGCCCAGACGGAAGA
ATCCTCAAGAGGGCCTGTATAATGAGCTGCAGAAAGACAAGATGGCCGAGGCCTAC
AGCGAGATCGGAATGAAGGGCGAGCGCAGAAGAGGCAAGGGACACGATGGACTGT
ACCAGGGCCTGAGCACCGCCACCAAGGATACCTATGATGCCCTGCACATGCAGGCC
CTGCCTCCAAGATAA
SEQ ID NO: 149 GRP78-G4S3-B7H3-CD8α-CAR (78.B7H3-CAR) cytoplasmic domain
RSKRSRLLHSDYMNMTPRRPGPTRKHYQPYAPPRDFAAYRSRVKFSRSADAPAYQQGQ
NQLYNELNLGRREEYDVLDKRRGRDPEMGGKPRRKNPQEGLYNELQKDKMAEAYSEI
GMKGERRRGKGHDGLYQGLSTATKDTYDALHMQALPPR
SEQ ID NO: 150 GRP78-G4S3-B7H3-CD8α-CAR (78.B7H3-CAR) full length
ATGGACTGGATTTGGCGGATCCTGTTTCTCGTGGGAGCCGCCACAGGCGCCCATTCT
TGTACTGTGGCCCTTCCTGGTGGATACGTTAGAGTGTGCGAATTCGGAGGCGGAGGA
TCAGGCGGCGGAGGAAGTGGGGGAGGCGGATCTGAAGTTCAGCTGGTTGAGTCTGG
CGGCGGACTGGTTCAACCAGGCGGATCTCTGAGACTGAGCTGTGCCGCCAGCGGCT
TCACCTTTAGCAGCTTTGGCATGCACTGGGTCCGACAGGCCCCTGGCAAAGGACTTG
AGTGGGTCGCCTACATCAGCAGCGATAGCAGCGCCATCTACTACGCCGACACCGTG
AAGGGCAGATTCACCATCAGCCGGGACAACGCCAAGAACAGCCTGTACCTGCAGAT
GAACTCCCTGCGCGACGAGGATACCGCCGTGTACTATTGTGGCAGAGGCAGAGAGA
ACATCTATTACGGCAGCAGACTGGACTACTGGGGCCAGGGAACAACCGTGACAGTC
TCTTCTGGTGGCGGAGGAAGCGGAGGCGGAGGTTCAGGCGGCGGAGGATCTGATAT
TCAGCTGACTCAGAGCCCCAGCTTCCTGAGCGCCTCTGTGGGAGACAGAGTGACCAT
CACATGCAAGGCCAGCCAGAACGTGGACACCAACGTGGCCTGGTATCAGCAGAAGC
CTGGCAAGGCTCCCAAGGCTCTGATCTACAGCGCCAGCTACAGATACAGCGGCGTG
CCCAGCAGATTTTCTGGCAGCGGCTCTGGCACCGACTTCACCCTGACCATATCTAGC
CTGCAGCCTGAGGACTTCGCCACCTACTACTGCCAGCAGTACAACAACTACCCCTTC
ACCTTCGGCCAGGGCACCAAGCTGGAAATCAAGACCACCACACCAGCTCCTCGGCC
TCCAACTCCTGCTCCTACAATTGCCAGCCAGCCTCTGTCTCTGAGGCCCGAAGCTTG
TAGACCTGCTGCTGGCGGAGCCGTGCATACAAGAGGACTGGATTTCGCCTGCGACAT
CTACATCTGGGCCCCTCTGGCTGGAACATGTGGCGTTCTGCTGCTGAGCCTGGTCAT
CACCCTGTACTGCCGGTCCAAGAGAAGCAGACTGCTGCACAGCGACTACATGAACA
TGACCCCTAGACGGCCCGGACCTACCAGAAAGCACTACCAGCCTTACGCTCCTCCTA
GAGATTTCGCCGCCTACCGGTCCAGAGTGAAGTTCTCCAGATCCGCCGATGCTCCCG
CCTATCAGCAGGGACAGAACCAGCTGTACAACGAGCTGAACCTGGGGAGAAGAGA
AGAGTACGACGTGCTGGACAAGCGGAGAGGCAGGGATCCTGAGATGGGCGGCAAG
CCCAGACGGAAGAATCCTCAAGAGGGCCTGTATAATGAGCTGCAGAAAGACAAGAT
GGCCGAGGCCTACAGCGAGATCGGAATGAAGGGCGAGCGCAGAAGAGGCAAGGGA
CACGATGGACTGTACCAGGGCCTGAGCACCGCCACCAAGGATACCTATGATGCCCT
GCACATGCAGGCCCTGCCTCCAAGATAA
SEQ ID NO: 151 GRP78-G4S3-B7H3-CD8α-CAR (78.B7H3-CAR) full length
MDWIWRILFLVGAATGAHSCTVALPGGYVRVCEFGGGGSGGGGSGGGGSEVQLVESG
GGLVQPGGSLRLSCAASGFTFSSFGMHWVRQAPGKGLEWVAYISSDSSAIYYADTVKG
RFTISRDNAKNSLYLQMNSLRDEDTAVYYCGRGRENIYYGSRLDYWGQGTTVTVSSGG
GGSGGGGSGGGGSDIQLTQSPSFLSASVGDRVTITCKASQNVDTNVAWYQQKPGKAPK
ALIYSASYRYSGVPSRFSGSGSGTDFTLTISSLQPEDFATYYCQQYNNYPFTFGQGTKLEI
KTTTPAPRPPTPAPTIASQPLSLRPEACRPAAGGAVHTRGLDFACDIYIWAPLAGTCGVL
LLSLVITLYCRSKRSRLLHSDYMNMTPRRPGPTRKHYQPYAPPRDFAAYRSRVKFSRSA
DAPAYQQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR

1. A polynucleotide encoding a chimeric antigen receptor (CAR) comprising
a) an extracellular antigen-binding domain comprising one or more glucose-regulated-protein 78 (GRP78)-binding moieties and a CD123-binding moiety;
b) a transmembrane domain; and
c) a cytoplasmic domain comprising a signaling domain.

2. The polynucleotide of claim 1, wherein the GRP78-binding moiety comprises a GRP78-binding peptide comprising the amino acid sequence CTVALPGGYVRVC (SEQ ID NO: 92) or a variant thereof, and/or wherein the nucleotide sequence encoding the GRP78-binding peptide comprises the sequence TGTACTGTGGCCCTTCCTGGTGGATACGTTAGAGTGTGC (SEQ ID NO: 93), or a nucleotide sequence having at least 80% sequence identity thereof.

3-4. (canceled)

5. The polynucleotide of claim 1, wherein the extracellular antigen-binding domain comprises one, two or three GRP78-binding moieties.

6-7. (canceled)

8. The polynucleotide of claim 1, wherein the CD123-binding moiety is an antibody or antibody fragment.

9. The polynucleotide of claim 8, wherein the CD123-binding moiety is a single chain variable fragment (scFv) derived from antibody 26292 (scFV (292), wherein:
a) the scFV (292) comprises a heavy chain variable domain (VH) comprising the amino acid sequence of SEQ ID NO: 94, or an amino acid sequence having at least 80% identity thereof, and/or a light chain variable domain (VL) comprising the amino acid sequence of SEQ ID NO: 96, or an amino acid sequence having at least 80% identity thereof,
b) the nucleotide sequence encoding scFV (292) comprises a nucleotide encoding the VH and comprising the nucleotide sequence of SEQ ID NO: 95, or a nucleotide having at least 80% identity thereof, and/or a nucleotide encoding the VL and comprising the nucleotide sequence of SEQ ID NO: 97, or a nucleotide sequence having at least 80% identity thereof,
c) the scFV (292) comprises an amino acid sequence of SEQ ID NO: 98, or an amino acid sequence having at least 80% identity thereof, and/or
d) the nucleotide sequence encoding scFV (292) comprises the nucleotide sequence of SEQ ID NO: 99, or a nucleotide sequence having at least 80% identity thereof.

10-12. (canceled)

13. The polynucleotide of claim 9, wherein the VH and the VL are linked via a linker sequence.

14. The polynucleotide of claim 9, wherein the polynucleotide comprises three heavy chain complementarity determining regions (CDRs) (HCDR1, HCDR2 and HCDR3) contained within the VH sequence of SEQ ID NO: 94; and/or three light chain CDRs (LCDR1, LCDR2 and LCDR3) contained within the VL sequence of SEQ ID NO: 96.

15-16. (canceled)

17. The polynucleotide of claim 1, wherein the extracellular antigen-binding domain further comprises a linker sequence between the one or more GRP78-binding moieties and the CD123-binding moiety.

18. (canceled)

19. The polynucleotide of claim 17, wherein the linker sequence comprises a (G4S)3 linker (SEQ ID NO: 9), a β2M linker (SEQ ID NO: 12), a mutated IgG4 linker (SEQ ID NO: 14), or a GPcPcPc linker (SEQ ID NO: 16), or an amino acid sequence having at least 80% identity thereof, or wherein the linker sequence is encoded by any one of SEQ ID NOs: 10, 11, 13, 15, or 17, or a nucleotide sequence having at least 80% sequence identity thereof.

20. (canceled)

21. The polynucleotide of claim 19, wherein the linker sequence comprises a (G4S)3 linker (SEQ ID NO: 9), or is encoded by SEQ ID NOs: 10 or 11.

22. The polynucleotide of claim 1, wherein the extracellular antigen-binding domain further comprises a leader sequence derived from human immunoglobulin (IgG) heavy chain variable region or CD8α.

23-25. (canceled)

26. The polynucleotide of claim 1, wherein the CAR further comprises a hinge domain between the extracellular target binding domain and the transmembrane domain, wherein the hinge domain is derived from CD8α, CD28, or an IgG, and wherein when the hinge domain is derived from CD28, the CD28 hinge domain comprises the amino acid sequence of SEQ ID NO: 37, or an amino acid sequence having at least 80% sequence identity thereof, and/or the nucleotide sequence encoding the CD28 hinge domain comprises the sequence of SEQ ID NO: 38, or a nucleotide sequence having at least 80% sequence identity thereof.

27-30. (canceled)

31. The polynucleotide of claim 1, wherein the transmembrane domain is derived from CD8α, CD28, CD8, CD4, CD3ζ, CD40, CD134 (OX-40), NKG2A/C/D/E or CD7,
wherein when the transmembrane domain is derived from CD28, the CD28 transmembrane domain comprises the amino acid sequence SEQ ID NO: 43, or an amino acid sequence having at least 80% sequence identity thereof, and/or the nucleotide sequence encoding the CD28 transmembrane domain comprises the sequence SEQ ID NO: 44 or 45, or a nucleotide sequence having at least 80% sequence identity thereof.

32-34. (canceled)

35. The polynucleotide of claim 1, wherein the signaling domain is derived from CD3ζ, DAP10, DAP12, Fc ε receptor I γ chain (FCER1G), CD3δ, CD3ε, CD3γ, CD226, NKG2D, or CD79A,
wherein when the signaling domain is derived from CD3ζ, the CD3ζ signaling domain comprises the amino acid sequence SEQ ID NO: 69, or an amino acid sequence having at least 80% sequence identity thereof, and/or the nucleotide sequence encoding the CD3ζ signaling domain comprises the sequence SEQ ID NO: 70 or 71, or a nucleotide sequence having at least 80% sequence identity thereof.

36-38. (canceled)

39. The polynucleotide of claim 1, wherein the cytoplasmic domain further comprises one or more costimulatory domains derived from CD28, CD27, CD40, CD134, CD226, CD79A, ICOS, 4-1BB, OX40 or MyD88, or any combination thereof,
wherein when the cytoplasmic domain comprises a CD28 costimulatory domain, the CD28 costimulatory domain comprises the amino acid sequence of SEQ ID NO: 54, or an amino acid sequence having at least 80% sequence identity thereof, and/or the nucleotide sequence encoding the CD28 costimulatory domain comprises the sequence of SEQ ID NO: 55 or 56, or a nucleotide sequence having at least 80% sequence identity thereof.

40-43. (canceled)

44. The polynucleotide of claim 1, wherein the CAR comprises the amino acid sequence of any one of SEQ ID NOs: 110, 112, 114, or 116, or an amino acid sequence having at least 80% sequence identity thereof.

45. The polynucleotide of claim 1, wherein the nucleotide sequence encoding the CAR comprises the sequence of any one of SEQ ID NOs: 113, 115, 117 or 119, or a nucleotide sequence having at least 80% sequence identity thereof.

46-62. (canceled)

63. A chimeric antigen receptor (CAR) encoded by the polynucleotide of claim 1.

64. A recombinant vector comprising the polynucleotide of claim 1.

65-69. (canceled)

70. An isolated host cell comprising the polynucleotide of claim 1, optionally wherein the isolated host cell is a CD8+ T cell, a CD4+ T cell, a cytotoxic T cell, an αβ T cell receptor (TCR) T cell, an invariant natural killer T (iNKT) cell, a γδ T cell, a memory T cell, a memory stem T cell (TSCM), a naïve T cell, an effector T cell, a T-helper cell, or a regulatory T cell (Treg).

71-86. (canceled)

87. A pharmaceutical composition comprising the host cell of claim 70, and a pharmaceutically acceptable carrier and/or excipient.

88. A method of generating the isolated host cell of claim 70, said method comprising genetically modifying the host cell with the polynucleotide.

89-92. (canceled)

93. A method for killing a cancer cell expressing GRP78 and/or CD123, said method comprising contacting said cell with the host cell(s) of claim 70.

94. A method for treating a cancer in a subject in need thereof, wherein one or more cells of the tumor express GRP78 and/or CD123, said method comprising administering to the subject a therapeutically effective amount of the host cell(s) of claim 70.

95-98. (canceled)

99. A polynucleotide encoding a chimeric antigen receptor (CAR) comprising
a. an extracellular antigen-binding domain comprising one or more glucose-regulated-protein 78 (GRP78)-binding moieties and a B7H3-binding moiety;
b. a transmembrane domain; and
c. a cytoplasmic domain comprising a signaling domain.

100-203. (canceled)
